(19) United States
(12) Patent Application Publication
(10) Pub. No.: US 2009/0298820 A1
(43) Pub. Date: Dec. 3, 2009

(54) 3-SUBSTITUTED-1H-PYRROLO[2,3-B]PYRIDINE AND 3-SUBSTITUTED-1H-PYRROLO[3,2-B]PYRIDINE COMPOUNDS, THEIR USE AS MTOR KINASE AND PI3 KINASE INHIBITORS, AND THEIR SYNTHESES

(75) Inventors: Hwei-Ru Tsou, New City, NY (US); Gary Harold Birnberg, Tuxedo Park, NY (US); Gloria Jean MacEwan, Monroe, NY (US); Semiramis Ayral-Kaloustian, Tarrytown, NY (US); Matthew Gregory Bursavich, Salt Lake City, UT (US); Sabrina Lombardi, New Haven, CT (US); Nan Zhang, Bayside, NY (US); Adam Matthew Gilbert, Congers, NY (US); George Theordore Grosu, Pearl River, NY (US); Natasja Brooijmans, New York, NY (US); Thai Hiep Nguyen, Fair Lawn, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/473,658

(22) Filed: May 28, 2009

Related U.S. Application Data
(60) Provisional application No. 61/056,679, filed on May 28, 2008.

(51) Int. Cl. A61K 31/437; A61K 31/5375; A61K 31/5377; A61K 31/496; C07D 471/04; A61P 35/00; C07D 498/08; A61P 29/00; A61P 17/00; A61P 19/00

(52) U.S. Cl. 514/230.5; 514/234.5; 514/253.04; 514/300; 544/105; 544/127; 544/362; 546/113

(57) ABSTRACT

The invention relates to 3-substituted-1H-pyrrolo[2,3-b]pyridine, and 3-substituted-1H-pyrrolo[3,2-b]pyridine compounds of the Formula 1:

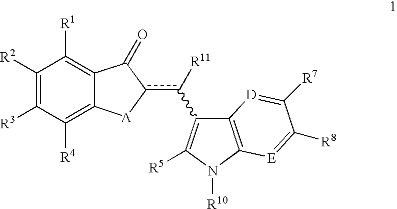

or a pharmaceutically acceptable salt thereof, wherein the constituent variables are as defined herein, compositions comprising the compounds, and methods for making and using the compounds.

### FIELD OF THE INVENTION

The invention relates to 3-substituted-1H-pyrrolo[2,3-b]pyridine, and 3-substituted-1H-pyrrolo[3,2-b]pyridine compounds, compositions comprising such compounds, methods of synthesizing such compounds, and methods for treating mTOR-related diseases comprising the administration of an effective amount of such a compound. The invention also relates to methods for treating PI3K-related diseases comprising the administration of an effective amount of such a compound.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267 (1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,2′:4,5]furo[3,2-d]pyrimidin-2-yl)phenol) inhibits PI3K_{α} and PI3K_{γ} as well as the mTOR complexes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4, 6-dimorpholino-1,3,5-triazine) inhibits PI3K_{α} and PI3K_{γ} but not the mTOR enzymes with IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth that originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{α} and PI3K_{γ} as well as the mTOR enzyme with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut. 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,
Although it seems clear that inhibition of the a isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over-activated in numerous cancers and may account for the widespread response from various cancers to mTOR inhibitors. The over-activation of the upstream pathway would normally cause mTOR kinase to be over-activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. On Mar. 30, 2009, the Food and Drug Administration (FDA) approved Everolimus (AFINITOR™) for the treatment of patients with advanced renal cell carcinoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. The instant invention is directed to these and other important ends.

### SUMMARY OF THE INVENTION

In one aspect, the invention provides compounds of the Formula 1:

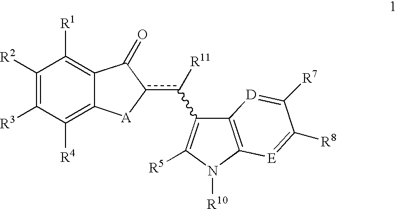

or a pharmaceutically acceptable salt thereof, wherein the constituent variables are as defined below. In other aspects, the invention provides compositions comprising a compound of the invention, and methods for making compounds of the invention. In further aspects, the invention provides methods for inhibiting PI3K and mTOR in a subject, and methods for treating PI3K-related and mTOR-related disorders in a mammal in need thereof.

### DETAILED DESCRIPTION OF THE INVENTION

In one aspect, the invention provides compounds of the Formula: 1:

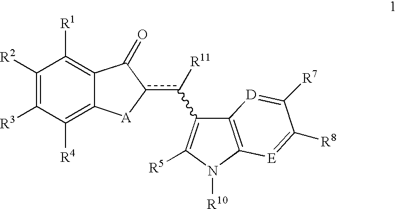

or a geometric isomer thereof or a pharmaceutically acceptable salt thereof, wherein:

A is oxygen, sulfur, or CH₂;

[symbol] represents an optional second carbon-to-carbon bond;

D is C—R⁶ or N;

E is C—R⁹ or N;

with the proviso that at least one of D and E must be N;

R¹, R², R³, and R⁴ are independently H; C₁-C₆alkoxy optionally substituted with from 1 to 3 substituents independently selected from H₂N—, (C₁-C₆alkyl)N—, and (C₁-C₆alkyl)(C₁-C₆alkyl)N—; C₁-C₆alkyl; (C₁-C₆alkoxy)carbonyl; R¹²R¹³N—; R¹²R¹³NC(O)NH—; R¹²C(O)NH—; R¹⁴OC(O)NH—; halo; or hydroxyl;

R¹² and R¹³ are each independently H; C₁-C₆alkyl optionally substituted with from 1 to 3 substituents independently selected from halo, H₂N—, (C₁-C₆alkyl)NH—, (C₁-C₆alkyl)(C₁-C₆alkyl)N-optionally substituted by C₁-C₆alkoxy, C₁-C₉heterocyclyl optionally substituted by C₁-C₆alkyl, or C₁-C₉heteroaryl optionally substituted by C₁-C₆alkyl; perfluoro(C₁-C₆)alkyl; C₁-C₉heteroaryl optionally substituted with from 1 to 3 substituents independently selected from C₁-C₆alkyl, halo, C₁-C₉heterocyclyl optionally substituted by C₁-C₆alkyl, C₁-C₆alkoxy optionally substituted with (C₁-C₆alkyl)(C₁-C₆alkyl)N—, C₁-C₉heterocyclyl-O—, heterocyclyl(C₁-C₆alkyl), and perfluoro(C₁-C₆)alkyl; C₁-C₉heterocyclyl optionally substituted by C₁-C₆alkyl or C₁-C₆acyl optionally substituted with from 1 to 3 independently selected halogens; C₆-C₁₄aryl optionally substituted with from 1 to 3 substituents independently selected from C₁-C₆alkyl, halo, C₁-C₉heterocyclyl optionally substituted by C₁-C₆alkyl, C₁-C₆alkoxy optionally substituted with (C₁-C₆alkyl)(C₁-C₆alkyl)N—, C₁-C₉heterocyclyl-O—, heterocyclyl(C₁-C₆alkyl), and perfluoro(C₁-C₆)alkyl; or C₃-C₈cycloalkyl;

R¹⁴ is independently C₁-C₆alkyl, C₁-C₆hydroxylalkyl-, or C₆-C₁₄aryl;

R⁵ is H; C₁-C₆alkyl; C₆-C₁₄aryl; C₃-C₈cycloalkyl; halo; C₁-C₉heteroaryl; C₁-C₆heterocyclylalkyl; C₁-C₆perfluoroalkyl-; R¹⁵R¹⁶NC(O)—; (C₁-C₆alkoxy)carbonyl; or CO₂H;

R¹⁵ and R¹⁶ are each independently H; C₁-C₆alkyl optionally substituted with from 1 to 3 substituents independently selected from H₂N—, (C₁-C₆alkyl)NH—, (C₁-C₆alkyl)(C₁-C₆alkyl)N—, or C₁-C₉heteroaryl; C₁-C₉heteroaryl; C₆-C₁₄aryl optionally substituted with from 1 to 3 substituents independently selected from C₁-C₆alkyl, halo, perfluoro(C₁-C₆)alkyl; C₃-C₈cycloalkyl;

or R¹⁵ and R¹⁶ when taken together with the nitrogen to which they are attached can form a 3- to 7-membered nitrogen-containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;

R⁶-R⁹ are each independently:

(a) H; (b) C₁-C₆alkoxy; (c) C₁-C₆alkyl optionally substituted by C₆-C₁₄aryl; (d) C₂-C₆alkenyl optionally substituted by C₆-C₁₄aryl; (e) C₂-C₆alkynyl optionally substituted by C₆-C₁₄aryl; (f) (C₁-C₆alkyl)amido-; (g) C₁-C₆alkylcarboxy; (h) (C₁-C₆alkyl)carboxyamido; (i) (C₁-C₆alkyl)SO₂—; (j) C₆-C₁₄aryl optionally substituted with from 1 to 3 substituents independently selected from: (i) C₁-C₈acyl, (ii) C₁-C₆alkyl, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl optionally substituted by C₁-C₆alkyl, (iii) (C₁-C₆alkyl)amido-, (iv) (C₁-C₆alkyl)carboxy, (v) (C₁-C₆alkyl)carboxyamido, (vi) C₁-C₆alkoxy- optionally substituted by C₁-C₆alkoxy- or C₁-C₉heteroaryl, (vi) (C₁-C₆alkoxy)carbonyl, (viii) (C₆-C₁₄aryl)oxy, (ix) C₃-C₈cycloalkyl, (x) halo, (xi) C₁-C₆haloalkyl-, (xii) C₁-C₉heterocyclyl optionally substituted by C₁-C₆alkyl or C₁-C₆hydroxylalkyl-, (xiii) hydroxyl, (xiv) C₁-C₆hydroxylalkyl-, (xv) C₁-C₆perfluoroalkyl-, (xvi) C₁-C₆perfluoroalkyl-O—, (xvii) R¹⁷R¹⁸N—, (xviii) CN, (xix) —COOH, (xx) R¹⁷R¹⁸NC(O)—, (xxi) R¹⁷C(O)NH—, (xxii) R¹⁷R¹⁸NS(O)₂— (XXiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) C₁-C₉heteroaryl, (xxviii) —C(═N—(OR¹⁷))—(NR¹⁷R¹⁸), and (xxix) —NO₂; (k) (C₆-C₁₄aryl)alkyl-O—; (l) halo; (m) C₁-C₉heteroaryl optionally substituted with from 1 to 3 substituents independently selected from: (i) C₁-C₈acyl, (ii) C₁-C₆alkyl, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl optionally substituted by C₁-C₆alkyl, (iii) (C₁-C₆alkyl)amido-, (iv) (C₁-C₆alkyl)carboxy, (v) (C₁-C₆alkyl)carboxyamido, (vi) C₁-C₆alkoxy- optionally substituted by C₁-C₆alkoxy- or C₁-C₉heteroaryl, (vii) (C₁-C₆alkoxy)carbonyl, (viii) (C₆-C₁₄aryl)oxy, (ix) C₃-C₈cycloalkyl, (x) halo, (xi) C₁-C₆haloalkyl-, (xii) C₁-C₉heterocyclyl- optionally substituted by C₁-C₆alkyl or C₁-C₆hydroxylalkyl-, (xiii) hydroxyl, (xiv) C₁-C₆hydroxylalkyl-, (xv) C₁-C₆perfluoroalkyl-, (xvi) C₁-C₆perfluoroalkyl-O—, (xvii) R¹⁷R¹⁸N—, (xviii) CN, (xix) —COOH, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) C₁-C₉heteroaryl, (xxviii) —C(═N—(OR¹⁷))—(NR¹⁷R¹⁸), and (xxix) —NO₂; (n) hydroxyl; (o)C₁-C₉heterocyclyl optionally substituted with from 1 to 3 substituents independently selected from: (i) C₁-C₆alkyl, which is optionally substituted with from 1 to 3 substituents independently selected from: A) H₂N—, B) (C₁-C₆alkyl)NH—, and C) (C₁-C₆alkyl)(C₁-C₆alkyl)N—, (ii) R¹⁷R¹⁸NC(O)—, (iii) (C₁-C₆)alkyl-O—(C₁-C₆)alkylene-, (iv) hydroxyl, and (v) R¹⁷R¹⁸N—; (p) C₁-C₆perfluoroalkyl-; (q) CN; (r) (C₁-C₆alkoxy)carbonyl; (s) CO₂H; or (t) NO₂;

R¹⁷ and R¹⁸ are each independently H; C₁-C₆alkyl optionally substituted with from 1 to 3 substituents independently selected from C₁-C₆alkoxy-, hydroxyl-, H₂N—, (C₁-C₆alkyl)NH—, or (C₁-C₆alkyl)(C₁-C₆alkyl)N—; C₁-C₆alkoxy-; C₂-C₆alkenyl; C₂-C₆alkynyl; C₁-C₆-carboxyamidoalkyl-; C₁-C₉heteroaryl optionally substituted with from 1 to 3 substituents independently selected from C₁-C₆alkyl, halo, or perfluoro(C₁-C₆)alkyl; C₁-C₉heterocyclyl- optionally substituted by C₁-C₆alkyl; C₆-C₁₄aryl optionally substituted with from 1 to 3 substituents independently selected from C₁-C₆alkyl, halo, or perfluoro(C₁-C₆)alkyl; heterocyclyl(C₁-C₆alkyl); (C₆-C₁₄aryl)alkyl optionally substituted by C₁-C₆alkyl or C₁-C₆alkoxy-; (C₁-C₉heteroaryl)alkyl optionally substituted by C₁-C₆alkyl or C₁-C₆alkoxy-; or C₃-C₈cycloalkyl;

or R¹⁷ and R¹⁸ when taken together with the nitrogen to which they are attached can form a nitrogen-containing monocyclic, bicyclic, or bridged non-aromatic C₁-C₉heterocycle wherein up to two of the carbon atoms of the C₁-C₉heterocycle can be replaced with —N(H)—, —N(C₁-C₆alkyl)-, —N(C₆-C₁₄aryl)-, —S—, —SO—, —S(O)₂, or —O—, which is also optionally substituted with from 1 to 3 substituents independently selected from C₁-C₆alkyl, C₁-C₆alkoxy-, hydroxyl, C₁-C₆hydroxylalkyl-, H₂N—, (C₁-C₆alkyl)NH—, (C₁-C₆alkyl)(C₁-C₆alkyl)N—, or (C₁-C₆)alkyl-O—(C₁-C₆)alkylene-;

R¹⁹ is C₁-C₆alkyl or C₆-C₁₄aryl;

or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;

R¹⁰ is H; C₁-C₆alkyl optionally substituted with from 1 to 3 substituents independently selected from halogen, H₂N—, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)N(C₁-C₆alkyl)(C₁-C₆alkyl), —CN, hydroxyl, C₁-C₆alkoxy-, C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂; C₂-C₁₀ alkenyl; C₆-C₁₄aryl; C₃-C₈cycloalkyl; C₁-C₉heteroaryl; or C₁-C₆heterocyclylalkyl group optionally substituted with from 1 to 3 substituents independently selected from halogen, H₂N—, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy-, C₁-C₆alkyl, —C(O)OH, —C(O)O(C₁-C₆alkyl), —C(O)(C₁-C₆alkyl), 4- to 7-membered monocyclic heterocycle, C₆-C₁₄aryl, C₁-C₉heteroaryl, C₁-C₆heterocyclylalkyl, or C₃-C₈cycloalkyl;

R¹¹ is H or C₁-C₆alkyl.

In one aspect, the invention provides compounds of the Formula 1:

In one embodiment, A is oxygen.

In one embodiment, R¹ is hydroxyl.

In one embodiment, R² is H.

In one embodiment, R³ is hydroxyl.

In one embodiment, R⁴ is H.

In one embodiment, R⁵ is H.

In one embodiment, R⁶ is C₆-C₁₄aryl, optionally independently substituted with from 1 to 3 substituents as specified in Formula 1 or C₁-C₉heterocyclyl.

In one embodiment, R⁷ is H.

In one embodiment, R⁸ is H.

In one embodiment, E is N.

In one embodiment, R¹⁰ is C₁-C₆alkyl.

In one embodiment, R¹⁰ is methyl.

In one embodiment, R¹¹ is H.

In one embodiment, R¹═R³=hydroxyl and R²═R⁴═H.

In one embodiment, R⁵═R⁷═R⁸═H and R¹⁰ is CH₃.

In one embodiment, R⁶ is C₆-C₁₄aryl, optionally independently substituted with from 1 to 3 substituents as specified in Formula 1, E is N, and R¹¹ is H.

In one embodiment, R¹═R³=hydroxyl, R²═R⁴═R⁵═R⁷═R⁸═R¹¹═H, R⁶ is C₆-C₁₄aryl, optionally independently substituted with from 1 to 3 substituents as specified in Formula 1, E is N, and R¹⁰ is CH₃.

In another aspect, the invention provides compounds of the Formula 2:

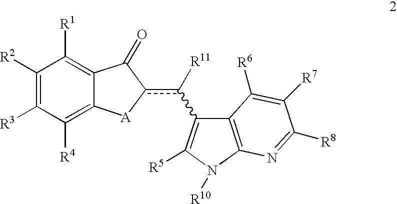

or a geometric isomer thereof or a pharmaceutically acceptable salt thereof, wherein:

the constituent variables are as defined for Formula 1.

In one embodiment, A is oxygen.

In one embodiment, R¹ is hydroxyl.

In one embodiment, R² is H.

In one embodiment, R³ is hydroxyl.

In one embodiment, R⁴ is H.

In one embodiment, R⁵ is H.

In one embodiment, R⁶ is C₆-C₁₄aryl, optionally independently substituted with from 1 to 3 substituents as specified in Formula 2 or C₁-C₉heterocyclyl.

In one embodiment, R⁷ is H.

In one embodiment, R⁸ is H.

In one embodiment, R¹⁰ is C₁-C₆alkyl.

In one embodiment, R¹⁰ is methyl.

In one embodiment, R¹¹ is H.

In one embodiment, R¹═R³=hydroxyl and R²═R⁴═H.

In one embodiment, R⁵═R⁷═R⁸═H and R¹⁰ is CH₃.

In one embodiment, R⁶ is C₆-C₁₄aryl, optionally independently substituted with from 1 to 3 substituents as specified in Formula 2 and R¹¹ is H.

In one embodiment, R¹═R³=hydroxyl, R²═R⁴═R⁵═R⁷═R⁸═R¹¹═H, R⁶ is C₆-C₁₄aryl, optionally independently substituted with from 1 to 3 substituents as specified in Formula 2, and R¹⁰ is CH₃.

In another aspect, the invention provides compounds of the Formula 3:

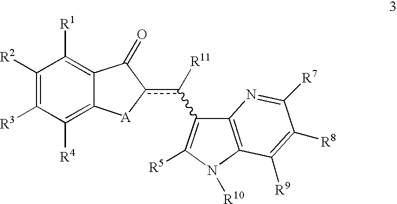

or a geometric isomer thereof or a pharmaceutically acceptable salt thereof, wherein:

the constituent variables are as defined for Formula 1.

In one embodiment, A is oxygen.

In one embodiment, R¹ is hydroxyl.

In one embodiment, R² is H.

In one embodiment, R³ is hydroxyl.

In one embodiment, R⁴ is H.

In one embodiment, R⁵ is H.

In one embodiment, R⁷ is C₁-C₆alkoxy.

In one embodiment, R⁷ is methoxy.

In one embodiment, R⁸ is H.

In one embodiment, R⁹ is H.

In one embodiment, R¹⁰ is H or C₁-C₆alkyl.

In one embodiment, R¹⁰ is methyl.

In one embodiment, R¹¹ is H.

In one embodiment, R¹═R³=hydroxyl and R²═R⁴═H.

In one embodiment, R⁵═R⁸═R⁹═H, R⁷ is methoxy, and R¹⁰ is CH₃.

In one embodiment, R¹═R³=hydroxyl, R²═R⁴═R⁵═R⁸═R⁹═R¹⁰═R¹¹═H and R⁷ is methoxy.

In another aspect, the invention provides compounds of the Formula 4:

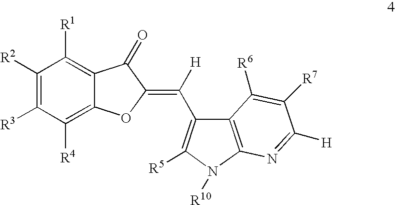

or a geometric isomer thereof or a pharmaceutically acceptable salt thereof, wherein:

the constituent variables are as defined for Formula 1.

In one embodiment, R¹ is hydroxyl.

In one embodiment, R² is H.

In one embodiment, R³ is hydroxyl.

In one embodiment, R⁴ is H.

In one embodiment, R⁵ is H.

In one embodiment, R⁶ is C₆-C₁₄aryl, optionally independently substituted with from 1 to 3 substituents as specified in Formula 4 or C₁-C₉heterocyclyl.

In one embodiment, R⁷ is H.

In one embodiment, R¹⁰ is C₁-C₆alkyl.

In one embodiment, R¹⁰ is methyl.

In one embodiment, R¹═R³=hydroxyl and R²═R⁴═H.

In one embodiment, R⁵═R⁷═H and R¹⁰ is CH₃.

In one embodiment, R⁶ is C₆-C₁₄aryl, optionally independently substituted with from 1 to 3 substituents as specified in Formula 4.

In one embodiment, R¹═R³=hydroxyl, R²═R⁴═R⁵═R⁷═H, R⁶ is C₆-C₁₄aryl, optionally independently substituted with from 1 to 3 substituents as specified in Formula 4, and R¹⁰ is CH₃.

Additional illustrative compounds of formula 4 are set forth below:
4,6-dihydroxy-2-[(1-methyl-1H-pyrrolo[2,3-b]pyridin-3-yl)methylene]-1-benzofuran-3(2H)-one;
6,7-dihydroxy-2-[(1-methyl-1H-pyrrolo[2,3-b]pyridin-3-yl)methylene]-1-benzofuran-3(2H)-one;
4,6-dihydroxy-2-({1-[3-(4-methylpiperazin-1-yl)propyl]-1H-pyrrolo[2,3-b]pyridin-3-yl}methylene)-1-benzofuran-3(2H)-one;
4,6-dihydroxy-2-{[1-(3-morpholin-4-ylpropyl)-1H-pyrrolo[2,3-b]pyridin-3-yl]methylene}-1-benzofuran-3(2H)-one;
6,7-dihydroxy-2-({1-[3-(4-methylpiperazin-1-yl)propyl]-1H-pyrrolo[2,3-b]pyridin-3-yl}methylene)-1-benzofuran-3(2H)-one;
6,7-dihydroxy-2-{[1-(3-morpholin-4-ylpropyl)-1H-pyrrolo[2,3-b]pyridin-3-yl]methylene}-1-benzofuran-3(2H)-one;
6,7-dihydroxy-2-[(1-{3-[4-(2-hydroxyethyl)piperazin-1-yl]propyl}-1H-pyrrolo[2,3-b]pyridin-3-yl)methylene]-1-benzofuran-3(2H)-one;
4,6-dihydroxy-2-(1H-pyrrolo[2,3-b]pyridin-3-ylmethylene)-1-benzofuran-3(2H)-one;
(2Z)-2-{[4-(2-chlorophenyl)-1-methyl-1H-pyrrolo[2,3-b]pyridin-3-yl]methylene}-4,6-dihydroxy-1-benzofuran-3(2H)-one;
(2Z)-2-{[4-(3-chlorophenyl)-1-methyl-1H-pyrrolo[2,3-b]pyridin-3-yl]methylene}-4,6-dihydroxy-1-benzofuran-3(2H)-one;
(2Z)-4,6-dihydroxy-2-{[4-(2-methoxyphenyl)-1-methyl-1H-pyrrolo[2,3-b]pyridin-3-yl]methylene}-1-benzofuran-3(2H)-one;
(2Z)-4,6-dihydroxy-2-({1-methyl-4-[4-(trifluoromethoxy)phenyl]-1H-pyrrolo[2,3-b]pyridin-3-yl}methylene)-1-benzofuran-3(2H)-one;
(2Z)-4,6-dihydroxy-2-[(4-{4-[2-(1H-imidazol-1-yl)ethoxy]phenyl}-1-methyl-1H-pyrrolo[2,3-b]pyridin-3-yl)methylene]-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}benzenesulfonamide;
N-(3-{3-[(Z)-(4,6-dihydroxy-3-oxo-1-benzofuran-2(3H)-ylidene)methyl]-1-methyl-1H-pyrrolo[2,3-b]pyridin-4-yl}phenyl)methanesulfonamide;
(2Z)-4,6-dihydroxy-2-{[4-(4-hydroxyphenyl)-1-methyl-1H-pyrrolo[2,3-b]pyridin-3-yl]methylene}-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}-N′-hydroxybenzenecarboximidamide;
3-{3-[(Z)-(4-hydroxy-3-oxo-1-benzofuran-2(3H)-ylidene)methyl]-1-methyl-1H-pyrrolo[2,3-b]pyridin-4-yl}benzenesulfonamide;
(2Z)-2-({4-[4-(5,6-dihydro-4H-1,3-oxazin-2-yl)phenyl]-1-methyl-1H-pyrrolo[2,3- b]pyridin-3-yl}methylidene)-4-hydroxy-1-benzofuran-3(2H)-one;
(2Z)-4-hydroxy-2-{[1-methyl-4-(1H-pyrrol-2-yl)-1H-pyrrolo[2,3-b]pyridin-3-yl]methylidene}-1-benzofuran-3(2H)-one;
(2Z)-4,6-dihydroxy-2-[(1-methyl-4-phenyl-1H-pyrrolo[2,3-b]pyridin-3-yl)methylene]-1-benzofuran-3(2H)-one;
(2Z)-2-({4-[4-(dimethylamino)phenyl]-1-methyl-1H-pyrrolo[2,3-b]pyridin-3-yl}methylene)-4,6-dihydroxy-1-benzofuran-3(2H)-one;
(2Z)-4,6-dihydroxy-2-[(1-methyl-2-phenyl-1H-pyrrolo[2,3-b]pyridin-3-yl)methylene]-1-benzofuran-3(2H)-one;
(2Z)-6-hydroxy-2-[(1-methyl-2-phenyl-1H-pyrrolo[2,3-b]pyridin-3-yl)methylene]-1-benzofuran-3(2H)-one;
(2Z)-4-hydroxy-2-[(1-methyl-2-phenyl-1H-pyrrolo[2,3-b]pyridin-3-yl)methylene]-1-benzofuran-3(2H)-one;
(2Z)-6-hydroxy-2-[(1-methyl-4-phenyl-1H-pyrrolo[2,3-b]pyridin-3-yl)methylene]-1-benzofuran-3(2H)-one;
(2Z)-4-hydroxy-2-[(1-methyl-4-phenyl-1H-pyrrolo[2,3-b]pyridin-3-yl)methylene]-1-benzofuran-3(2H)-one;
(2Z)-4-fluoro-6-hydroxy-2-[(1-methyl-4-phenyl-1H-pyrrolo[2,3-b]pyridin-3-yl)methylene]-1-benzofuran-3(2H)-one;
(2Z)-4,6-dihydroxy-2-[(1-methyl-4-pyridin-3-yl-1H-pyrrolo[2,3-b]pyridin-3-yl)methylene]-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}benzamide;
N-(4-{3-[(Z)-(4,6-dihydroxy-3-oxo-1-benzofuran-2(3H)-ylidene)methyl]-1-methyl-1H-pyrrolo[2,3-b]pyridin-4-yl}phenyl)acetamide;
N-(4-{3-[(Z)-(4,6-dihydroxy-3-oxo-1-benzofuran-2(3H)-ylidene)methyl]-1-methyl-1H-pyrrolo[2,3-b]pyridin-4-yl}phenyl)methanesulfonamide;
(2Z)-5-bromo-2-[(1-methyl-4-phenyl-1H-pyrrolo[2,3-b]pyridin-3-yl)methylene]-1-benzofuran-3(2H)-one;
1-methyl-3-{(2Z)-2-[(1-methyl-4-phenyl-1H-pyrrolo[2,3-b]pyridin-3-yl)methylene]-3-oxo-2,3-dihydro-1-benzofuran-5-yl}urea;
(2Z)-4,6-dihydroxy-2-{[1-methyl-4-(phenylethynyl)-1H-pyrrolo[2,3-b]pyridin-3-yl]methylene}-1-benzofuran-3(2H)-one;
(2Z)-4,6-dihydroxy-2-[(1-methyl-4-piperidin-1-yl-1H-pyrrolo[2,3-b]pyridin-3-yl)methylene]-1-benzofuran-3(2H)-one;
(2Z)-2-{[4-(4-aminophenyl)-1-methyl-1H-pyrrolo[2,3-b]pyridin-3-yl]methylene}-4,6-dihydroxy-1-benzofuran-3(2H)-one;
(2Z)-5-hydroxy-2-[(1-methyl-4-phenyl-1H-pyrrolo[2,3-b]pyridin-3-yl)methylene]-1-benzofuran-3(2H)-one;
(2Z)-4,6-dihydroxy-2-({1-methyl-4-[4-(methylamino)phenyl]-1H-pyrrolo[2,3-b]pyridin-3-yl}methylene)-1-benzofuran-3(2H)-one;
(2Z)-4,6-dihydroxy-2-{[1-methyl-4-(4-methylpiperazin-1-yl)-1H-pyrrolo[2,3-b]pyridin-3-yl]methylene}-1-benzofuran-3(2H)-one;
(2Z)-4,6-dihydroxy-2-[(1-methyl-4-morpholin-4-yl-1H-pyrrolo[2,3-b]pyridin-3-yl)methylene]-1-benzofuran-3(2H)-one;
(2Z)-4,6-dihydroxy-2-[(1-methyl-4-pyrrolidin-1-yl-1H-pyrrolo[2,3-b]pyridin-3-yl)methylene]-1-benzofuran-3(2H)-one;
(2Z)-4-hydroxy-2-{[1-methyl-4-(4-methylpiperazin-1-yl)-1H-pyrrolo[2,3-b]pyridin-3-yl]methylene}-1-benzofuran-3(2H)-one;
(2Z)-4-hydroxy-2-[(1-methyl-4-morpholin-4-yl-1H-pyrrolo[2,3-b]pyridin-3-yl)methylene]-1-benzofuran-3(2H)-one;
(2Z)-4,6-dihydroxy-2-{[1-methyl-4-(4-morpholin-4-ylphenyl)-1H-pyrrolo[2,3-b]pyridin-3-yl]methylene}-1-benzofuran-3(2H)-one;
(2Z)-4,6-dihydroxy-2-{[4-(4-hydroxypiperidin-1-yl)-1-methyl-1H-pyrrolo[2,3-b]pyridin-3-yl]methylene}-1-benzofuran-3(2H)-one;
(2Z)-4,6-dihydroxy-2-({1-methyl-4-[(E)-2-phenylvinyl]-1H-pyrrolo[2,3-b]pyridin-3-yl}methylene)-1-benzofuran-3(2H)-one;
(2Z)-2-({4-[3-(dimethylamino)phenyl]-1-methyl-1H-pyrrolo[2,3-b]pyridin-3-yl}methylene)-4,6-dihydroxy-1-benzofuran-3(2H)-one;
(2Z)-4,6-dihydroxy-2-{[4-(4-methoxyphenyl)-1-methyl-1H-pyrrolo[2,3-b]pyridin-3-yl]methylene}-1-benzofuran-3(2H)-one;
(2Z)-4,6-dihydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one;
(2Z)-4-hydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one;
(2Z)-4,6-dihydroxy-2-({1-methyl-4-[4-(4-methylpiperazin-1-yl)phenyl]-1H-pyrrolo[2,3-b]pyridin-3-yl}methylene)-1-benzofuran-3(2H)-one;
(2Z)-4-hydroxy-2-({1-methyl-4-[4-(4-methylpiperazin-1-yl)phenyl]-1H-pyrrolo[2,3-b]pyridin-3-yl}methylene)-1-benzofuran-3(2H)-one;
(2Z)-5-amino-2-[(1-methyl-4-phenyl-1H-pyrrolo[2,3-b]pyridin-3-yl)methylene]-1-benzofuran-3(2H)-one;
(2Z)-5-bromo-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one;
2-hydroxyethyl {(2Z)-2-[(1-methyl-4-phenyl-1H-pyrrolo[2,3-b]pyridin-3-yl)methylene]-3-oxo-2,3-dihydro-1-benzofuran-5-yl}carbamate;
1-ethyl-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]Urea;
(2Z)-4,6-dihydroxy-2-[(1-methyl-4-pyrimidin-5-yl-1H-pyrrolo[2,3-b]pyridin-3-yl)methylene]-1-benzofuran-3(2H)-one;
(2Z)-2-{[4-(6-chloropyridin-3-yl)-1-methyl-1H-pyrrolo[2,3-b]pyridin-3-yl]methylene}-4,6-dihydroxy-1-benzofuran-3(2H)-one;
(2Z)-2-{[4-(4-chlorophenyl)-1-methyl-1H-pyrrolo[2,3-b]pyridin-3-yl]methylene}-4,6-dihydroxy-1-benzofuran-3(2H)-one;
(2Z)-2-[(4-azepan-1-yl-1-methyl-1H-pyrrolo[2,3-b]pyridin-3-yl)methylene]-4,6-dihydroxy-1-benzofuran-3(2H)-one;
(2Z)-4,6-dihydroxy-2-{[4-(3-methoxyphenyl)-1-methyl-1H-pyrrolo[2,3-b]pyridin-3-yl]methylene}-1-benzofuran-3(2H)-one;
(2Z)-4,6-dihydroxy-2-({4-[4-(hydroxymethyl)phenyl]-1-methyl-1H-pyrrolo[2,3-b]pyridin-3-yl}methylene)-1-benzofuran-3(2H)-one;
2,2-dimethyl-N-{(2Z)-2-[(1-methyl-4-phenyl-1H-pyrrolo[2,3-b]pyridin-3-yl)methylene]-3-oxo-2,3-dihydro-1-benzofuran-4-yl}propanamide;
(2Z)-4-hydroxy-2-{[1-methyl-4-(6-morpholin-4-ylpyridin-3-yl)-1H-pyrrolo[2,3-b]pyridin-3-yl]methylene}-1-benzofuran-3(2H)-one;
(2Z)-4-hydroxy-2-{[1-methyl-4-(6-piperidin-1-ylpyridin-3-yl)-1H-pyrrolo[2,3-b]pyridin-3-yl]methylene}-1-benzofuran-3(2H)-one;
(2Z)-4-hydroxy-2-{[4-(6-methoxypyridin-3-yl)-1-methyl-1H-pyrrolo[2,3-b]pyridin-3-yl]methylene}-1-benzofuran-3(2H)-one;
(2Z)-4,6-dihydroxy-2-({1-methyl-4-[4-(methylsulfonyl)phenyl]-1H-pyrrolo[2,3-b]pyridin-3-yl}methylene)-1-benzofuran-3(2H)-one;
(2Z)-4-hydroxy-2-({1-methyl-4-[4-(methylsulfonyl)phenyl]-1H-pyrrolo[2,3-b]pyridin-3-yl}methylene)-1-benzofuran-3(2H)-one;
(2Z)-4,6-dihydroxy-2-({1-methyl-4-[3-(methylsulfonyl)phenyl]-1H-pyrrolo[2,3-b]pyridin-3-yl}methylene)-1-benzofuran-3(2H)-one;
(2Z)-4-hydroxy-2-({1-methyl-4-[3-(methylsulfonyl)phenyl]-1H-pyrrolo[2,3-b]pyridin-3-yl}methylene)-1-benzofuran-3(2H)-one;
(2Z)-4-hydroxy-2-({4-[6-(hydroxymethyl)pyridin-3-yl]-1-methyl-1H-pyrrolo[2,3-b]pyridin-3-yl}methylene)-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}-N-methylbenzenesulfonamide;
(2Z)-4,6-dihydroxy-2-{[1-methyl-4-(1,2,3,6-tetrahydropyridin-4-yl)-1H-pyrrolo[2,3-b]pyridin-3-yl]methylene}-1-benzofuran-3(2H)-one;
(2Z)-4,6-dihydroxy-2-({1-methyl-4-[3-(pyrrolidin-1-ylcarbonyl)phenyl]-1H-pyrrolo[2,3-b]pyridin-3-yl}methylene)-1-benzofuran-3(2H)-one;
(2Z)-4,6-dihydroxy-2-({1-methyl-4-[3-(piperidin-1-ylsulfonyl)phenyl]-1H-pyrrolo[2,3-b]pyridin-3-yl}methylene)-1-benzofuran-3(2H)-one;
(2Z)-4-hydroxy-2-({4-[4-methoxy-3-(morpholin-4-ylsulfonyl)phenyl]-1-methyl-1H-pyrrolo[2,3-b]pyridin-3-yl}methylene)-1-benzofuran-3(2H)-one;
N-cyclopropyl-4-{3-[(Z)-(4-hydroxy-3-oxo-1-benzofuran-2(3H)-ylidene)methyl]-1-methyl-1H-pyrrolo[2,3-b]pyridin-4-yl}benzenesulfonamide;
N-(4-fluorophenyl)-4-{3-[(Z)-(4-hydroxy-3-oxo-1-benzofuran-2(3H)-ylidene)methyl]-1-methyl-1H-pyrrolo[2,3-b]pyridin-4-yl}benzamide;
(2Z)-4-hydroxy-2-{[1-methyl-4-(4-phenoxyphenyl)-1H-pyrrolo[2,3-b]pyridin-3-yl]methylene}-1-benzofuran-3(2H)-one;
(2Z)-2-{[4-(1-benzofuran-2-yl)-1-methyl-1H-pyrrolo[2,3-b]pyridin-3-yl]methylene}-4-hydroxy-1-benzofuran-3(2H)-one;
(2Z)-4-hydroxy-2-({4-[4-methoxy-3-(pyrrolidin-1-ylsulfonyl)phenyl]-1-methyl-1H-pyrrolo[2,3-b]pyridin-3-yl}methylene)-1-benzofuran-3(2H)-one;
(2Z)-4,6-dihydroxy-2-{[1-methyl-4-(4-phenoxyphenyl)-1H-pyrrolo[2,3-b]pyridin-3-yl]methylene}-1-benzofuran-3(2H)-one;
(2Z)-4,6-dihydroxy-2-({4-[4-methoxy-3-(morpholin-4-ylsulfonyl)phenyl]-1-methyl-1H-pyrrolo[2,3-b]pyridin-3-yl}methylene)-1-benzofuran-3(2H)-one;
(2Z)-4,6-dihydroxy-2-({4-[4-methoxy-3-(pyrrolidin-1-ylsulfonyl)phenyl]-1-methyl-1H-pyrrolo[2,3-b]pyridin-3-yl}methylene)-1-benzofuran-3(2H)-one;
N-benzyl-4-{3-[(Z)-(4-hydroxy-3-oxo-1-benzofuran-2(3H)-ylidene)methyl]-1-methyl-1H-pyrrolo[2,3-b]pyridin-4-yl}benzenesulfonamide;
(2Z)-4-hydroxy-2-({1-methyl-4-[4-(pyrrolidin-1-ylcarbonyl)phenyl]-1H-pyrrolo[2,3-b]pyridin-3-yl}methylene)-1-benzofuran-3(2H)-one;
(2Z)-4,6-dihydroxy-2-({1-methyl-4-[4-(pyrrolidin-1-ylcarbonyl)phenyl]-1H-pyrrolo[2,3-b]pyridin-3-yl}methylene)-1-benzofuran-3(2H)-one;
(2Z)-2-{[4-(2,6-dimethylmorpholin-4-yl)-1-methyl-1H-pyrrolo[2,3-b]pyridin-3-yl]methylene}-4-hydroxy-1-benzofuran-3(2H)-one;
(2Z)-4,6-dihydroxy-2-[(1-methyl-4-phenoxy-1H-pyrrolo[2,3-b]pyridin-3-yl)methylene]-1-benzofuran-3(2H)-one;
(2Z)-4-hydroxy-2-[(4-hydroxy-1-methyl-1H-pyrrolo[2,3-b]pyridin-3-yl)methylene]-1-benzofuran-3(2H)-one;
N-(4-fluorophenyl)-3-{3-[(Z)-(4-hydroxy-3-oxo-1-benzofuran-2(3H)-ylidene)methyl]-1-methyl-1H-pyrrolo[2,3-b]pyridin-4-yl}benzamide;
3-{3-[(Z)-(4,6-dihydroxy-3-oxo-1-benzofuran-2(3H)-ylidene)methyl]-1-methyl-1H-pyrrolo[2,3-b]pyridin-4-yl}-N-(4-fluorophenyl)benzamide;
5-{3-[(Z)-(4,6-dihydroxy-3-oxo-1-benzofuran-2(3H)-ylidene)methyl]-1-methyl-1H-pyrrolo[2,3-b]pyridin-4-yl}-2-fluoro-N-phenylbenzamide;
1-methyl-3-{(2Z)-2-[(1-methyl-4-phenyl-1H-pyrrolo[2,3-b]pyridin-3-yl)methylene]-3-oxo-2,3-dihydro-1-benzofuran-4-yl}urea;
(2Z)-2-{[4-(3,5-dimethylpiperidin-1-yl)-1-methyl-1H-pyrrolo[2,3-b]pyridin-3-yl]methylene}-4-hydroxy-1-benzofuran-3(2H)-one;
(2Z)-2-{[4-(2,5-diazabicyclo[2.2.1]hept-2-yl)-1-methyl-1H-pyrrolo[2,3-b]pyridin-3-yl]methylene}-4-hydroxy-1-benzofuran-3(2H)-one;
(2Z)-4-hydroxy-2-[(1-methyl-4-thiomorpholin-4-yl-1H-pyrrolo[2,3-b]pyridin-3-yl)methylene]-1-benzofuran-3(2H)-one;
(2Z)-4-hydroxy-2-({1-methyl-4-[3-(pyrrolidin-1-ylcarbonyl)phenyl]-1H-pyrrolo[2,3-b]pyridin-3-yl}methylene)-1-benzofuran-3(2H)-one;
(2Z)-4-hydroxy-2-({1-methyl-4-[4-(pyrrolidin-1-ylcarbonyl)piperazin-1-yl]-1H-pyrrolo[2,3-b]pyridin-3-yl}methylene)-1-benzofuran-3(2H)-one;
(2Z)-4-hydroxy-2-{[1-methyl-4-(1,3,3-trimethyl-6-azabicyclo[3.2.1]oct-6-yl)-1H-pyrrolo[2,3-b]pyridin-3-yl]methylene}-1-benzofuran-3(2H)-one;
(2Z)-4-hydroxy-2-({1-methyl-4-[4-(trifluoromethyl)phenyl]-1H-pyrrolo[2,3-b]pyridin-3-yl}methylene)-1-benzofuran-3(2H)-one;
(2Z)-4-hydroxy-2-({1-methyl-4-[3-(trifluoromethyl)phenyl]-1H-pyrrolo[2,3-b]pyridin-3-yl}methylene)-1-benzofuran-3(2H)-one;
(2Z)-4-amino-2-[(1-methyl-4-phenyl-1H-pyrrolo[2,3-b]pyridin-3-yl)methylene]-1-benzofuran-3(2H)-one;
(2Z)-4-hydroxy-2-({1-methyl-4-[3-(pyrrolidin-1-ylcarbonyl)piperidin-1-yl]-1H-pyrrolo[2,3-b]pyridin-3-yl}methylene)-1-benzofuran-3(2H)-one;
(2Z)-4-hydroxy-2-({1-methyl-4-[3-(trifluoromethoxy)phenyl]-1H-pyrrolo[2,3-b]pyridin-3-yl}methylene)-1-benzofuran-3(2H)-one;
4-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzamide;
(2Z)-2-({4-[3-(dimethylamino)pyrrolidin-1-yl]-1-methyl-1H-pyrrolo[2,3-b]pyridin-3-yl}methylene)-4-hydroxy-1-benzofuran-3(2H)-one;
(2Z)-2-[(4-{4-[(dimethylamino)methyl]phenyl}-1-methyl-1H-pyrrolo[2,3-b]pyridin-3-yl)methylene]-4-hydroxy-1-benzofuran-3(2H)-one;
1-{3-[(Z)-(4-hydroxy-3-oxo-1-benzofuran-2(3H)-ylidene)methyl]-1-methyl-1H-pyrrolo[2,3-b]pyridin-4-yl}piperidine-4-carboxamide;
(2Z)-2-({4-[4-(aminomethyl)piperidin-1-yl]-1-methyl-1H-pyrrolo[2,3-b]pyridin-3-yl}methylene)-4-hydroxy-1-benzofuran-3(2H)-one;
(2Z)-2-[(1-ethyl-4-phenyl-1H-pyrrolo[2,3-b]pyridin-3-yl)methylene]-4-hydroxy-1-benzofuran-3(2H)-one;
(2Z)-4-hydroxy-2-{[1-methyl-4-(2-oxa-5-azabicyclo[2.2.1]hept-5-yl)-1H-pyrrolo[2,3-b]pyridin-3-yl]methylene}-1-benzofuran-3(2H)-one;
(2Z)-2-[(4-{4-[3-(dimethylamino)propyl]piperazin-1-yl}-1-methyl-1H-pyrrolo[2,3-b]pyridin-3-yl)methylene]-4-hydroxy-1-benzofuran-3(2H)-one;
1-{(2Z)-2-[(5-methoxy-1H-pyrrolo[2,3-b]pyridin-3-yl)methylene]-3-oxo-2,3-dihydro-1-benzofuran-5-yl}-3-methylurea;
1-[(2Z)-2-({5-methoxy-1-[2-(4-methylpiperazin-1-yl)ethyl]-1H-pyrrolo[2,3-b]pyridin-3-yl}methylene)-3-oxo-2,3-dihydro-1-benzofuran-5-yl]-3-methylurea;
3-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzamide;
(2Z)-2-[(4-{3-[(dimethylamino)methyl]phenyl}-1-methyl-1H-pyrrolo[2,3-b]pyridin-3-yl)methylene]-4-hydroxy-1-benzofuran-3(2H)-one;
(2Z)-4-hydroxy-2-({4-[4-methoxy-3-(piperidin-1-ylsulfonyl)phenyl]-1-methyl-1H-pyrrolo[2,3-b]pyridin-3-yl}methylene)-1-benzofuran-3(2H)-one;
(2Z)-4-hydroxy-2-[(1-methyl-4-{4-[(4-methylpiperazin-1-yl)carbonyl]phenyl}-1H-pyrrolo[2,3-b]pyridin-3-yl)methylene]-1-benzofuran-3(2H)-one;
(2Z)-4-hydroxy-2-{[4-(3-hydroxyphenyl)-1-methyl-1H-pyrrolo[2,3-b]pyridin-3-yl]methylene}-1-benzofuran-3(2H)-one;
(2Z)-4-hydroxy-2-{[1-methyl-4-(4-methyl-3,4-dihydro-2H-1,4-benzoxazin-7-yl)-1H-pyrrolo[2,3-b]pyridin-3-yl]methylene}-1-benzofuran-3(2H)-one;
(2Z)-2-({4-[4-(2-furyl)phenyl]-1-methyl-1H-pyrrolo[2,3-b]pyridin-3-yl}methylene)-4-hydroxy-1-benzofuran-3(2H)-one;
(2Z)-4-hydroxy-2-({1-methyl-4-[4-(morpholin-4-ylcarbonyl)phenyl]-1H-pyrrolo[2,3-b]pyridin-3-yl}methylene)-1-benzofuran-3(2H)-one;
(2Z)-4-hydroxy-2-({1-methyl-4-[4-(4-methylpiperazin-1-yl)piperidin-1-yl]-1H-pyrrolo[2,3-b]pyridin-3-yl}methylene)-1-benzofuran-3(2H)-one;
(2Z)-4-hydroxy-2-({1-methyl-4-[4-(morpholin-4-ylmethyl)phenyl]-1H-pyrrolo[2,3-b]pyridin-3-yl}methylene)-1-benzofuran-3(2H)-one;
1-methyl-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
4-{3-[(Z)-(4-hydroxy-3-oxo-1-benzofuran-2(3H)-ylidene)methyl]-1-methyl-1H-pyrrolo[2,3-b]pyridin-4-yl}-N-methylbenzamide;
1-(4-{3-[(Z)-(4-hydroxy-3-oxo-1-benzofuran-2(3H)-ylidene)methyl]-1-methyl-1H-pyrrolo[2,3-b]pyridin-4-yl}phenyl)-3-methylurea;
(2Z)-2-{[4-(4-acetylphenyl)-1-methyl-1H-pyrrolo[2,3-b]pyridin-3-yl]methylene}-4-hydroxy-1-benzofuran-3(2H)-one;
(2Z)-4-hydroxy-2-{[1-methyl-4-(4-piperazin-1-ylphenyl)-1H-pyrrolo[2,3-b]pyridin-3-yl]methylene}-1-benzofuran-3(2H)-one;
(2Z)-4-hydroxy-2-({1-methyl-4-[4-(piperazin-1-ylcarbonyl)phenyl]-1H-pyrrolo[2,3-b]pyridin-3-yl}methylene)-1-benzofuran-3(2H)-one;
2-fluoro-5-{3-[(Z)-(4-hydroxy-3-oxo-1-benzofuran-2(3H)-ylidene)methyl]-1-methyl-1H-pyrrolo[2,3-b]pyridin-4-yl}-N-phenylbenzamide;
3-{3-[(Z)-(4-hydroxy-3-oxo-1-benzofuran-2(3H)-ylidene)methyl]-1-methyl-1H-pyrrolo[2,3-b]pyridin-4-yl}-N-phenylbenzamide;
(2Z)-2-{[4-(4-aminopiperidin-1-yl)-1-methyl-1H-pyrrolo[2,3-b]pyridin-3-yl]methylene}-4-hydroxy-1-benzofuran-3(2H)-one;
3-(4-{3-[(Z)-(4-hydroxy-3-oxo-1-benzofuran-2(3H)-ylidene)methyl]-1-methyl-1H-pyrrolo[2,3-b]pyridin-4-yl}phenyl)-1,1-dimethylurea;
2-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzamide;
(2Z)-4-hydroxy-2-{[1-methyl-4-(4-piperidin-1-ylphenyl)-1H-pyrrolo[2,3-b]pyridin-3-yl]methylidene}-1-benzofuran-3(2H)-one;
(2Z)-4-hydroxy-2-{[1-methyl-4-(4-pyrrolidin-1-ylphenyl)-1H-pyrrolo[2,3-b]pyridin-3-yl]methylidene}-1-benzofuran-3(2H)-one;
(2Z)-4-hydroxy-2-({1-methyl-4-[4-(8-oxa-3-azabicyclo[3.2.1]oct-3-ylcarbonyl)phenyl]-1H-pyrrolo[2,3-b]pyridin-3-yl}methylidene)-1-benzofuran-3(2H)-one;
1-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylpiperidine-4-carboxamide;
(2Z)-4-hydroxy-2-{[4-(4-{[4-(2-hydroxyethyl)piperazin-1-yl]carbonyl}phenyl)-1-methyl-1H-pyrrolo[2,3-b]pyridin-3-yl]methylidene}-1-benzofuran-3(2H)-one;
4-{3-[(Z)-(4-hydroxy-3-oxo-1-benzofuran-2(3H)-ylidene)methyl]-1-methyl-1H-pyrrolo[2,3-b]pyridin-4-yl}-N-(3-hydroxypropyl)benzamide;
N-(2-hydroxyethyl)-1-{3-[(Z)-(4-hydroxy-3-oxo-1-benzofuran-2(3H)-ylidene)methyl]-1-methyl-1H-pyrrolo[2,3-b]pyridin-4-yl}piperidine-4-carboxamide;
4-{3-[(Z)-(4-hydroxy-3-oxo-1-benzofuran-2(3H)-ylidene)methyl]-1-methyl-1H-pyrrolo[2,3-b]pyridin-4-yl}-N-(2-methoxyethyl)benzamide;
(2Z)-4-hydroxy-2-({1-methyl-4-[4-(morpholin-4-ylcarbonyl)piperidin-1-yl]-1H-pyrrolo[2,3-b]pyridin-3-yl}methylidene)-1-benzofuran-3(2H)-one;
(2Z)-4-hydroxy-2-{[1-methyl-4-(3-oxa-8-azabicyclo[3.2.1]oct-8-yl)-1H-pyrrolo[2,3-b]pyridin-3-yl]methylidene}-1-benzofuran-3(2H)-one;
(2Z)-4-hydroxy-2-({1-methyl-4-[4-(pyrrolidin-1-ylcarbonyl)piperidin-1-yl]-1H-pyrrolo[2,3-b]pyridin-3-yl}methylidene)-1-benzofuran-3(2H)-one;
(2Z)-4-hydroxy-2-{[1-methyl-4-(5-methyl-2,5-diazabicyclo[2.2.1]hept-2-yl)-1H-pyrrolo[2,3-b]pyridin-3-yl]methylidene}-1-benzofuran-3(2H)-one;
N-{(2Z)-2-[(1-methyl-4-phenyl-1H-pyrrolo[2,3-b]pyridin-3-yl)methylidene]-3-oxo-2,3-dihydro-1-benzofuran-5-yl}acetamide;
N-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]acetamide;
2,2,2-trifluoro-N-{(2Z)-2-[(1-methyl-4-phenyl-1H-pyrrolo[2,3-b]pyridin-3-yl)methylidene]-3-oxo-2,3-dihydro-1-benzofuran-5-yl}acetamide;
(2Z)-4-hydroxy-2-({1-methyl-4-[4-(8-oxa-3-azabicyclo[3.2.1]oct-3-yl)phenyl]-1H-pyrrolo[2,3-b]pyridin-3-yl}methylidene)-1-benzofuran-3(2H)-one;
N-[2-(dimethylamino)ethyl]-4-{3-[(Z)-(4-hydroxy-3-oxo-1-benzofuran-2(3H)-ylidene)methyl]-1-methyl-1H-pyrrolo[2,3-b]pyridin-4-yl}benzamide;
(2Z)-4-[2-(dimethylamino)ethoxy]-2-[(1-methyl-4-phenyl-1H-pyrrolo[2,3-b]pyridin-3-yl)methylidene]-1-benzofuran-3(2H)-one;
ethyl 4-{3-[(Z)-(4-hydroxy-3-oxo-1-benzofuran-2(3H)-ylidene)methyl]-1-methyl-1H-pyrrolo[2,3-b]pyridin-4-yl}benzoate;
1,1-dimethyl-3-{(2Z)-2-[(1-methyl-4-phenyl-1H-pyrrolo[2,3-b]pyridin-3-yl)methylidene]-3-oxo-2,3-dihydro-1-benzofuran-5-yl}urea;
N-{(2Z)-2-[(1-methyl-4-phenyl-1H-pyrrolo[2,3-b]pyridin-3-yl)methylidene]-3-oxo-2,3-dihydro-1-benzofuran-5-yl}methanesulfonamide;
N,N-dimethyl-1-{1-methyl-3-[(Z)-{3-oxo-5-[(trifluoroacetyl)amino]-1-benzofuran-2(3H)-ylidene}methyl]-1H-pyrrolo[2,3-b]pyridin-4-yl}piperidine-4-carboxamide;
(2Z)-4-hydroxy-2-[(1-methyl-4-{4-[(4-methylpiperazin-1-yl)carbonyl]piperidin-1-yl}-1H-pyrrolo[2,3-b]pyridin-3-yl)methylidene]-1-benzofuran-3(2H)-one;
(2Z)-2-{[4-(6,8-dioxa-3-azabicyclo[3.2.1]oct-3-yl)-1-methyl-1H-pyrrolo[2,3-b]pyridin-3-yl]methylidene}-4-hydroxy-1-benzofuran-3(2H)-one;
1-[(2Z)-2-{[4-(4-{[4-(2-hydroxyethyl)piperazin-1-yl]carbonyl}phenyl)-1-methyl-1H-pyrrolo[2,3-b]pyridin-3-yl]methylidene}-3-oxo-2,3-dihydro-1-benzofuran-5-yl]-3-methylurea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-pyridin-3-ylurea;
N,N-dimethyl-1-{1-methyl-3-[(Z)-{5-[(methylcarbamoyl)amino]-3-oxo-1-benzofuran-2(3H)-ylidene}methyl]-1H-pyrrolo[2,3-b]pyridin-4-yl}piperidine-4-carboxamide;
1-{3-[(Z)-(5-bromo-3-oxo-1-benzofuran-2(3H)-ylidene)methyl]-1-methyl-1H-pyrrolo[2,3-b]pyridin-4-yl}-N,N-dimethylpiperidine-4-carboxamide;
N-[(2Z)-2-({4-[4-(dimethylcarbamoyl)piperidin-1-yl]-1-methyl-1H-pyrrolo[2,3-b]pyridin-3-yl}methylidene)-3-oxo-2,3-dihydro-1-benzofuran-5-yl]morpholine-4-carboxamide;
ethyl[(2Z)-2-({4-[4-(dimethylcarbamoyl)piperidin-1-yl]-1-methyl-1H-pyrrolo[2,3-b]pyridin-3-yl}methylidene)-3-oxo-2,3-dihydro-1-benzofuran-5-yl]carbamate;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-phenylurea;
1-[(2Z)-2-({1-methyl-4-[4-(morpholin-4-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
1-methyl-3-[(2Z)-2-({1-methyl-4-[4-(morpholin-4-ylcarbonyl)piperidin-1-yl]-1H-pyrrolo[2,3-b]pyridin-3-yl}methylidene)-3-oxo-2,3-dihydro-1-benzofuran-5-yl]urea;
4-{3-[(Z)-(4-hydroxy-3-oxo-1-benzofuran-2(3H)-ylidene)methyl]-1-methyl-1H-pyrrolo[2,3-b]pyridin-4-yl}-N-(2-methoxyethyl)-N-methylbenzamide;
(2Z)-2-[(1-methyl-4-phenyl-1H-pyrrolo[2,3-b]pyridin-3-yl)methylidene]-3-oxo-2,3-dihydro-1-benzofuran-5-carboxylic acid;
1-[(2Z)-2-{[1-ethyl-4-(4-{[4-(2-hydroxyethyl)piperazin-1-yl]carbonyl}phenyl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-({1-methyl-4-[4-(morpholin-4-ylcarbonyl)phenyl]-1H-pyrrolo[2,3-b]pyridin-3-yl}methylidene)-3-oxo-2,3-dihydro-1-benzofuran-5-yl]-3-pyridin-3-ylurea;
1-methyl-3-[(2Z)-2-({1-methyl-4-[4-(morpholin-4-ylcarbonyl)phenyl]-1H-pyrrolo[2,3-b]pyridin-3-yl}methylidene)-3-oxo-2,3-dihydro-1-benzofuran-5-yl]urea;
4-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzenesulfonamide;
(2Z)-4-hydroxy-2-({1-methyl-4-[4-(piperidin-1-ylsulfonyl)phenyl]-1H-pyrrolo[2,3-b]pyridin-3-yl}methylidene)-1-benzofuran-3(2H)-one;
N-(2-hydroxyethyl)-4-{3-[(Z)-(4-hydroxy-3-oxo-1-benzofuran-2(3H)-ylidene)methyl]-1-methyl-1H-pyrrolo[2,3-b]pyridin-4-yl}benzamide;
1-{(2Z)-2-[(1-methyl-4-phenyl-1H-pyrrolo[2,3-b]pyridin-3-yl)methylidene]-3-oxo-2,3-dihydro-1-benzofuran-5-yl}-3-pyridin-3-ylurea;
1-[(2Z)-2-{[1-methyl-4-(4-methylpiperazin-1-yl)-1H-pyrrolo[2,3-b]pyridin-3-yl]methylidene}-3-oxo-2,3-dihydro-1-benzofuran-5-yl]-3-pyridin-3-ylurea;
1-{(2Z)-2-[(1-methyl-4-morpholin-4-yl-1H-pyrrolo[2,3-b]pyridin-3-yl)methylidene]-3-oxo-2,3-dihydro-1-benzofuran-5-yl}-3-pyridin-3-ylurea;
(2Z)-2-{[1-ethyl-4-(4-{[4-(2-hydroxyethyl)piperazin-1-yl]carbonyl}phenyl)-1H-pyrrolo[2,3-b]pyridin-3-yl]methylidene}-4-hydroxy-1-benzofuran-3(2H)-one;
(2Z)-N-[2-(dimethylamino)ethyl]-2-[(1-methyl-4-phenyl-1H-pyrrolo[2,3-b]pyridin-3-yl)methylidene]-3-oxo-2,3-dihydro-1-benzofuran-5-carboxamide;
4-methyl-N-{(2Z)-2-[(1-methyl-4-phenyl-1H-pyrrolo[2,3-b]pyridin-3-yl)methylidene]-3-oxo-2,3-dihydro-1-benzofuran-5-yl}piperazine-1-carboxamide;
N,N-dimethyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
(2Z)-4-hydroxy-2-({1-methyl-4-[4-(pyrrolidin-1-ylsulfonyl)phenyl]-1H-pyrrolo[2,3-b]pyridin-3-yl}methylidene)-1-benzofuran-3(2H)-one;
(2Z)-4-hydroxy-2-({1-methyl-4-[4-(morpholin-4-ylsulfonyl)phenyl]-1H-pyrrolo[2,3-b]pyridin-3-yl}methylidene)-1-benzofuran-3(2H)-one;
4-{3-[(Z)-(4-hydroxy-3-oxo-1-benzofuran-2(3H)-ylidene)methyl]-1-methyl-1H-pyrrolo[2,3-b]pyridin-4-yl}-N-methoxybenzamide;
1-[2-(dimethylamino)ethyl]-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[2-(4-methylpiperazin-1-yl)ethyl]urea;
N-(2-hydroxyethyl)-4-{3-[(Z)-(4-hydroxy-3-oxo-1-benzofuran-2(3H)-ylidene)methyl]-1-methyl-1H-pyrrolo[2,3-b]pyridin-4-yl}-N-methylbenzamide;
4-{3-[(Z)-(4-hydroxy-3-oxo-1-benzofuran-2(3H)-ylidene)methyl]-1-methyl-1H-pyrrolo[2,3-b]pyridin-4-yl}-N-methoxy-N-methylbenzamide;
(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-carboxylic acid;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-pyridin-4-ylurea;
N-(3-methoxypropyl)-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-(3-methoxypropyl)-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-[2-(dimethylamino)-2-oxoethyl]-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-(2-amino-2-oxoethyl)-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(2-pyrrolidin-1-ylethyl)piperidine-4-carboxamide;
1-[2-({4-[4-(2,5-dihydro-1H-pyrrol-1-ylcarbonyl)piperidin-1-yl]-1-methyl-1H-pyrrolo[2,3-b]pyridin-3-yl}methylidene)-3-oxo-2,3-dihydro-1-benzofuran-5-yl]-3-pyridin-3-ylurea;
N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(1-methylpyrrolidin-3-yl)piperidine-4-carboxamide;
N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-[(3R)-pyrrolidin-3-yl]piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-[(3R)-pyrrolidin-3-yl]piperidine-4-carboxamide;
N-methyl-1-[1-methyl-3-({3-oxo-5-[(pyridin-3-ylcarbamoyl)amino]-1-benzofuran-2(3H)-ylidene}methyl)-H-pyrrolo[2,3-b]pyridin-4-yl]-N-[(3S)-pyrrolidin-3-yl]piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-[(3S)-pyrrolidin-3-yl]piperidine-4-carboxamide;
1-(2-{[4-(4-{[3-(dimethylamino)pyrrolidin-1-yl]carbonyl}piperidin-1-yl)-1-methyl-1H-pyrrolo[2,3-b]pyridin-3-yl]methylidene}-3-oxo-2,3-dihydro-1-benzofuran-5-yl)-3-pyridin-3-ylurea;
N-(1,1-dioxidotetrahydrothiophen-3-yl)-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-(1,1-dioxidotetrahydrothiophen-3-yl)-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(tetrahydro-2H-pyran-4-yl)piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(tetrahydro-2H-pyran-4-yl)piperidine-4-carboxamide;
N-benzyl-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N,N-dimethyl-1-(1-methyl-3-{(Z)-[3-oxo-5-({[4-(piperidin-1-ylmethyl)phenyl]carbamoyl}amino)-1-benzofuran-2(3H)-ylidene]methyl}-1H-pyrrolo[2,3-b]pyridin-4-yl)piperidine-4-carboxamide;
N,N-dimethyl-1-(1-methyl-3-{(Z)-[5-{[(6-morpholin-4-ylpyridin-3-yl)carbamoyl]amino}-3-oxo-1-benzofuran-2(3H)-ylidene]methyl}-1H-pyrrolo[2,3-b]pyridin-4-yl)piperidine-4-carboxamide;
N,N-dimethyl-1-(1-methyl-3-{(Z)-[5-({[4-(4-methylpiperazin-1-yl)phenyl]carbamoyl}amino)-3-oxo-1-benzofuran-2(3H)-ylidene]methyl}-1H-pyrrolo[2,3-b]pyridin-4-yl)piperidine-4-carboxamide;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-(6-morpholin-4-ylpyridin-3-yl)urea;
1-{4-[2-(dimethylamino)ethoxy]phenyl}-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-(4-methylpiperazin-1-yl)urea;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-[(3-methylpyridin-4-yl)methyl]piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-[(2-methylpyridin-4-yl)methyl]piperidine-4-carboxamide;
N-[(4-methoxypyridin-2-yl)methyl]-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-[(6-methylpyridin-2-yl)methyl]piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-prop-2-yn-1-ylpiperidine-4-carboxamide;
N-cyclopentyl-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N,N-dimethyl-1-(1-methyl-3-{(Z)-[5-{[(4-methylpiperazin-1-yl)carbamoyl]amino}-3-oxo-1-benzofuran-2(3H)-ylidene]methyl}-1H-pyrrolo[2,3-b]pyridin-4-yl)piperidine-4-carboxamide;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[4-(4-methylpiperazin-1-yl)phenyl]urea;
N,N-dimethyl-1-(1-methyl-3-{(Z)-[5-({[6-(4-methylpiperazin-1-yl)pyridin-3-yl]carbamoyl}amino)-3-oxo-1-benzofuran-2(3H)-ylidene]methyl}-1H-pyrrolo[2,3-b]pyridin-4-yl)piperidine-4-carboxamide;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[6-(4-methylpiperazin-1-yl)pyridin-3-yl]urea;
1-[(1-methyl-1H-imidazol-5-yl)methyl]-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-({1-methyl-4-[4-(2-oxa-5-azabicyclo[2.2.2]oct-5-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
1-[(2Z)-2-{[1-methyl-4-(piperidin-1-ylcarbonyl)-1H-pyrrolo[2,3-b]pyridin-3-yl]methylidene}-3-oxo-2,3-dihydro-1-benzofuran-5-yl]-3-pyridin-3-ylurea;
1-(1-methylethyl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-cyclohexyl-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-cyclopropyl-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-({1-methyl-4-[(3S)-3-methylmorpholin-4-yl]-1H-pyrrolo[2,3-b]pyridin-3-yl}methylidene)-3-oxo-2,3-dihydro-1-benzofuran-5-yl]-3-pyridin-3-ylurea;
1-{(2Z)-2-[(4-{4-[(2R,6S)-2,6-dimethylmorpholin-4-yl]piperidin-1-yl}-1-methyl-1H-pyrrolo[2,3-b]pyridin-3-yl)methylidene]-3-oxo-2,3-dihydro-1-benzofuran-5-yl}-3-pyridin-3-ylurea;
1-(3-chloropropyl)-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
1-[3-(dimethylamino)propyl]-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
1-{3-[(2-methoxyethyl)(methyl)amino]propyl}-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
N-ethyl-N-(2-hydroxyethyl)-4-{3-[(Z)-(4-hydroxy-3-oxo-1-benzofuran-2(3H)-ylidene)methyl]-1-methyl-1H-pyrrolo[2,3-b]pyridin-4-yl}benzamide;
N,N-diethyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
(2Z)-4-hydroxy-2-[(4-{4-[(3-hydroxypyrrolidin-1-yl)carbonyl]phenyl}-1-methyl-1H-pyrrolo[2,3-b]pyridin-3-yl)methylene]-1-benzofuran-3(2H)-one;
N-(2-methoxyethyl)-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-(2-methoxyethyl)-N-methyl-4-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}benzamide;
N-methoxy-4-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}benzamide;
(2Z)-4-hydroxy-2-[(4-{4-[(3-methoxypyrrolidin-1-yl)carbonyl]phenyl}-1-methyl-1H-pyrrolo[2,3-b]pyridin-3-yl)methylene]-1-benzofuran-3(2H)-one;
1-[(2Z)-2-({1-methyl-4-[4-(8-oxa-3-azabicyclo[3.2.1]oct-3-ylcarbonyl)piperidin-1-yl]-1H-pyrrolo[2,3-b]pyridin-3-yl}methylene)-3-oxo-2,3-dihydro-1-benzofuran-5-yl]-3-pyridin-3-ylurea;
N,N-bis(2-methoxyethyl)-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
1-{(2Z)-2-[(1-methyl-4-{4-[(4-methylpiperazin-1-yl)carbonyl]piperidin-1-yl}-1H-pyrrolo[2,3-b]pyridin-3-yl)methylidene]-3-oxo-2,3-dihydro-1-benzofuran-5-yl}-3-pyridin-3-ylurea;
1-[(2Z)-2-({1-methyl-4-[4-(pyrrolidin-1-ylcarbonyl)piperidin-1-yl]-1H-pyrrolo[2,3-b]pyridin-3-yl}methylene)-3-oxo-2,3-dihydro-1-benzofuran-5-yl]-3-pyridin-3-ylurea;
N-(2-hydroxyethyl)-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-4-ylmethyl)piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-3-ylmethyl)piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-2-ylmethyl)piperidine-4-carboxamide;
1-[(2Z)-2-({1-methyl-4-[4-(piperidin-1-ylcarbonyl)piperidin-1-yl]-1H-pyrrolo[2,3-b]pyridin-3-yl}methylene)-3-oxo-2,3-dihydro-1-benzofuran-5-yl]-3-pyridin-3-ylurea;
1-{(2Z)-2-[(4-{4-[(3-methoxypyrrolidin-1-yl)carbonyl]phenyl}-1-methyl-1H-pyrrolo[2,3-b]pyridin-3-yl)methylene]-3-oxo-2,3-dihydro-1-benzofuran-5-yl}-3-pyridin-3-ylurea;
N-(2-methoxyethyl)-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-[2-(dimethylamino)ethyl]-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-pyridin-3-ylpiperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-pyridin-4-ylpiperidine-4-carboxamide;
1-[(2Z)-2-({4-[4-(2,5-diazabicyclo[2.2.1]hept-2-ylcarbonyl)piperidin-1-yl]-1-methyl-1H-pyrrolo[2,3-b]pyridin-3-yl}methylene)-3-oxo-2,3-dihydro-1-benzofuran-5-yl]-3-pyridin-3-ylurea;
1-{(2Z)-2-[(4-{4-[(2,6-dimethylmorpholin-4-yl)carbonyl]piperidin-1-yl}-1-methyl-1H-pyrrolo[2,3-b]pyridin-3-yl)methylene]-3-oxo-2,3-dihydro-1-benzofuran-5-yl}-3-pyridin-3-ylurea;
N-[2-(dimethylamino)ethyl]-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N,N-dimethyl-1-(1-methyl-3-{(Z)-[3-oxo-5-({[6-(tetrahydro-2H-pyran-4-yloxy)pyridin-3-yl]carbamoyl}amino)-1-benzofuran-2(3H)-ylidene]methyl}-1H-pyrrolo[2,3-b]pyridin-4-yl)piperidine-4-carboxamide;
N,N-dimethyl-1-(1-methyl-3-{(Z)-[5-{[(4-morpholin-4-ylphenyl)carbamoyl]amino}-3-oxo-1-benzofuran-2(3H)-ylidene]methyl}-1H-pyrrolo[2,3-b]pyridin-4-yl)piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-pyridin-3-ylpiperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-3-ylmethyl)piperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-4-ylmethyl)piperidine-4-carboxamide;
1-{(2Z)-2-[(1-methyl-4-{4-[(5-methyl-2,5-diazabicyclo[2.2.1]hept-2-yl)carbonyl]piperidin-1-yl}-1H-pyrrolo[2,3-b]pyridin-3-yl)methylidene]-3-oxo-2,3-dihydro-1-benzofuran-5-yl}-3-pyridin-3-ylurea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-(4-morpholin-4-ylphenyl)urea;
1-{3-[(Z)-{5-[({4-[2-(dimethylamino)ethoxy]phenyl}carbamoyl)amino]-3-oxo-1-benzofuran-2(3H)-ylidene}methyl]-1-methyl-1H-pyrrolo[2,3-b]pyridin-4-yl}-N,N-dimethylpiperidine-4-carboxamide;
1-{(2Z)-2-[(4-{4-[(3-methoxypyrrolidin-1-yl)carbonyl]piperidin-1-yl}-1-methyl-1H-pyrrolo[2,3-b]pyridin-3-yl)methylidene]-3-oxo-2,3-dihydro-1-benzofuran-5-yl}-3-pyridin-3-ylurea;
1-[(2Z)-2-{[1-methyl-4-(3-oxa-9-azabicyclo[3.3.1]non-9-yl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[1-methyl-4-(2-oxa-5-azabicyclo[2.2.2]oct-5-yl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[4-(piperidin-1-ylmethyl)phenyl]urea;
1-methyl-3-[(2Z)-2-{[1-methyl-4-(2-oxa-5-azabicyclo[2.2.2]oct-5-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(3-oxa-8-azabicyclo[3.2.1]oct-8-yl)-1H-pyrrolo[2,3-b]pyridin-3-yl]methylidene}-3-oxo-2,3-dihydro-1-benzofuran-5-yl]-3-pyridin-3-ylurea;
1-methyl-3-[(2Z)-2-{[1-methyl-4-(3-oxa-8-azabicyclo[3.2.1]oct-8-yl)-1H-pyrrolo[2,3-b]pyridin-3-yl]methylidene}-3-oxo-2,3-dihydro-1-benzofuran-5-yl]urea;
1-[(2Z)-2-{[4-(6,8-dioxa-3-azabicyclo[3.2.1]oct-3-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[1-methyl-4-(2-oxa-5-azabicyclo[2.2.1]hept-5-yl)-1H-pyrrolo[2,3-b]pyridin-3-yl]methylidene}-3-oxo-2,3-dihydro-1-benzofuran-5-yl]-3-pyridin-3-ylurea;
1-methyl-3-[(2Z)-2-{[1-methyl-4-(2-oxa-5-azabicyclo[2.2.1]hept-5-yl)-1H-pyrrolo[2,3-b]pyridin-3-yl]methylidene}-3-oxo-2,3-dihydro-1-benzofuran-5-yl]urea;
1-azetidin-3-yl-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[1-(trifluoroacetyl)piperidin-4-yl]urea;
1-{3-[(Z)-{5-[(azetidin-3-ylcarbamoyl)amino]-3-oxo-1-benzofuran-2(3H)-ylidene}methyl]-1-methyl-1H-pyrrolo[2,3-b]pyridin-4-yl}-N,N-dimethylpiperidine-4-carboxamide;
N,N-dimethyl-1-(1-methyl-3-{(Z)-[5-{[(1-methylethyl)carbamoyl]amino}-3-oxo-1-benzofuran-2(3H)-ylidene]methyl}-1H-pyrrolo[2,3-b]pyridin-4-yl)piperidine-4-carboxamide;
1-(1-methyl-1H-benzimidazol-2-yl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
N,N-dimethyl-1-(1-methyl-3-{(Z)-[5-{[(1-methyl-1H-benzimidazol-2-yl)carbamoyl]amino}-3-oxo-1-benzofuran-2(3H)-ylidene]methyl}-1H-pyrrolo[2,3-b]pyridin-4-yl)piperidine-4-carboxamide;
1-{3-[(Z)-{5-[(cyclopropylcarbamoyl)amino]-3-oxo-1-benzofuran-2(3H)-ylidene}methyl]-1-methyl-1H-pyrrolo[2,3-b]pyridin-4-yl}-N,N-dimethylpiperidine-4-carboxamide;
1-[(2Z)-2-({1-methyl-4-[4-(3-oxa-8-azabicyclo[3.2.1]oct-8-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
1-[(2Z)-2-{[1-methyl-4-(9-oxa-3,7-diazabicyclo[3.3.1]non-3-yl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[4-(3,6-dihydro-2H-pyran-4-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[4-(2,2-diethylmorpholin-4-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-({4-[(2R,6S)-2,6-dimethylmorpholin-4-yl]-1-methyl-1H-pyrrolo[2,3-b]pyridin-3-yl}methylidene)-3-oxo-2,3-dihydro-1-benzofuran-5-yl]-3-pyridin-3-ylurea;
1-[(2Z)-2-({4-[2-(2-methoxyethyl)morpholin-4-yl]-1-methyl-1H-pyrrolo[2,3-b]pyridin-3-yl}methylidene)-3-oxo-2,3-dihydro-1-benzofuran-5-yl]-3-pyridin-3-ylurea;
1-(6-fluoropyridin-3-yl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-(6-chloropyridin-3-yl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
N,N-dimethyl-1-(1-methyl-3-{(Z)-[3-oxo-5-({[1-(trifluoroacetyl)piperidin-4-yl]carbamoyl}amino)-1-benzofuran-2(3H)-ylidene]methyl}-1H-pyrrolo[2,3-b]pyridin-4-yl)piperidine-4-carboxamide;
1-[(2Z)-2-{[4-(2,2-dimethylmorpholin-4-yl)-1-methyl-1H-pyrrolo[2,3-b]pyridin-3-yl]methylidene}-3-oxo-2,3-dihydro-1-benzofuran-5-yl]-3-pyridin-3-ylurea;
(2Z)-2-{[4-(4-{[(2R,6S)-2,6-dimethylmorpholin-4-yl]carbonyl}piperidin-1-yl)-1-methyl-1H-pyrrolo[2,3-b]pyridin-3-yl]methylidene}-4-hydroxy-1-benzofuran-3(2H)-one;
(2Z)-2-{[4-(4-{[(2R,6S)-2,6-dimethylmorpholin-4-yl]carbonyl}phenyl)-1-methyl-1H-pyrrolo[2,3-b]pyridin-3-yl]methylidene}-4-hydroxy-1-benzofuran-3(2H)-one;
(2Z)-4-hydroxy-2-{[4-(4-{[4-(2-methoxyethyl)piperazin-1-yl]carbonyl}phenyl)-1-methyl-1H-pyrrolo[2,3-b]pyridin-3-yl]methylidene}-1-benzofuran-3(2H)-one;
(2Z)-4-hydroxy-2-{[4-(4-{[2-(2-methoxyethyl) morpholin-4-yl]carbonyl}phenyl)-1-methyl-1H-pyrrolo[2,3-b]pyridin-3-yl]methylidene}-1-benzofuran-3(2H)-one;
(2Z)-4-hydroxy-2-({1-methyl-4-[4-(8-oxa-3-azabicyclo[3.2.1]oct-3-ylcarbonyl)piperidin-1-yl]-1H-pyrrolo[2,3-b]pyridin-3-yl}methylidene)-1-benzofuran-3(2H)-one;
(2Z)-4-hydroxy-2-{[4-(4-{[4-(2-hydroxyethyl)piperazin-1-yl]carbonyl}piperidin-1-yl)-1-methyl-1H-pyrrolo[2,3-b]pyridin-3-yl]methylidene}-1-benzofuran-3(2H)-one;
1-[(2Z)-2-{[4-(4-{[2-(methoxymethyl)morpholin-4-yl]carbonyl}piperidin-1-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-({1-methyl-4-[4-(3-oxa-9-azabicyclo[3.3.1]non-9-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
(2Z)-4-hydroxy-2-{[4-(4-{[2-(methoxymethyl)morpholin-4-yl]carbonyl}phenyl)-1-methyl-1H-pyrrolo[2,3-b]pyridin-3-yl]methylidene}-1-benzofuran-3(2H)-one;
(2Z)-4-hydroxy-2-{[4-(4-{[2-(methoxymethyl)morpholin-4-yl]carbonyl}piperidin-1-yl)-1-methyl-1H-pyrrolo[2,3-b]pyridin-3-yl]methylidene}-1-benzofuran-3(2H)-one; and
(2Z)-4-hydroxy-2-({1-methyl-4-[4-(3-oxa-9-azabicyclo[3.3.1]non-9-ylcarbonyl)piperidin-1-yl]-1H-pyrrolo[2,3-b]pyridin-3-yl}methylidene)-1-benzofuran-3(2H)-one.

In another aspect, the invention provides compounds of the Formula 5:

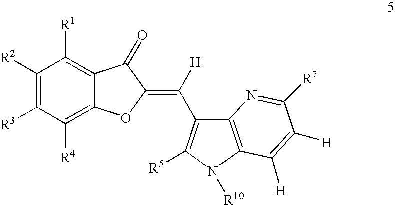

or a geometric isomer thereof or a pharmaceutically acceptable salt thereof, wherein:

the constituent variables are as defined for Formula 1.

In one embodiment, R¹ is hydroxyl.

In one embodiment, R² is H.

In one embodiment, R³ is hydroxyl.

In one embodiment, R⁴ is H.

In one embodiment, R⁵ is H.

In one embodiment, R⁷ is C₁-C₆alkoxy.

In one embodiment, R⁷ is methoxy.

In one embodiment, R¹⁰ is H or C₁-C₆alkyl.

In one embodiment, R¹⁰ is methyl.

In one embodiment, R¹═R³=hydroxyl and R²═R⁴═H.

In one embodiment, R⁵ is H. R⁷ is methoxy, and R¹⁰ is CH₃.

In one embodiment, R¹═R³=hydroxyl, R²═R⁴═R⁵═R¹⁰═H and R⁷ is methoxy.

Additional illustrative compounds of formula 5 are set forth below:
4,6-dihydroxy-2-[(5-methoxy-1-methyl-1H-pyrrolo[3,2-b]pyridin-3-yl)methylene]-1-benzofuran-3(2H)-one;
6,7-dihydroxy-2-[(5-methoxy-1-methyl-1H-pyrrolo[3,2-b]pyridin-3-yl)methylene]-1-benzofuran-3(2H)-one;
4-hydroxy-2-[(5-methoxy-1-methyl-1H-pyrrolo[3,2-b]pyridin-3-yl)methylene]-1-benzofuran-3(2H)-one; and (2Z)-4,6-dihydroxy-2-[(5-methoxy-1H-pyrrolo[3,2-b]pyridin-3-yl)methylene]-1-benzofuran-3(2H)-one.

In other aspects, the invention provides pharmaceutical compositions comprising compounds or pharmaceutically acceptable salts of the compounds of the present Formula 1 and a pharmaceutically acceptable carrier.

In other aspects, the invention provides that the pharmaceutically acceptable carrier suitable for oral administration and the composition comprises an oral dosage form.

In other aspects, the invention provides a composition comprising a compound of Formula 1; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

In other aspects, the second compound is Avastin.

In other aspects, the invention provides a method of treating a PI3K-related disorder, comprising administering to a mammal in need thereof a compound of Formula 1 in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating an mTOR-related disorder, comprising administering to a mammal in need thereof a compound of Formula 1 in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating advanced renal cell carcinoma, comprising administering to a mammal in need thereof a compound of Formula 1 in an amount effective to treat advanced renal cell carcinoma.

In other aspects, the invention provides a method of treating acute lymphoblastic leukemia, comprising administering to a mammal in need thereof a compound of Formula 1 in an amount effective to treat acute lymphoblastic leukemia.

In other aspects, the invention provides a method of treating acute malignant melanoma, comprising administering to a mammal in need thereof a compound of Formula 1 in an amount effective to treat malignant melanoma.

In other aspects, the invention provides a method of treating soft-tissue or bone sarcoma, comprising administering to a mammal in need thereof a compound of Formula 1 in an amount effective to treat soft-tissue or bone sarcoma.

In other aspects, the invention provides a method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition comprising a compound of Formula 1; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier. in an amount effective to treat the cancer.

In other aspects, the invention provides a method of inhibiting mTOR in a subject, comprising administering to a subject in need thereof a compound of Formula 1 in an amount effective to inhibit mTOR.

In other aspects, the invention provides a method of inhibiting PI3K in a subject, comprising administering to a subject in need thereof a compound of Formula 1 in an amount effective to inhibit PI3K.

In other aspects, the invention provides a method of inhibiting mTOR and PI3K together in a subject, comprising administering to a subject in need thereof a compound of Formula 1 in an amount effective to inhibit mTOR and PI3K.

In other aspects, the invention provides a method of synthesizing a compound of Formula 1′, comprising:

a) condensing a compound of the formula XVIII with a compound of formula XIX:

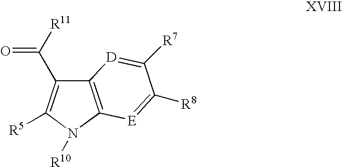

under acidic conditions, and A, D, E, and R¹-R¹¹ are as defined in formula 1

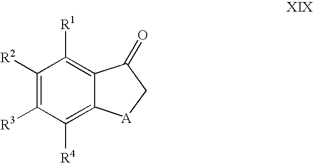

thereby producing a compound of formula 1′:

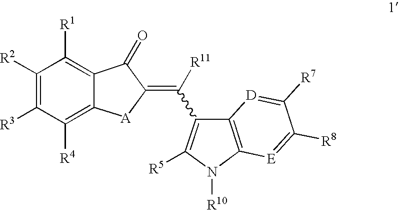

b) optionally reducing the compound of formula 1′ and thereby producing a compound of formula 1″:

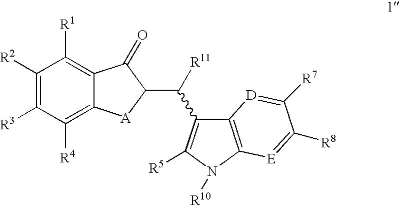

or a pharmaceutically acceptable salt thereof.

A method further comprising:

a) acylation with R¹¹C(O)X, wherein X is halogen, or Vilsmeier-Haack formylation, of a compound of formula XVI:

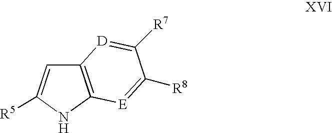

thereby producing a compound of formula XVII:

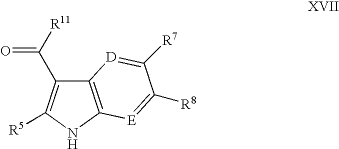

b) optionally alkylating the compound of formula XVII with R¹⁰Cl, thereby producing a compound of Formula XVIII.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), trieth iodide, tromethamine (2-amino-2-(hydroxymethyl)-1,3-propanediol), valerate, and zinc salts.

Some compounds within the present invention possess one or more chiral centers, and the present invention includes each separate enantiomer of such compounds as well as mixtures of the enantiomers. Where multiple chiral centers exist in compounds of the present invention, the invention includes each combination as well as mixtures thereof. All chiral, diastereomeric, and racemic forms of a structure are intended, unless the specific stereochemistry or isomeric form is specifically indicated. It is well known in the art how to prepare optically active forms, such as by resolution of racemic forms or by synthesis from optically active starting materials.

The compounds within the present invention possess double bonds connecting the fused indole to the benzofuran or benzothiophene nucleolus. These double bonds can exist as geometric isomers, and the invention includes both E and Z isomers of such double bonds. All such stable isomers are contemplated in the present invention.

An “effective amount” when used in connection a compound of the present invention of this invention is an amount effective for inhibiting mTOR or PI3K in a subject.

### DEFINITIONS

The following definitions are used in connection with the compounds of the present invention unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (C₆-C₁₄aryl)-(C₁-C₆alkyl)-O—C(O)—. It is understood that the above definitions are not intended to include impermissible substitution patterns (e.g., methyl substituted with 5 fluoro groups). Such impermissible substitution patterns are well known to the skilled artisan.

Acyl” refers to from 1 to 8 carbon atoms of a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C₁-C₈acyl group include acetyl-, benzoyl-, nicotinoyl, propionyl-, isobutyryl-, oxalyl-, and the like. Lower-acyl refers to acyl groups containing one to four carbons. An acyl group can be unsubstituted or substituted with one or more of the following groups: halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“Alkenyl” refer to a straight or branched chain unsaturated hydrocarbon containing 2-10 carbon atoms, and containing at least one double bond. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. A C₂-C₁₀alkenyl group can be unsubstituted or substituted with one or more of the following groups: halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Alkoxy” refers to the group R—O— where R is an alkyl group, as defined below. Exemplary C₁-C₆alkoxy groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂.

“(Alkoxy)carbonyl” refers to the group alkyl-O—C(O)—. Exemplary (C₁-C₆alkoxy)carbonyl groups include but are not limited to methoxy, ethoxy, n-propoxy, 1-propoxy, n-butoxy and t-butoxy. An (alkoxy)carbonyl group can be unsubstituted or substituted with one or more of the following groups: halogen, hydroxyl, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂.

“Alkyl” refers to a hydrocarbon chain that may be a straight chain or branched chain, containing the indicated number of carbon atoms. For example, C_{I}-C_{I0} indicates that the group may have from 1 to 10 (inclusive) carbon atoms in it. In the absence of any numerical designation, “alkyl” is a chain (straight or branched) having 1 to 6 (inclusive) carbon atoms in it. Examples of C₁-C₆ alkyl groups include, but are not limited to, methyl, ethyl, propyl, butyl, pentyl, hexyl, isopropyl, isobutyl, sec-butyl, tert-butyl, isopentyl, neopentyl, and isohexyl. An alkyl group can be unsubstituted or substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂.

The carbon number as used in the definitions herein refers to carbon backbone and carbon branching, but does not include carbon atoms of the substituents, such as alkoxy substitutions and the like.

“(Alkyl)amido-” refers to a —C(O)NH— group in which the nitrogen atom of said group is attached to a alkyl group, as defined above. Representative examples of a (C₁-C₆alkyl)amido group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an —NH group, the nitrogen atom of said group being attached to a alkyl group, as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂.

“Alkylcarboxy” refers to an alkyl group, defined above, attached to the parent structure through the oxygen atom of a carboxyl (C(O)—O—) functionality. Examples of (C₁-C₆alkyl)carboxy include acetoxy, ethylcarboxy, propylcarboxy, and isopentylcarboxy.

“(Alkyl)carboxyamido-” refers to a —NHC(O)— group in which the carbonyl carbon atom of said group is attached to a alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“Alkylene”, “alkenylene”, and “alkynylene” refers to the subsets of alkyl, alkenyl and alkynyl groups, as defined above, including the same residues as alkyl, alkenyl, and alkynyl, but having two points of attachment within a chemical structure. Examples of C₁-C₆alkylene include ethylene (—CH₂CH₂—), propylene (—CH₂CH₂CH₂—), and dimethylpropylene (—CH₂C(CH₃)₂CH₂—). Likewise, examples of C₂-C₆alkenylene include ethenylene (—CH═CH— and propenylene (—CH═CH—CH₂—). Examples of C₂-C₆alkynylene include ethynylene (—C≡C—) and propynylene (—C≡C—CH₂—).

“Alkylthio” refers to groups of straight chain or branched chain with 1 to 6 carbon atoms, attached to the parent structure through a sulfur atom. Examples include methylthio, ethylthio, n-propylthio, i-propylthio, n-butylthio, i-butylthio, s-butylthio, t-butylthio, n-pentylthio and n-hexylthio.

“Alkynyl” refers to a straight or branched chain unsaturated hydrocarbon containing 2-10 carbon atoms, and containing at least one triple bond. Examples of a C₂-C₁₀alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne. An alkynyl group can be unsubstituted or substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Amido(aryl)-” refers to an aryl group, as defined below, wherein one of the aryl group's hydrogen atoms has been replaced with one or more —C(O)NH₂ groups. Representative examples of an amido(C₆-C₁₄aryl)- group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Aminoalkyl-” refers to an alkyl group, as defined above, wherein one or more of the alkyl group's hydrogen atoms has been replaced with —NH₂. Representative examples of an C₁-C₆-aminoalkyl- group include, but are not limited to —CH₂NH₂, —CH₂CH₂NH₂, —CH₂CH₂CH₂ NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂), —CH₂CH₂CH₂CH₂CH₂NH₂, and —CH₂CH₂CH(NH₂)CH₂CH₃. An aminoalkyl- group can be unsubstituted or substituted with one or two of the following groups C₁-C₆alkoxy, C₆-C₁₄aryl, C₁-C₉heteroaryl, C₃-C₈cycloalkyl, and C₁-C₆alkyl.

Aryl refers to an aromatic hydrocarbon group containing 6-14 carbon ring atoms. “C₆-C₁₄Aryl” refers to a phenyl, naphthyl, biphenyl, anthryl, tetrahydronaphthyl, fluorenyl, indanyl, biphenylenyl, and acenaphthenyl, groups. Examples of an C₆-C₁₄aryl group include, but are not limited to, phenyl, 1-naphthyl, 2-naphthyl, and 3-biphen-1-yl. An aryl group can be unsubstituted or substituted with one or more of the following groups: C₁-C₆alkyl, halo, haloalkyl-, hydroxyl, hydroxyl(C₁-C₆alkyl)-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Aryl)alkyl” refers to an alkyl group, as defined above, wherein one or more of the alkyl group's hydrogen atoms has been replaced with an C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, —NH₂, hydroxyl, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂.

“(Aryl)amino” refers to a radical of formula (C₆-C₁₄aryl)-NH—, wherein “C₆-C₁₄aryl” is as defined above. Examples of (C₆-C₁₄aryl)amino radicals include, but are not limited to, phenylamino (anilido), 1-naphthlamino, 2-naphthlamino and the like. An (C₆-C₁₄aryl)amino group can be unsubstituted or substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“(Aryl)oxy” refers to the group Ar—O— where Ar is an C₆-C₁₄aryl group, as defined above. Exemplary (C₆-C₁₄aryl)oxy groups include but are not limited to phenyloxy, α-naphthyloxy, and β-naphthyloxy. A (C₆-C₁₄aryl)oxy group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆-aminoalkyl-, -dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Cycloalkyl” refers to a monocyclic, non-aromatic, saturated hydrocarbon ring containing 3-8 carbon atoms. Representative examples of a C₃-C₈cycloalkyl include, but are not limited to, cyclopropyl, cyclobutyl, cyclopentyl, cyclohexyl, cycloheptyl and cyclooctyl. A cycloalkyl can be unsubstituted or independently substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

A “Bicyclic cycloalkyl” refers to a bicyclic, non-aromatic, saturated hydrocarbon ring system containing 6-10 carbon atoms. Representative examples of a C₆-C₁₀bicyclic cycloalkyl include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), carboxyamidoalkyl-, or —NO₂. Additionally, each of any two hydrogen atoms on the same carbon atom of the bicyclic cycloalkyl rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

A “Carboxyamidoalkyl-” refers to a primary carboxyamide (CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound by an C₁-C₆alkylene group as defined above. Exemplary C₁-C₆-carboxyamidoalkyl- groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl” refers to monocyclic, non-aromatic carbocyclic rings containing 3-10 carbon atoms with one or more carbon-to-carbon double bonds within the ring system. The “cycloalkenyl” may be a single ring or may be multi-ring. Multi-ring structures may be bridged or fused ring structures. A C₃-C₁₀cycloalkenyl can be unsubstituted or independently substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂ Additionally, each of any two hydrogen atoms on the same carbon atom of the C₃-C₁₀cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms. Examples of C₃-C₁₀cycloalkenyls include, but are not limited to, cyclopropenyl, cyclobutenyl, cyclopentenyl, cyclohexenyl, 4,4a-octalin-3-yl, and cyclooctenyl.

“Di(alkyl)amino-” refers to a nitrogen atom which has attached to it two alkyl groups, as defined above. Each alkyl group can be independently selected. Representative examples of an di(C₁-C₆alkyl)amino- group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen-containing heterocycle wherein up to two of the carbon atoms of the heterocycle can be replaced with —N(R)—, —O—, or —S(O)ₚ—. R is hydrogen, C₁-C₆alkyl, C₃-C₈cycloalkyl, C₆-C₁₄aryl, C₁-C₉heteroaryl, C₁-C₆-aminoalkyl-, or arylamino. Variable p is 0, 1, or 2.

“Halo” or “halogen” is —F, —Cl, —Br or —I.

“Haloalkyl-” refers to a alkyl group, as defined above, wherein one or more of the C₁-C₆alkyl group's hydrogen atoms has been replaced with —F, —Cl, —Br, or —I. Each substitution can be independently selected from —F, —Cl, —Br, or —I. Representative examples of an C₁-C₆haloalkyl- group include, but are not limited to, —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂ CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Heteroaryl” refers to 5-10-membered mono and bicyclic aromatic groups containing at least one heteroatom selected from oxygen, sulfur and nitrogen. Examples of monocyclic C₁-C₉heteroaryl radicals include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic heteroaryl radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆-aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Heteroaryl)oxy” refers to the group Het-O— where Het is a heteroaryl group, as defined above. Exemplary (C₁-C₉heteroaryl)oxy groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆-aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

The term “heteroatom” refers to a sulfur, nitrogen, or oxygen atom.

“Heterocycle” or “heterocyclyl” refers to 3-10-membered mono, bicyclic, and bridged groups containing at least one heteroatom selected from oxygen, sulfur and nitrogen. A heterocycle may be saturated or partially saturated. Exemplary C₁-C₉heterocyclyl groups include but are not limited to 1,2-oxaziridin-2-yl, aziridine, oxirane, oxirene, 1H-tetrazole, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, oxazine, 5,6-dihydro-4H-1,3-oxazin-2-yl, 2,5-diazabicyclo[2.2.1]hept-2-yl, 3-oxa-9-azabicyclo[3.3.1]non-9-yl, 2-oxa-5-azabicyclo[2.2.2]oct-5-yl, 8-oxa-3-azabicyclo[3.2.1]octan-3-yl, 2-methyl-2,5-diazabicyclo[2.2.1]heptane-5-yl, 3-oxa-8-azabicyclo[3.2.1]oct-8-yl, 6,8-dioxa-3-azabicyclo[3.2.1]oct-3-yl, 2-oxa-5-azabicyclo[2.2.1]hept-5-yl, 9-oxa-3,7-diazabicyclo[3.3.1]non-3-yl, 1,3,3-trimethyl-6-azabicyclo[3.2.1]oct-6-yl, 4-methyl-3,4-dihydro-2H-1,4-benzoxazin-7-yl, thiazine, dithiane, and dioxane.

“Heterocyclyl(alkyl)” refers to an alkyl group, as defined above, wherein one or more of the alkyl group's hydrogen atoms has been replaced with a heterocycle group as defined above. Heterocyclyl(C₁-C₆alkyl) moieties include 2-pyridylmethyl, 1-piperazinylethyl, 1-pyrrolidinylethyl, 4-morpholinylpropyl, 6-piperazinylhexyl, and the like. A heterocyclyl(alkyl) group can be unsubstituted or substituted with one or more of the following groups: halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), 4- to 7-membered monocyclic heterocycle, C₆-C₁₄aryl, C₁-C₉heteroaryl, or C₃-C₈cycloalkyl.

“Hydroxylalkyl-” refers to a alkyl group, as defined above, wherein one or more of the C₁-C₆alkyl group's hydrogen atoms has been replaced with hydroxyl groups. Examples of C₁-C₆hydroxylalkyl- moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Hydroxylalkenyl-” refers to an alkenyl group, defined above, and substituted on one or more sp³ carbon atoms with a hydroxyl group. Examples of C₃-C₆hydroxylalkenyl- moieties include chemical groups such as —CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

The term “monocyclic heterocycle” refers to a monocyclic 3- to 7-membered aromatic, cycloalkyl, or cycloalkenyl in which 1-4 of the ring carbon atoms have been independently replaced with an N, O or S atom. The monocyclic heterocyclic ring can be attached via a nitrogen, sulfur, or carbon atom. Representative examples of a 3- to 7-membered monocyclic heterocycle group include, but are not limited to, piperidinyl, 1,2,5,6-tetrahydropyridinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl. A monocyclic heterocycle group can be unsubstituted or substituted with one or more of the following groups: C₁-C₈acyl, C₁-C₆alkyl, heterocyclyl(C₁-C₆alkyl), (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂.

“Bicyclic heterocycle” refers to a bicyclic cycloalkyl or bicyclic cycloalkenyl in which 1-4 of the ring carbon atoms have been independently replaced with an N, O or S atom. The bicyclic heterocyclic ring can be attached via a nitrogen, sulfur, or carbon atom. Representative examples of a 6- to 10-membered bicyclic heterocycle group include, but are not limited to, indolinyl, indazolyl, tetrahydroquinolinyl, perhydroquinazolinyl, 5,6-dihydro-4H-1,3-oxazin-2-yl, 8-oxa-3-azabicyclo[3.2.1]octan-3-yl, 2-methyl-2,5-diazabicyclo[2.2.1]heptane-5-yl, and indazolyl. A bicyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Nitrogen-containing heteroaryl” refers to 5-10-membered mono and bicyclic aromatic groups containing at least one nitrogen atom and optionally additional heteroatoms selected from oxygen and sulfur. Examples of nitrogen-containing monocyclic C₁-C₉heteroaryl radicals include, but are not limited to, oxazinyl, thiazinyl, diazinyl, triazinyl, tetrazinyl, imidazolyl, tetrazolyl, isoxazolyl, furazanyl, oxazolyl, thiazolyl, pyrazolyl, triazolyl, pyrimidinyl, N-pyridyl, 2-pyridyl, 3-pyridyl and 4-pyridyl. Examples of nitrogen-containing bicyclic C₁-C₉heteroaryl radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A nitrogen-containing C₁-C₉heteroaryl group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆-aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Perfluoroalkyl-” refers to alkyl group, defined above, having two or more fluorine atoms. Examples of a C₁-C₆perfluoroalkyl-group include CF₃, CH₂CF₃, CF₂CF₃ and CH(CF₃)₂.

The term “optionally substituted” as used herein means that at least one hydrogen atom of the optionally substituted group has been substituted with halogen, —NH₂, (C₁-C₆alkyl)N—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

A “subject” is a mammal, e.g., a human, mouse, rat, guinea pig, dog, cat, horse, cow, pig, or non-human primate, such as a monkey, chimpanzee, baboon or gorilla.

The compounds of the present invention exhibit an mTOR inhibitory activity and, therefore, can be utilized to inhibit abnormal cell growth in which mTOR plays a role. Thus, the compounds of the present invention are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention exhibit a PI3 kinase inhibitory activity and, therefore, can be utilized in order to inhibit abnormal cell growth in which PI3 kinases play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of Formula 1 will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be made into tablets, placed in a hard gelatin capsule in powder or pellet form, or in the form of a troche or lozenge. The solid carrier may contain conventional excipients such as binding agents, fillers, lubricants used to make tablets, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formula I directly in parenteral formulations. The pharmaceutical compositions are prepared by conventional techniques appropriate to the desired preparation containing appropriate amounts of the active ingredient, that is, the compound of Formula I according to the invention. See, for example, Remington: The Science and Practice of Pharmacy, 20th Edition. Baltimore, Md.: Lippincott Williams & Wilkins, 2000.

The dosage of the compounds of Formula 1 to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of Formula 1 or pharmaceutical composition thereof for a mammal, including man, suffering from, or likely to suffer from any condition as described herein is an amount of active ingredient from about 0.01 .mg/kg to 10 mg/kg body weight. For parenteral administration, the dose may be in the range of 0.1 .mg/kg to 1 mg/kg body weight for intravenous administration. For oral administration, the dose may be in the range about 0.1 .mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In one embodiment, the compound of the present invention or a pharmaceutically acceptable salt thereof is administered concurrently with another therapeutic agent.

In one embodiment, a composition comprising an effective amount of a compound of the present invention or a pharmaceutically acceptable salt thereof and an effective amount of another therapeutic agent within the same composition can be administered.

Effective amounts of the other therapeutic agents are well known to those skilled in the art. However, it is well within the skilled artisan's purview to determine the other therapeutic agent's optimal effective amount range. The compound of the present invention or a pharmaceutically acceptable salt thereof and the other therapeutic agent can act additively or, in one embodiment, synergistically. In one embodiment, of the invention, where another therapeutic agent is administered to an animal, the effective amount of the compound of the present invention or a pharmaceutically acceptable salt thereof is less than its effective amount would be where the other therapeutic agent is not administered. In this case, without being bound by theory, it is believed that the compound of the present invention or a pharmaceutically acceptable salt thereof and the other therapeutic agent act synergistically.

Procedures used to synthesize the compounds of the present invention are described in Schemes 1-24 and are illustrated in the examples. Reasonable variations of the described procedures, which would be evident to one skilled in the art, are intended to be within the scope of the present invention:

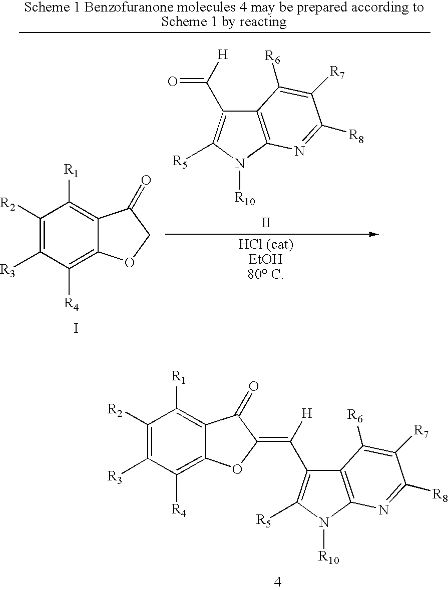

benzofuranone compounds I with heteroaryl aldehydes II in EtOH with catalytic amounts of HCl at 80 C. Benzofuranone compounds I and heteroaryl aldehydes II can be purchased commercially or prepared synthetically via standard organic chemistry protocols.

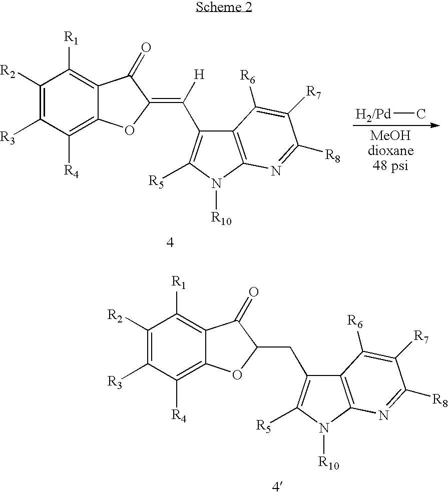

2-Methylbenzofuranone molecules 4′ may be prepared according to Scheme 2 by reduction of 2-methylenebenzofuranones 4 with Pd/C in MeOH/dioxane under 48 psi atmosphere of hydrogen.

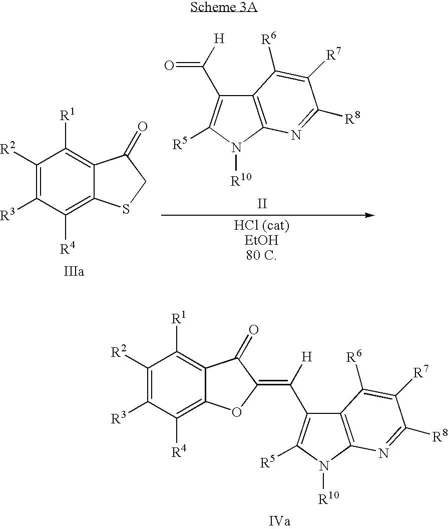

Benzothiophenone molecules IVa may be prepared according to Scheme 3A by reacting benzothiophenone IIIa with the heteroaryl aldehydes II in benzene with catalytic amounts of piperidine at 85° C. Benzothiophenone IIIa and heteroaryl aldehydes II can be purchased commercially or prepared synthetically via standard organic chemistry protocols.

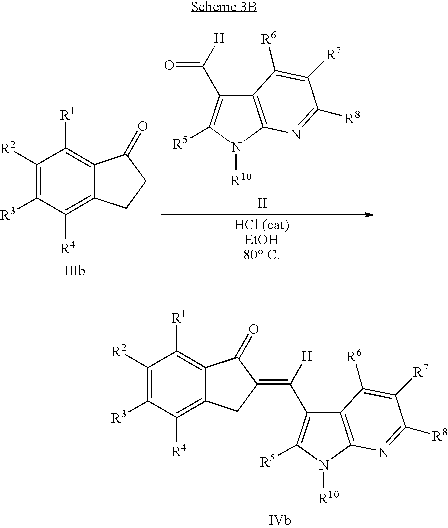

Indenone molecules IVb may be prepared according to Scheme 3B by reacting indenone IIIb with the heteroaryl aldehydes II in benzene with catalytic amounts of piperidine at 85° C. Indenone IIIb and heteroaryl aldehydes II can be purchased commercially or prepared synthetically via standard organic chemistry protocols.

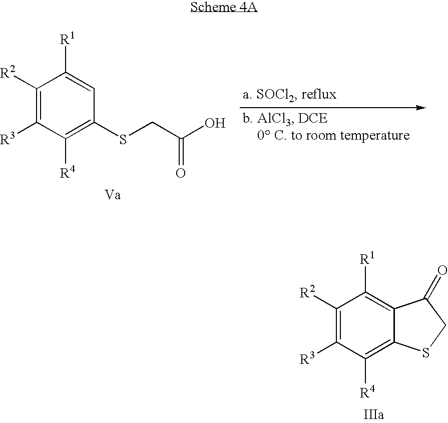

Benzothiophenone compounds IIIa as described in Scheme 4A can be obtained from the corresponding acids Va using known literature procedures. To the acid (15.6 mmole) is added SOCl₂ (10 mL). After heating the resulting suspension to 85° C. for 1 hour, the reaction is concentrated in vacuo and placed under vacuum for 30 minutes. To the reaction is added methylene chloride (30 mL) and cooled on an ice-salt bath for 15 minutes. AlCl₃ (2.5 g) is added in portions over 20 minutes. The reaction is stirred with cooling for 15 minutes and then allowed to stir for 45 minutes at room temperature. The reaction is quenched with ice water, extracted with methylene chloride and concentrated in vacuo to afford the desired compound without further purification.

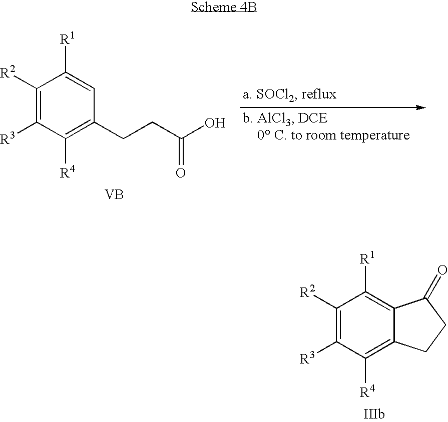

Indenone IIIb as described in Scheme 4B can be obtained from the corresponding acids Vb using known literature procedures. To the acid (15.6 mmole) is added SOCl₂ (10 mL). After heating the resulting suspension to 85° C. for 1 hour, the reaction is concentrated in vacuo and placed under vacuum for 30 minutes. To the reaction is added methylene chloride (30 mL) and cooled on an ice-salt bath for 15 minutes. AlCl₃ (2.5 g) is added in portions over 20 minutes. The reaction is stirred with cooling for 15 minutes and then allowed to stir for 45 minutes at room temperature. The reaction is quenched with ice water, extracted with methylene chloride and concentrated in vacuo to afford the desired compound without further purification.

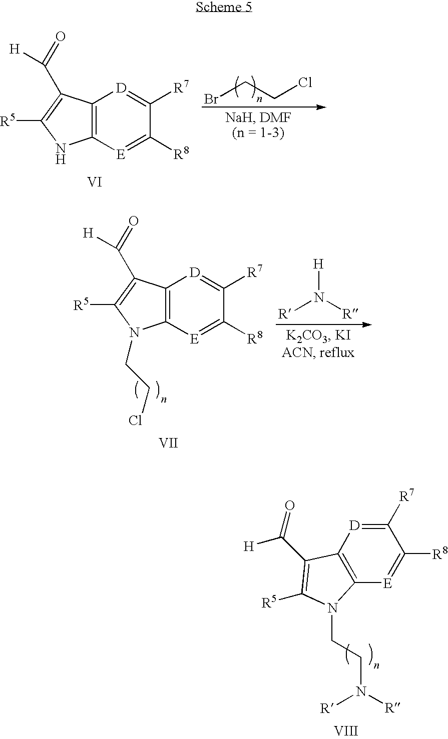

3-Indolecarboxaldehydes as described by Scheme 5 can be obtained by alkylation of the 3-indolecarboxaldehydes VI using the corresponding ω-bromochloroalkanes and NaH in DMF under standard literature conditions. The resulting alkyl chloride was then reacted with the desired secondary amine using potassium carbonate and potassium iodide in ACN at 80° C. under standard literature conditions.

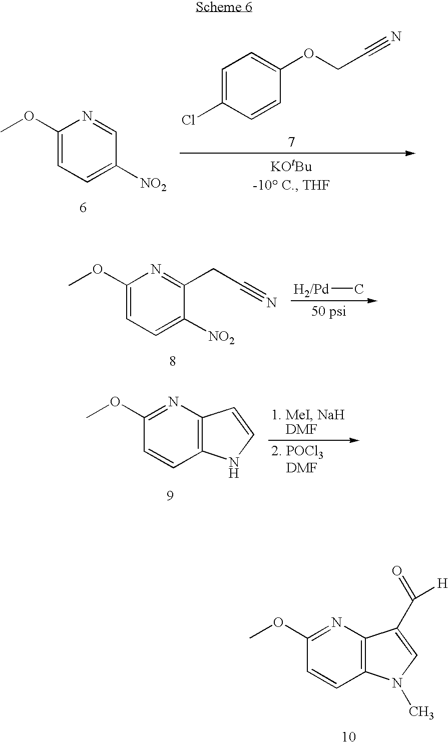

5-Methoxy-1-methyl-1H-pyrrolo[3,2-b]pyridine-3-carbaldehyde (10) as described in Scheme 6 can be obtained by first generating 5-methoxy-1H-pyrrolo[3,2-b]pyridine from 2-methoxy-5-nitro-pyridine 6 using literature procedures described in Eur. J. Med. Chem. 2004, 39, 515. The azaindole was then converted into 5-methoxy-1-methyl-1H-pyrrolo[3,2-b]pyridine-3-carbaldehyde using Vilsmeier-Haack methods.

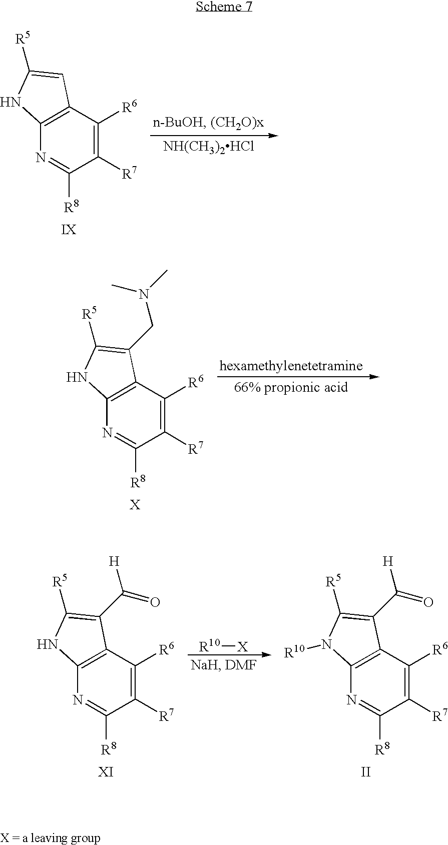

7-Aza-3-indole carboxaldehyde compounds II as described in Scheme 7 can be obtained by first generating 7-azagramine from 7-azaindole IX, paraformaldehyde, and dimethylamine, by Mannich reaction followed by hydrolysis using literature procedures described in JACS 1955, 77, 457. This was followed by methylation using MeI and NaH in DMF under standard literature conditions.

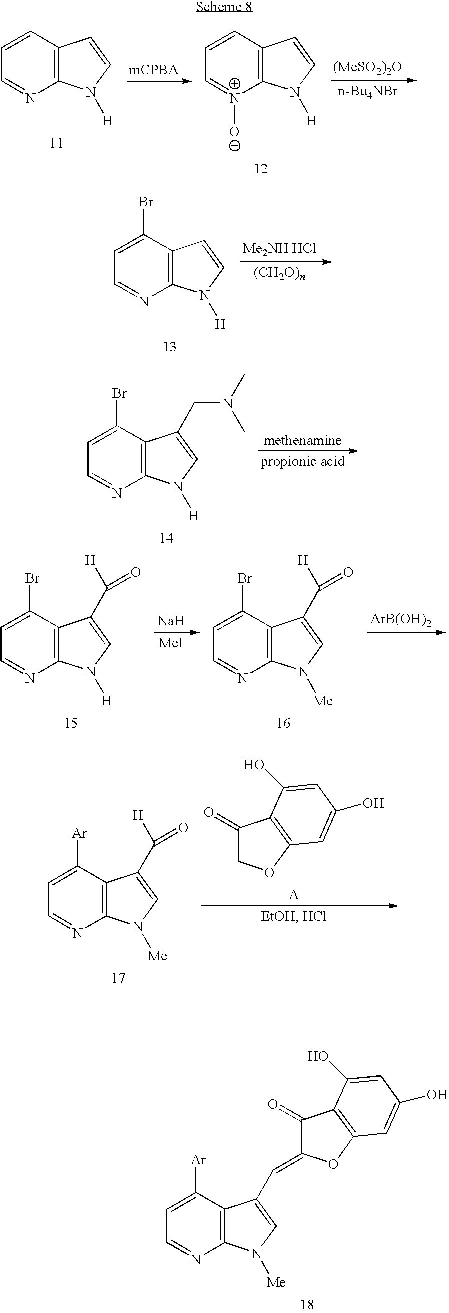

Preparation of (2Z)-2-[(4-aryl-1-methyl-1H-pyrrolo[2,3-b]pyridine-3-yl)methylene]-4,6-dihydroxy-1-benzofuran-3(2H)-one (18). 4-Bromo-1H-pyrrolo[2,3-b]pyridine was prepared by a modified N-oxide rearrangement procedure. The 7-azagramine 14 was obtained from 7-azaindole 13, paraformaldehyde, and dimethylamine, by Mannich reaction followed by hydrolysis. This was followed by methylation using MeI and NaH.

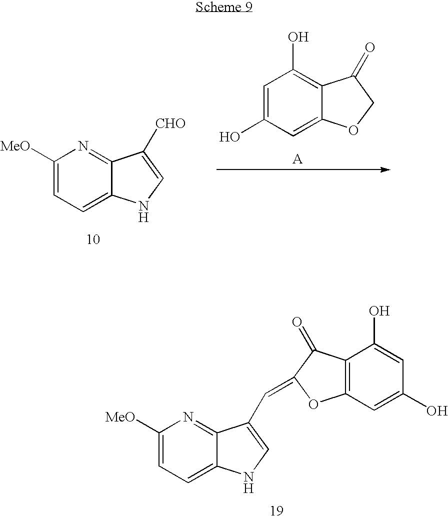

Preparation of (2Z)-4,6-dihydroxy-2-[(5-methoxy-1H-pyrrolo[3,2-b]pyridin-3-yl)methylene]-1-benzofuran-3(2H)-one (19). 5-Methoxy-1H-pyrrolo[3,2-b]pyridine-3-carbaldehyde (10) was condensed with furanone A, which proceeded smoothly.

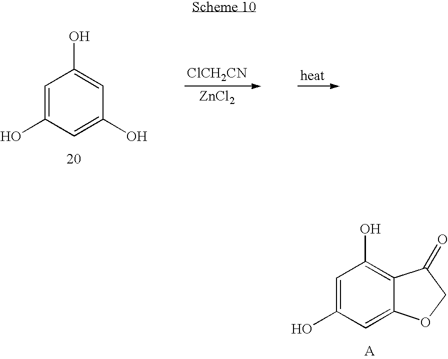

Preparation of 4,6-dihydroxybenzofuranone (Compound A) from phloroglucinol by thermal cyclization of the intermediate phenoxyacetonitrile, as shown in Scheme 10.

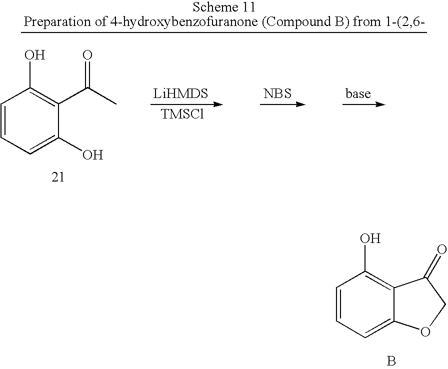

dihydroxyphenyl)ethanone (21) by bromination of the enol ether followed by base-induced cyclization, as shown in Scheme 11.

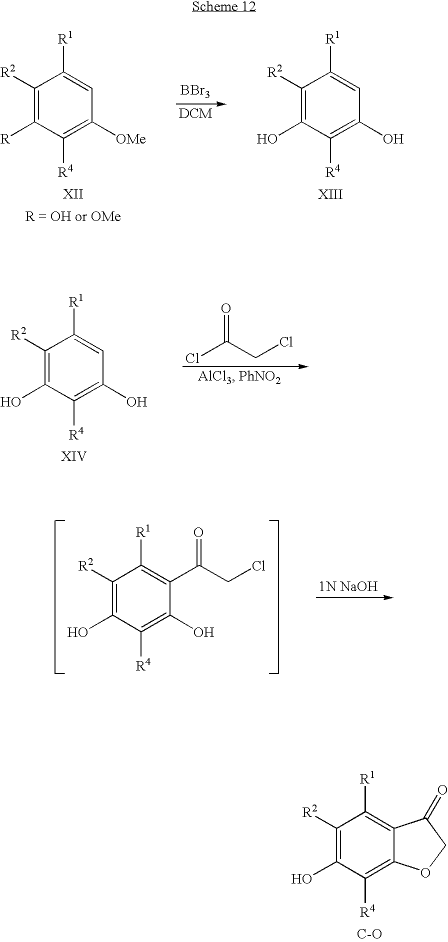

Preparation of monosubstituted 6-hydroxy benzofuranone compounds (Compounds C—O) from anisole compounds XII as shown in Scheme 12.

| Benzofuranone | R₁ | R₂ | R₄ |
|---|---|---|---|
| C | Me | H | H |
| D | H | Me | H |
| E | H | H | Me |
| F | F | H | H |
| G | H | F | H |
| H | H | H | F |
| I | Cl | H | H |
| L | H | Cl | H |
| M | H | H | Cl |
| N | H | Br | H |
| O | Br | H | H |

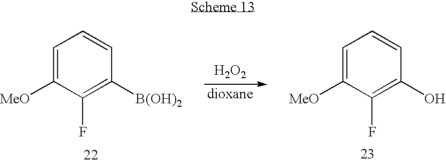

Preparation of 2-fluoro-3-methoxy-phenol (27) as shown in Scheme 13.

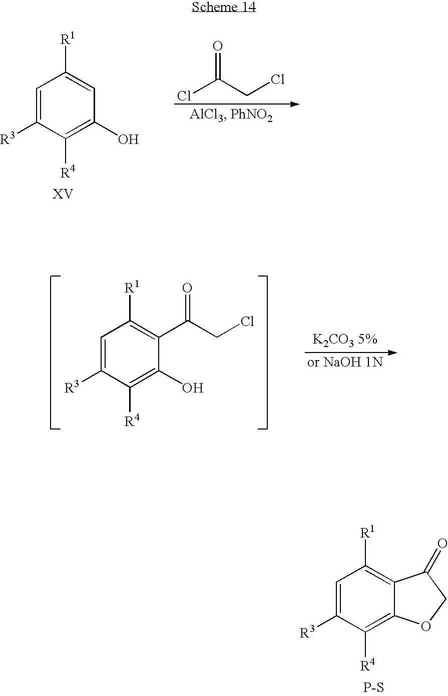

Preparation of other commercially non-available benzofuranone compounds (Compounds P—S) as shown in Scheme 14.

| Benzofuranone | R₁ | R₂ | R₄ |
|---|---|---|---|
| P | OMe | OMe | H |
| Q | H | H | Br |
| R | OMe | OH | H |
| S | H | Br | H |

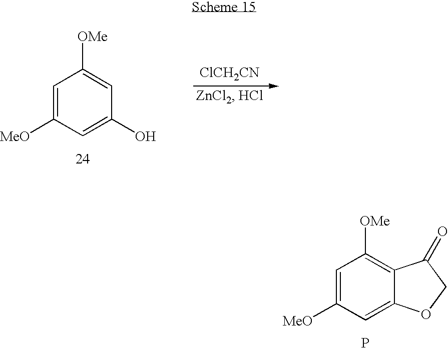

Preparation of 4,6-dimethoxybenzofuran-3(2H)-one (Compound P) as shown above in Scheme 15 by a one-step alkylation-cyclization process.

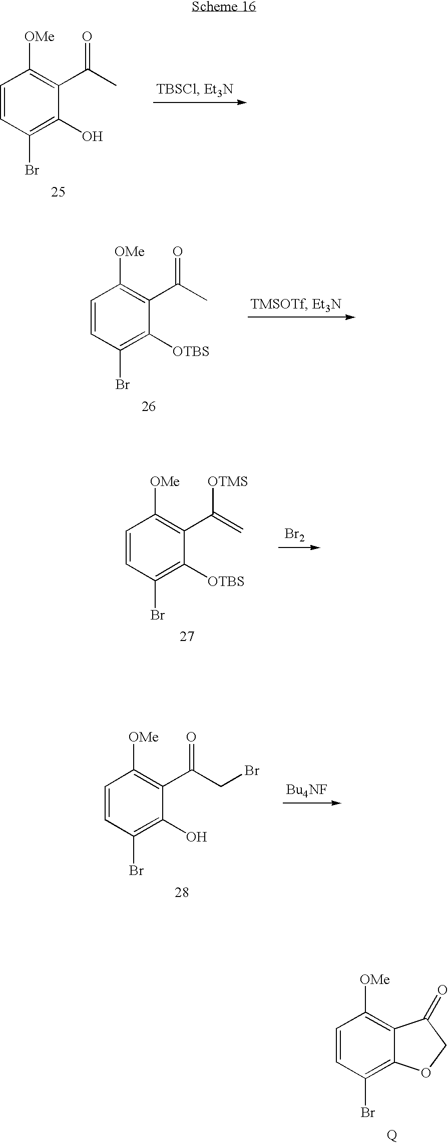

Preparation of 7-bromo-4-methoxybenzofuran-3(2H)-one (Compound Q) from 1-(3-bromo-2-hydroxy-6-methoxyphenyl)ethanone by bromination of the enol ether followed by fluoride-induced cyclization, as shown in Scheme 16.

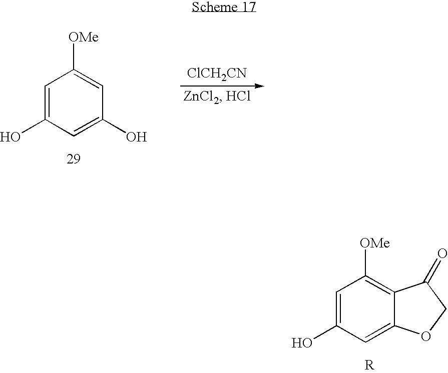

Preparation of 6-hydroxy-4-methoxybenzofuran-3(2H)-one (Compound R) as shown above in Scheme 17 by a one-step alkylation-cyclization process.

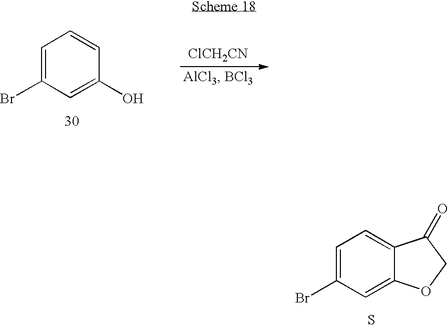

Preparation of 6-bromobenzofuran-3(2H)-one (Compound S) as shown above in Scheme 18 by another one-step alkylation-cyclization process.

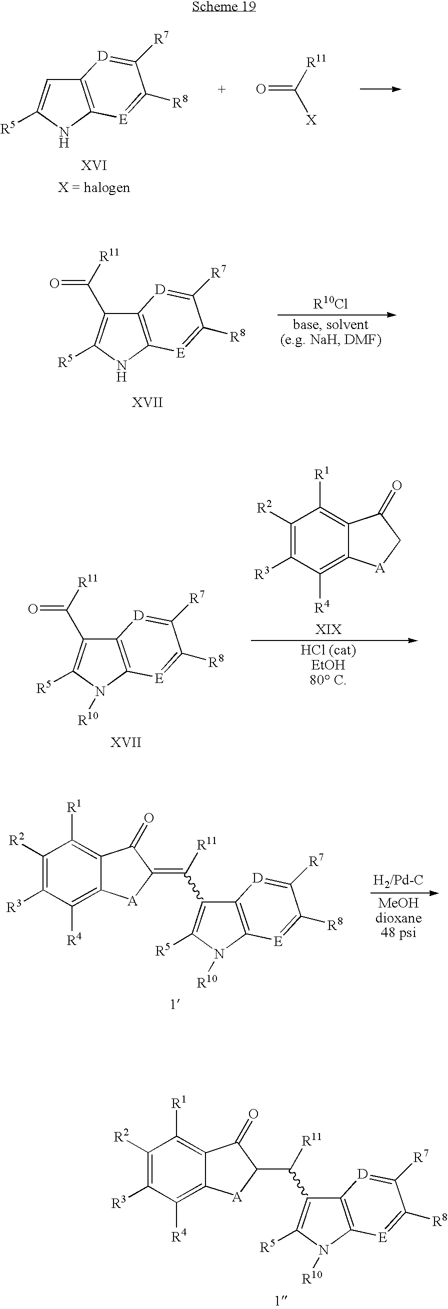

A general synthesis of the 1H-pyrrolopyridin-3-yl)methylene compounds of Formula 1′ (compounds of Formula 1 with [symbol] a second carbon to carbon bond) and of the reduced pyrrolopyridin-3-yl)methyl compounds 1″ (compounds of Formula 1 with [symbol] absent) is shown in Scheme 19.

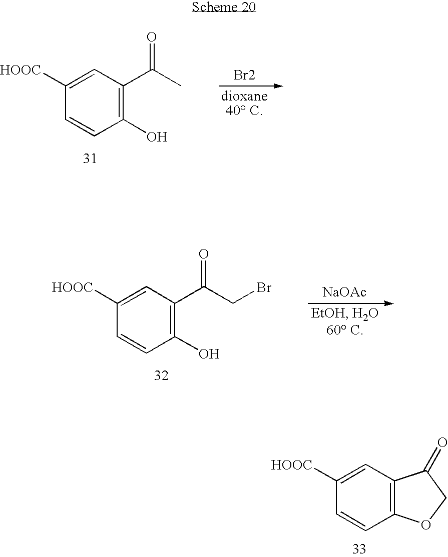

Preparation of 3-oxo-2,3-dihydrobenzofuran-5-carboxylic acid (Compound 33) as shown above in Scheme 20 by a two-step bromination-cyclization process.

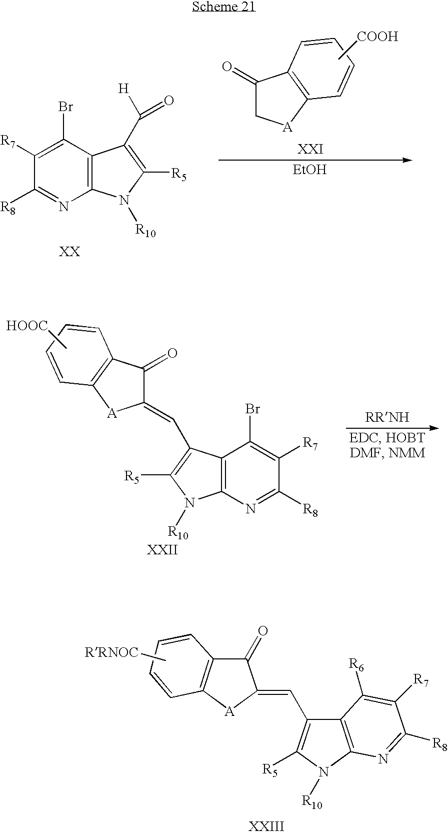

Condensation of 3-oxo-2,3-dihydrobenzofuran carboxylic acids XXI with 1H-pyrrolo[2,3-b]pyridine-3-carbaldehydes XX as shown above in Scheme 21.

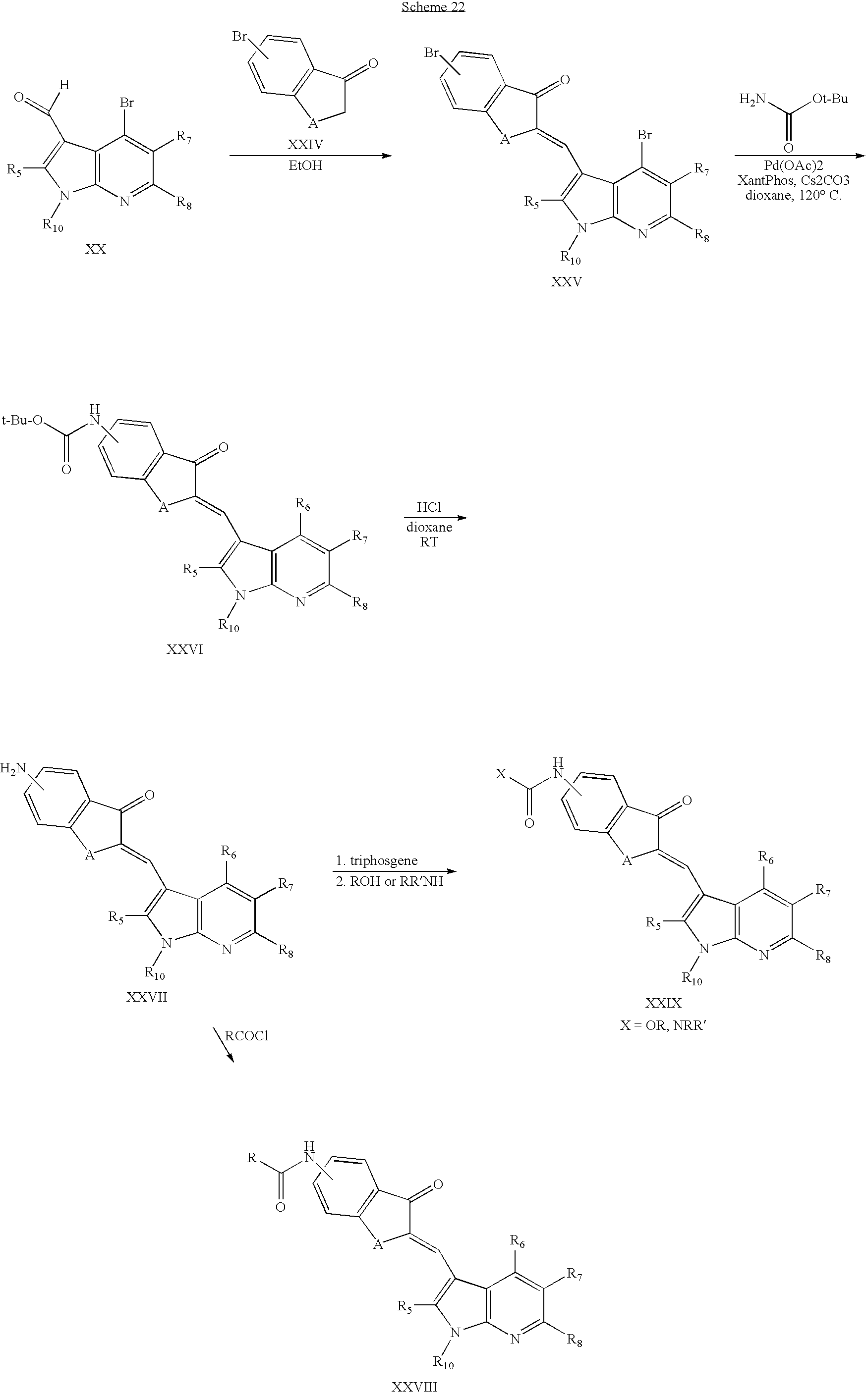

Condensation of bromo-3-oxo-2,3-dihydrobenzofuran XXIV with 1H-pyrrolo[2,3-b]pyridine-3-carbaldehydes XX as shown above in Scheme 22.

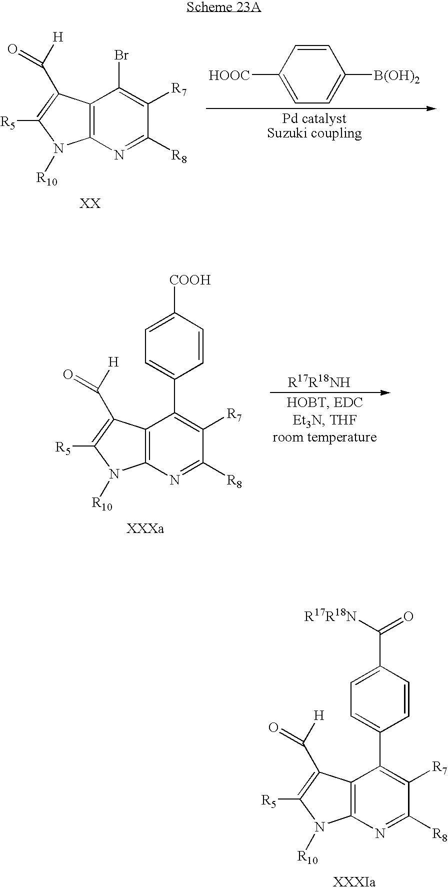

Preparation of 4-(3-formyl-1H-pyrrolo[2,3-b]pyridin-4-yl)benzamide intermediates Compound XXXIa as shown above in Scheme 23A by Suzuki coupling on the 4-bromo-3-formyl-1H-pyrrolo[2,3-b]pyridin-4-yl)benzamide XX, followed by coupling with amine.

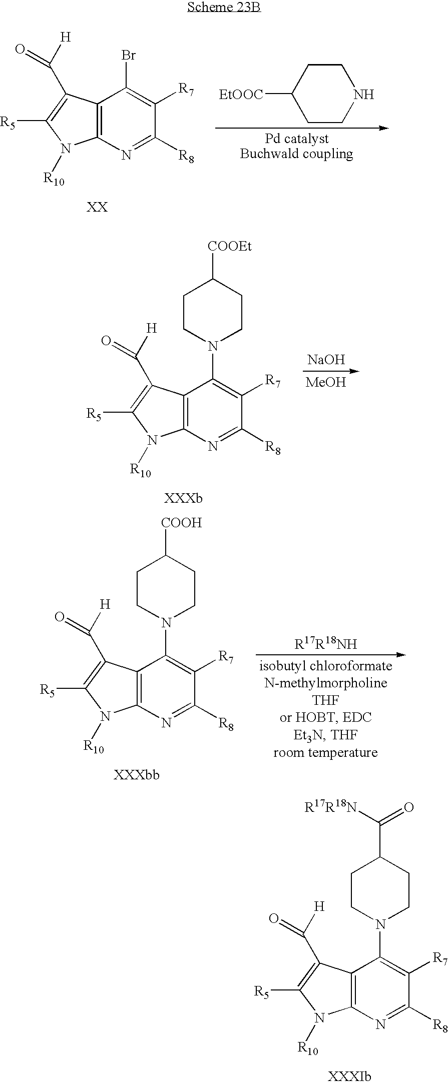

Preparation of 4-(3-formyl-1H-pyrrolo[2,3-b]pyridin-4-yl)piperidylamide intermediate compounds XXXIb as shown above in Scheme 23B by Buchwald coupling on the 4-bromo-3-formyl-1H-pyrrolo[2,3-b]pyridin-4-yl)piperidylamide XX, followed by hydrolysis and coupling with amine.

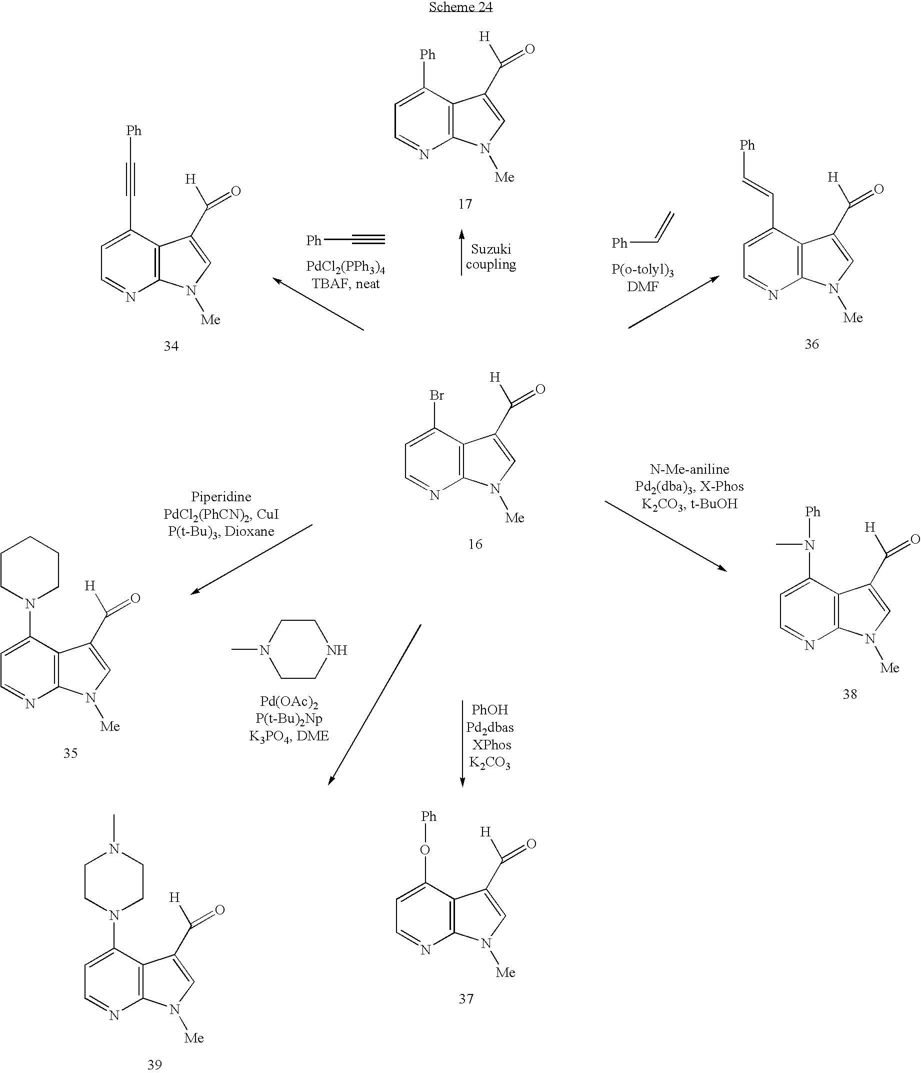

Scheme 24 summarizes the synthesis of various 1-methyl-1-H-pyrrolo[2,3-b]pyridine-3-carbaldehyde intermediates from 4-bromo-1-methyl-1-H-pyrrolo[2,3-b]pyridine-3-carbaldehyde 16.

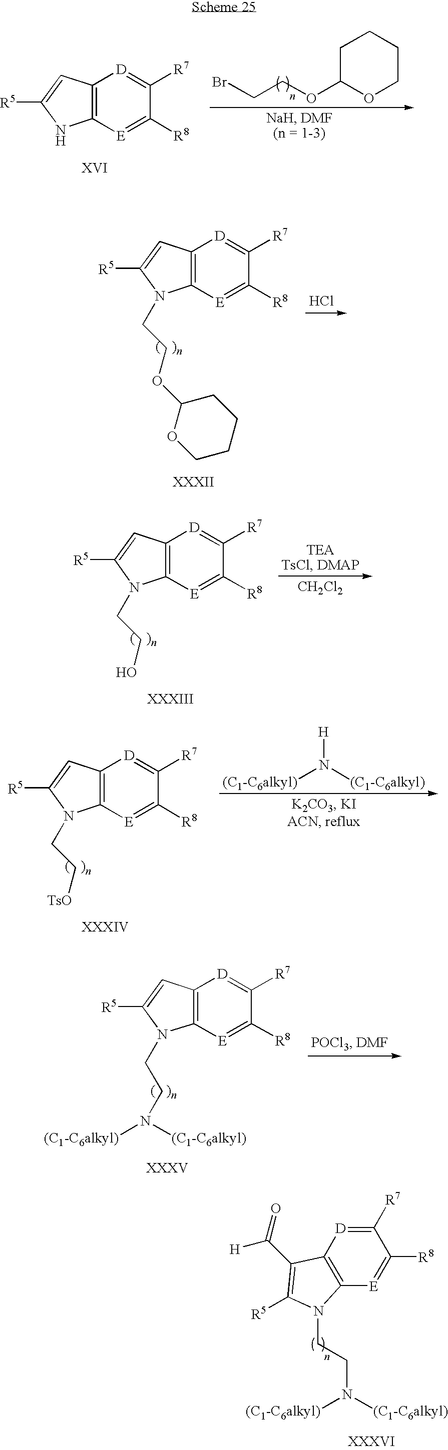

3-Indolecarbaldehydes as described by Scheme 25 can be obtained by alkylation of the indole XVI using the 2-(bromoalkoxy) tetrahydropyran and NaH in DMF under standard conditions. The resulting alkyl ether XXXII was then deprotected with HCl to generate the alcohol XXXIII, which was then converted into the corresponding tosylate XXIV under standard conditions. The tosylate XXXIV was then displaced with the desired secondary amine using potassium carbonate and potassium iodide in acetonitrile at 80° C. under standard literature conditions to generate the amine XXXV. The azaindole XXXV was converted into the corresponding 3-carbaldehyde XXXVI using Vilsmeier-Haack methods.

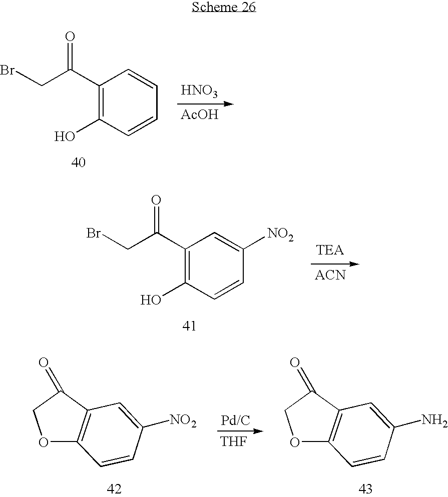

Preparation of 5-aminobenzofuran-3(2H)-one 43 is shown in Scheme 26. Nitration of the bromide 40 followed by cyclization yielded 5-nitrobenzofuran-3(2H)-one 42. Reduction of the nitro compound 42 generated the desired 5-aminobenzofuran-3(2H)-one 43.

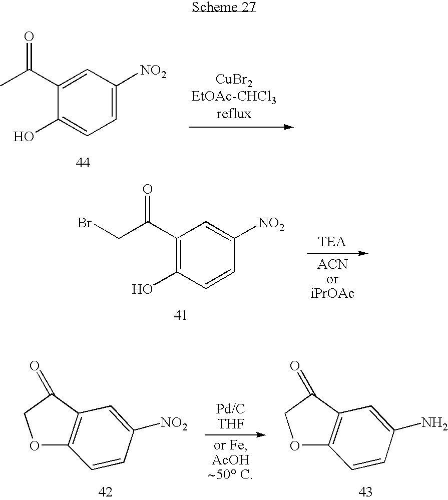

Alternate synthesis of 5-aminobenzofuran-3(2H)-one 43 is shown in Scheme 27 starting with 2-hydroxy-5-nitroacetophenone 44. Bromination of 44 using cupric bromide yielded the bromide 41, which was then converted into the desired 5-aminobenzofuran-3(2H)-one 43 as shown in Scheme 26.

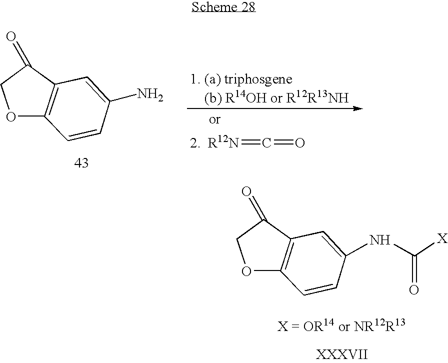

Conversion of amine 43 to carbamates and ureas XXXVII was accomplished either by treatment with triphosgene, followed by addition of alcohols or amines, or by treatment with isocyanate reagents as shown in Scheme 28.

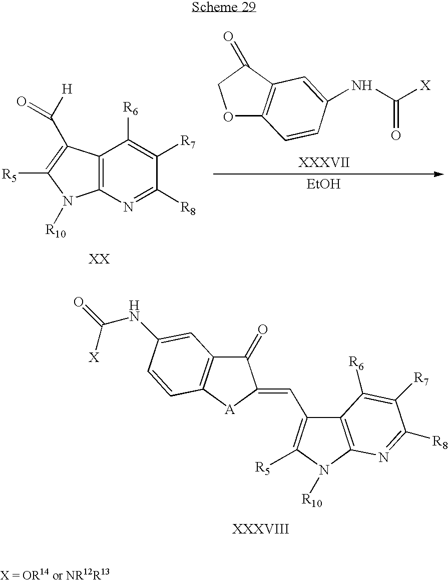

Condensation of the 5-substituted 3-oxo-2,3-dihydrobenzofuran XXXVII with 1H-pyrrolo[2,3-b]pyridine-3-carbaldehydes XX is shown above in Scheme 29.

One of skill in the art will recognize that Schemes 1-29 can be adapted to produce the other compounds of Formula I and pharmaceutically acceptable salts of compounds of Formula I according to the present invention.

### EXAMPLES

The following abbreviations are used herein and have the indicated definitions: ACN is acetonitrile, AcOH is acetic acid. ATP is adenosine triphosphate. Biotage Initiator™ 60 is a 60-position sample microwave synthesizer. Initiator™ is a registered trademark of Biotage AB, Uppsala, Sweden. BOC is t-butoxycarbonyl. Celite™ is flux-calcined diatomaceous earth. Celite™ is a registered trademark of World Minerals Inc. CHAPS is (3-[(3-cholamidopropyl)dimethylammonio]-1-propanesulfonic acid, DEAD is diethyl azodicarboxylate, DIAD is diisopropylazodicarboxylate, DMAP is dimethyl aminopyridine, DME is 1,2-dimethoxyethane, DMF is N,N-dimethylformamide, DMF-DMA is dimethylformamide dimethyl acetal, and DMSO is dimethylsulfoxide. DPBS is Dulbecco's Phosphate Buffered Saline Formulation. EDCl is 3′-dimethylaminopropyl)carbodiimide or water-soluble carbodiimide, EDTA is ethylenediaminetetraacetic acid, ESI stands for Electrospray Ionization, EtOAc is ethyl acetate, and EtOH is ethanol. HBTU is O-benzotriazole-N,N,N′,N′-tetramethyl-uronium-hexafluoro-phosphate, HEPES is 4-(2-hydroxyethyl)-1-piperazineethanesulfonic acid, GMF is glass microfiber, HOBT is N-hydroxybenzotriazole, Hunig's Base is diisopropylethylamine, HPLC is high-pressure liquid chromatography, LPS is lipopolysaccharide. MeCN is acetonitrile, MeOH is methanol, MS is mass spectrometry, and NEt₃ is triethylamine. Ni(R^{a}) is Raney™ nickel, a sponge-metal catalyst produced when a block of nickel-aluminum alloy is treated with concentrated sodium hydroxide. Raney™ is a registered trademark of W. R. Grace and Company. NMP is N-methylpyrrolidone, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is trichloroacetic acid, TFA is trifluoroacetic acid, THF is tetrahydrofuran, THP is tetrahydro-2H-pyran-2-yl. TLC is thin-layer chromatography and TRIS is tris(hydroxymethyl)aminomethane.

### Synthetic Methods

The following methods outline the synthesis of the Examples of the present invention.

### I. Synthesis of Benzofuranone Intermediates

### Preparation of 4,6-dihydroxybenzofuranone (Compound A)

To a solution of phloroglucinol (2 g, 16 mmol, 1 eq.) in ethyl ether (20 mL), ClCH₂CN (10 mL), ZnCl₂ (0.2 g, 1.6 mmol, 0.1 eq.) and 10% HCl/Et₂O (15 mL) were added. The mixture was stirred at room temperature overnight. The yellow precipitate (imine hydrochloride) was filtered off and washed three times with ethyl ether. Then, it was dissolved in 25 mL of water and heated at 100° C. overnight. The red solid was filtered off, washed three times with water and dried to give pure 4,6-dihydroxy-benzofuran-3-one. Yield: 70%. MS (m/z): 167.2 (MH+).

### Preparation of 4-hydroxybenzofuranone (Compound B)

LiHMDS (1M solution in THF, 3.1 mL, 3.1 mmol, 3.6 eq.) was slowly added to a solution of 2′,6′-dihydroxyacetophenone (131 mg, 0.86 mmol, 1 eq.) in anhydrous THF (4.5 mL) under argon atmosphere at −78° C. After 30 min, TMSCl (0.65 mL, 5.16 mmol, 6 eq.) was added and the resulting mixture was stirred for 4 hours. Then NBS (171 mg, 0.95 mmol, 1.1 eq.) was slowly added and the solution was stirred for 1 hour at −78° C. and for 10 min at rt. 1M NaOH (2 mL) was added and the resulting solution was stirred until complete disappearance of the starting material. The reaction was quenched by adding 1M HCl until pH 4. The aqueous layer was extracted with EtOAc and the collected organic extracts were washed with brine, dried on anhydrous Na₂SO₄ and evaporated under reduced pressure. The oily crude mixture was purified by silica gel column chromatography (eluent: EtOAc/petroleum ether 15:85). The title compound was obtained as a pale yellow solid. Yield: 46%. MS (m/z): 151.5 (MH+).

### Preparation of Monosubstituted 6-hydroxy benzofuranone Compounds (Compounds C—O)

### Preparation of 2-fluoro-3-methoxy-phenol

Hydrogen peroxide (35% in water, 5 mL) was added to a solution of 2-fluoro-3-methoxyphenylboronic acid (500 mg, 2.94 mmole) in dioxane (5 mL). The reaction mixture was stirred at 100° C. for 2.5 hours and then allowed to cool to rt. Water was added and the aqueous layer was extracted with methylene chloride. The combined organic layers were dried on Na₂SO₄ and evaporated affording the title compound as dark oil. Yield: 71%. MS (m/z): 143.1 (MH+).

General Procedure for the Demethylation with BBr₃

To a solution of the methoxy-derivative (8.7 mmole) in methylene chloride (40 mL), cooled to −78° C., BBr₃ (1 M in methylene chloride, 4 eq. for each methoxy group) was added in drops. The reaction was stirred overnight allowing to the cooling bath to expire. The mixture was cooled again to −78° C. and quenched by addition of water in drops. The aqueous layer was extracted with EtOAc. The combined organic layers were dried on Na₂SO₄ and evaporated. The residue was triturated with EtOAc to give crude resorcinol that was used for the following reaction without further purification. This procedure was used to obtain the following compounds:

### 2-Fluoro-benzene-1,3-diol

Yield: 93%. MS (m/z): 129.1 (MH+).

### 5-Fluoro-benzene-1,3-diol

Yield: 97%. MS (m/z): 129.2 (MH+).

### 5-Chloro-benzene-1,3-diol

Yield: 87%. MS (m/z): 145.4 (MH+).

General Procedure for the Preparation of 6-hydroxybenzofuranones.

Chloroacetyl chloride (0.33 mL, 4.15 mmol, 1.2 eq.) was added to a suspension of AlCl₃ (2.3 g, 17.3 mmol, 5 eq.) in nitrobenzene (6 mL), cooled to 0° C. The selected resorcinol (3.46 mmol, 1 eq.) was dissolved in nitrobenzene (6 mL) and added at 0° C. to the reaction mixture. The reaction was stirred at room temperature overnight, then poured into ice and extracted with EtOAc. The organic layer was extracted with 1 N NaOH; the separated aqueous layer was acidified with HCl and extracted with EtOAc. The combined organic layers were dried on Na₂SO₄ and evaporated. The crude mixture was triturated with Acute or methylene chloride to give pure benzofuranone compounds. This procedure was used to obtain the following compounds:

### 6-Hydroxy-4-methyl-benzofuran-3-one (C)

Yield: 17%. MS (m/z): 165.1 (MH+).

### 6-Hydroxy-5-methyl-benzofuran-3-one (D)

Yield: 69%. MS (m/z): 165.1 (MH+).

### 6-Hydroxy-7-methyl-benzofuran-3-one (E)

Yield: 22%. MS (m/z): 165.2 (MH+).

### 4-Fluoro-6-hydroxy-benzofuran-3-one (F)

Yield: 27%. MS (m/z): 169.1 (MH+)

### 5-Fluoro-6-hydroxy-benzofuran-3-one (G)

Yield: 28%. MS (m/z): 169.1 (MH+).

### 7-Fluoro-6-hydroxy-benzofuran-3-one (H)

Yield: 29%. MS (m/z): 169.2 (MH+).

### 4-Chloro-6-hydroxy-benzofuran-3-one (I)

Yield: 9%. MS (m/z): 185.1 (MH+).

### 5-Chloro-6-hydroxy-benzofuran-3-one (L)

Yield: 38%. MS (m/z): 185.1 (MH+).

### 7-Chloro-6-hydroxy-benzofuran-3-one (M)

Yield: 30%. MS (m/z): 185.3 (MH+).

### 5-Bromo-6-hydroxy-benzofuran-3-one (N)

Yield: 51%. MS (m/z): 228.9 (MH+).

### 4-Bromo-6-hydroxy-benzofuran-3-one (O)

Yield: 20%. MS (m/z): 229.0 (MH+).

### Preparation of 4,6-dimethoxybenzofuran-3(2H)-one (Compound P)

To a mixture of 3,5-dimethoxyphenol (47.1 g, 306 mmole), 2-chloroacetonitrile (23.07 g, 306 mmole) and zinc chloride (22.90 g, 168 mmole) in ether (450 mL) was bubbled through Hydrochloric acid gas over 2 hours. An oil separates, this mixture was allowed to stir overnight. The ether was decanted from the now solidified oil, the solid rinsed with fresh ether and the ether decanted. To the solid was added 400 mL of water and the mixture boiled for 1 hour, cooled to RT, filtered, washed with water. The solid was mixed with 50 grams of sodium acetate and 400 mL ethanol and the mixture refluxed for 5 hours and cooled. The solid was collected and washed with ethanol. The solid was washed with dichloromethan. The washes were evaporated and the solid isolated with ethyl acetate to give 4,6-dimethoxybenzofuran-3(2H)-one (7.85 g, 40.4 mmol, 13.23% yield).

### Preparation of 7-bromo-4-methoxybenzofuran-3(2H)-one (Compound Q)

To a solution of 1-(3-bromo-2-hydroxy-6-methoxyphenyl)ethanone (6.49 g, 26.5 mmole) in triethylamine (17 mL) and dichloromethane (120 mL) was added TBSCl (4.29 g, 28.5 mmole). This solution was stirred overnight. Reaction mixture was evaporated in-vacuo and treated with 150 mL water, stirred 1 hour, extracted with ether (3×75 mL). The combined ether extracts were combined, washed with 2N hydrochloric acid, water, dried over sodium sulfate, filtered, evaporated and the resulting semi-solid 1-[3-bromo-2-(tert-butyldimethylsilyloxy)-6-methoxyphenyl]ethanone (9.35 g, 26.0 mmol, 98% yield), used as is in the next step.

To a solution of 1-(3-bromo-2-(tert-butyldimethylsilyloxy)-6-methoxyphenyl)ethanone (9.35 g, 26.0 mmole) in TEA (17 mL) and dichloromethane (120 mL) was added TMSOTf (5.64 mL, 31.2 mmole), cooled with an ice bath. This solution was stirred overnight and allowed to warm to RT. Chloroform was added, 120 mL, and the mixture extracted with brine (2×150 mL). The organic layer was dried over sodium sulfate, filtered and evaporated to give a dark brown semi-solid, placed under high-vacuum to remove volatiles, 1-[3-bromo-2-(tert-butyldimethylsilyloxy)-6-methoxyphenyl]vinyloxytrimethylsilane (12.18 g, 26.0 mmol, 100% yield), assumed to be 92% pure, used as is for the next step.

To a solution of 1-[3-bromo-2-(tert-butyldimethylsilyloxy)-6-methoxyphenyl]vinyloxytrimethylsilane (12.18 g, 26.0 mmole) in carbon tetrachloride (120 mL), (some dark oil does not dissolve) cooled in an ice-bath, was added bromine (1.512 mL, 29.3 mmole) in 25 mL carbon tetrachloride in drops over 15 minutes. This was stirred at ice bath temp for 30 minutes then the ice bath was removed and the reaction allowed to warm to room temperature. Reaction mixture was treated with 200 mL water, layers separated. Aqueous extracted with concentrated hydrochloric acid (2×50 mL). Combined organic layers washed with aqueous Na₂S₂O₃, dried over sodium sulfate, filtered through a little Magnesol™, evaporated to give an orange oil, 11.38 g, 2-bromo-1-[3-bromo-2-(tert-butyldimethylsilyloxy)-6-methoxyphenyl]ethanone, used as is in the next step.

To a solution of 2-bromo-1-[3-bromo-2-(tert-butyldimethylsilyloxy)-6-methoxyphenyl]ethanone (11.38 g, 26.0 mmole) in tetrahydrofuran (100 mL), cooled in an ice-bath, was added tetrabutylammonium fluoride (29 ml, 29.0 mmole) (1M in tetrahydrofuran). This was stirred at ice bath temp for 10 minutes then the ice bath was removed and the reaction allowed to warm to room temperature, stirred for 30 minutes. Reaction mixture was quenched with 30 mL saturated ammonium chloride solution. The tetrahydrofuran was removed in-vacuo; water and ether were added. The aqueous layer was extracted with ether (2×25 mL). Combined ether layers washed with water, brine, dried over sodium sulfate, filtered and evaporated to give a yellow residue, purified by chromatography using a hexane-ethyl acetate gradient the product peak was collected, evaporated and the solid isolated with 1:1 hexanes-ethyl acetate, washed with fresh solvent and dried to give a pale yellow solid, 7-bromo-4-methoxybenzofuran-3(2H)-one (587 mg, 9.30% yield).

### Preparation of 6-hydroxy-4-methoxybenzofuran-3(2H)-one (Compound R)

A mixture of 5-methoxybenzene-1,3-diol (10.05 g, 71.7 mmole), 2-chloroacetonitrile (5.41 g, 71.7 mmole), zinc chloride (5.38 g, 39.4 mmole) and ether (100 mL) was stirred in a 500 mL 3N Morton flask. Dry hydrogen chloride gas was bubbled through, solids dissolved and were replaced by a dark oil. After an hour of bubbling hydrochloric acid gas through the mixture the oil became a salmon-colored solid. Hydrochloric acid gas is bubbled through for an additional hour. The mixture was stirred overnight. The mixture was filtered, and the flask rinsed with ether and this ether was used as a wash. Any solids remaining in the flask are left there. The solids were transferred back to the flask and treated with 100 mL of 2N hydrochloric acid and the mixture stirred and brought to reflux. All solids dissolved after heating for a while some solid precipitates. Heated for 2 hours and cooled, the salmon colored solid collected and washed well with water and dried, 9.73 g. A one gram portion of this was purified by chromatography using a hexane-ethyl acetate gradient; the product peak was collected, evaporated to give a yellow solid, 180 mg, MS (m/z) 181.2 (MH+), used as is for the next step.

### Preparation of 6-bromo-1-benzofuran-3(2H)-one (Compound S)

To a stirred solution of boron trichloride in methylene chloride (1.0 M, 6 mL, 6.0 mmole) at 0° C. was added a mixture of 3-bromophenol (870 mg, 5 mmole) in 2 mL of methylene chloride followed by chloroacetonitrile (0.38 mL, 6 mmole) and aluminum chloride (334 mg, 2.5 mmole). The mixture was stirred at room temperature for 20 hours. Then, ice and hydrochloric acid (2N, 4 mL, 8 mmole) were added and the mixture was stirred for 30 min. The mixture was extracted with methylene chloride (×3) and the organic layer was washed with saturated sodium chloride solution, dried over magnesium sulfate, and concentrated. The residue was purified by chromatography over silica, eluting with hexanes to 5% ethyl acetate in hexanes. The desired 1-(4-bromo-2-hydroxyphenyl)-2-chloroethanone was obtained as a mixture with the starting material 3-bromophenol, and was used without further purification. MS (m/z): 246.9 (MH−).

The crude product in the previous step was dissolved in 20 mL of acetonitrile and 3 mL of triethylamine was added. The mixture was stirred at room temperature for 40 min, and concentrated. The residue was purified by chromatography over silica, eluting with hexanes to 2% ethyl acetate in hexanes. The desired 6-bromo-1-benzofuran-3(2H)-one was obtained as a yellow solid (350 mg). MS (m/z): 213.0 (MH+).

### II. Synthesis of Pyrrolopyridine-3-carbaldehyde Intermediates

### Preparation of 5-Methoxy-1H-pyrrolo[3,2-b]pyridine-3-carbaldehyde (Formula 10)

2-Methoxy-5-nitro-pyridine (Formula 6, 4 g, 25.6 mmole) and 4-chlorophenoxyacetonitrile (Formula 7, 4.8 g, 28.5 mmole) were dissolved in THF (58 mL). The resulting solution was slowly added to a solution of t-BuOK (6.3 g, 56.3 mmole) in THF dry (60 mL) at −10° C. The reaction mixture was stirred for 3 h at −10° C., and then water was added. The aqueous layer was extracted with EtOAc. The combined organic layers were dried on Na₂SO₄ and evaporated to give a crude that was purified by silica gel column chromatography (eluent: petroleum ether/EtOAc 8:2) to give (6-methoxy-3-nitro-pyridin-2-yl)-acetonitrile (Formula 8, Yield: 50%. MS (m/z): 194.1 (MH+)).

To a solution of (6-methoxy-3-nitro-pyridin-2-yl)-acetonitrile (Formula 8, 1 g, 5.18 mmole) in EtOH (30 mL), 10% Pd/C was added. The mixture was hydrogenated at 45 psi at room temperature overnight. The catalyst was filtered off and the solvent was evaporated. The crude product was purified by silica gel column chromatography (eluent: petroleum ether/EtOAc 8:2) to give 5-methoxy-1H-pyrrolo[3,2-b]pyridine (Formula 9, Yield: 64%. MS (m/z): 149 (MH+).

To a solution of 5-methoxy-1H-pyrrolo[3,2-b]pyridine (Formula 9, 498 mg, 3.36 mmole) in 33% acetic acid (5.2 mL), hexamethylenetetramine (714 mg, 5.05 mmole) was added. The reaction mixture was refluxed for 4 hours. After cooling, the reaction was extracted with EtOAc. The combined organic layers were dried on Na₂SO₄ and evaporated to give a crude that was purified twice by silica gel column chromatography (eluent: methylene chloride/MeOH 95:5) to give 5-methoxy-1H-pyrrolo[3,2-b]pyridine-3-carbaldehyde (Formula 10, Yield: 27%. MS (m/z): 177.17 (MH+)).

### Preparation of 1-methyl-4-phenyl-1H-pyrrolo[2,3-b]pyridine-3-carbaldehyde (Formula 17)

A solution of 70% mCPBA (11.54 g, 66.87 mmole) in ethyl acetate (25 mL) was added by drops to a solution of 7-azaindole (Formula 11, 5 g, 42.3 mmole) in ethyl acetate (40 mL) at 0° C. with a good stirrer. After addition was completed, the mixture was stirred at room temperature for 1 to 2 hours until no starting material left. The mixture was cooled, filtered, and washed with ethyl acetate to give a solid. It was dissolved in 50 mL of water and treated with 30% K₂CO₃ solution (˜16 mL) to pH 9.5-10.5 to give a precipitate. It was stirred at room temperature for 1 hour, cooled, filtered, and washed with a small amount of cold water to give 2.484 g of 1H-pyrrolo[2,3-b]pyridine 7-oxide as a white crystal (Formula 12, 43.8% yield). MS (m/z): 135.1 (MH+).

A solution of methanesulfonic anhydride (6.066 g, 34.82 mmole) and acetonitrile (11.7 mL) was added by drops to a solution of 1H-pyrrolo[2,3-b]pyridine 7-oxide (Formula 12, 2.333 g, 17.41 mmole), tetramethyl ammonium bromide (4.023 g, 26.12 mmole) in DMF (11.7 mL) at 0° C. After stirring at 0° C. for 45 min, additional DMF (11.7 mL) was added in drops to the thick mixture at 0° C., and then stirred at room temperature overnight. To the mixture was added ice water (35 mL), followed by 10 N NaOH (˜4.66 mL) to pH 7. After stirring at the room temperature, a precipitate formed. It was filtered and washed with water to give 1.891 g of 4-bromo-1H-pyrrolo[2,3-b]pyridine as a pale peach solid (Formula 13, 55% yield). MS (m/z): 197 (MH+). NMR (DMSO-d6) showed 6˜9% impurity which is likely to be the 4,6-dibromo compound based on LC/MS analysis.

A mixture of 4-bromo-1H-pyrrolo[2,3-b]pyridine (Formula 13, 197 mg, 1 mmole), dimethylamine hydrochloride (88 mg, 1.079 mmole), and paraformaldehyde (33 mg, 1.1 mmole) in n-butanol (2 mL) was heated at 120° C. for 1.25 hours. After removal of the solvent, the residue was treated with ice water and three drops of con. HCl. After washing with ether, the aqueous layer was basified with sat. K₂CO₃ solution and extracted with methylene chloride. The organic layer was dried over sodium sulfate, filtered, and the solvent dried to give 106 mg of 1-(4-bromo-1H-pyrrolo[2,3-b]pyridin-3-yl)-N,N-dimethylmethanamine as a light pink solid (Formula 14, 42%). MS (m/z): 254.2 (MH+).

A solution of 1-(4-bromo-1H-pyrrolo[2,3-b]pyridin-3-yl)-N,N-dimethylmethanamine (Formula 14, 341 mg, 1.34 mmole) and hexamethylenetetramine (190 mg, 1.34 mmole) in 66% propionic acid was added by drops to a refluxing solution of hexamethylenetetramine (190 mg, 1.34 mmole) in 66% propionic acid (0.8 mL) at 120° C. The reaction mixture was heated for 2-4 hours, and monitored by MS. It was cooled, treated with water (4 mL), and filtered to give 238 mg of 4-bromo-1H-pyrrolo[2,3-b]pyridine-3-carbaldehyde as a beige solid (Formula 15, 79%). MS (m/z): 225.0 (MH+).

Sodium hydride (60%, 27.4 mg, 0.686 mmole) was added in portions to a suspension of 4-bromo-1H-pyrrolo[2,3-b]pyridine-3-carbaldehyde (Formula 15, 128.6 mg, 0.572 mmole) in 5 mL of DMF and 1 mL of THF at 0° C. After stirring at 0° C. for 20 min, methyl iodide (39.2 μL, 0.6292 mmole) was added by drops into the mixture and warmed up to room temperature for 2.5 hours. The solvents were evaporated and the residue was treated with methylene chloride, filtered, and dried. This was further treated with hexane. The mixture was filtered again and washed with hexane to give a beige solid, which was recrystallized from chloroform and hexane to yield 102 mg of 4-bromo-1-methyl-1H-pyrrolo[2,3-b]pyridine-3-carbaldehyde as crystals (Formula 16, 74%). MS (ESI): m/z 239 (M+H).

A mixture of 4-bromo-1-methyl-1H-pyrrolo[2,3-b]pyridine-3-carbaldehyde (Formula 16, 38 mg, 0.159 mmole), phenyl boronic acid (38.8 mg, 0.318 mmole), and tetrakis(triphenylphosphine)palladium (0) (27.6 mg, 0.0238 mmole) in saturated sodium carbonate (0.37 mL) and 1,2-dimethoxylethane (1.4 mL) was heated at 120° C. in microwave for 20 min. It was filtered through a pad of silica gel and washed with 5% MeOH in ethyl acetate. After the solvent was evaporated, acetonitrile was added to the residue, and filtered to remove a bright yellow solid.

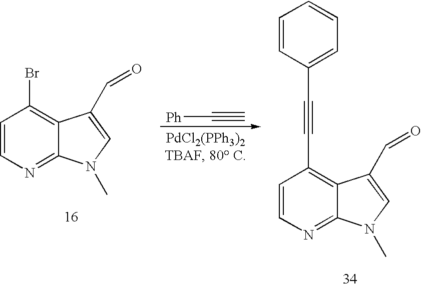

The filtrate was concentrated to yield 51.4 mg of 1-methyl-4-phenyl-1H-pyrrolo[2,3-b]pyridine-3-carbaldehyde as white crystals (Formula 17, Ar=phenyl, 76%). MS (ESI): m/z 237(M+H).

### Synthesis of 1-methyl-4-phenylethynyl-1H-pyrrolo[2,3-b]pyridine-3-carbaldehyde (34)

4-Bromo-1-methyl-1-H-pyrrolo[2,3-b]pyridine-3-carbaldehyde (0.10 g, 0.42 mmole) was combined with phenylacetylene (0.051 g, 0.5 mmole), bis(triphenylphosphine) palladium (II) chloride (8.8 mg, 0.126 mmole) and tetrabutylammonium fluoride (0.33 g, 1.26 mmole) and heated to 80° C. overnight. The thick black solution was diluted with water and extracted with ethyl acetate. The organic layer was washed with brine, dried over MgSO₄, concentrated and purified via silica gel (50% EtOAc: Hex gradient) to produce 79 mg (72%) 1-methyl-4-phenylethynyl-1H-pyrrolo[2,3-b]pyridine-3-carbaldehyde as an off-white solid. Reference: JOC, 2006 (71) 379.

### Synthesis of 1-methyl-4-piperidin-1-yl-1-H-pyrrolo[2,3-b]pyridine-3-carbaldehyde (35)

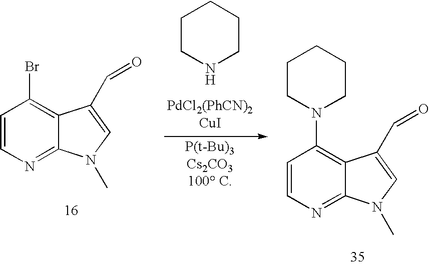

To a mixture of 4-bromo-1-methyl-1-H-pyrrolo[2,3-b]pyridine-3-carbaldehyde (0.06 g, 0.25 mmole) in dioxane (5 mL) was added piperidine (0.12 mL, 1.25 mmole), bis(benzonitrile)dichloro palladium (1.4 mg, 0.0038 mmole), copper(I)iodide (1.4 mg, 0.0075 mmole), tri-tert-butyl phosphine (2.3 mg, 0.011 mmole) and cesium carbonate (0.16 g, 0.5 mmole) and was heated to 100° C. overnight. The reaction was concentrated and purified on silica using a 50% EtOAc/Hex gradient to produce 0.035 g (54%) of 1-methyl-4-piperidin-1-yl-1-H-pyrrolo[2,3-b]pyridine-3-carbaldehyde as an off white solid. Reference: Synlett, 2001 (5) p. 609.

### Synthesis of 1-methyl-4-styryl-1H-pyrrolo[2,3-b]pyridine-3-carbaldehyde (36)

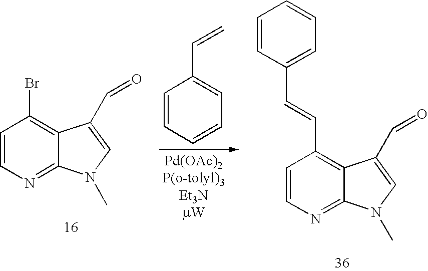

To a mixture of 4-bromo-1-methyl-1-H-pyrrolo[2,3-b]pyridine-3-carbaldehyde (0.08 g, 0.33 mmole) in DMF (2 mL) in a 2-5 mL Biotage microwave vial was added palladium acetate (6 mg, 0.027 mmole), tri-o-tolylphosphine (23.4 mg, 0.077 mmole), triethyl amine (0.19 mL, 1.34 mmole) and styrene (0.077 mL, 0.67 mmole). It was irradiated at 160° C. for 45 min (Biotage Initiator™ 60). The solution was stripped to dryness and purified on silica gel (50% EtOAc/Hex gradient) to give 0.045 g (51%) 1-methyl-4-styryl-1H-pyrrolo[2,3-b]pyridine-3-carbaldehyde. Ref: Synlett, 2001 (5) p. 609.

### Synthesis of 1-methyl-4-phenoxy-1H-pyrrolo[2,3-b]pyridine-3-carbaldehyde (37A) and 4-hydroxy-1-methyl-1-H-pyrrolo[2,3-b]pyridine-3-carbaldehyde (37B)

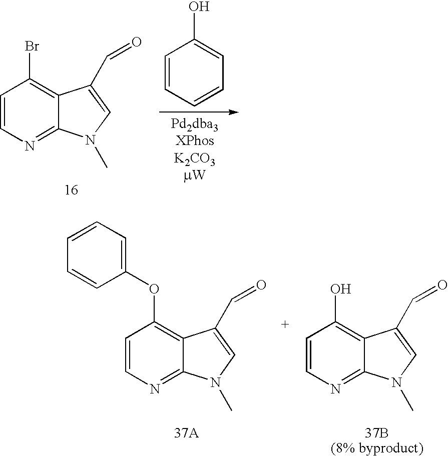

To a mixture of 4-bromo-1-methyl-1-H-pyrrolo[2,3-b]pyridine-3-carbaldehyde (0.2 g, 0.84 mmole) in toluene (2 mL) was added phenol (0.12 g, 1.25 mmole), tris(dibenzylideneacetone)dipalladium (0.04 g, 0.042 mmole), 2-(dicyclohexylphosphino)-2′,4′,6′-triisopropyl-1,1′-biphenyl (0.04 g, 0.084 mmole) (X-Phos), potassium carbonate (0.26 g, 1.85 mmole) and degassed in a 2-5 mL microwave tube. The mixture was irradiated to 130° C. for 3 hours (Biotage Initiator™ 60), cooled, filtered through a Whatman 45 micron filter and concentrated. Purification on silica gel using a 50% EtOAc/Hex gradient afforded 0.095 g (45%) of 1-methyl-4-phenoxy-1H-pyrrolo[2,3-b]pyridine-3-carbaldehyde as a white solid. Ref: Synthesis, 2006 (4) p. 629. By this process, 0.017 g (8%) of 4-hydroxy-1-methyl-1-H-pyrrolo[2,3-b]pyridine-3-carbaldehyde was isolated as a minor by-product.

### 1-Methyl-4-[methyl(phenyl)amino]-1-H-indole-3-carbaldehyde (38)

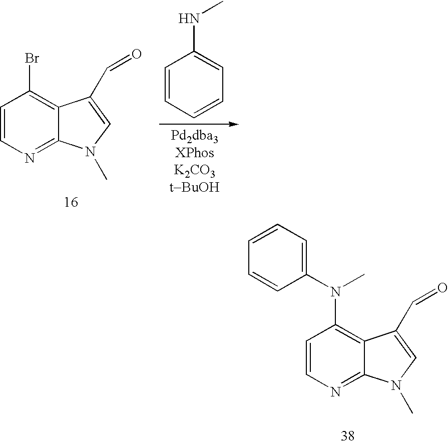

To a mixture of 4-bromo-1-methyl-1-H-pyrrolo[2,3-b]pyridine-3-carbaldehyde (0.05 g, 0.21 mmole) in t-butanol (1 mL) was added N-methyl aniline (0.026 g, 0.24 mmole), tris(dibenzylideneacetone)dipalladium (0.01 g, 0.042 mmole), 2-(dicyclohexylphosphino)-2′,4′,6′-triisopropyl-1,1′-biphenyl (0.011 g, 0.023 mmole) (X-Phos), potassium carbonate (0.064 g, 0.46 mmole) and degassed in a pressure tube. The mixture was heated at 100° C. for 20 hours, cooled, diluted with 20 mL of methylene chloride, filtered through Celite™ and concentrated. Purification on a preparative LC a 50% EtOAc/Hex gradient afforded 0.033 g (29%) of 1-methyl-4-[methyl(phenyl)amino]-1-H-indole-3-carbaldehyde as a pale yellow solid. Ref: Synthesis, 2006 (4) p. 629.

### Synthesis of 1-methyl-2-phenyl-1H-pyrrolo[2,3-b]pyridine (40)

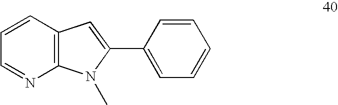

A mixture of 1-methyl-1H-pyrrolo[2,3-b]pyridine (0.919 g, 6.96 mmole), palladium acetate (7.8 mg, 0.035 mmole), triphenylphosphine (36.5 mg, 0.139 mmole), phenyl iodide (0.935 mL, 8.352 mmole), cesium acetate (2.645 g, 13.78 mmole) in dimethyl acetamide (0.92 mL) was heated at 125° C. for 14.5 hours. It was filtered through a pad of silica gel and washed with ethyl acetate. After the solvents were removed, the residue was purified by preparative TLC (developed by 40% ethyl acetate in hexane) to yield 0.767 g (53%) of 1-methyl-2-phenyl-1H-pyrrolo[2,3-b]pyridine as a light yellow oil: MS (ESI) m/z 209.2 (M+H)⁺.

### Synthesis of N,N-dimethyl-1-(1-methyl-2-phenyl-1H-pyrrolo[2,3-b]pyridin-3-yl)methanami ne (41)

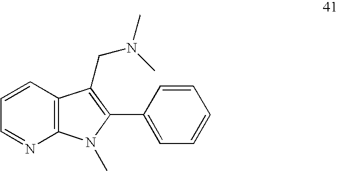

A mixture of 1-methyl-2-phenyl-1H-pyrrolo[2,3-b]pyridine (320 mg, 1.536 mmole), dimethylamine hydrochloride (135 mg, 1.658 mmole) and paraldehyde (50.6 mg, 1.69 mmole) in n-butanol (3 mL) was heated at 120° C. for 1.5 hours. After removal of the solvent, the residue was treated with ice, 3 drops of concentrated HCl, and ether. The aqueous layer was separated and treated with potassium carbonate, followed by treatment with methylene chloride. The organic layer was dried to yield 0.3315 g (81%) of the title compound as a yellow oil: MS (ESI) m/z 266.3 (M+H)⁺.

### Synthesis of 1-methyl-2-phenyl-1H-pyrrolo[2,3-b]pyridine-3-carbaldehyde (42)

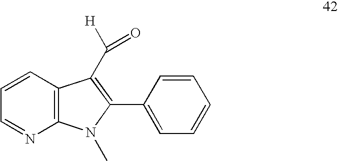

A solution of N,N-dimethyl-1-(1-methyl-2-phenyl-1H-pyrrolo[2,3-b]pyridin-3-yl)methanamine (295 mg, 1.11 mmole), hexamethylenetetramine (156 mg, 1.11 mmole) and 66% propionic acid (1.2 mL) was added in drops to a refluxing solution of hexamethylenetetramine (156 mg, 1.11 mmole) and 66% propionic acid (0.7 mL). After refluxing for 27 hours, it was treated with ice water and methylene chloride. The organic layer was purified by chromatography on a silica gel column and eluted with 40% ethyl acetate in hexane. The fractions were collected and dried to give 0.158 g (60%) of the title compound as a white solid: MS (ESI) m/z 237.2 (M+H)⁺.

### III. Condensation of pyrrolopyridine-3-carbaldehydes and Benzofurans 4,6-dihydroxy-2-(1H-pyrrolo[2,3-b]pyridin-3-ylmethylene)-1-benzofuran-3(2H)-one (Compound II)

To 1H-pyrrolo[2,3-b]pyridin-3-carbaldehyde (0.158 g) in EtOH (3 mL) was added 4,6-dihydroxy-benzofuran-3-one (70 mgs) and HCl (12N, 8 drops). The reaction mixture was heated to 90° C. and stirred for 2.5 hours—LCMS indicated no remaining benzofuranone and product formation. The reaction was allowed to cool. Concentration of the solution in a Speed-Vac and purification via preparative HPLC afforded the title compound. LCMS retention time=1.78 MS=295.1.

Using the procedure above Compounds 1-10 were also prepared. In some cases the reaction suspension was filtered and the solid recrystallized, if necessary, from EtOH. Otherwise the reaction was concentrated via Speed-Vac and purified via preparative HPLC to afford the desired compounds. Compound and analytical data are show in Table I below.

TABLE I
Compounds Prepared According the Procedure of Example 1.

| Cmpd | Name | Time (min) | Mass | Ion | LC/MS conditions |
|---|---|---|---|---|---|
| 1 | 4,6-dihydroxy-2-[(1-methyl-1H-pyrrolo[2,3- | 2.01 | 309.1 | M + H | std method w/formic |
| | b]pyridin-3-yl)methylene]-1-benzofuran- | | | | |
| | 3(2H)-one | | | | |
| 2 | 6,7-dihydroxy-2-[(1-methyl-1H-pyrrolo[2,3- | 1.99 | 309.1 | M + H | std method w/formic |
| | b]pyridin-3-yl)methylene]-1-benzofuran- | | | | |
| | 3(2H)-one | | | | |
| 3 | 4,6-dihydroxy-2-[(5-methoxy-1-methyl-1H- | 2.01 | 339.1 | M + H | std method w/formic |
| | pyrrolo[3,2-b]pyridin-3-yl)methylene]-1- | | | | |
| | benzofuran-3(2H)-one | | | | |
| 4 | 6,7-dihydroxy-2-[(5-methoxy-1-methyl-1H- | 1.97 | 339.1 | M + H | std method w/formic |
| | pyrrolo[3,2-b]pyridin-3-yl)methylene]-1- | | | | |
| | benzofuran-3(2H)-one | | | | |
| 5 | 4-hydroxy-2-[(5-methoxy-1-methyl-1H- | 2.2 | 323.1 | M + H | std method w/formic |
| | pyrrolo[3,2-b]pyridin-3-yl)methylene]-1- | | | | |
| | benzofuran-3(2H)-one | | | | |
| 6 | 4,6-dihydroxy-2-({1-[3-(4-methylpiperazin-1- | 1.84 | 435.2 | M + H | std method w/formic |
| | yl)propyl]-1H-pyrrolo[2,3-b]pyridin-3- | | | | |
| | yl}methylene)-1-benzofuran-3(2H)-one | | | | |
| 7 | 4,6-dihydroxy-2-{[1-(3-morpholin-4-ylpropyl)- | 1.76 | 422.2 | M + H | std method w/formic |
| | 1H-pyrrolo[2,3-b]pyridin-3-yl]methylene}-1- | | | | |
| | benzofuran-3(2H)-one | | | | |
| 8 | 6,7-dihydroxy-2-({1-[3-(4-methylpiperazin-1- | 1.84 | 435.2 | M + H | std method w/formic |
| | yl)propyl]-1H-pyrrolo[2,3-b]pyridin-3- | | | | |
| | yl}methylene)-1-benzofuran-3(2H)-one | | | | |
| 9 | 6,7-dihydroxy-2-{[1-(3-morpholin-4-ylpropyl)- | 1.78 | 422.2 | M + H | std method w/formic |
| | 1H-pyrrolo[2,3-b]pyridin-3-yl]methylene}-1- | | | | |
| | benzofuran-3(2H)-one | | | | |
| 10 | 6,7-dihydroxy-2-[(1-{3-[4-(2- | 1.79 | 465.2 | M + H | std method |
| | hydroxyethyl)piperazin-1-yl]propyl}-1H- | | | | w/formic@215 nm |
| | pyrrolo[2,3-b]pyridin-3-yl)methylene]-1- | | | | |
| | benzofuran-3(2H)-one | | | | |
| 11 | 4,6-dihydroxy-2-(1H-pyrrolo[2,3-b]pyridin-3- | 1.78 | 295.1 | M + H | std method |
| | ylmethylene)-1-benzofuran-3(2H)-one | | | | w/NH₄OAc |

### Preparative Reverse-Phase HPLC(RP-HPLC):

Compounds were in dissolved in 2 mL of 1:1 DMSO:MeCN, filtered through a 0.45 μm GMF, and purified on a Gilson HPLC, using a Phenomenex LUNA C₁₈ column: 60 mm×21.2 mm I.D., 5 um particle size: with ACN/H₂O (containing 0.2% TFA) gradient elution (95:5 H₂O:MeCN to 10:90 H₂O:MeCN; 8 min run

### LCMS Conditions: Standard Method w/ Formic

HPLC Conditions: Instrument—Agilent 1100, Column: Thermo Aquasil C18, 50×2.1 mm, 5 um, Mobile Phase A: 0.1% Formic Acid in water, B: 0.1% Formic Acid in CAN, Flow Rate: 0.800 mL/min, Column Temperature: 40° C., Injection Volume: 5 mL, UV: monitor 215, 230, 254, 280, and 300 nm, Purity is reported at 254 nm unless otherwise noted.

Gradient Table:

| Time (min) | % B |
|---|---|
| 0 | 0 |
| 2.5 | 100 |
| 4.0 | 100 |
| 4.1 | 0 |
| 5.5 | 0 |

MS Conditions: Instrument: Agilent MSD; Ionization Mode: API-ES; Gas Temperature: 350° C.; Drying Gas: 11.0 L/min.; Nebulizer Pressure: 55 psig; Polarity: 50% positive, 50% negative; VCap: 3000V (positive), 2500V (negative); Fragmentor: 80 (positive), 120 (negative); Mass Range: 100-1000 m/z; Threshold: 150; Step size: 0.15; Gain: 1; Peak width: 0.15 min.

LCMS Conditions: Standard Method w/NH₄OAC

HPLC Conditions: Instrument—Agilent 1100, Column: Thermo Aquasil C18, 50×2.1 mm, 5 um, Mobile Phase A: 0.1% Ammonium Acetate in water, B: 0.1% Ammonium Acetate in CAN, Flow Rate: 0.800 mL/min, Column Temperature: 40° C., Injection Volume: 5 mL, UV: monitor 215, 230, 254, 280, and 300 nm. Purity is reported at 254 nm unless otherwise noted.

Gradient Table:

| Time (min) | % B |
|---|---|
| 0 | 0 |
| 2.5 | 100 |
| 4.0 | 100 |
| 4.1 | 0 |
| 5.5 | 0 |

MS Conditions: Instrument: Agilent MSD; Ionization Mode: API-ES; Gas Temperature: 350° C.; Drying Gas: 11.0 L/min.; Nebulizer Pressure: 55 psig; Polarity: 50% positive, 50% negative; VCap: 3000V (positive), 2500V (negative); Fragmentor: 80 (positive), 120 (negative); Mass Range: 100-1000 m/z; Threshold: 150; Step size: 0.15; Gain: 1; Peak width: 0.15 min.

### Preparation of (2Z)-4,6-dihydroxy-2-[(1-methyl-4-phenyl-1H-pyrrolo[2,3-b]pyridin-3-yl)methylene]-1-benzofuran-3(2H)-one (Compound 24)

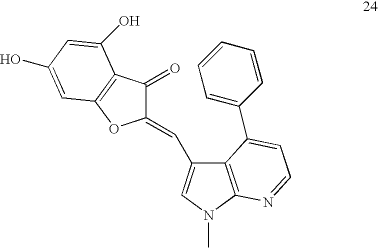

A mixture of 1-methyl-4-phenyl-1H-pyrrolo[2,3-b]pyridine-3-carbaldehyde (Formula 17, Ar=phenyl, 18 mg, 0.076 mmole), 4,6-dihydroxycoumaranone (12.7 mg, 0.076 mmole), ethanol (0.36 mL), and conc. HCl (0.061 mL) was heated at 80° C. After it was turn into a solution, a precipitate formed. After heating for 3 hours, the precipitate was filtered and washed with ethanol to yield 19.8 mg of (2Z)-4,6-dihydroxy-2-[(1-methyl-4-phenyl-1H-pyrrolo[2,3-b]pyridin-3-yl)methylene]-1-benzofuran-3(2H)-one as a yellow solid (Formula 18, Ar=phenyl, 67%). MS (m/z): 385.2 (MH+). ¹H NMR (400 MHz, DMSO-d6) δ ppm 3.99 (s, 3H), 6.03 (s, 1H), 6.22 (s, 1H), 6.29 (s, 1H), 7.11 (d, J=5.1 Hz, 1H), 7.55 (m, 5H), 8.37 (s, 1H), 8.40 (d, J=5.1 Hz, 1H).

### Preparation of (2Z)-4,6-dihydroxy-2-[(5-methoxy-1H-pyrrolo[3,2-b]pyridin-3-yl)methylene]-1-benzofuran-3(2H)-one (Compound 26)

To a solution of 5-methoxy-1H-pyrrolo[3,2-b]pyridine-3-carbaldehyde (10) and 4,6-dihydroxy-benzofuran-3-one A (664 mg, 4 mmol, 1 eq.) in EtOH (16 mL), a catalytic amount of 12 N HCl was added. The resulting mixture was stirred at 85° C. until disappearance of the starting materials and then allowed to cool to room temperature. The formed solid was recovered by filtration, washed with ethyl ether and dried under vacuum. The product was obtained by filtration. Yield: 62%. MS (m/z): 325.19 (MH+).

### Synthesis of (2Z)-4,6-dihydroxy-2-[(1-methyl-2-phenyl-1H-pyrrolo[2,3-b]pyridin-3-yl)methylene]-1-benzofuran-3(2H)-one (Compound 27)

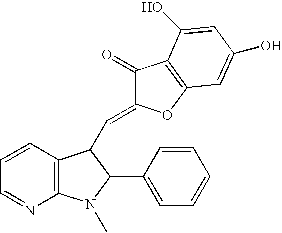

A mixture of 1-methyl-2-phenyl-1H-pyrrolo[2,3-b]pyridine-3-carbaldehyde (70 mg, 0.296 mmole), 4-hydroxy-1-benzofuran-3(2H)-one (49 mg, 0.296 mmole), ethanol (2.18 mL) and conc. HCl (0.235 mL) was heated to 80° C. After heating 3 hours, the formed precipitate was filtered and washed with ethanol to yield 94 mg (82%) of the title compound as a pale yellow solid: MS (ESI) m/z 385.2 (M+H)⁺. ¹H NMR (400 MHz, DMSO-d6) δ ppm 3.73 (s, 3H), 6.10 (s, 1H), 6.27 (s, 1H), 6.47 (s, 1H), 7.36 (ds, J=8.1, 4.6 Hz, 1H), 7.63 (m, 5H), 8.43 (dd, J=4.2, 1.8 Hz, 1H), 8.80 (dd, J=9, 2.5 Hz, 1H), 10.81 (bd, 2H).

### (2Z)-4,6-Dihydroxy-2-[(1-methyl-4-piperidin-1-yl-1H-pyrrolo[2,3-b]pyridin-3-yl)methylene]-1-benzofuran-3(2H)-one (Compound 40)

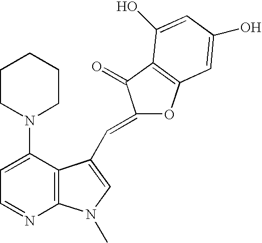

¹H NMR (400 MHz, DMSO-d6) δ ppm 1.66 (m, 2H), 1.80 (m, 4H), 3.23 (m, 4H), 3.96 (s, 3H), 6.11 (s, 1H), 6.26 (s, 1H), 6.92 (d, J=6.3 Hz, 1H), 6.96 (s, 1H), 8.17 (s, 1H), 8.25 (d, J=6.3 Hz, 1H).

### Synthesis of 2-(4-bromo-1-methyl-1-H-pyrrolo[2,3-b]pyridin-3-ylmethylene)-4,6-dihydroxy-benzofuran-3-one (43)

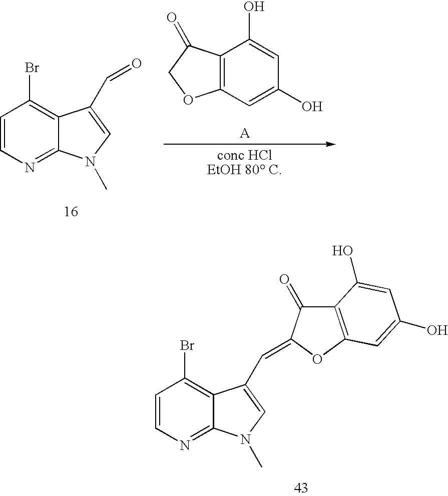

A mixture of 4-bromo-1-methyl-1-H-pyrrolo[2,3-b]pyridine-3-carbaldehyde (1.0 g, 4.2 mmole), 4-hydroxy-1-benzofuran-3(2H)-one (0.69 g, 4.2 mmole), ethanol (50 mL) and conc. HCl (0.25 mL) was heated to 80° C. After heating 6 hours, the formed precipitate was filtered and rinsed with ethanol to yield 0.66 g (41%) of a deep orange solid.

### Synthesis of (2Z)-4,6-dihydroxy-2-{[4-(4-methoxyphenyl)-1-methyl-1H-pyrrolo[2,3-b]pyridin-3-yl]methylene}-1-benzofuran-3(2H)-one (Compound 53)

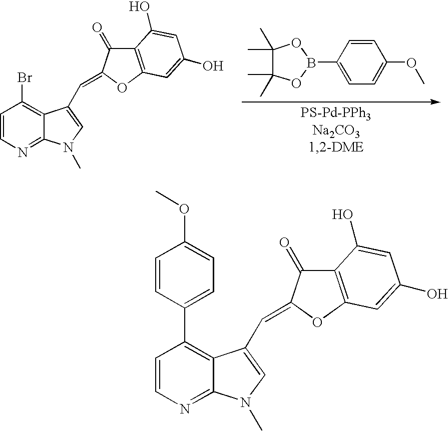

A mixture of 2-(4-bromo-1-methyl-1-H-pyrrolo[2,3-b]pyridin-3-ylmethylene)-4,6-dihydroxy-benzofuran-3-one (0.08 g, 0.21 mmole), 4-methoxyphenylboronic acid pinacol ester (0.1 g, 0.413 mmole), polymer supported palladium triphenylphosphine catalyst (Biotage, 0.11 mmol/g, 19 mg, 0.0021 mmole), in saturated sodium carbonate (0.5 mL) and 1,2-dimethoxyethane (2 mL) was heated to 120° C. in the microwave (Biotage Initiator 60) for 45 min. The slurry was filtered through a Whatman 45 micron filter, rinsed with methanol and concentrated. It was then purified on HPLC to afford 33 mg (38%) of mustard colored solids.

### Synthesis of (2Z)-4,6-dihydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one (Compound 54)

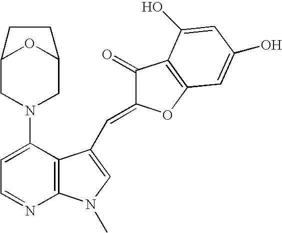

A mixture of 4-(8-oxa-3-azabicyclo[3.2.1]octan-3-yl)-1-methyl-1H-pyrrolo[2,3-b]pyridine-3-carbaldehyde (37 mg, 0.136 mmole), 4,6-dihydroxy-1-benzofuran-3(2H)-one (23 mg, 0.139 mmole), ethanol (1 mL) and conc. HCl (0.11 mL) was heated to 80° C. After heating 21 hours, the formed precipitate was filtered and washed with ethanol to yield 42 mg (74%) of the title compound as an orange solid: MS (ESI) m/z 420.3 (M+H)⁺. ¹H NMR (400 MHz, DMSO-d6) δ ppm 1.89 (bs, 2H), 2.25 (d, J=7.1, 2H), 3.15 (d, J=11.5, 2H), 3.47 (d, J=11.5, 2H), 4.41 (bs, 2H), 6.08 (d, J=1.8, 2H), 6.25 (d, J=1.8, 2H), 6.98 (s, 1H), 7.01 (d, J=6.5 Hz, 1H), 8.17 (s, 1H), 8.22 (d, J=6.5 Hz, 1H), 10.9 (bd, 2H).

### Synthesis of (2Z)-4-hydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one (Compound 55)

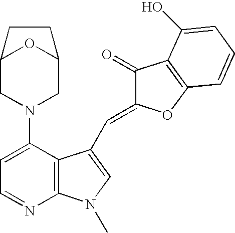

A mixture of 4-(8-oxa-3-azabicyclo[3.2.1]octan-3-yl)-1-methyl-1H-pyrrolo[2,3-b]pyridine-3-carbaldehyde (37 mg, 0.136 mmole), 4-hydroxy-1-benzofuran-3(2H)-one (20.9 mg, 0.139 mmole), ethanol (1 mL) and conc. HCl (0.11 mL) was heated to 80° C. After heating 21 hours, the formed precipitate was filtered and washed with ethanol to yield 48 mg (88%) of the title compound as a yellow solid: MS (ESI) m/z 404.3 (M+H)⁺. ¹H NMR (400 MHz, DMSO-d6) δ ppm 1.92 (bs, 2H), 2.25 (d, J=7.1, 2H), 3.17 (d, J=11.4, 2H), 3.46 (d, J=11.4, 2H), 4.42 (bs, 2H), 6.65 (d, J=8.3, 1H), 6.85 (d, J=8.0, 2H), 7.0 (d, J=6 Hz, 1H), 7.14 (s, 1H), 7.54 (t, J=8.3 Hz, 1H), 8.23 (d, J=6.3 Hz, 1H), 8.25 (s, 1H), 11.1 (bd, 1H).

### 4-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzamide (Compound 111)

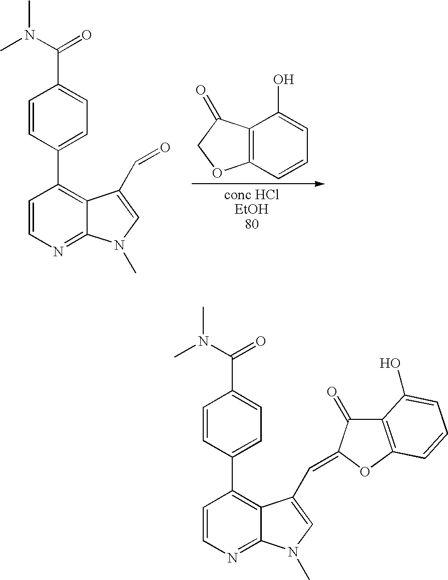

A mixture of 4-(3-formyl-1-methyl-1-H-pyrrolo[2,3-b]pyridin-4-yl)-N,N-dimethyl-benzamide (0.78 g, 0.256 mmole),), 4-hydroxy-1-benzofuran-3(2H)-one (0.038 g, 0.256 mmole), ethanol (5 mL), and conc. HCl (0.025 mL) was heated to 80° C. After heating 6 hours, the formed precipitate was filtered and rinsed with ethanol to yield 0.078 g (69%) of an orange solid. HRMS (ESI) m/e calcd for C₂₆H₂₁N₃O₄ 439.1605, found 439.1603 (M+H)⁺¹; ¹H NMR (400 MHz, DMSO-D6) δ ppm 3.10 (d, J=10.8 Hz, 1H), 4.01 (s, 3H), 6.37 (s, 1H), 6.57 (d, J=8.4 Hz, 1H), 6.83 (d, J=8.4 Hz, 1H), 7.18 (d, J=4.2 Hz, 1H), 7.48 (t, J=8.4 Hz, 1H), 7.61 (s, 4H), 8.43-8.45 (m, 2H), 11.87 (broad, 1H).

### 1-(2-(5-Methoxy-1-[2-(4-methyl-piperazin-1-yl)-ethyl]-1H-pyrrolo[2,3-b]pyridin-3-ylmethylene)-3-oxo-2,3-dihydro-benzofuran-5-yl)-3-methyl-urea (Compound 120)

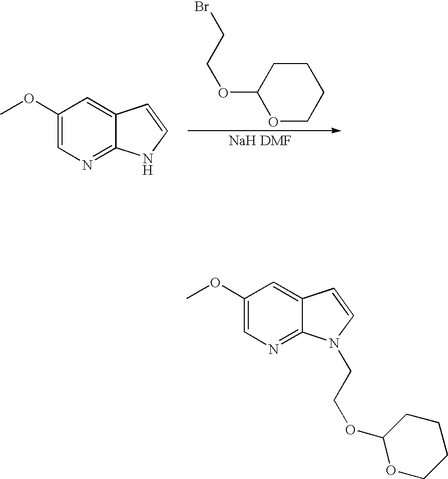

### 5-Methoxy-1-[2-(tetrahydro-pyran-2-yloxy)-ethyl]-1-H-pyrrolo[2,3-b]pyridine

Into a solution of compound I (296 mg, 2.0 mmol) in DMF (40 mL) was added NaH (160 mg, 4 mmol). The mixture was stirred at room temperature for 10 min. 2-(2-bromoethoxy)tetrahydropyran (1.5 mL, 10 mmol) was then added into the above mixture. After being continuously stirred at room temperature for 12 hours, the reaction was quenched with water (1 mL) to give a crude mixture of the desired product, which was used directly in the next step.

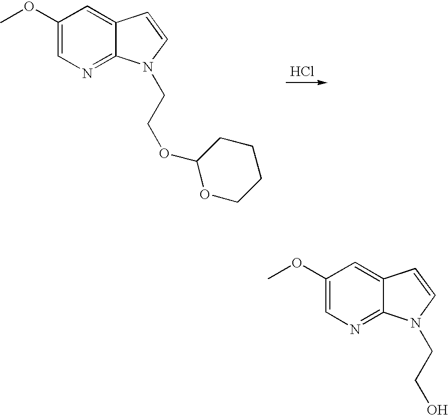

### 2-(5-Methoxy-pyrrolo[2,3-b]pyridin-1-yl)-ethanol

Into the above crude mixture of compound II, was added HCl (37%, 1.5 mL). After being stirred at room temperature for 1 hour, the resulting reaction mixture was poured into water. The mixture was adjusted to pH=9 by adding Na₂CO₃ solid. The mixture was extracted with ethyl acetate (2×), washed with saturated aqueous sodium chloride solution, dried over MgSO₄, suction filtered, concentrated, and chromatographed over a 40 g silica column (eluting with a solution mixture of hexane:ethyl acetate=1:1) to provide 300 mg of the desired product as a white solid. MS: M+H=193.1.

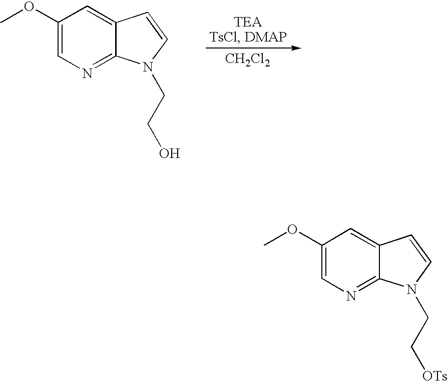

### Toluene-4-sulfonic acid 2-(5-methoxy-pyrrolo[2,3-b]pyridine-1-yl)-ethyl ester

Into a solution of compound III (300 mg, 1.56 mmol) in dichloromethane (20 mL) were added triethylamine (310 μL, 2.34 mmol), DMAP (38 mg, 0.31 mmol) and TsCl (356.9 mg, 1.87 mmol). After being stirred at room temperature for 12 hours, the resulting reaction mixture was then diluted with dichloromethane, washed with water and with saturated NaCl aqueous solution, dried over MgSO₄, suction filtered, concentrated, and dried further in vacuo to give the desired product, which was used directly in the next step without further purification. MS: M+H=347.0.

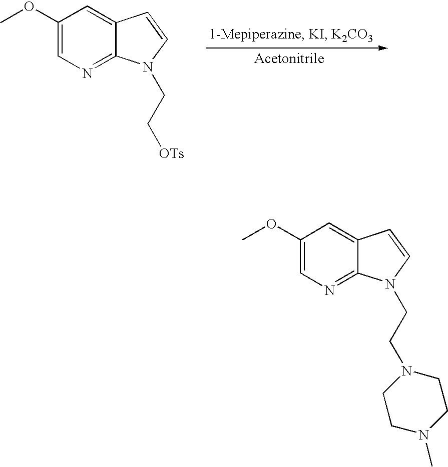

### 5-Methoxy-1-[2-(4-methyl-piperazin-1-yl)-ethyl]-1H-pyrrolo[2,3-b]pyridine

Into a solution of compound IV above in acetonitrile (30 ml) were added 1-methylpiperazine (1.38 ml, 12.44 mmol), K₂CO₃ (1.07 mg, 7.8 mmol) and KI (647.8 mg, 3.9 mmol). The reaction mixture was stirred at 80° C. for 12 hours. The resulting reaction mixture was partitioned between water and ethyl acetate. The organic layer was washed with saturated aqueous sodium chloride solution, dried over MgSO₄, suction filtered, concentrated, and chromatographed over a 40 g silica column (eluting with a solution mixture of CH₂Cl₂:MeOH=95:5) to provide 260.5 mg of the desired product as a light yellow solid. MS: M+H=275.2.

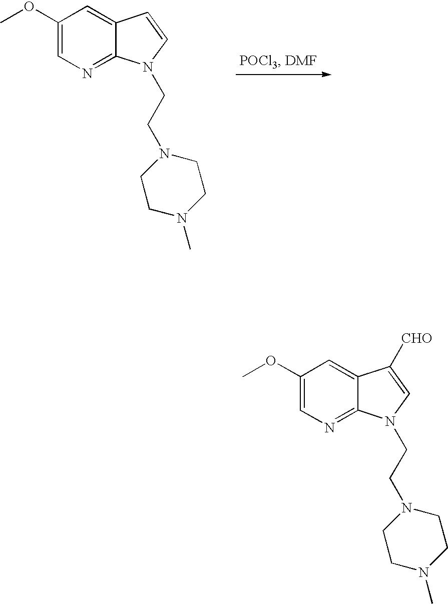

### 5-Methoxy-1-[2-(4-methyl-piperazin-1-yl)-ethyl]-1H-pyrrolo[2,3-b]pyridine-3-carbaldehyde

Into DMF (12 ml) was dropwise added POCl₃ (0.91 mL) with stirring. The mixture was stirred at room temperature for 20 minutes. Into the above mixture was added compound V (260 mg, 0.95 mmol) in DMF (2 mL). The reaction mixture was stirred at room temperature for 14 hours, and then poured into cold water. The mixture was adjusted to pH=9 by adding Na₂CO₃ solid. The mixture was extracted with ethyl acetate, washed with saturated aqueous sodium chloride solution, dried over MgSO₄, suction filtered, concentrated and dried further in vacuo to give the desired product (179.5 mg) as a white solid. MS: M+H=303.1.

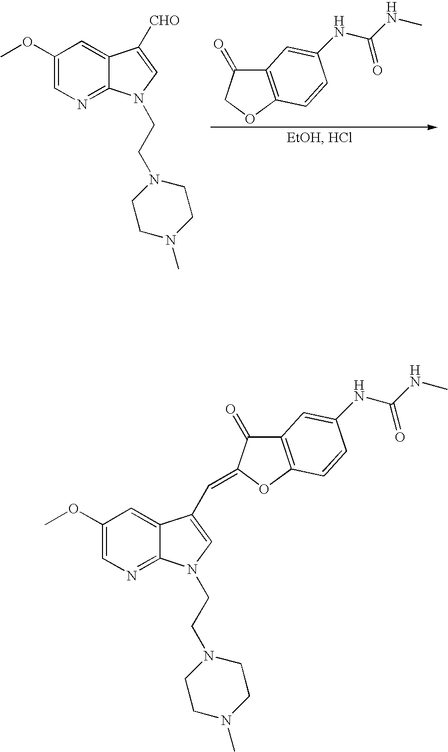

### 1-(2-(5-Methoxy-1-[2-(4-methyl-piperazin-1-yl)-ethyl]-1H-pyrrolo[2,3-b]pyridin-3-ylmethylene)-3-oxo-2,3-dihydro-benzofuran-5-yl)-3-methyl-urea (Compound 120)

Into a solution mixture of HCl (37%, 100 μL) in ethanol (4 mL) were added compound VI (65 mg, 0.21 mmol) and 5-methylurea benzofuranone (53.2 mg, 0.26 mmol). The reaction mixture was stirred at room temperature for 14 hours then suction filtered. The filtered cake was washed with dichloromethane, and then dried in vacuo to give 1-(2-(5-Methoxy-1-[2-(4-methyl-piperazin-1-yl)-ethyl]-1H-pyrrolo[2,3-b]pyridin-3-ylmethylene)-3-oxo-2,3-dihydro-benzofuran-5-yl)-3-methyl-urea (95.1 mg) as a yellow solid. MS: M+H=491.3.

### (Z)-4-Hydroxy-2-((1-methyl-4-(4-(morpholine-4-carbonyl)phenyl)-1H-pyrrolo[2,3-b]pyridin-3-yl)methylene)benzofuran-3(2H)-one (Compound 128)

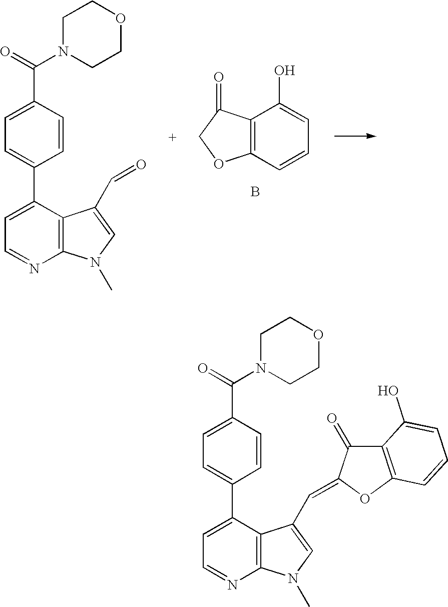

1-Methyl-4-(4-(morpholine-4-carbonyl)phenyl)-1-H-pyrrolo[2,3-b]pyridine-3 carbaldehyde (94.5 mg, 0.27 mmole) and 4-hydroxybenzofuran-3(2H)-one (42.6 mg, 0.285 mmole) were stirred in absolute EtOH (2.2 mL), followed by addition of 0.22 mL of concentrated HCl. It was heated at 80° C. After 4.5 hours, the reaction mixture was cooled in ice bath and filtered. The solid was washed with 4 mL of cold absolute EtOH, dried in vacuum, gave 99.0 mg (76%) of the title product, as a pale yellow solid. HRMS (ESI) m/e calcd for C₂₈H₂₃N₃O₅481.16385, found 482.17066 (M+H)⁺¹; ¹H NMR (400 MHz, DMSO-d6) δ ppm 2.50 (d, J=2.0 Hz, 4H), 3.70 (bs, 4H), 4.00 (s, 3H), 6.28 (s, 1H), 6.58 (d, J=2.0 Hz, 1H), 6.82 (d, J=2.0 Hz, 1H), 7.17 (d, J=2.0 Hz, 1H), 7.45 (t, J=2.0 Hz, 1H), 7.59 (s, 3H), 8.33 (s, 1H), 8.42 (d, J=2.0 Hz, 1H), 10.89 (bs, 1H).

### (Z)-1-(2-((4-(8-oxa-3-azabicyclo[3.2.1]octan-3-yl)-1-methyl-1H-pyrrolo[2,3-b]pyridin-3-yl)methylene)-3-oxo-2,3-dihydrobenzofuran-5-yl)-3-methyl urea (Compound 131)

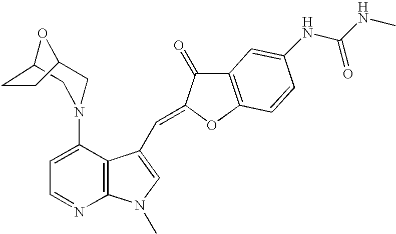

To a mixture of 4-(8-oxa-3-azabicyclo[3.2.1]octan-3-yl)-1-methyl-1H-pyrrolo[2,3-b]pyridine-3-carbaldehyde (57 mg, 0.21 mmole), 1-methyl-3-(3-oxo-2,3-dihydrobenzofuran-5-yl)urea (43.3 mg, 0.21 mmole) and EtOH (4 mL) was added 3 drops of concentrated hydrochloric acid. This was heated to 80° C. and stirred overnight. The reaction mixture was cooled and the yellow solid collected by filtration, washed with ethanol, washed with ether, air dried and vacuum dried to give (Z)-1-(2-((4-(8-oxa-3-azabicyclo[3.2.1]octan-3-yl)-1-methyl-1H-pyrrolo[2,3-b]pyridin-3-yl)methylene)-3-oxo-2,3-dihydrobenzofuran-5-yl)-3-methylurea (57 mg, 59% yield) mp 247-55 (dec) (begins to soften at 230° C.). HRMS (ESI) m/e calcd for C₂₅H₂₅N₅O₄ 460.19793, found 460.19674 (M+H)⁺¹; ¹H NMR (400 MHz, DMSO-D6) δ ppm 1.91-1.99 (m, 2H) 2.31-2.39 (m, 2H) 2.66 (s, 3H) 3.05-3.13 (m, 2H) 3.32-3.40 (m, 2H) 3.94 (s, assumed to be 3H, overlap with H₂O) 4.40-4.46 (m, 2H) 5.92-6.32 (obs, 1H) 6.96 (d, J=5.9, 1H) 7.38 (s, 1H) 7.42 (d, J=8.9, 1H) 7.67 (dd, J=8.9, 2.4, 1H) 7.87 (d, J=2.4, 1H) 8.24 (d, J=5.9, 1H) 8.83 (s, 1H).

### (2Z)-4-hydroxy-2-({1-methyl-4-[4-(8-oxa-3-azabicyclo[3.2.1]oct-3-ylcarbonyl)phenyl]-1H-pyrrolo[2,3-b]pyridin-3-yl}methylidene)-1-benzofuran-3(2H)-one (Compound 144)

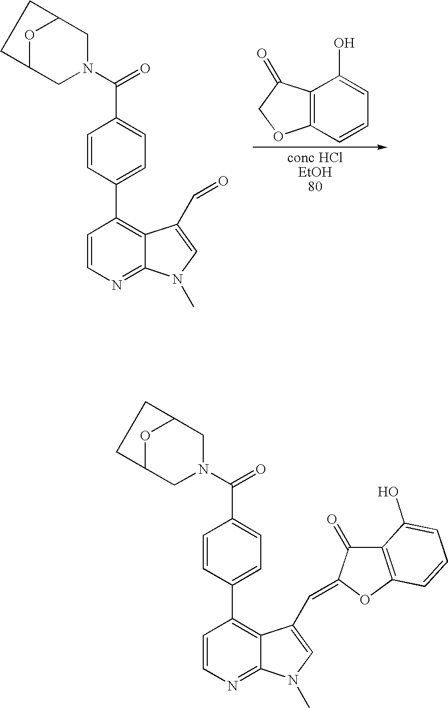

A mixture of 1-methyl-4-[4-(8-oxa-3-azabicyclo[3.2.1]octane-3-carbonyl)-phenyl]-1H-pyrrolo[2,3-b]pyridine-3-carbaldehyde (0.093 g, 0.25 mmole), 4-hydroxy-1-benzofuran-3(2H)-one (0.037 g, 0.25 mmole), ethanol (5 mL) and conc. HCl (0.025 mL) was heated to 80° C. After heating 6 hours, the formed precipitate was filtered and rinsed with ethanol to yield 0.089 g (70%) of a yellow solid. HRMS (ESI) m/e calcd for C₃₀H₂₅N₃O₅ 508.1867, found 508.1864 (M+H)⁺¹; ¹H NMR (400 MHz, DMSO-D6) δ ppm 1.65-1.9 (m, 4H), 3.04 (d, J=12.1 Hz, 1H), 3.51 (d, J=12.1 Hz, 1H), 4.00 (s, 3H), 4.03-4.3 (m, assume 2H, overlapping with water), 4.42 (s, 2H), 6.26 (s, 1H); 6.60 (d, J=8.2 Hz, 1H), 6.82 (d, J=8.2 Hz, 1H), 7.17 (d, J=5.8 Hz, 1H), 7.43 (t, J=8.2 Hz, 1H), 7.57 (m, 4H), 8.41 (m, 2H), 11.1 (broad, 1H).

### 1-{3-[(Z)-(4-hydroxy-3-oxo-1-benzofuran-2(3H)-ylidene)methyl]-1-methyl-1H-pyrrolo[2,3-b]pyridin-4-yl}-N,N-dimethyl piperidine-4-carboxamide (Compound 145)

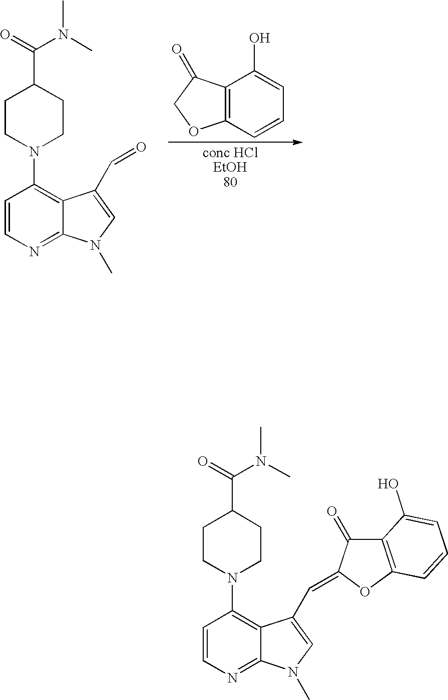

A mixture of 1-(3-formyl-1-methyl-1H-pyrrolo[2,3-b]pyridin-4-yl)-piperidine-4-carboxylic acid dimethylamide (0.072 g, 0.23 mmole),), 4-hydroxy-1-benzofuran-3(2H)-one (0.034 g, 0.23 mmole), ethanol (5 mL) and conc. HCl (0.025 mL) was heated to 80° C. After heating 6 hours, the solution was cooled and concentrated to half volume. The solids were filtered and rinsed with acetonitrile to yield 0.036 g (35%) of a yellow solid. HRMS (ESI) m/e calcd for C₂₅H₂₆N₄O₄ 447.2027, found 447.2032 (M+H)⁺¹; ¹H NMR (400 MHz, DMSO-D6) δ ppm 1.83 (d, J=12.4 Hz, 2H), 1.94 (q, J=12.4 Hz, 2H), 2.86 (s, 3H), 3.06 (s, 3H), 2.82-3.07 (m, assume 3H buried), 3.71 (d, J=12.4 Hz, 2H), 3.96 (s, 3H), 6.65 (d, J=8.5 Hz, 1H); 6.86 (d, J=7.6 Hz, 1H), 6.94 (d, J=7.6 Hz, 1H), 7.08 (s, 1H), 7.53 (t, J=8.5 Hz, 1H), 8.26 (d, J=7.6 Hz, 2H), 11.02 (broad, 1H).

### (Z)-4-hydroxy-2-((4-(4-(4-(2-hydroxyethyl)piperazine-1-carbonyl)phenyl)-1-methyl-1H-pyrrolo[2,3-b]pyridin-3-yl)methylene)benzofuran-3(2m-one (Compound 146)

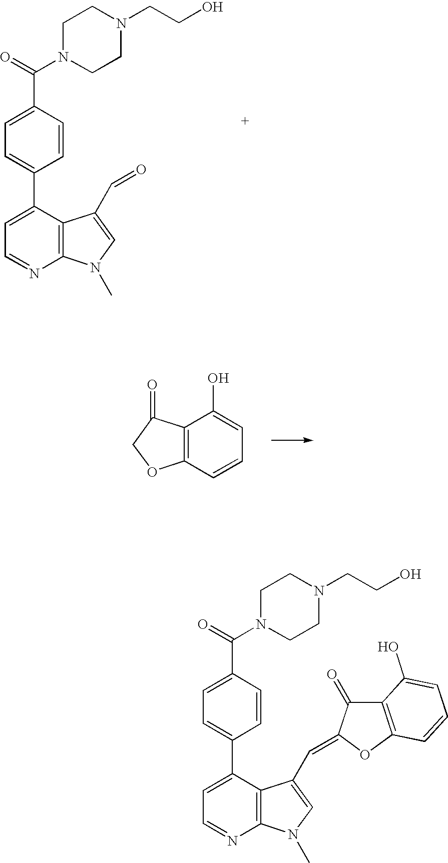

4-(4-(4-(2-Hydroxyethyl)piperazine-1-carbonyl)phenyl)-1-methyl-1H-pyrrolo[2,3-b]pyridine-3-carbaldehyde (160 mg, 0.408 mmole) and 4-hydroxybenzofuran-3(2H)-one (64.2 mg., 0.428 mmole) were stirred in absolute EtOH (3.2 mL), followed by addition of 0.34 mL of concentrated HCl. It was heated at 80° C. After 5.2 hours, the reaction mixture was cooled in ice bath and filtered. The solid washed with 4 mL of cold absolute EtOH, dried in vacuum, gave 154.0 mg (72%) of the title product, as a pale yellow solid. MS (ESI) m/e calcd for C₃₀H₂₈N₄O₅ 524.2, found 525.2 (M+H)⁺¹. ¹H NMR (400 MHz, DMSO-d6) δ ppm 3.24 (bd, J=3.0 Hz, 2H), 3.62-3.65 (bd, J=3.0 Hz, 2H), 3.81-3.83 (m, 4H), 3.86 (m, 4H), 6.24 (s, 1H), 6.63 (d, J=2.0 Hz, 1H), 6.83 (d, J=2.0 Hz, 1H), 7.20 (d, J=2.0 Hz, 1H), 7.50 (t, J=2.0 Hz, 1H), 7.61-7.67 (m, 3H), 8.42 (s, 1H), 8.45 (d, J=1.0 Hz, 1H), 10.98 (bs, 1H).

### (Z)-4-hydroxy-2-((1-methyl-4-(4-(morpholine-4-carbonyl)piperidin-1-yl)-1H-pyrrolo[2,3-b]pyridin-3-yl)methylene)benzofuran-3(2H)-one (Compound 150)

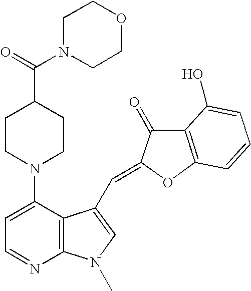

To a mixture of 1-methyl-4-(4-(morpholine-4-carbonyl)piperidin-1-yl)-1H-pyrrolo[2,3-b]pyridine-3-carbaldehyde (89 mg, 0.25 mmole-4-hydroxybenzofuran-3(2H)-one (38 mg, 0.25 mmole) and EtOH (5 mL) was added 3 drops of concentrated hydrochloric acid. This was heated to 80° C. and stirred overnight. The reaction mixture was cooled and the orange solid collected by filtration, washed with ethanol, washed with ether, air dried and vacuum dried to give (Z)-4-hydroxy-2-((1-methyl-4-(4-(morpholine-4-carbonyl)piperidin-1-yl)-1H-pyrrolo[2,3-b]pyridin-3-yl)methylene)benzofuran-3(2H)-one (82 mg, 67% yield) mp 358-61 (dec). Mol Ion: M+H 489.2; ¹H NMR (400 MHz, DMSO-D6) δ ppm 1.76-1.87 (m, 2H), 1.87-2.01 (m, 2H), 2.86-2.96 (m, 1H), 2.96-3.07 (m, 2H), 3.46-3.71 (m, 10H), 3.95 (s, 3H), 6.63 (d, J=8.3 Hz, 1H), 6.86 (d, J=8.1 Hz, 1H), 6.92 (d, J=5.9 Hz, 1H), 7.11 (s, 1H) 7.58 (t, J=8.2 Hz, 1H) 8.25 (d, J=5.9 Hz, 1H) 8.26 (s, 1H) 11.01 (obs, 1H).

### (Z)-4-hydroxy-2-((1-methyl-4-(4-(pyrrolidine-1-carbonyl)piperidin-1-yl)-1H-pyrrolo[2,3-b]pyridin-3-yl)methylene)benzofuran-3(2H)-one (Compound 152)

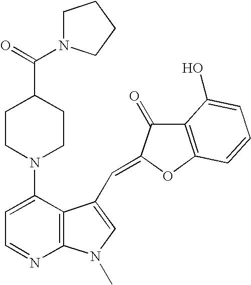

To a mixture of 1-methyl-4-(4-(pyrrolidine-1-carbonyl)piperidin-1-yl)-1H-pyrrolo[2,3-b]pyridine-3-carbaldehyde (55 mg, 0.162 mmole 4-hydroxybenzofuran-3(2H)-one (24 mg, 0.162 mmole) and EtOH (3 mL) was added 2 drops of concentrated hydrochloric acid. This was heated to 80° C. and stirred overnight. The reaction mixture was cooled and the yellow solid collected by filtration, washed with ethanol, washed with ether, air dried and vacuum dried to give (Z)-4-hydroxy-2-((1-methyl-4-(4-(pyrrolidine-1-carbonyl)piperidin-1-yl)-1H-pyrrolo[2,3-b]pyridin-3-yl)methylene)benzofuran-3(2H)-one (36 mg, 46% yield) mp 191-207 (dec). Mol Ion: M+H 473.2; ¹H NMR (400 MHz, DMSO-D6) δ ppm 1.74-2.02 (m, 8H), 2.63-2.73 (m, 1H), 2.87-2.98 (m, 2H), 3.30 (t, J=6.9 Hz, 2H), 3.51 (t, J=6.9 Hz, 2H), 3.58-3.67 (m, 2H), 3.94 (s, 3H), 6.62 (d, J=8.2 Hz, 1H), 6.86 (d, J=8.1 Hz, 1H), 6.89 (d, J=5.9 Hz, 1H), 7.16 (s, 1H) 7.52 (t, J=8.2 Hz, 1H) 8.23 (d, J=5.7 Hz, 1H) 8.26 (s, 1H) 10.97 (obs, 1H).

### Synthesis of (Z)-N,N-dimethyl-1-(1-methyl-3-((3-oxo-5-(3-pyridin-3-yl ureido)benzofuran-2(3H)-ylidene)methyl)-1H-pyrrolo[2,3-b]pyridin-4-yl)piperidine-4-carboxamide (Compound 189)

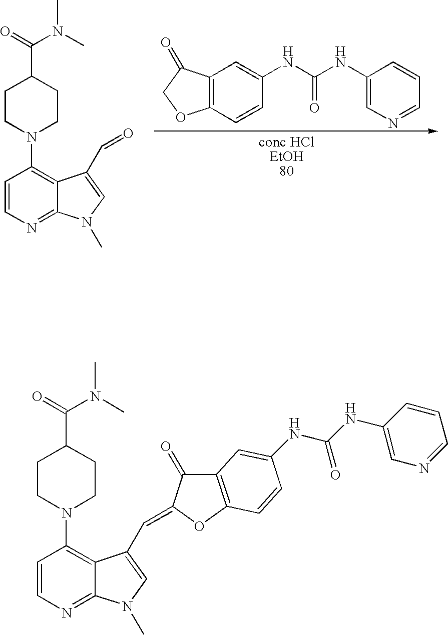

A mixture of 1-(3-formyl-1-methyl-1H-pyrrolo[2,3-b]pyridin-4-yl)-piperidine-4-carboxylic acid dimethylamide (0.09 g, 0.29 mmole), 1-(3-oxo-2,3-dihydrobenzofuran-5-yl)-3-(pyridin-3-yl)urea (0.078 g, 0.29 mmole), ethanol (5 mL) and conc. HCl (0.025 mL) was heated to 80° C. After heating 6 hours, the solution was cooled; the solids formed upon cooling were filtered, rinsed with ethanol and acetone and dried in vacuo to afford 0.14 g (85%) of a yellowish solid. HRMS (ESI) m/e calcd for C₃₁H₃₁N₇O₄ 566.2150, found 566.2514 (M+H)⁺¹.

### Synthesis of 1-(3-formyl-1-methyl-1H-pyrrolo[2,3-b]pyridin-4-yl)-N,N-dimethyl piperidine-4-carboxamide

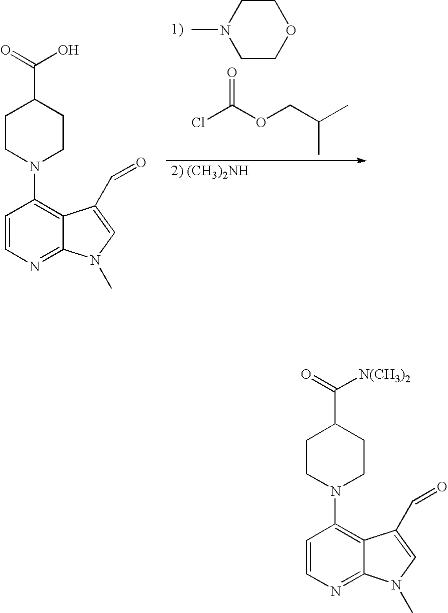

To a suspension of 0.070 g (0.24 mmole) of 1-(3-formyl-1-methyl-1H-pyrrolo[2,3-b]pyridin-4-yl)piperidine-4-carboxylic acid in 1.2 ml of DMF and 1.2 mL THF was added 0.08 mL (0.074 g, 0.73 mmole) N-methylmorpholine then was cooled to 0° C. 0.05 g (0.365 mmole) of isobutyl chloroformate was added dropwise and allowed to react for 20 minutes before adding 0.24 mL (0.488 mmole) of 2M dimethylamine in THF. The reaction was allowed to warm to room temperature overnight. The reaction was concentrated in vacuo, diluted with water and extracted with ethyl acetate 2 times. The organic layers were combined, washed with brine and concentrated. Silica gel purification using 100% hexane:ethyl acetate gradient afforded 0.05 g (65% yield) white solids. (M+H)⁺ 315.4

### Synthesis of 1-(3-formyl-1-methyl-1H-pyrrolo[2,3-b]pyridin-4-yl)piperidine-4-carboxylic acid

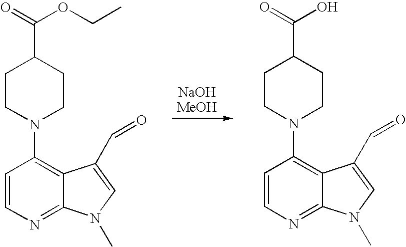

To a solution of 1.0 g (3.17 mmole) of ethyl 1-(3-formyl-1-methyl-1H-pyrrolo[2,3-b]pyridin-4-yl)piperidine-4-carboxylate in 20 mL methanol was added 8 mL 2N NaOH. The solution was allowed to react at room temperature overnight. The solution was concentrated, taken up in water and washed with ethyl acetate two times. The aqueous layer was cooled and acidified with 2N HCl. Solids formed which were then filtered, washed with water and dried in vacuo to afford 0.75 g (82%) off white solids. (M+H)⁺ 288.3

### Synthesis of ethyl 1-(3-formyl-1-methyl-1H-pyrrolo[2,3-b]pyridin-4-yl)piperidine-4-carboxylate

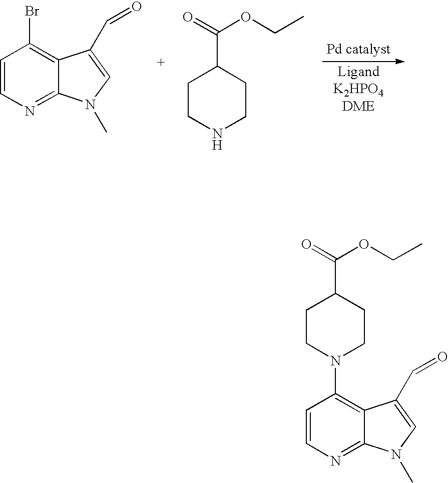

Heat a suspension of 4-bromo-1-methyl-1H-pyrrolo[2,3-b]pyridine-3-carbaldehyde (1.2 g, 5 mmole), 2′-(dicyclohexylphosphino)-N,N-dimethylbiphenyl-2-amine (0.148 g, 0.376 mmole), tris(dibenzylideneacetone)dipalladium (0) (0.115 g, 0.125 mmole), potassium hydrogen phosphate (1.749 g, 10 mmole) in 50 mL of dimethoxyethane to 80° C. under nitrogen. Then ethyl piperidine-4-carboxylate (1.578 g, 10 mmole) was added. The flask was sealed and heated to 110° C. overnight. The reaction mixture was cooled and absorbed onto silica gel and purified on the ISCO 40 g column using 60% B gradient (EtOAc/10% MeOH in EtOAc). The desired fractions were collected and dried to give 1.0 g (63%) of ethyl 1-(3-formyl-1-methyl-1H-pyrrolo[2,3-b]pyridin-4-yl)piperidine-4-carboxylate.

### Synthesis of methyl 4-(3-formyl-1-methyl-1H-pyrrolo[2,3-b]pyridin-4-yl)benzoate

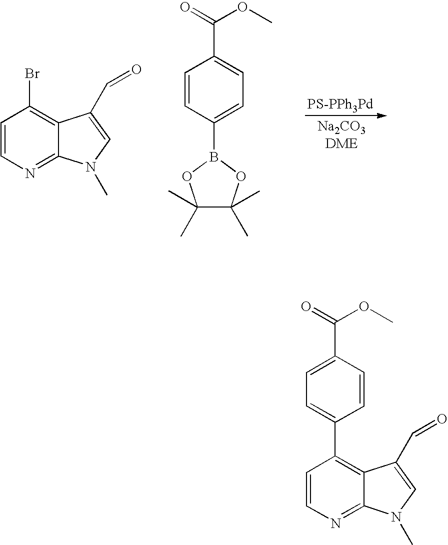

In a 20 ml microwave vial was combined 4-bromo-1-methyl-1-H-pyrrolo[2,3-b]pyridine-3-carbaldehyde (0.5 g, 2.1 mmole), dimethoxyethane (8 mL), methyl 4-(4,4,5,5-tetramethyl-1,3,2-dioxaborolan-2-yl)benzoate (1.1 g, 4.2 mmole), saturated sodium carbonate (4.6 mL) and polymer supported triphenylphosphine palladium (0.38 g, 0.042 mmole; Biotage, 0.11 mmol/g). The mixture was then heated to 130° C. under microwave irradiation for 45 minutes, cooled then filtered through Celite™ and rinsed with warm methanol and water. The solution was concentrated to half the volume when solids formed. The solids were filtered, washed with water, dried in vacuo to afford 0.46 g (75%) of an off white solid. (M+H)⁺ 295.4.

The following pyrrolopyridine compounds were prepared according to the above procedures.

TABLE 2

| | | | | HRMS |
|---|---|---|---|---|
| Cmpd | Name | MP ° C. | MS | ESI [M + H]⁺ |
| 1 | 4,6-dihydroxy-2-[(1-methyl-1H-pyrrolo[2,3-b]pyridin- | | 309.1 | |
| | 3-yl)methylene]-1-benzofuran-3(2H)-one | | | |
| 2 | 6,7-dihydroxy-2-[(1-methyl-1H-pyrrolo[2,3-b]pyridin- | | 309.1 | |
| | 3-yl)methylene]-1-benzofuran-3(2H)-one | | | |
| 3 | 4,6-dihydroxy-2-[(5-methoxy-1-methyl-1H- | | 339.1 | |
| | pyrrolo[3,2-b]pyridin-3-yl)methylene]-1-benzofuran- | | | |
| | 3(2H)-one | | | |
| 4 | 6,7-dihydroxy-2-[(5-methoxy-1-methyl-1H- | | 339.1 | |
| | pyrrolo[3,2-b]pyridin-3-yl)methylene]-1-benzofuran- | | | |
| | 3(2H)-one | | | |
| 5 | 4-hydroxy-2-[(5-methoxy-1-methyl-1H-pyrrolo[3,2- | | 323.1 | |
| | b]pyridin-3-yl)methylene]-1-benzofuran-3(2H)-one | | | |
| 6 | 4,6-dihydroxy-2-({1-[3-(4-methylpiperazin-1- | | 435.2 | |
| | yl)propyl]-1H-pyrrolo[2,3-b]pyridin-3-yl}methylene)- | | | |
| | 1-benzofuran-3(2H)-one | | | |
| 7 | 4,6-dihydroxy-2-{[1-(3-morpholin-4-ylpropyl)-1H- | | 422.2 | |
| | pyrrolo[2,3-b]pyridin-3-yl]methylene}-1-benzofuran- | | | |
| | 3(2H)-one | | | |
| 8 | 6,7-dihydroxy-2-({1-[3-(4-methylpiperazin-1- | | 435.2 | |
| | yl)propyl]-1H-pyrrolo[2,3-b]pyridin-3-yl}methylene)- | | | |
| | 1-benzofuran-3(2H)-one | | | |
| 9 | 6,7-dihydroxy-2-{[1-(3-morpholin-4-ylpropyl)-1H- | | 422.2 | |
| | pyrrolo[2,3-b]pyridin-3-yl]methylene}-1-benzofuran- | | | |
| | 3(2H)-one | | | |
| 10 | 6,7-dihydroxy-2-[(1-{3-[4-(2-hydroxyethyl)piperazin- | | 465.2 | |
| | 1-yl]propyl}-1H-pyrrolo[2,3-b]pyridin-3-yl)methylene]- | | | |
| | 1-benzofuran-3(2H)-one | | | |
| 11 | 4,6-dihydroxy-2-(1H-pyrrolo[2,3-b]pyridin-3- | | 295.1 | |
| | ylmethylene)-1-benzofuran-3(2H)-one | | | |
| 12 | (2Z)-2-{[4-(2-chlorophenyl)-1-methyl-1H-pyrrolo[2,3- | | 419.1 | |
| | b]pyridin-3-yl]methylene}-4,6-dihydroxy-1- | | | |
| | benzofuran-3(2H)-one | | | |
| 13 | (2Z)-2-{[4-(3-chlorophenyl)-1-methyl-1H-pyrrolo[2,3- | | 419.1 | |
| | b]pyridin-3-yl]methylene}-4,6-dihydroxy-1- | | | |
| | benzofuran-3(2H)-one | | | |
| 14 | (2Z)-4,6-dihydroxy-2-{[4-(2-methoxyphenyl)-1- | | 415.1 | |
| | methyl-1H-pyrrolo[2,3-b]pyridin-3-yl]methylene}-1- | | | |
| | benzofuran-3(2H)-one | | | |
| 15 | (2Z)-4,6-dihydroxy-2-({1-methyl-4-[4- | | 467.1 | |
| | (trifluoromethoxy)phenyl]-1H-pyrrolo[2,3-b]pyridin-3- | | | |
| | yl}methylene)-1-benzofuran-3(2H)-one | | | |
| 16 | (2Z)-4,6-dihydroxy-2-[(4-{4-[2-(1H-imidazol-1- | | 495.2 | |
| | yl)ethoxy]phenyl}-1-methyl-1H-pyrrolo[2,3-b]pyridin- | | | |
| | 3-yl)methylene]-1-benzofuran-3(2H)-one | | | |
| 17 | 4-{3-[(Z)-(4,6-dihydroxy-3-oxo-1-benzofuran-2(3H)- | | 464.1 | |
| | ylidene)methyl]-1-methyl-1H-pyrrolo[2,3-b]pyridin-4- | | | |
| | yl}benzenesulfonamide | | | |
| 18 | N-(3-{3-[(Z)-(4,6-dihydroxy-3-oxo-1-benzofuran- | | 478.1 | |
| | 2(3H)-ylidene)methyl]-1-methyl-1H-pyrrolo[2,3- | | | |
| | b]pyridin-4-yl}phenyl)methanesulfonamide | | | |
| 19 | (2Z)-4,6-dihydroxy-2-{[4-(4-hydroxyphenyl)-1- | | 401.1 | |
| | methyl-1H-pyrrolo[2,3-b]pyridin-3-yl]methylene}-1- | | | |
| | benzofuran-3(2H)-one | | | |
| 20 | 4-{3-[(Z)-(4,6-dihydroxy-3-oxo-1-benzofuran-2(3H)- | | 443.1 | |
| | ylidene)methyl]-1-methyl-1H-pyrrolo[2,3-b]pyridin-4- | | | |
| | yl}-N′-hydroxybenzenecarboximidamide | | | |
| 21 | 3-{3-[(Z)-(4-hydroxy-3-oxo-1-benzofuran-2(3H)- | | 448.1 | |
| | ylidene)methyl]-1-methyl-1H-pyrrolo[2,3-b]pyridin-4- | | | |
| | yl}benzenesulfonamide | | | |
| 22 | (2Z)-2-({4-[4-(5,6-dihydro-4H-1,3-oxazin-2- | | 470 | |
| | yl)phenyl]-1-methyl-1H-pyrrolo[2,3-b]pyridin-3- | | | |
| | yl}methylidene)-4-hydroxy-1-benzofuran-3(2H)-one | | | |
| 23 | (2Z)-4-hydroxy-2-{[1-methyl-4-(1H-pyrrol-2-yl)-1H- | | 555.2 | |
| | pyrrolo[2,3-b]pyridin-3-yl]methylidene}-1- | | | |
| | benzofuran-3(2H)-one | | | |
| 24 | (2Z)-4,6-dihydroxy-2-[(1-methyl-4-phenyl-1H- | | | calcd for |
| | pyrrolo[2,3-b]pyridin-3-yl)methylene]-1-benzofuran- | | | C23H16N2O4 + H⁺, |
| | 3(2H)-one | | | 385.11828; found |
| | | | | 385.1179; |
| 25 | (2Z)-2-({4-[4-(dimethylamino)phenyl]-1-methyl-1H- | | | calcd for |
| | pyrrolo[2,3-b]pyridin-3-yl}methylene)-4,6-dihydroxy- | | | C25H21N3O4 + H⁺, |
| | 1-benzofuran-3(2H)-one | | | 428.16048; found |
| | | | | 428.1604; |
| 26 | (2Z)-4,6-dihydroxy-2-[(5-methoxy-1H-pyrrolo[3,2- | | | |
| | b]pyridin-3-yl)methylene]-1-benzofuran-3(2H)-one | | | |
| 27 | (2Z)-4,6-dihydroxy-2-[(1-methyl-2-phenyl-1H- | | 385.3 | calcd for |
| | pyrrolo[2,3-b]pyridin-3-yl)methylene]-1-benzofuran- | | | C23H16N2O4 + H⁺, |
| | 3(2H)-one | | | 385.11828; found |
| | | | | 385.1183; |
| 28 | (2Z)-6-hydroxy-2-[(1-methyl-2-phenyl-1H- | | 369.3 | calcd for |
| | pyrrolo[2,3-b]pyridin-3-yl)methylene]-1-benzofuran- | | | C23H16N2O3 + H⁺, |
| | 3(2H)-one | | | 369.12337; found |
| | | | | 369.1235; |
| 29 | (2Z)-4-hydroxy-2-[(1-methyl-2-phenyl-1H- | | 369.3 | calcd for |
| | pyrrolo[2,3-b]pyridin-3-yl)methylene]-1-benzofuran- | | | C23H16N2O3 + H⁺, |
| | 3(2H)-one | | | 369.12337; found |
| | | | | 369.1235; |
| 30 | (2Z)-6-hydroxy-2-[(1-methyl-4-phenyl-1H- | 314-6 dec | 369.3 | calcd for |
| | pyrrolo[2,3-b]pyridin-3-yl)methylene]-1-benzofuran- | | | C23H16N2O3 + H⁺, |
| | 3(2H)-one | | | 369.12337; found |
| | | | | 369.1236; |
| 31 | (2Z)-4-hydroxy-2-[(1-methyl-4-phenyl-1H- | 274-5 | 369.3 | calcd for |
| | pyrrolo[2,3-b]pyridin-3-yl)methylene]-1-benzofuran- | | | C23H16N2O3 + H⁺, |
| | 3(2H)-one | | | 369.12337; found |
| | | | | 369.1238; |
| 32 | (2Z)-4-fluoro-6-hydroxy-2-[(1-methyl-4-phenyl-1H- | 325-7 dec | 387.3 | calcd for |
| | pyrrolo[2,3-b]pyridin-3-yl)methylene]-1-benzofuran- | | | C23H15FN2O3 + H⁺, |
| | 3(2H)-one | | | 387.11395; |
| | | | | found 387.1147; |
| 33 | (2Z)-4,6-dihydroxy-2-[(1-methyl-4-pyridin-3-yl-1H- | | | calcd for |
| | pyrrolo[2,3-b]pyridin-3-yl)methylene]-1-benzofuran- | | | C22H15N3O4 + H⁺, |
| | 3(2H)-one | | | 386.11353; found |
| | | | | 386.1141; |
| 34 | 4-{3-[(Z)-(4,6-dihydroxy-3-oxo-1-benzofuran-2(3H)- | | | |
| | ylidene)methyl]-1-methyl-1H-pyrrolo[2,3-b]pyridin-4- | | | |
| | yl}benzamide | | | |
| 35 | N-(4-{3-[(Z)-(4,6-dihydroxy-3-oxo-1-benzofuran- | | 442.2 | calcd for |
| | 2(3H)-ylidene)methyl]-1-methyl-1H-pyrrolo[2,3- | | | C25H19N3O5 + H⁺, |
| | b]pyridin-4-yl}phenyl)acetamide | | | 442.13975; found |
| | | | | 442.1404; |
| 36 | N-(4-{3-[(Z)-(4,6-dihydroxy-3-oxo-1-benzofuran- | | 478.2 | calcd for |
| | 2(3H)-ylidene)methyl]-1-methyl-1H-pyrrolo[2,3- | | | C24H19N3O6S + H⁺, |
| | b]pyridin-4-yl}phenyl)methanesulfonamide | | | 478.10673; |
| | | | | found 478.1070; |
| 37 | (2Z)-5-bromo-2-[(1-methyl-4-phenyl-1H-pyrrolo[2,3- | 227-9 | | calcd for |
| | b]pyridin-3-yl)methylene]-1-benzofuran-3(2H)-one | | | C23H15BrN2O2 + H⁺, |
| | | | | 431.03896; |
| | | | | found 431.0391; |
| 38 | 1-methyl-3-{(2Z)-2-[(1-methyl-4-phenyl-1H- | 265-70 | | calcd for |
| | pyrrolo[2,3-b]pyridin-3-yl)methylene]-3-oxo-2,3- | dec | | C25H20N4O3 + H⁺, |
| | dihydro-1-benzofuran-5-yl}urea | | | 425.16082; found |
| | | | | 425.1610; |
| 39 | (2Z)-4,6-dihydroxy-2-{[1-methyl-4-(phenylethynyl)- | 308-310 | | calcd for |
| | 1H-pyrrolo[2,3-b]pyridin-3-yl]methylene}-1- | | | C25H16N2O4 + H⁺, |
| | benzofuran-3(2H)-one | | | 409.11828; found |
| | | | | 409.1185; |
| 40 | (2Z)-4,6-dihydroxy-2-[(1-methyl-4-piperidin-1-yl-1H- | 322-324 | | calcd for |
| | pyrrolo[2,3-b]pyridin-3-yl)methylene]-1-benzofuran- | | | C22H21N3O4 + H⁺, |
| | 3(2H)-one | | | 392.16048; found |
| | | | | 392.1611; |
| 41 | (2Z)-2-{[4-(4-aminophenyl)-1-methyl-1H-pyrrolo[2,3- | | | calcd for |
| | b]pyridin-3-yl]methylene}-4,6-dihydroxy-1- | | | C23H17N3O4 + H⁺, |
| | benzofuran-3(2H)-one | | | 400.12918; found |
| | | | | 400.1291; |
| 42 | (2Z)-5-hydroxy-2-[(1-methyl-4-phenyl-1H- | 352-4 dec | 369.2 | calcd for |
| | pyrrolo[2,3-b]pyridin-3-yl)methylene]-1-benzofuran- | | | C23H16N2O3 + H⁺, |
| | 3(2H)-one | | | 369.12337; found |
| | | | | 369.1238; |
| 43 | (2Z)-4,6-dihydroxy-2-({1-methyl-4-[4- | | 414.1 | |
| | (methylamino)phenyl]-1H-pyrrolo[2,3-b]pyridin-3- | | | |
| | yl}methylene)-1-benzofuran-3(2H)-one | | | |
| 44 | (2Z)-4,6-dihydroxy-2-{[1-methyl-4-(4- | | | calcd for |
| | methylpiperazin-1-yl)-1H-pyrrolo[2,3-b]pyridin-3- | | | C22H22N4O4 + H⁺, |
| | yl]methylene}-1-benzofuran-3(2H)-one | | | 407.17138; found |
| | | | | 407.1721; |
| 45 | (2Z)-4,6-dihydroxy-2-[(1-methyl-4-morpholin-4-yl- | | | calcd for |
| | 1H-pyrrolo[2,3-b]pyridin-3-yl)methylene]-1- | | | C21H19N3O5 + H⁺, |
| | benzofuran-3(2H)-one | | | 394.13975; found |
| | | | | 394.1404; |
| 46 | (2Z)-4,6-dihydroxy-2-[(1-methyl-4-pyrrolidin-1-yl-1H- | 312-315 | 378.3 | calcd for |
| | pyrrolo[2,3-b]pyridin-3-yl)methylene]-1-benzofuran- | | | C21H19N3O4 + H⁺, |
| | 3(2H)-one | | | 378.14483; found |
| | | | | 378.1455; |
| 47 | (2Z)-4-hydroxy-2-{[1-methyl-4-(4-methylpiperazin-1- | | | calcd for |
| | yl)-1H-pyrrolo[2,3-b]pyridin-3-yl]methylene}-1- | | | C22H22N4O3 + H⁺, |
| | benzofuran-3(2H)-one | | | 391.17647; found |
| | | | | 391.1770; |
| 48 | (2Z)-4-hydroxy-2-[(1-methyl-4-morpholin-4-yl-1H- | | | calcd for |
| | pyrrolo[2,3-b]pyridin-3-yl)methylene]-1-benzofuran- | | | C21H19N3O4 + H⁺, |
| | 3(2H)-one | | | 378.14483; found |
| | | | | 378.1452; |
| 49 | (2Z)-4,6-dihydroxy-2-{[1-methyl-4-(4-morpholin-4- | | 470.2 | |
| | ylphenyl)-1H-pyrrolo[2,3-b]pyridin-3-yl]methylene}-1- | | | |
| | benzofuran-3(2H)-one | | | |
| 50 | (2Z)-4,6-dihydroxy-2-{[4-(4-hydroxypiperidin-1-yl)-1- | | 408.3 | calcd for |
| | methyl-1H-pyrrolo[2,3-b]pyridin-3-yl]methylene}-1- | | | C22H21N3O5 + H⁺, |
| | benzofuran-3(2H)-one | | | 408.15540; found |
| | | | | 408.1562; |
| 51 | (2Z)-4,6-dihydroxy-2-({1-methyl-4-[(E)-2- | decomp | 411.3 | calcd for |
| | phenylvinyl]-1H-pyrrolo[2,3-b]pyridin-3- | at 295-315 | | C25H18N2O4 + H⁺, |
| | yl}methylene)-1-benzofuran-3(2H)-one | | | 411.13393; found |
| | | | | 411.1347; |
| 52 | (2Z)-2-({4-[3-(dimethylamino)phenyl]-1-methyl-1H- | | 428.3 | calcd for |
| | pyrrolo[2,3-b]pyridin-3-yl}methylene)-4,6-dihydroxy- | | | C25H21N3O4 + H⁺, |
| | 1-benzofuran-3(2H)-one | | | 428.16048; found |
| | | | | 428.1611; |
| 53 | (2Z)-4,6-dihydroxy-2-{[4-(4-methoxyphenyl)-1- | decomp | | calcd for |
| | methyl-1H-pyrrolo[2,3-b]pyridin-3-yl]methylene}-1- | begins at | | C24H18N2O5 + H⁺, |
| | benzofuran-3(2H)-one | 230 | | 415.12885; found |
| | | | | 415.1288; |
| 54 | (2Z)-4,6-dihydroxy-2-{[1-methyl-4-(8-oxa-3- | | | calcd for |
| | azabicyclo[3.2.1]oct-3-yl)-1H-pyrrolo[2,3-b]pyridin-3- | | | C23H21N3O5 + H⁺, |
| | yl]methylene}-1-benzofuran-3(2H)-one | | | 420.15540; found |
| | | | | 420.1553; |
| 55 | (2Z)-4-hydroxy-2-{[1-methyl-4-(8-oxa-3- | | | calcd for |
| | azabicyclo[3.2.1]oct-3-yl)-1H-pyrrolo[2,3-b]pyridin-3- | | | C23H21N3O4 + H⁺, |
| | yl]methylene}-1-benzofuran-3(2H)-one | | | 404.16048; found |
| | | | | 404.1605; |
| 56 | (2Z)-4,6-dihydroxy-2-({1-methyl-4-[4-(4- | | 483.4 | calcd for |
| | methylpiperazin-1-yl)phenyl]-1H-pyrrolo[2,3- | | | C28H26N4O4 + H⁺, |
| | b]pyridin-3-yl}methylene)-1-benzofuran-3(2H)-one | | | 483.20268; found |
| | | | | 483.20156; |
| 57 | (2Z)-4-hydroxy-2-({1-methyl-4-[4-(4-methylpiperazin- | | 467.4 | calcd for |
| | 1-yl)phenyl]-1H-pyrrolo[2,3-b]pyridin-3- | | | C28H26N4O3 + H⁺, |
| | yl}methylene)-1-benzofuran-3(2H)-one | | | 467.20777; found |
| | | | | 467.2066; |
| 58 | (2Z)-5-amino-2-[(1-methyl-4-phenyl-1H-pyrrolo[2,3- | 232-9 | 368.3 | calcd for |
| | b]pyridin-3-yl)methylene]-1-benzofuran-3(2H)-one | | | C23H17N3O2 + H⁺, |
| | | | | 368.13935; found |
| | | | | 368.1397; |
| 59 | (2Z)-5-bromo-2-{[1-methyl-4-(8-oxa-3- | | 466.2 | calcd for |
| | azabicyclo[3.2.1]oct-3-yl)-1H-pyrrolo[2,3-b]pyridin-3- | | | C23H20BrN3O3 + H⁺, |
| | yl]methylene}-1-benzofuran-3(2H)-one | | | 466.07608; |
| | | | | found 466.07493; |
| 60 | 2-hydroxyethyl {(2Z)-2-[(1-methyl-4-phenyl-1H- | 233-5 | | calcd for |
| | pyrrolo[2,3-b]pyridin-3-yl)methylene]-3-oxo-2,3- | | | C26H21N3O5 + H⁺, |
| | dihydro-1-benzofuran-5-yl}carbamate | | | 456.15540; found |
| | | | | 456.15442; |
| 61 | 1-ethyl-3-[(2Z)-2-{[1-methyl-4-(8-oxa-3- | | | calcd for |
| | azabicyclo[3.2.1]oct-3-yl)-1H-pyrrolo[2,3-b]pyridin-3- | | | C26H27N5O4 + H⁺, |
| | yl]methylene}-3-oxo-2,3-dihydro-1-benzofuran-5- | | | 474.21358; found |
| | yl]urea | | | 474.21329; |
| 62 | (2Z)-4,6-dihydroxy-2-[(1-methyl-4-pyrimidin-5-yl-1H- | decomp | 385.2 | calcd for |
| | pyrrolo[2,3-b]pyridin-3-yl)methylene]-1-benzofuran- | 340 | | C21H14N4O4 + H⁺, |
| | 3(2H)-one | | | 387.1088; found |
| | | | | 387.1086; |
| 63 | (2Z)-2-{[4-(6-chloropyridin-3-yl)-1-methyl-1H- | decomp | 420.1 | calcd for |
| | pyrrolo[2,3-b]pyridin-3-yl]methylene}-4,6-dihydroxy- | 340 | | C22H14CIN3O4 + H⁺, |
| | 1-benzofuran-3(2H)-one | | | 420.07456; |
| | | | | found 420.0746; |
| 64 | (2Z)-2-{[4-(4-chlorophenyl)-1-methyl-1H-pyrrolo[2,3- | decomposes | 419.2 | calcd for |
| | b]pyridin-3-yl]methylene}-4,6-dihydroxy-1- | 345 | | C23H15CIN2O4 + H⁺, |
| | benzofuran-3(2H)-one | | | 419.07931; |
| | | | | found 419.0794; |
| 65 | (2Z)-2-[(4-azepan-1-yl-1-methyl-1H-pyrrolo[2,3- | decomp | 406.2 | calcd for |
| | b]pyridin-3-yl)methylene]-4,6-dihydroxy-1- | 297-300 | | C23H23N3O4 + H⁺, |
| | benzofuran-3(2H)-one | | | 406.17613; found |
| | | | | 406.1760; |
| 66 | (2Z)-4,6-dihydroxy-2-{[4-(3-methoxyphenyl)-1- | decomp | 415.2 | calcd for |
| | methyl-1H-pyrrolo[2,3-b]pyridin-3-yl]methylene}-1- | 297-300 | | C24H18N2O5 + H⁺, |
| | benzofuran-3(2H)-one | | | 415.12885; found |
| | | | | 415.1297; |
| 67 | (2Z)-4,6-dihydroxy-2-({4-[4-(hydroxymethyl)phenyl]- | >330 | 415.2 | calcd for |
| | 1-methyl-1H-pyrrolo[2,3-b]pyridin-3-yl}methylene)-1- | | | C24H18N2O5 + H⁺, |
| | benzofuran-3(2H)-one | | | 415.12885; found |
| | | | | 415.1293; |
| 68 | 2,2-dimethyl-N-{(2Z)-2-[(1-methyl-4-phenyl-1H- | 251-3 | 452.3 | calcd for |
| | pyrrolo[2,3-b]pyridin-3-yl)methylene]-3-oxo-2,3- | | | C28H25N3O3 + H⁺, |
| | dihydro-1-benzofuran-4-yl}propanamide | | | 452.19687; found |
| | | | | 452.19678; |
| 69 | (2Z)-4-hydroxy-2-{[1-methyl-4-(6-morpholin-4- | | 455.3 | calcd for |
| | ylpyridin-3-yl)-1H-pyrrolo[2,3-b]pyridin-3- | | | C26H22N4O4 + H⁺, |
| | yl]methylene}-1-benzofuran-3(2H)-one | | | 455.17138; found |
| | | | | 455.17003; |
| 70 | (2Z)-4-hydroxy-2-{[1-methyl-4-(6-piperidin-1- | | 453.3 | calcd for |
| | ylpyridin-3-yl)-1H-pyrrolo[2,3-b]pyridin-3- | | | C27H24N4O3 + H⁺, |
| | yl]methylene}-1-benzofuran-3(2H)-one | | | 453.19212; found |
| | | | | 453.19037; |
| 71 | (2Z)-4-hydroxy-2-{[4-(6-methoxypyridin-3-yl)-1- | 242-4 | 400.1 | calcd for |
| | methyl-1H-pyrrolo[2,3-b]pyridin-3-yl]methylene}-1- | | | C23H17N3O4 + H⁺, |
| | benzofuran-3(2H)-one | | | 400.12918; found |
| | | | | 400.1287; |
| 72 | (2Z)-4,6-dihydroxy-2-({1-methyl-4-[4- | decomp | 463.2 | calcd for |
| | (methylsulfonyl)phenyl]-1H-pyrrolo[2,3-b]pyridin-3- | 276-286 | | C24H18N2O6S + H⁺, |
| | yl}methylene)-1-benzofuran-3(2H)-one | | | 463.09583; |
| | | | | found 463.0958; |
| 73 | (2Z)-4-hydroxy-2-({1-methyl-4-[4- | decomp | 447.2 | calcd for |
| | (methylsulfonyl)phenyl]-1H-pyrrolo[2,3-b]pyridin-3- | 266- | | C24H18N2O5S + H⁺, |
| | yl}methylene)-1-benzofuran-3(2H)-one | | | 447.10092; |
| | | | | found 447.1010; |
| 74 | (2Z)-4,6-dihydroxy-2-({1-methyl-4-[3- | decomp | 463 | calcd for |
| | (methylsulfonyl)phenyl]-1H-pyrrolo[2,3-b]pyridin-3- | 315-320 | | C24H18N2O6S + H⁺, |
| | yl}methylene)-1-benzofuran-3(2H)-one | | | 463.09583; |
| | | | | found 463.0953; |
| 75 | (2Z)-4-hydroxy-2-({1-methyl-4-[3- | decomp | 447.1 | calcd for |
| | (methylsulfonyl)phenyl]-1H-pyrrolo[2,3-b]pyridin-3- | 310-318 | | C24H18N2O5S + H⁺, |
| | yl}methylene)-1-benzofuran-3(2H)-one | | | 447.10092; |
| | | | | found (ESI, |
| | | | | [[M + H]+), 447.1006; |
| 76 | (2Z)-4-hydroxy-2-({4-[6-(hydroxymethyl)pyridin-3-yl]- | decomp | 400 | calcd for |
| | 1-methyl-1H-pyrrolo[2,3-b]pyridin-3-yl}methylene)-1- | 270-275 | | C23H17N3O4 + H⁺, |
| | benzofuran-3(2H)-one | | | 400.12918; found |
| | | | | 400.1285; |
| 77 | 4-{3-[(Z)-(4,6-dihydroxy-3-oxo-1-benzofuran-2(3H)- | | | calcd for |
| | ylidene)methyl]-1-methyl-1H-pyrrolo[2,3-b]pyridin-4- | | | C24H19N3O6S + H⁺, |
| | yl}-N-methylbenzenesulfonamide | | | 478.10673; |
| | | | | found (EST-FTMS, |
| | | | | [M + H]1+), |
| | | | | 478.10689; |
| 78 | (2Z)-4,6-dihydroxy-2-{[1-methyl-4-(1,2,3,6- | | | calcd for |
| | tetrahydropyridin-4-yl)-1H-pyrrolo[2,3-b]pyridin-3- | | | C22H19N3O4 + H⁺, |
| | yl]methylene}-1-benzofuran-3(2H)-one | | | 390.14483; found |
| | | | | (EST-FTMS, |
| | | | | [M + H]1+), |
| | | | | 390.14495; |
| 79 | (2Z)-4,6-dihydroxy-2-({1-methyl-4-[3-(pyrrolidin-1- | | | calcd for |
| | ylcarbonyl)phenyl]-1H-pyrrolo[2,3-b]pyridin-3- | | | C28H23N3O5 + H⁺, |
| | yl}methylene)-1-benzofuran-3(2H)-one | | | 482.17105; found |
| | | | | (EST-FTMS, |
| | | | | [M + H]1+), |
| | | | | 482.17116; |
| 80 | (2Z)-4,6-dihydroxy-2-({1-methyl-4-[3-(piperidin-1- | | | calcd for |
| | ylsulfonyl)phenyl]-1H-pyrrolo[2,3-b]pyridin-3- | | | C28H25N3O6S + H⁺, |
| | yl}methylene)-1-benzofuran-3(2H)-one | | | 532.15368; |
| | | | | found (EST-FTMS, |
| | | | | [M + H]1+), |
| | | | | 532.15325; |
| 81 | (2Z)-4-hydroxy-2-({4-[4-methoxy-3-(morpholin-4- | | | calcd for |
| | ylsulfonyl)phenyl]-1-methyl-1H-pyrrolo[2,3-b]pyridin- | | | C28H25N3O7S + H⁺, |
| | 3-yl}methylene)-1-benzofuran-3(2H)-one | | | 548.14860; |
| | | | | found 548.14717; |
| 82 | N-cyclopropyl-4-{3-[(Z)-(4-hydroxy-3-oxo-1- | | | calcd for |
| | benzofuran-2(3H)-ylidene)methyl]-1-methyl-1H- | | | C26H21N3O5S + H⁺, |
| | pyrrolo[2,3-b]pyridin-4-yl}benzenesulfonamide | | | 488.12747; |
| | | | | found 488.12632; |
| 83 | N-(4-fluorophenyl)-4-{3-[(Z)-(4-hydroxy-3-oxo-1- | | | calcd for |
| | benzofuran-2(3H)-ylidene)methyl]-1-methyl-1H- | | | C30H20FN3O4 + H⁺, |
| | pyrrolo[2,3-b]pyridin-4-yl}benzamide | | | 506.15106; |
| | | | | found 506.1496; |
| 84 | (2Z)-4-hydroxy-2-{[1-methyl-4-(4-phenoxyphenyl)- | | | calcd for |
| | 1H-pyrrolo[2,3-b]pyridin-3-yl]methylene}-1- | | | C29H20N2O4 + H⁺, |
| | benzofuran-3(2H)-one | | | 461.14958; found |
| | | | | 461.14861; |
| 85 | (2Z)-2-{[4-(1-benzofuran-2-yl)-1-methyl-1H- | | | calcd for |
| | pyrrolo[2,3-b]pyridin-3-yl]methylene}-4-hydroxy-1- | | | C25H16N2O4 + H⁺, |
| | benzofuran-3(2H)-one | | | 409.11828; found |
| | | | | 409.11775; |
| 86 | (2Z)-4-hydroxy-2-({4-[4-methoxy-3-(pyrrolidin-1- | | | calcd for |
| | ylsulfonyl)phenyl]-1-methyl-1H-pyrrolo[2,3-b]pyridin- | | | C28H25N3O6S + H⁺, |
| | 3-yl}methylene)-1-benzofuran-3(2H)-one | | | 532.15368; |
| | | | | found 532.15249; |
| 87 | (2Z)-4,6-dihydroxy-2-{[1-methyl-4-(4- | | | calcd for |
| | phenoxyphenyl)-1H-pyrrolo[2,3-b]pyridin-3- | | | C29H20N2O5 + H⁺, |
| | yl]methylene}-1-benzofuran-3(2H)-one | | | 477.14450; found |
| | | | | 477.14359; |
| 87 | (2Z)-4,6-dihydroxy-2-({4-[4-methoxy-3-(morpholin-4- | | | calcd for |
| | ylsulfonyl)phenyl]-1-methyl-1H-pyrrolo[2,3-b]pyridin- | | | C28H25N3O8S + H⁺, |
| | 3-yl}methylene)-1-benzofuran-3(2H)-one | | | 564.14351; |
| | | | | found 564.14169; |
| 89 | (2Z)-4,6-dihydroxy-2-({4-[4-methoxy-3-(pyrrolidin-1- | | | calcd for |
| | ylsulfonyl)phenyl]-1-methyl-1H-pyrrolo[2,3-b]pyridin- | | | C28H25N3O7S + H⁺, |
| | 3-yl}methylene)-1-benzofuran-3(2H)-one | | | 548.14860; |
| | | | | found 548.14713; |
| 90 | N-benzyl-4-{3-[(Z)-(4-hydroxy-3-oxo-1-benzofuran- | | | calcd for |
| | 2(3H)-ylidene)methyl]-1-methyl-1H-pyrrolo[2,3- | | | C30H23N3O5S + H⁺, |
| | b]pyridin-4-yl}benzenesulfonamide | | | 538.14312; |
| | | | | found 538.14179; |
| 91 | (2Z)-4-hydroxy-2-({1-methyl-4-[4-(pyrrolidin-1- | | | calcd for |
| | ylcarbonyl)phenyl]-1H-pyrrolo[2,3-b]pyridin-3- | | | C28H23N3O4 + H⁺, |
| | yl}methylene)-1-benzofuran-3(2H)-one | | | 466.17613; found |
| | | | | 466.17515; |
| 92 | (2Z)-4,6-dihydroxy-2-({1-methyl-4-[4-(pyrrolidin-1- | | | calcd for |
| | ylcarbonyl)phenyl]-1H-pyrrolo[2,3-b]pyridin-3- | | | C28H23N3O5 + H⁺, |
| | yl}methylene)-1-benzofuran-3(2H)-one | | | 482.17105; found |
| | | | | 482.16974; |
| 93 | (2Z)-2-{[4-(2,6-dimethylmorpholin-4-yl)-1-methyl-1H- | decomp | 406.2 | calcd for |
| | pyrrolo[2,3-b]pyridin-3-yl]methylene}-4-hydroxy-1- | 280 | | C23H23N3O4 + H⁺, |
| | benzofuran-3(2H)-one | | | 406.17613; found |
| | | | | 406.1757; |
| 94 | (2Z)-4,6-dihydroxy-2-[(1-methyl-4-phenoxy-1H- | 330-332 | 401.1 | calcd for |
| | pyrrolo[2,3-b]pyridin-3-yl)methylene]-1-benzofuran- | | | C23H16N2O5 + H⁺, |
| | 3(2H)-one | | | 401.11320; found |
| | | | | 401.1128; |
| 95 | (2Z)-4-hydroxy-2-[(4-hydroxy-1-methyl-1H- | decomp | 309.1 | calcd for |
| | pyrrolo[2,3-b]pyridin-3-yl)methylene]-1-benzofuran- | 325 | | C17H12N2O4 + H⁺, |
| | 3(2H)-one | | | 309.08698; found |
| | | | | 309.0868; |
| 96 | N-(4-fluorophenyl)-3-{3-[(Z)-(4-hydroxy-3-oxo-1- | | | calcd for |
| | benzofuran-2(3H)-ylidene)methyl]-1-methyl-1H- | | | C30H20FN3O4 + H⁺, |
| | pyrrolo[2,3-b]pyridin-4-yl}benzamide | | | 506.15106; |
| | | | | found 506.15124; |
| 97 | 3-{3-[(Z)-(4,6-dihydroxy-3-oxo-1-benzofuran-2(3H)- | | | calcd for |
| | ylidene)methyl]-1-methyl-1H-pyrrolo[2,3-b]pyridin-4- | | | C30H20FN3O5 + H⁺, |
| | yl}-N-(4-fluorophenyl)benzamide | | | 522.14598; |
| | | | | found 522.14432; |
| 98 | 5-{3-[(Z)-(4,6-dihydroxy-3-oxo-1-benzofuran-2(3H)- | | | calcd for |
| | ylidene)methyl]-1-methyl-1H-pyrrolo[2,3-b]pyridin-4- | | | C30H20FN3O5 + H⁺, |
| | yl}-2-fluoro-N-phenylbenzamide | | | 522.14598; |
| | | | | found 522.14458; |
| 99 | 1-methyl-3-{(2Z)-2-[(1-methyl-4-phenyl-1H- | 273-5 | 425.3 | calcd for |
| | pyrrolo[2,3-b]pyridin-3-yl)methylene]-3-oxo-2,3- | | | C25H20N4O3 + H⁺, |
| | dihydro-1-benzofuran-4-yl}urea | | | 425.16082; found |
| | | | | 425.1611; |
| 100 | (2Z)-2-{[4-(3,5-dimethylpiperidin-1-yl)-1-methyl-1H- | decomp | 404.2 | calcd for |
| | pyrrolo[2,3-b]pyridin-3-yl]methylene}-4-hydroxy-1- | 235-240 | | C24H25N3O3 + H⁺, |
| | benzofuran-3(2H)-one | | | 404.19687; found |
| | | | | 404.1973; |
| 101 | (2Z)-2-{[4-(2,5-diazabicyclo[2.2.1]hept-2-yl)-1- | decomp | 389.3 | calcd for |
| | methyl-1H-pyrrolo[2,3-b]pyridin-3-yl]methylene}-4- | 325 | | C22H20N4O3 + H⁺, |
| | hydroxy-1-benzofuran-3(2H)-one | | | 389.16082; found |
| | | | | 389.1609; |
| 102 | (2Z)-4-hydroxy-2-[(1-methyl-4-thiomorpholin-4-yl- | decomp | 394.1 | calcd for |
| | 1H-pyrrolo[2,3-b]pyridin-3-yl)methylene]-1- | 285-290 | | C21H19N3O3S + H⁺, |
| | benzofuran-3(2H)-one | | | 394.12199; |
| | | | | found 394.1221; |
| 103 | (2Z)-4-hydroxy-2-({1-methyl-4-[3-(pyrrolidin-1- | 233-235 | 466.3 | calcd for |
| | ylcarbonyl)phenyl]-1H-pyrrolo[2,3-b]pyridin-3- | | | C28H23N3O4 + H⁺, |
| | yl}methylene)-1-benzofuran-3(2H)-one | | | 466.17613; found |
| | | | | 466.17476; |
| 104 | (2Z)-4-hydroxy-2-({1-methyl-4-[4-(pyrrolidin-1- | 223-225 | 474.3 | calcd for |
| | ylcarbonyl)piperazin-1-yl]-1H-pyrrolo[2,3-b]pyridin-3- | | | C26H27N5O4 + H⁺, |
| | yl}methylene)-1-benzofuran-3(2H)-one | | | 474.21358; found |
| | | | | 474.21324; |
| 105 | (2Z)-4-hydroxy-2-{[1-methyl-4-(1,3,3-trimethyl-6- | 220-223 | 444.2 | calcd for |
| | azabicyclo[3.2.1]oct-6-yl)-1H-pyrrolo[2,3-b]pyridin-3- | | | C27H29N3O3 + H⁺, |
| | yl]methylene}-1-benzofuran-3(2H)-one | | | 444.22817; found |
| | | | | 444.2283; |
| 106 | (2Z)-4-hydroxy-2-({1-methyl-4-[4- | decomp | 437.1 | calcd for |
| | (trifluoromethyl)phenyl]-1H-pyrrolo[2,3-b]pyridin-3- | 244-254 | | C24H15F3N2O3 + H⁺, |
| | yl}methylene)-1-benzofuran-3(2H)-one | | | 437.11075; |
| | | | | found 437.1107; |
| 107 | (2Z)-4-hydroxy-2-({1-methyl-4-[3- | 262-265 | 437.1 | calcd for |
| | (trifluoromethyl)phenyl]-1H-pyrrolo[2,3-b]pyridin-3- | | | C24H15F3N2O3 + H⁺, |
| | yl}methylene)-1-benzofuran-3(2H)-one | | | 437.11075; |
| | | | | found 437.1106; |
| 108 | (2Z)-4-amino-2-[(1-methyl-4-phenyl-1H-pyrrolo[2,3- | 262-8 | | calcd for |
| | b]pyridin-3-yl)methylene]-1-benzofuran-3(2H)-one | | | C23H17N3O2 + H⁺, |
| | | | | 368.13935; found |
| | | | | 368.13884; |
| 109 | (2Z)-4-hydroxy-2-({1-methyl-4-[3-(pyrrolidin-1- | 235-40 | | calcd for |
| | ylcarbonyl)piperidin-1-yl]-1H-pyrrolo[2,3-b]pyridin-3- | | | C27H28N4O4 + H⁺, |
| | yl}methylene)-1-benzofuran-3(2H)-one | | | 473.21833; found |
| | | | | 473.21653; |
| 110 | (2Z)-4-hydroxy-2-({1-methyl-4-[3- | 230-233 | 453.1 | calcd for |
| | (trifluoromethoxy)phenyl]-1H-pyrrolo[2,3-b]pyridin-3- | | | C24H15F3N2O4 + H⁺, |
| | yl}methylene)-1-benzofuran-3(2H)-one | | | 453.10567; |
| | | | | found 453.1055; |
| 111 | 4-{3-[(Z)-(4-hydroxy-3-oxo-1-benzofuran-2(3H)- | 212-218 | 440.2 | calcd for |
| | ylidene)methyl]-1-methyl-1H-pyrrolo[2,3-b]pyridin-4- | | | C26H21N3O4 + H⁺, |
| | yl}-N,N-dimethylbenzamide | | | 440.16048; found |
| | | | | 440.1603; |
| 112 | (2Z)-2-({4-[3-(dimethylamino)pyrrolidin-1-yl]-1- | decomp | 405.3 | calcd for |
| | methyl-1H-pyrrolo[2,3-b]pyridin-3-yl}methylene)-4- | 205 | | C23H24N4O3 + H⁺, |
| | hydroxy-1-benzofuran-3(2H)-one | | | 405.19212; found |
| | | | | 405.1920; |
| 113 | (2Z)-2-[(4-{4-[(dimethylamino)methyl]phenyl}-1- | decomp | 426.1 | calcd for |
| | methyl-1H-pyrrolo[2,3-b]pyridin-3-yl)methylene]-4- | 340-350 | | C26H23N3O3 + H⁺, |
| | hydroxy-1-benzofuran-3(2H)-one | | | 426.18122; found |
| | | | | 426.1815; |
| 114 | 1-{3-[(Z)-(4-hydroxy-3-oxo-1-benzofuran-2(3H)- | decomp | 419.1 | calcd for |
| | ylidene)methyl]-1-methyl-1H-pyrrolo[2,3-b]pyridin-4- | 280-287 | | C23H22N4O4 + H⁺, |
| | yl}piperidine-4-carboxamide | | | 419.17138; found |
| | | | | 419.1713; |
| 115 | (2Z)-2-({4-[4-(aminomethyl)piperidin-1-yl]-1-methyl- | decomp | 405.2 | calcd for |
| | 1H-pyrrolo[2,3-b]pyridin-3-yl}methylene)-4-hydroxy- | 230-238 | | C23H24N4O3 + H⁺, |
| | 1-benzofuran-3(2H)-one | | | 405.19212; found |
| | | | | 405.1922; |
| 116 | (2Z)-2-[(1-ethyl-4-phenyl-1H-pyrrolo[2,3-b]pyridin-3- | | 383.2 | calcd for |
| | yl)methylene]-4-hydroxy-1-benzofuran-3(2H)-one | | | C24H18N2O3 + H⁺, |
| | | | | 383.13902; found |
| | | | | 383.14011; |
| 117 | (2Z)-4-hydroxy-2-{[1-methyl-4-(2-oxa-5- | 258-63 | 390.2 | calcd for |
| | azabicyclo[2.2.1]hept-5-yl)-1H-pyrrolo[2,3-b]pyridin- | | | C22H19N3O4 + H⁺, |
| | 3-yl]methylene}-1-benzofuran-3(2H)-one | | | 390.14483; found |
| | | | | 390.14588; |
| 118 | (2Z)-2-[(4-{4-[3-(dimethylamino)propyl]piperazin-1- | 263-8 | 462.3 | calcd for |
| | yl}-1-methyl-1H-pyrrolo[2,3-b]pyridin-3- | | | C26H31N5O3 + H⁺, |
| | yl)methylene]-4-hydroxy-1-benzofuran-3(2H)-one | | | 462.24997; found |
| | | | | 462.25115; |
| 119 | 1-{(2Z)-2-[(5-methoxy-1H-pyrrolo[2,3-b]pyridin-3- | | 365 | |
| | yl)methylene]-3-oxo-2,3-dihydro-1-benzofuran-5-yl}- | | | |
| | 3-methylurea | | | |
| 120 | 1-[(2Z)-2-({5-methoxy-1-[2-(4-methylpiperazin-1- | | 491.3 | |
| | yl)ethyl]-1H-pyrrolo[2,3-b]pyridin-3-yl}methylene)-3- | | | |
| | oxo-2,3-dihydro-1-benzofuran-5-yl]-3-methylurea | | | |
| 121 | 3-{3-[(Z)-(4-hydroxy-3-oxo-1-benzofuran-2(3H)- | 220-223 | 440.1 | calcd for |
| | ylidene)methyl]-1-methyl-1H-pyrrolo[2,3-b]pyridin-4- | | | C26H21N3O4 + H⁺, |
| | yl}-N,N-dimethylbenzamide | | | 440.16048; found |
| | | | | 440.1602; |
| 122 | (2Z)-2-[(4-{3-[(dimethylamino)methyl]phenyl}-1- | decomp | 426.1 | calcd for |
| | methyl-1H-pyrrolo[2,3-b]pyridin-3-yl)methylene]-4- | 308-315 | | C26H23N3O3 + H⁺, |
| | hydroxy-1-benzofuran-3(2H)-one | | | 426.18122; found |
| | | | | 426.1808; |
| 123 | (2Z)-4-hydroxy-2-({4-[4-methoxy-3-(piperidin-1- | | | calcd for |
| | ylsulfonyl)phenyl]-1-methyl-1H-pyrrolo[2,3-b]pyridin- | | | C29H27N3O6S + H⁺, |
| | 3-yl}methylene)-1-benzofuran-3(2H)-one | | | 546.16933; |
| | | | | found 546.16879; |
| 124 | (2Z)-4-hydroxy-2-[(1-methyl-4-{4-[(4- | | | calcd for |
| | methylpiperazin-1-yl)carbonyl]phenyl}-1H- | | | C29H26N4O4 + H⁺, |
| | pyrrolo[2,3-b]pyridin-3-yl)methylene]-1-benzofuran- | | | 495.20268; found |
| | 3(2H)-one | | | 495.2015; |
| 125 | (2Z)-4-hydroxy-2-{[4-(3-hydroxyphenyl)-1-methyl- | | | calcd for |
| | 1H-pyrrolo[2,3-b]pyridin-3-yl]methylene}-1- | | | C23H16N2O4 + H⁺, |
| | benzofuran-3(2H)-one | | | 385.11828; found |
| | | | | 385.11795; |
| 126 | (2Z)-4-hydroxy-2-{[1-methyl-4-(4-methyl-3,4- | | | calcd for |
| | dihydro-2H-1,4-benzoxazin-7-yl)-1H-pyrrolo[2,3- | | | C26H21N3O4 + H⁺, |
| | b]pyridin-3-yl]methylene}-1-benzofuran-3(2H)-one | | | 440.16048; found |
| | | | | 440.16048; |
| 127 | (2Z)-2-({4-[4-(2-furyl)phenyl]-1-methyl-1H- | | | calcd for |
| | pyrrolo[2,3-b]pyridin-3-yl}methylene)-4-hydroxy-1- | | | C27H18N2O4 + H⁺, |
| | benzofuran-3(2H)-one | | | 435.13393; found |
| | | | | 435.13391; |
| 128 | (2Z)-4-hydroxy-2-({1-methyl-4-[4-(morpholin-4- | | | calcd for |
| | ylcarbonyl)phenyl]-1H-pyrrolo[2,3-b]pyridin-3- | | | C28H23N3O5 + H⁺, |
| | yl}methylene)-1-benzofuran-3(2H)-one | | | 482.17105; found |
| | | | | 482.17066; |
| 129 | (2Z)-4-hydroxy-2-({1-methyl-4-[4-(4-methylpiperazin- | 239-42 | 474.3 | calcd for |
| | 1-yl)piperidin-1-yl]-1H-pyrrolo[2,3-b]pyridin-3- | | | C27H31N5O3 + H⁺, |
| | yl}methylene)-1-benzofuran-3(2H)-one | | | 474.24997; found |
| | | | | 474.24861; |
| 130 | (2Z)-4-hydroxy-2-({1-methyl-4-[4-(morpholin-4- | | | calcd for |
| | ylmethyl)phenyl]-1H-pyrrolo[2,3-b]pyridin-3- | | | C28H25N3O4 + H⁺, |
| | yl}methylene)-1-benzofuran-3(2H)-one | | | 468.19178; found |
| | | | | 468.19254; |
| 131 | 1-methyl-3-[(2Z)-2-{[1-methyl-4-(8-oxa-3- | 247-55 | 460.2 | calcd for |
| | azabicyclo[3.2.1]oct-3-yl)-1H-pyrrolo[2,3-b]pyridin-3- | | | C28H25N3O4 + H⁺, |
| | yl]methylene}-3-oxo-2,3-dihydro-1-benzofuran-5- | | | 460.19793; found |
| | yl]urea | | | 460.19674; |
| 132 | 4-{3-[(Z)-(4-hydroxy-3-oxo-1-benzofuran-2(3H)- | decomp | 426.2 | calcd for |
| | ylidene)methyl]-1-methyl-1H-pyrrolo[2,3-b]pyridin-4- | 175 | | C25H19N3O4 + H⁺, |
| | yl}-N-methylbenzamide | | | 426.14483; found |
| | | | | 426.1447; |
| 133 | 1-(4-{3-[(Z)-(4-hydroxy-3-oxo-1-benzofuran-2(3H)- | dec 298-305 | | calcd for |
| | ylidene)methyl]-1-methyl-1H-pyrrolo[2,3-b]pyridin-4- | | | C25H20N4O4 + H⁺, |
| | yl}phenyl)-3-methylurea | | | 441.15573; found |
| | | | | 441.1556; |
| 134 | (2Z)-2-{[4-(4-acetylphenyl)-1-methyl-1H-pyrrolo[2,3- | 250-253 | | calcd for |
| | b]pyridin-3-yl]methylene}-4-hydroxy-1-benzofuran- | | | C25H18N2O4 + H⁺, |
| | 3(2H)-one | | | 411.13393; found |
| | | | | 411.1340; |
| 135 | (2Z)-4-hydroxy-2-{[1-methyl-4-(4-piperazin-1- | | 453.1 | calcd for |
| | ylphenyl)-1H-pyrrolo[2,3-b]pyridin-3-yl]methylene}-1- | | | C27H24N4O3 + H⁺, |
| | benzofuran-3(2H)-one | | | 453.19212; found |
| | | | | 453.19281; |
| 136 | (2Z)-4-hydroxy-2-({1-methyl-4-[4-(piperazin-1- | | | calcd for |
| | ylcarbonyl)phenyl]-1H-pyrrolo[2,3-b]pyridin-3- | | | C28H24N4O4 + H⁺, |
| | yl}methylene)-1-benzofuran-3(2H)-one | | | 481.18703; found |
| | | | | 481.18617; |
| 137 | 2-fluoro-5-{3-[(Z)-(4-hydroxy-3-oxo-1-benzofuran- | | | calcd for |
| | 2(3H)-ylidene)methyl]-1-methyl-1H-pyrrolo[2,3- | | | C30H20FN3O4 + H⁺, |
| | b]pyridin-4-yl}-N-phenylbenzamide | | | 506.15106; |
| | | | | found 506.15039; |
| 138 | 3-{3-[(Z)-(4-hydroxy-3-oxo-1-benzofuran-2(3H)- | | | calcd for |
| | ylidene)methyl]-1-methyl-1H-pyrrolo[2,3-b]pyridin-4- | | | C30H21N3O4 + H⁺, |
| | yl}-N-phenylbenzamide | | | 488.16048; found |
| | | | | 488.16022; |
| 139 | (2Z)-2-{[4-(4-aminopiperidin-1-yl)-1-methyl-1H- | 260-260 | 391.2 | calcd for |
| | pyrrolo[2,3-b]pyridin-3-yl]methylene}-4-hydroxy-1- | | | C22H22N4O3 + H⁺, |
| | benzofuran-3(2H)-one | | | 391.17647; found |
| | | | | 391.1768; |
| 140 | 3-(4-{3-[(Z)-(4-hydroxy-3-oxo-1-benzofuran-2(3H)- | 235-238 | 455.2 | calcd for |
| | ylidene)methyl]-1-methyl-1H-pyrrolo[2,3-b]pyridin-4- | | | C26H22N4O4 + H⁺, |
| | yl}phenyl)-1,1-dimethylurea | | | 455.17138; found |
| | | | | (ESI, [M + H]+), |
| | | | | 455.1712; |
| 141 | 2-{3-[(Z)-(4-hydroxy-3-oxo-1-benzofuran-2(3H)- | 293-5 | 440.2 | calcd for |
| | ylidene)methyl]-1-methyl-1H-pyrrolo[2,3-b]pyridin-4- | | | C26H21N3O4 + H⁺, |
| | yl}-N,N-dimethylbenzamide | | | 440.16048; found |
| | | | | (ESI, [M + H]+), |
| | | | | 440.1601; |
| 142 | (2Z)-4-hydroxy-2-{[1-methyl-4-(4-piperidin-1- | | | calcd for |
| | ylphenyl)-1H-pyrrolo[2,3-b]pyridin-3-yl]methylidene}- | | | C28H25N3O3 + H⁺, |
| | 1-benzofuran-3(2H)-one | | | 452.19687; found |
| | | | | 452.1969; |
| 143 | (2Z)-4-hydroxy-2-{[1-methyl-4-(4-pyrrolidin-1- | | | calcd for |
| | ylphenyl)-1H-pyrrolo[2,3-b]pyridin-3-yl]methylidene}- | | | C27H23N3O3 + H⁺, |
| | 1-benzofuran-3(2H)-one | | | 438.18122; found |
| | | | | 438.1811; |
| 144 | (2Z)-4-hydroxy-2-({1-methyl-4-[4-(8-oxa-3- | 296-298 | 508.1 | calcd for |
| | azabicyclo[3.2.1]oct-3-ylcarbonyl)phenyl]-1H- | | | C30H25N3O5 + H⁺, |
| | pyrrolo[2,3-b]pyridin-3-yl}methylidene)-1- | | | 508.18670; found |
| | benzofuran-3(2H)-one | | | 508.1864; |
| 145 | 1-{3-[(Z)-(4-hydroxy-3-oxo-1-benzofuran-2(3H)- | decomp | 447.1 | calcd for |
| | ylidene)methyl]-1-methyl-1H-pyrrolo[2,3-b]pyridin-4- | 185-195 | | C25H26N4O4 + H⁺, |
| | yl}-N,N-dimethylpiperidine-4-carboxamide | | | 447.20268; found |
| | | | | 447.2032; |
| 146 | (2Z)-4-hydroxy-2-{[4-(4-{[4-(2- | | 525.2 | calcd for |
| | hydroxyethyl)piperazin-1-yl]carbonyl}phenyl)-1- | | | C30H28N4O5 + H⁺, |
| | methyl-1H-pyrrolo[2,3-b]pyridin-3-yl]methylidene}-1- | | | 525.21325; found |
| | benzofuran-3(2H)-one | | | 525.2126; |
| 147 | 4-{3-[(Z)-(4-hydroxy-3-oxo-1-benzofuran-2(3H)- | | 470.1 | calcd for |
| | ylidene)methyl]-1-methyl-1H-pyrrolo[2,3-b]pyridin-4- | | | C27H23N3O5 + H⁺, |
| | yl}-N-(3-hydroxypropyl)benzamide | | | 470.17105; found |
| | | | | 470.1703; |
| 148 | N-(2-hydroxyethyl)-1-{3-[(Z)-(4-hydroxy-3-oxo-1- | 232-42 | 463.1 | calcd for |
| | benzofuran-2(3H)-ylidene)methyl]-1-methyl-1H- | | | C25H26N4O5 + H⁺, |
| | pyrrolo[2,3-b]pyridin-4-yl}piperidine-4-carboxamide | | | 463.19760; found |
| | | | | 463.19675; |
| 149 | 4-{3-[(Z)-(4-hydroxy-3-oxo-1-benzofuran-2(3H)- | | 470.2 | |
| | ylidene)methyl]-1-methyl-1H-pyrrolo[2,3-b]pyridin-4- | | | |
| | yl}-N-(2-methoxyethyl)benzamide | | | |
| 150 | (2Z)-4-hydroxy-2-({1-methyl-4-[4-(morpholin-4- | 358-61 | 489.2 | |
| | ylcarbonyl)piperidin-1-yl]-1H-pyrrolo[2,3-b]pyridin-3- | | | |
| | yl}methylidene)-1-benzofuran-3(2H)-one | | | |
| 151 | (2Z)-4-hydroxy-2-{[1-methyl-4-(3-oxa-8- | 284-91 | 404.2 | |
| | azabicyclo[3.2.1]oct-8-yl)-1H-pyrrolo[2,3-b]pyridin-3- | | | |
| | yl]methylidene}-1-benzofuran-3(2H)-one | | | |
| 152 | (2Z)-4-hydroxy-2-({1-methyl-4-[4-(pyrrolidin-1- | 191-207 | 473.2 | |
| | ylcarbonyl)piperidin-1-yl]-1H-pyrrolo[2,3-b]pyridin-3- | | | |
| | yl}methylidene)-1-benzofuran-3(2H)-one | | | |
| 153 | (2Z)-4-hydroxy-2-{[1-methyl-4-(5-methyl-2,5- | dec 220 | 403.2 | calcd for |
| | diazabicyclo[2.2.1]hept-2-yl)-1H-pyrrolo[2,3- | | | C23H22N4O3 + H⁺, |
| | b]pyridin-3-yl]methylidene}-1-benzofuran-3(2H)-one | | | 403.17647; found |
| | | | | 403.1765; |
| 154 | N-{(2Z)-2-[(1-methyl-4-phenyl-1H-pyrrolo[2,3- | 332-335 | 410.2 | calcd for |
| | b]pyridin-3-yl)methylidene]-3-oxo-2,3-dihydro-1- | | | C25H19N3O3 + H⁺, |
| | benzofuran-5-yl}acetamide | | | 410.14992; found |
| | | | | 410.1498; |
| 155 | N-[(2Z)-2-{[1-methyl-4-(8-oxa-3-azabicyclo[3.2.1]oct- | dec 275-285 | 445.2 | calcd for |
| | 3-yl)-1H-pyrrolo[2,3-b]pyridin-3-yl]methylidene}-3- | | | C25H24N4O4 + H⁺, |
| | oxo-2,3-dihydro-1-benzofuran-5-yl]acetamide | | | 445.18703; found |
| | | | | 445.1868; |
| 156 | 2,2,2-trifluoro-N-{(2Z)-2-[(1-methyl-4-phenyl-1H- | 295-302 | 464.1 | calcd for |
| | pyrrolo[2,3-b]pyridin-3-yl)methylidene]-3-oxo-2,3- | | | C25H16F3N3O3 + H⁺, |
| | dihydro-1-benzofuran-5-yl}acetamide | | | 464.12165; |
| | | | | found 464.1212; |
| 157 | (2Z)-4-hydroxy-2-({1-methyl-4-[4-(8-oxa-3- | dec 252-260 | 480.1 | calcd for |
| | azabicyclo[3.2.1]oct-3-yl)phenyl]-1H-pyrrolo[2,3- | | | C29H25N3O4 + H⁺, |
| | b]pyridin-3-yl}methylidene)-1-benzofuran-3(2H)-one | | | 480.19178; found |
| | | | | 480.1914; |
| 158 | N-[2-(dimethylamino)ethyl]-4-{3-[(Z)-(4-hydroxy-3- | | 483.1 | calcd for |
| | oxo-1-benzofuran-2(3H)-ylidene)methyl]-1-methyl- | | | C28H26N4O4 + H⁺, |
| | 1H-pyrrolo[2,3-b]pyridin-4-yl}benzamide | | | 483.20268; found |
| | | | | 483.2024; |
| 159 | (2Z)-4-[2-(dimethylamino)ethoxy]-2-[(1-methyl-4- | | 440.2 | |
| | phenyl-1H-pyrrolo[2,3-b]pyridin-3-yl)methylidene]-1- | | | |
| | benzofuran-3(2H)-one | | | |
| 160 | ethyl 4-{3-[(Z)-(4-hydroxy-3-oxo-1-benzofuran- | | 441.2 | |
| | 2(3H)-ylidene)methyl]-1-methyl-1H-pyrrolo[2,3- | | | |
| | b]pyridin-4-yl}benzoate | | | |
| 161 | 1,1-dimethyl-3-{(2Z)-2-[(1-methyl-4-phenyl-1H- | 275-280 | 439.2 | calcd for |
| | pyrrolo[2,3-b]pyridin-3-yl)methylidene]-3-oxo-2,3- | | | C26H22N4O3 + H⁺, |
| | dihydro-1-benzofuran-5-yl}urea | | | 439.17647; found |
| | | | | 439.1763; |
| 162 | N-{(2Z)-2-[(1-methyl-4-phenyl-1H-pyrrolo[2,3- | 257-262 | 446.1 | calcd for |
| | b]pyridin-3-yl)methylidene]-3-oxo-2,3-dihydro-1- | | | C24H19N3O4S + H⁺, |
| | benzofuran-5-yl}methanesulfonamide | | | 446.11690; |
| | | | | found 446.1169; |
| 163 | N,N-dimethyl-1-{1-methyl-3-[(Z)-{3-oxo-5- | 236-240 | 542.2 | calcd for |
| | [(trifluoroacetyl)amino]-1-benzofuran-2(3H)- | | | C27H26F3N5O4 + H⁺, |
| | ylidene}methyl]-1H-pyrrolo[2,3-b]pyridin-4- | | | 542.20096; |
| | yl}piperidine-4-carboxamide | | | found 542.2012; |
| 164 | (2Z)-4-hydroxy-2-[(1-methyl-4-{4-[(4- | 239-44 | 502.3 | calcd for |
| | methylpiperazin-1-yl)carbonyl]piperidin-1-yl}-1H- | | | C28H31N5O4 + H⁺, |
| | pyrrolo[2,3-b]pyridin-3-yl)methylidene]-1- | | | 502.24488; found |
| | benzofuran-3(2H)-one | | | 502.24425; |
| 165 | (2Z)-2-{[4-(6,8-dioxa-3-azabicyclo[3.2.1]oct-3-yl)-1- | — | 406.2 | calcd for |
| | methyl-1H-pyrrolo[2,3-b]pyridin-3-yl]methylidene}-4- | | | C22H19N3O5 + H⁺, |
| | hydroxy-1-benzofuran-3(2H)-one | | | 406.13975; found |
| | | | | 406.13912; |
| 166 | 1-[(2Z)-2-{[4-(4-{[4-(2-hydroxyethyl)piperazin-1- | — | 581.4 | calcd for |
| | yl]carbonyl}phenyl)-1-methyl-1H-pyrrolo[2,3- | | | C32H32N6O5 + H⁺, |
| | b]pyridin-3-yl]methylidene}-3-oxo-2,3-dihydro-1- | | | 581.25070; found |
| | benzofuran-5-yl]-3-methylurea | | | 581.2492; |
| 167 | 1-[(2Z)-2-{[1-methyl-4-(8-oxa-3-azabicyclo[3.2.1]oct- | 226-30 | 523.3 | calcd for |
| | 3-yl)-1H-pyrrolo[2,3-b]pyridin-3-yl]methylidene}-3- | | | C29H26N6O4 + H⁺, |
| | oxo-2,3-dihydro-1-benzofuran-5-yl]-3-pyridin-3- | | | 523.20883; found |
| | ylurea | | | 523.20794; |
| 168 | N,N-dimethyl-1-{1-methyl-3-[(Z)-{5- | decomp | 503.3 | calcd for |
| | [(methylcarbamoyl)amino]-3-oxo-1-benzofuran- | 203 | | C27H30N6O4 + H⁺, |
| | 2(3H)-ylidene}methyl]-1H-pyrrolo[2,3-b]pyridin-4- | | | 503.24013; found |
| | yl}piperidine-4-carboxamide | | | 503.2400; |
| 169 | 1-{3-[(Z)-(5-bromo-3-oxo-1-benzofuran-2(3H)- | decomp | 509.2 | calcd for |
| | ylidene)methyl]-1-methyl-1H-pyrrolo[2,3-b]pyridin-4- | 241 | | C25H25BrN4O3 + H⁺, |
| | yl}-N,N-dimethylpiperidine-4-carboxamide | | | 509.11828; |
| | | | | found 509.1183; |
| 170 | N-[(2Z)-2-({4-[4-(dimethylcarbamoyl)piperidin-1-yl]- | decomp | 559.3 | calcd for |
| | 1-methyl-1H-pyrrolo[2,3-b]pyridin-3-yl}methylidene)- | 128 | | C30H34N6O5 + H⁺, |
| | 3-oxo-2,3-dihydro-1-benzofuran-5-yl]morpholine-4- | | | 559.26635; found |
| | carboxamide | | | 559.2663; |
| 171 | ethyl [(2Z)-2-({4-[4-(dimethylcarbamoyl)piperidin-1- | 162-167 | 518.3 | calcd for |
| | yl]-1-methyl-1H-pyrrolo[2,3-b]pyridin-3- | | | C28H31N5O5 + Na+, |
| | yl}methylidene)-3-oxo-2,3-dihydro-1-benzofuran-5- | | | 540.22174; |
| | yl]carbamate | | | found (ESI, [M + Na]+ |
| | | | | Calc'd), 540.2217; |
| 172 | 1-[(2Z)-2-{[1-methyl-4-(8-oxa-3-azabicyclo[3.2.1]oct- | — | 522.3 | calcd for |
| | 3-yl)-1H-pyrrolo[2,3-b]pyridin-3-yl]methylidene}-3- | — | 522.3 | C30H27N5O4 + H⁺, |
| | oxo-2,3-dihydro-1-benzofuran-5-yl]-3-phenylurea | | | 522.21358; found |
| | | | | 522.2119; |
| 173 | 1-[(2Z)-2-({1-methyl-4-[4-(morpholin-4- | 246-51 | 608.4 | calcd for |
| | ylcarbonyl)piperidin-1-yl]-1H-pyrrolo[2,3-b]pyridin-3- | | | C33H33N7O5 + H⁺, |
| | yl}methylidene)-3-oxo-2,3-dihydro-1-benzofuran-5- | | | 608.26159; found |
| | yl]-3-pyridin-3-ylurea | | | 608.26108; |
| 174 | 1-methyl-3-[(2Z)-2-({1-methyl-4-[4-(morpholin-4- | 235-40 | 545.3 | calcd for |
| | ylcarbonyl)piperidin-1-yl]-1H-pyrrolo[2,3-b]pyridin-3- | | | C29H32N6O5 + H⁺, |
| | yl}methylidene)-3-oxo-2,3-dihydro-1-benzofuran-5- | | | 545.25070; found |
| | yl]urea | | | 545.24909; |
| 175 | 4-{3-[(Z)-(4-hydroxy-3-oxo-1-benzofuran-2(3H)- | — | — | calcd for |
| | ylidene)methyl]-1-methyl-1H-pyrrolo[2,3-b]pyridin-4- | | | C28H25N3O5 + H⁺, |
| | yl}-N-(2-methoxyethyl)-N-methylbenzamide | | | 484.18670; found |
| | | | | 484.18654; |
| 176 | (2Z)-2-[(1-methyl-4-phenyl-1H-pyrrolo[2,3-b]pyridin- | 329-42 | 397.2 | calcd for |
| | 3-yl)methylidene]-3-oxo-2,3-dihydro-1-benzofuran-5- | | | C24H16N2O4 + H⁺, |
| | carboxylic acid | | | 397.11828; found |
| | | | | 397.118; |
| 177 | 1-[(2Z)-2-{[1-ethyl-4-(4-{[4-(2- | | 658 | calcd for |
| | hydroxyethyl)piperazin-1-yl]carbonyl}phenyl)-1H- | | | C37H35N7O5 + H⁺, |
| | pyrrolo[2,3-b]pyridin-3-yl]methylidene}-3-oxo-2,3- | | | 658.27724; found |
| | dihydro-1-benzofuran-5-yl]-3-pyridin-3-ylurea | | | 658.27562; |
| 178 | 1-[(2Z)-2-({1-methyl-4-[4-(morpholin-4- | — | 601.4 | calcd for |
| | ylcarbonyl)phenyl]-1H-pyrrolo[2,3-b]pyridin-3- | | | C34H28N6O5 + H⁺, |
| | yl}methylidene)-3-oxo-2,3-dihydro-1-benzofuran-5- | | | 601.21940; found |
| | yl]-3-pyridin-3-ylurea | | | 601.21851; |
| 179 | 1-methyl-3-[(2Z)-2-({1-methyl-4-[4-(morpholin-4- | — | 538.3 | calcd for |
| | ylcarbonyl)phenyl]-1H-pyrrolo[2,3-b]pyridin-3- | | | C30H27N5O5 + H⁺, |
| | yl}methylidene)-3-oxo-2,3-dihydro-1-benzofuran-5- | | | 538.20850; found |
| | yl]urea | | | 538.20789; |
| 180 | 4-{3-[(Z)-(4-hydroxy-3-oxo-1-benzofuran-2(3H)- | — | 474.2 | calcd for |
| | ylidene)methyl]-1-methyl-1H-pyrrolo[2,3-b]pyridin-4- | | | C25H21N3O5S + H⁺, |
| | yl}-N,N-dimethylbenzenesulfonamide | | | 476.12747; |
| | | | | found 476.12741; |
| 181 | (2Z)-4-hydroxy-2-({1-methyl-4-[4-(piperidin-1- | — | — | calcd for |
| | ylsulfonyl)phenyl]-1H-pyrrolo[2,3-b]pyridin-3- | | | C28H25N3O5S + H⁺, |
| | yl}methylidene)-1-benzofuran-3(2H)-one | | | 516.15877; |
| | | | | found 516.15811; |
| 182 | N-(2-hydroxyethyl)-4-{3-[(Z)-(4-hydroxy-3-oxo-1- | — | 454.2 | calcd for |
| | benzofuran-2(3H)-ylidene)methyl]-1-methyl-1H- | | | C26H21N3O5 + H⁺, |
| | pyrrolo[2,3-b]pyridin-4-yl}benzamide | | | 456.15540; found |
| | | | | 456.15528; |
| 183 | 1-{(2Z)-2-[(1-methyl-4-phenyl-1H-pyrrolo[2,3- | — | 488.3 | calcd for |
| | b]pyridin-3-yl)methylidene]-3-oxo-2,3-dihydro-1- | | | C29H21N5O3 + H⁺, |
| | benzofuran-5-yl}-3-pyridin-3-ylurea | | | 488.17172; found |
| | | | | 488.1714; |
| 184 | 1-[(2Z)-2-{[1-methyl-4-(4-methylpiperazin-1-yl)-1H- | — | — | calcd for |
| | pyrrolo[2,3-b]pyridin-3-yl]methlidene}-3-oxo-2,3- | | | C28H27N7O3 + H⁺, |
| | dihydro-1-benzofuran-5-yl]-3-pyridin-3-ylurea | | | 510.22481; found |
| | | | | 510.22434; |
| 185 | 1-{(2Z)-2-[(1-methyl-4-morpholin-4-yl-1H- | — | — | calcd for |
| | pyrrolo[2,3-b]pyridin-3-yl)methylidene]-3-oxo-2,3- | | | C27H24N6O4 + H⁺, |
| | dihydro-1-benzofuran-5-yl}-3-pyridin-3-ylurea | | | 497.19318; found |
| | | | | 497.19251; |
| 186 | (2Z)-2-{[1-ethyl-4-(4-{[4-(2-hydroxyethyl)piperazin-1- | — | 539.3 | calcd for |
| | yl]carbonyl}phenyl)-1H-pyrrolo[2,3-b]pyridin-3- | | | C31H30N4O5 + H⁺, |
| | yl]methylidene}-4-hydroxy-1-benzofuran-3(2H)-one | | | 539.22890; found |
| | | | | 539.22778; |
| 187 | (2Z)—N-[2-(dimethylamino)ethyl]-2-[(1-methyl-4- | 255-6 | 467.3 | — |
| | phenyl-1H-pyrrolo[2,3-b]pyridin-3-yl)methylidene]-3- | | | |
| | oxo-2,3-dihydro-1-benzofuran-5-carboxamide | | | |
| 188 | 4-methyl-N-{(2Z)-2-[(1-methyl-4-phenyl-1H- | 211-213 | 494.4 | calcd for |
| | pyrrolo[2,3-b]pyridin-3-yl)methylidene]-3-oxo-2,3- | | | C29H27N5O3 + H⁺, |
| | dihydro-1-benzofuran-5-yl}piperazine-1- | | | 494.21867; found |
| | carboxamide | | | 494.2187; |
| 189 | N,N-dimethyl-1-{1-methyl-3-[(Z)-{3-oxo-5-[(pyridin-3- | dec 165-175 | 566.4 | calcd for |
| | ylcarbamoyl)amino]-1-benzofuran-2(3H)- | | | C31H31N7O4 + H⁺, |
| | ylidene}methyl]-1H-pyrrolo[2,3-b]pyridin-4- | | | 566.25103; found |
| | yl}piperidine-4-carboxamide | | | 566.2510; |
| 190 | (2Z)-4-hydroxy-2-({1-methyl-4-[4-(pyrrolidin-1- | — | 502.3 | — |
| | ylsulfonyl)phenyl]-1H-pyrrolo[2,3-b]pyridin-3- | | | |
| | yl}methylidene)-1-benzofuran-3(2H)-one | | | |
| 191 | (2Z)-4-hydroxy-2-({1-methyl-4-[4-(morpholin-4- | — | 518.3 | — |
| | ylsulfonyl)phenyl]-1H-pyrrolo[2,3-b]pyridin-3- | | | |
| | yl}methylidene)-1-benzofuran-3(2H)-one | | | |
| 192 | 4-{3-[(Z)-(4-hydroxy-3-oxo-1-benzofuran-2(3H)- | — | 442.1 | — |
| | ylidene)methyl]-1-methyl-1H-pyrrolo[2,3-b]pyridin-4- | | | |
| | yl}-N-methoxybenzamide | | | |
| 193 | 1-[2-(dimethylamino)ethyl]-3-[(2Z)-2-{[1-methyl-4-(8- | 175-80 | 517.2 | — |
| | oxa-3-azabicyclo[3.2.1]oct-3-yl)-1H-pyrrolo[2,3- | | | |
| | b]pyridin-3-yl]methylidene}-3-oxo-2,3-dihydro-1- | | | |
| | benzofuran-5-yl]urea | | | |
| 194 | 1-[(2Z)-2-{[1-methyl-4-(8-oxa-3-azabicyclo[3.2.1]oct- | 170-2 | 572.2 | — |
| | 3-yl)-1H-pyrrolo[2,3-b]pyridin-3-yl]methylidene}-3- | | | |
| | oxo-2,3-dihydro-1-benzofuran-5-yl]-3-[2-(4- | | | |
| | methylpiperazin-1-yl)ethyl]urea | | | |
| 195 | N-(2-hydroxyethyl)-4-{3-[(Z)-(4-hydroxy-3-oxo-1- | — | 470.2 | — |
| | benzofuran-2(3H)-ylidene)methyl]-1-methyl-1H- | | | |
| | pyrrolo[2,3-b]pyridin-4-yl}-N-methylbenzamide | | | |
| 196 | 4-{3-[(Z)-(4-hydroxy-3-oxo-1-benzofuran-2(3H)- | — | 456.3 | — |
| | ylidene)methyl]-1-methyl-1H-pyrrolo[2,3-b]pyridin-4- | | | |
| | yl}-N-methoxy-N-methylbenzamide | | | |
| 197 | (2Z)-2-{[1-methyl-4-(8-oxa-3-azabicyclo[3.2.1]oct-3- | 285-89 | 432.2 | — |
| | yl)-1H-pyrrolo[2,3-b]pyridin-3-yl]methylidene}-3-oxo- | | | |
| | 2,3-dihydro-1-benzofuran-5-carboxylic acid | | | |
| 198 | 1-[(2Z)-2-{[1-methyl-4-(8-oxa-3-azabicyclo[3.2.1]oct- | 257-9 | 523.4 | — |
| | 3-yl)-1H-pyrrolo[2,3-b]pyridin-3-yl]methylidene}-3- | | | |
| | oxo-2,3-dihydro-1-benzofuran-5-yl]-3-pyridin-4- | | | |
| | ylurea | | | |
| 199 | N-(3-methoxypropyl)-N-methyl-1-[1-methyl-3-({3- | | 624.3 | |
| | oxo-5-[(pyridin-3-ylcarbamoyl)amino]-1-benzofuran- | | | |
| | 2(3H)-ylidene}methyl)-1H-pyrrolo[2,3-b]pyridin-4- | | | |
| | yl]piperidine-4-carboxamide | | | |
| 200 | N-(3-methoxypropyl)-1-[1-methyl-3-({3-oxo-5- | | 610.3 | |
| | [(pyridin-3-ylcarbamoyl)amino]-1-benzofuran-2(3H)- | | | |
| | ylidene}methyl)-1H-pyrrolo[2,3-b]pyridin-4- | | | |
| | yl]piperidine-4-carboxamide | | | |
| 201 | N-[2-(dimethylamino)-2-oxoethyl]-N-methyl-1-[1- | | 635.3 | |
| | methyl-3-({3-oxo-5-[(pyridin-3-ylcarbamoyl)amino]-1- | | | |
| | benzofuran-2(3H)-ylidene}methyl)-1H-pyrrolo[2,3- | | | |
| | b]pyridin-4-yl]piperidine-4-carboxamide | | | |
| 202 | N-(2-amino-2-oxoethyl)-1-[1-methyl-3-({3-oxo-5- | | 595.2 | |
| | [(pyridin-3-ylcarbamoyl)amino]-1-benzofuran-2(3H)- | | | |
| | ylidene}methyl)-1H-pyrrolo[2,3-b]pyridin-4- | | | |
| | yl]piperidine-4-carboxamide | | | |
| 203 | 1-[1-methyl-3-({3-oxo-5-[(pyridin-3- | | 635.3 | |
| | ylcarbamoyl)amino]-1-benzofuran-2(3H)- | | | |
| | ylidene}methyl)-1H-pyrrolo[2,3-b]pyridin-4-yl]-N-(2- | | | |
| | pyrrolidin-1-ylethyl)piperidine-4-carboxamide | | | |
| 204 | 1-[2-({4-[4-(2,5-dihydro-1H-pyrrol-1- | | 590.4 | |
| | ylcarbonyl)piperidin-1-yl]-1-methyl-1H-pyrrolo[2,3- | | | |
| | b]pyridin-3-yl}methylidene)-3-oxo-2,3-dihydro-1- | | | |
| | benzofuran-5-yl]-3-pyridin-3-ylurea | | | |
| 205 | N-methyl-1-[1-methyl-3-({3-oxo-5-[(pyridin-3- | | 635.4 | |
| | ylcarbamoyl)amino]-1-benzofuran-2(3H)- | | | |
| | ylidene}methyl)-1H-pyrrolo[2,3-b]pyridin-4-yl]-N-(1- | | | |
| | methylpyrrolidin-3-yl)piperidine-4-carboxamide | | | |
| 206 | N-methyl-1-[1-methyl-3-({3-oxo-5-[(pyridin-3- | | 621.3 | |
| | ylcarbamoyl)amino]-1-benzofuran-2(3H)- | | | |
| | ylidene}methyl)-1H-pyrrolo[2,3-b]pyridin-4-yl]-N- | | | |
| | [(3R)-pyrrolidin-3-yl]piperidine-4-carboxamide | | | |
| 207 | 1-[1-methyl-3-({3-oxo-5-[(pyridin-3- | | 607.3 | |
| | ylcarbamoyl)amino]-1-benzofuran-2(3H)- | | | |
| | ylidene}methyl)-1H-pyrrolo[2,3-b]pyridin-4-yl]-N- | | | |
| | [(3R)-pyrrolidin-3-yl]piperidine-4-carboxamide | | | |
| 208 | N-methyl-1-[1-methyl-3-({3-oxo-5-[(pyridin-3- | | 621.3 | |
| | ylcarbamoyl)amino]-1-benzofuran-2(3H)- | | | |
| | ylidene}methyl)-1H-pyrrolo[2,3-b]pyridin-4-yl]-N- | | | |
| | [(3S)-pyrrolidin-3-yl]piperidine-4-carboxamide | | | |
| 209 | 1-[1-methyl-3-({3-oxo-5-[(pyridin-3- | | 607.3 | |
| | ylcarbamoyl)amino]-1-benzofuran-2(3H)- | | | |
| | ylidene}methyl)-1H-pyrrolo[2,3-b]pyridin-4-yl]-N- | | | |
| | [(3S)-pyrrolidin-3-yl]piperidine-4-carboxamide | | | |
| 210 | 1-(2-{[4-(4-{[3-(dimethylamino)pyrrolidin-1- | | 635.3 | |
| | yl]carbonyl}piperidin-1-yl)-1-methyl-1H-pyrrolo[2,3- | | | |
| | b]pyridin-3-yl]methylidene}-3-oxo-2,3-dihydro-1- | | | |
| | benzofuran-5-yl)-3-pyridin-3-ylurea | | | |
| 211 | N-(1,1-dioxidotetrahydrothiophen-3-yl)-N-methyl-1- | | 668.2 | |
| | [1-methyl-3-({3-oxo-5-[(pyridin-3- | | | |
| | ylcarbamoyl)amino]-1-benzofuran-2(3H)- | | | |
| | ylidene}methyl)-1H-pyrrolo[2,3-b]pyridin-4- | | | |
| | yl]piperidine-4-carboxamide | | | |
| 212 | N-(1,1-dioxidotetrahydrothiophen-3-yl)-1-[1-methyl | | 656.2 | |
| | 3-({3-oxo-5-[(pyridin-3-ylcarbamoyl)amino]-1- | | | |
| | benzofuran-2(3H)-ylidene}methyl)-1H-pyrrolo[2,3- | | | |
| | b]pyridin-4-yl]piperidine-4-carboxamide | | | |
| 213 | N-methyl-1-[1-methyl-3-({3-oxo-5-[(pyridin-3- | | 636.3 | |
| | ylcarbamoyl)amino]-1-benzofuran-2(3H)- | | | |
| | ylidene}methyl)-1H-pyrrolo[2,3-b]pyridin-4-yl]-N- | | | |
| | (tetrahydro-2H-pyran-4-yl)piperidine-4-carboxamide | | | |
| 214 | 1-[1-methyl-3-({3-oxo-5-[(pyridin-3- | | 622.3 | |
| | ylcarbamoyl)amino]-1-benzofuran-2(3H)- | | | |
| | ylidene}methyl)-1H-pyrrolo[2,3-b]pyridin-4-yl]-N- | | | |
| | (tetrahydro-2H-pyran-4-yl)piperidine-4-carboxamide | | | |
| 215 | N-benzyl-N-methyl-1-[1-methyl-3-({3-oxo-5-[(pyridin- | | 642.3 | |
| | 3-ylcarbamoyl)amino]-1-benzofuran-2(3H)- | | | |
| | ylidene}methyl)-1H-pyrrolo[2,3-b]pyridin-4- | | | |
| | yl]piperidine-4-carboxamide | | | |
| 216 | N,N-dimethyl-1-(1-methyl-3-{(Z)-[3-oxo-5-({[4- | | 662.3 | |
| | (piperidin-1-ylmethyl)phenyl]carbamoyl}amino)-1- | | | |
| | benzofuran-2(3H)-ylidene]methyl}-1H-pyrrolo[2,3- | | | |
| | b]pyridin-4-yl)piperidine-4-carboxamide | | | |
| 217 | N,N-dimethyl-1-(1-methyl-3-{(Z)-[5-{[(6-morpholin-4- | | 651.3 | |
| | ylpyridin-3-yl)carbamoyl]amino}-3-oxo-1- | | | |
| | benzofuran-2(3H)-ylidene]methyl}-1H-pyrrolo[2,3- | | | |
| | b]pyridin-4-yl)piperidine-4-carboxamide | | | |
| 218 | N,N-dimethyl-1-(1-methyl-3-{(Z)-[5-({[4-(4- | | 663.3 | |
| | methylpiperazin-1-yl)phenyl]carbamoyl}amino)-3- | | | |
| | oxo-1-benzofuran-2(3H)-ylidene]methyl}-1H- | | | |
| | pyrrolo[2,3-b]pyridin-4-yl)piperidine-4-carboxamide | | | |
| 219 | 1-[(2Z)-2-{[1-methyl-4-(8-oxa-3-azabicyclo[3.2.1]oct- | | 608.3 | |
| | 3-yl)-1H-pyrrolo[2,3-b]pyridin-3-yl]methylidene}-3- | | | |
| | oxo-2,3-dihydro-1-benzofuran-5-yl]-3-(6-morpholin- | | | |
| | 4-ylpyridin-3-yl)urea | | | |
| 220 | 1-{4-[2-(dimethylamino)ethoxy]phenyl}-3-[(2Z)-2-{[1- | | 609.3 | |
| | methyl-4-(8-oxa-3-azabicyclo[3.2.1]oct-3-yl)-1H- | | | |
| | pyrrolo[2,3-b]pyridin-3-yl]methylidene}-3-oxo-2,3- | | | |
| | dihydro-1-benzofuran-5-yl]urea | | | |
| 221 | 1-[(2Z)-2-{[1-methyl-4-(8-oxa-3-azabicyclo[3.2.1]oct- | | 544.3 | |
| | 3-yl)-1H-pyrrolo[2,3-b]pyridin-3-yl]methylidene}-3- | | | |
| | oxo-2,3-dihydro-1-benzofuran-5-yl]-3-(4- | | | |
| | methylpiperazin-1-yl)urea | | | |
| 222 | N-methyl-1-{1-methyl-3-[(Z)-{3-oxo-5-[(pyridin-3- | | 657.3 | |
| | ylcarbamoyl)amino]-1-benzofuran-2(3H)- | | | |
| | ylidene}methyl]-1H-pyrrolo[2,3-b]pyridin-4-yl}-N-[(3- | | | |
| | methylpyridin-4-yl)methyl]piperidine-4-carboxamide | | | |
| 223 | N-methyl-1-{1-methyl-3-[(Z)-{3-oxo-5-[(pyridin-3- | | 657.3 | |
| | ylcarbamoyl)amino]-1-benzofuran-2(3H)- | | | |
| | ylidene}methyl]-1H-pyrrolo[2,3-b]pyridin-4-yl}-N-[(2- | | | |
| | methylpyridin-4-yl)methyl]piperidine-4-carboxamide | | | |
| 224 | N-[(4-methoxypyridin-2-yl)methyl]-N-methyl-1-{1- | | 673.3 | |
| | methyl-3-[(Z)-{3-oxo-5-[(pyridin-3- | | | |
| | ylcarbamoyl)amino]-1-benzofuran-2(3H)- | | | |
| | ylidene}methyl]-1H-pyrrolo[2,3-b]pyridin-4- | | | |
| | yl}piperidine-4-carboxamide | | | |
| 225 | N-methyl-1-{1-methyl-3-[(Z)-{3-oxo-5-[(pyridin-3- | | 657.3 | |
| | ylcarbamoyl)amino]-1-benzofuran-2(3H)- | | | |
| | ylidene}methyl]-1H-pyrrolo[2,3-b]pyridin-4-yl}-N-[(6- | | | |
| | methylpyridin-2-yl)methyl]piperidine-4-carboxamide | | | |
| 226 | N-methyl-1-{1-methyl-3-[(Z)-{3-oxo-5-[(pyridin-3- | | 590.2 | |
| | ylcarbamoyl)amino]-1-benzofuran-2(3H)- | | | |
| | ylidene}methyl]-1H-pyrrolo[2,3-b]pyridin-4-yl}-N- | | | |
| | prop-2-yn-1-ylpiperidine-4-carboxamide | | | |
| 227 | N-cyclopentyl-N-methyl-1-{1-methyl-3-[(Z)-{3-oxo-5- | | 620.3 | |
| | [(pyridin-3-ylcarbamoyl)amino]-1-benzofuran-2(3H)- | | | |
| | ylidene}methyl]-1H-pyrrolo[2,3-b]pyridin-4- | | | |
| | yl}piperidine-4-carboxamide | | | |
| 228 | N,N-dimethyl-1-(1-methyl-3-{(Z)-[5-{[(4- | | 587.3 | |
| | methylpiperazin-1-yl)carbamoyl]amino}-3-oxo-1- | | | |
| | benzofuran-2(3H)-ylidene]methyl}-1H-pyrrolo[2,3- | | | |
| | b]pyridin-4-yl)piperidine-4-carboxamide | | | |
| 229 | 1-[(2Z)-2-{[1-methyl-4-(8-oxa-3-azabicyclo[3.2.1]oct- | | 620.3 | |
| | 3-yl)-1H-pyrrolo[2,3-b]pyridin-3-yl]methylidene}-3- | | | |
| | oxo-2,3-dihydro-1-benzofuran-5-yl]-3-[4-(4- | | | |
| | methylpiperazin-1-yl)phenyl]urea | | | |
| 230 | N,N-dimethyl-1-(1-methyl-3-{(Z)-[5-({[6-(4- | | 664.3 | |
| | methylpiperazin-1-yl)pyridin-3-yl]carbamoyl}amino)- | | | |
| | 3-oxo-1-benzofuran-2(3H)-ylidene]methyl}-1H- | | | |
| | pyrrolo[2,3-b]pyridin-4-yl)piperidine-4-carboxamide | | | |
| 231 | 1-[(2Z)-2-{[1-methyl-4-(8-oxa-3-azabicyclo[3.2.1]oct- | | 621.3 | |
| | 3-yl)-1H-pyrrolo[2,3-b]pyridin-3-yl]methylidene}-3- | | | |
| | oxo-2,3-dihydro-1-benzofuran-5-yl]-3-[6-(4- | | | |
| | methylpiperazin-1-yl)pyridin-3-yl]urea | | | |
| 232 | 1-[(1-methyl-1H-imidazol-5-yl)methyl]-3-[(2Z)-2-{[1- | | 540.2 | |
| | methyl-4-(8-oxa-3-azabicyclo[3.2.1]oct-3-yl)-1H- | | | |
| | pyrrolo[2,3-b]pyridin-3-yl]methylidene}-3-oxo-2,3- | | | |
| | dihydro-1-benzofuran-5-yl]urea | | | |
| 233 | 1-[(2Z)-2-({1-methyl-4-[4-(2-oxa-5- | | 634.3 | |
| | azabicyclo[2.2.2]oct-5-ylcarbonyl)piperidin-1-yl]-1H- | | | |
| | pyrrolo[2,3-b]pyridin-3-yl}methylidene)-3-oxo-2,3- | | | |
| | dihydro-1-benzofuran-5-yl]-3-pyridin-3-ylurea | | | |
| 234 | 1-[(2Z)-2-{[1-methyl-4-(piperidin-1-ylcarbonyl)-1H- | | 523.2 | |
| | pyrrolo[2,3-b]pyridin-3-yl]methylidene}-3-oxo-2,3- | | | |
| | dihydro-1-benzofuran-5-yl]-3-pyridin-3-ylurea | | | |
| 235 | 1-(1-methylethyl)-3-[(2Z)-2-{[1-methyl-4-(8-oxa-3- | | 488.2 | |
| | azabicyclo[3.2.1]oct-3-yl)-1H-pyrrolo[2,3-b]pyridin-3- | | | |
| | yl]methylidene}-3-oxo-2,3-dihydro-1-benzofuran-5- | | | |
| | yl]urea | | | |
| 236 | 1-cyclohexyl-3-[(2Z)-2-{[1-methyl-4-(8-oxa-3- | | 528.3 | |
| | azabicyclo[3.2.1]oct-3-yl)-1H-pyrrolo[2,3-b]pyridin-3- | | | |
| | yl]methylidene}-3-oxo-2,3-dihydro-1-benzofuran-5- | | | |
| | yl]urea | | | |
| 237 | 1-cyclopropyl-3-[(2Z)-2-{[1-methyl-4-(8-oxa-3- | | 468.2 | |
| | azabicyclo[3.2.1]oct-3-yl)-1H-pyrrolo[2,3-b]pyridin-3- | | | |
| | yl]methylidene}-3-oxo-2,3-dihydro-1-benzofuran-5- | | | |
| | yl]urea | | | |
| 238 | 1-[(2Z)-2-({1-methyl-4-[(3S)-3-methylmorpholin-4-yl]- | | 511.3 | |
| | 1H-pyrrolo[2,3-b]pyridin-3-yl}methylidene)-3-oxo- | | | |
| | 2,3-dihydro-1-benzofuran-5-yl]-3-pyridin-3-ylurea | | | |
| 239 | 1-{(2Z)-2-[(4-{4-[(2R,6S)-2,6-dimethylmorpholin-4- | | 608.4 | |
| | yl]piperidin-1-yl}-1-methyl-1H-pyrrolo[2,3-b]pyridin-3- | | | |
| | yl)methylidene]-3-oxo-2,3-dihydro-1-benzofuran-5- | | | |
| | yl}-3-pyridin-3-ylurea | | | |
| 240 | 1-(3-chloropropyl)-3-[(2Z)-2-{[1-methyl-4-(8-oxa-3- | 234-41 | 522.4 | calcd for |
| | azabicyclo[3.2.1]oct-3-yl)-1H-pyrrolo[2,3-b]pyridin-3- | | | C27H28ClN5O4 + H⁺, |
| | yl]methylene}-3-oxo-2,3-dihydro-1-benzofuran-5- | | | 522.19026; |
| | yl]urea | | | found 522.18839; |
| 241 | 1-[3-(dimethylamino)propyl]-3-[(2Z)-2-{[1-methyl-4- | 261-3 | 531.5 | calcd for |
| | (8-oxa-3-azabicyclo[3.2.1]oct-3-yl)-1H-pyrrolo[2,3- | | | C29H34N6O4 + H⁺, |
| | b]pyridin-3-yl]methylene}-3-oxo-2,3-dihydro-1- | | | 531.27143; found |
| | benzofuran-5-yl]urea | | | 531.271431; |
| 242 | 1-{3-[(2-methoxyethyl)(methyl)amino]propyl}-3-[(2Z)- | 158-63 | 575.5 | calcd for |
| | 2-{[1-methyl-4-(8-oxa-3-azabicyclo[3.2.1]oct-3-yl)- | | | C31H38N6O5 + H⁺, |
| | 1H-pyrrolo[2,3-b]pyridin-3-yl]methylene}-3-oxo-2,3- | | | 575.29764; found |
| | dihydro-1-benzofuran-5-yl]urea | | | 575.29933; |
| 243 | N-ethyl-N-(2-hydroxyethyl)-4-{3-[(Z)-(4-hydroxy-3- | | 484.1 | |
| | oxo-1-benzofuran-2(3H)-ylidene)methyl]-1-methyl- | | | |
| | 1H-pyrrolo[2,3-b]pyridin-4-yl}benzamide | | | |
| 244 | N,N-diethyl-1-{1-methyl-3-[(Z)-{3-oxo-5-[(pyridin-3- | | 594.6 | calcd for |
| | ylcarbamoyl)amino]-1-benzofuran-2(3H)- | | | C33H35N7O4 + H⁺, |
| | ylidene}methyl]-1H-pyrrolo[2,3-b]pyridin-4- | | | 594.28233; found |
| | yl}piperidine-4-carboxamide | | | 594.28326; |
| 245 | (2Z)-4-hydroxy-2-[(4-{4-[(3-hydroxypyrrolidin-1- | | | calcd for |
| | yl)carbonyl]phenyl}-1-methyl-1H-pyrrolo[2,3- | | | C28H23N3O5 + H⁺, |
| | b]pyridin-3-yl)methylene]-1-benzofuran-3(2H)-one | | | 482.17105; found |
| | | | | 482.1710; |
| 246 | N-(2-methoxyethyl)-N-methyl-1-{1-methyl-3-[(Z)-{3- | decomp | 610.2 | calcd for |
| | oxo-5-[(pyridin-3-ylcarbamoyl)amino]-1-benzofuran- | 282-285 | | C33H35N7O5 + H⁺, |
| | 2(3H)-ylidene}methyl]-1H-pyrrolo[2,3-b]pyridin-4- | | | 610.27724; found |
| | yl}piperidine-4-carboxamide | | | 610.2772; |
| 247 | N-(2-methoxyethyl)-N-methyl-4-{1-methyl-3-[(Z)-{3- | | 603.6 | calcd for |
| | oxo-5-[(pyridin-3-ylcarbamoyl)amino]-1-benzofuran- | | | C34H30N6O5 + H⁺, |
| | 2(3H)-ylidene}methyl]-1H-pyrrolo[2,3-b]pyridin-4- | | | 603.23505; found |
| | yl}benzamide | | | 603.23408; |
| 248 | N-methoxy-4-{1-methyl-3-[(Z)-{3-oxo-5-[(pyridin-3- | | | calcd for |
| | ylcarbamoyl)amino]-1-benzofuran-2(3H)- | | | C31H24N6O5 + H⁺, |
| | ylidene}methyl]-1H-pyrrolo[2,3-b]pyridin-4- | | | 561.18809; found |
| | yl}benzamide | | | 561.1876; |
| 249 | (2Z)-4-hydroxy-2-[(4-{4-[(3-methoxypyrrolidin-1- | | 496.18654 | calcd for |
| | yl)carbonyl]phenyl}-1-methyl-1H-pyrrolo[2,3- | | | C29H25N3O5 + H⁺, |
| | b]pyridin-3-yl)methylene]-1-benzofuran-3(2H)-one | | | 496.18670; found |
| | | | | 496.18654; |
| 250 | 1-[(2Z)-2-({1-methyl-4-[4-(8-oxa-3- | decomp | 634.4 | calcd for |
| | azabicyclo[3.2.1]oct-3-ylcarbonyl)piperidin-1-yl]-1H- | 260-265 | | C35H35N7O5 + H⁺, |
| | pyrrolo[2,3-b]pyridin-3-yl}methylene)-3-oxo-2,3- | | | 634.27724; found |
| | dihydro-1-benzofuran-5-yl]-3-pyridin-3-ylurea | | | 634.2772; |
| 251 | N,N-bis(2-methoxyethyl)-1-{1-methyl-3-[(Z)-{3-oxo- | 206-210 | 654.4 | calcd for |
| | 5-[(pyridin-3-ylcarbamoyl)amino]-1-benzofuran- | | | C35H39N7O6 + H⁺, |
| | 2(3H)-ylidene}methyl]-1H-pyrrolo[2,3-b]pyridin-4- | | | 676.2580; found |
| | yl}piperidine-4-carboxamide | | | 676.2854; |
| 252 | 1-{(2Z)-2-[(1-methyl-4-{4-[(4-methylpiperazin-1- | | | calcd for |
| | yl)carbonyl]piperidin-1-yl}-1H-pyrrolo[2,3-b]pyridin-3- | | | C34H36N8O4 + H⁺, |
| | yl)methylidene]-3-oxo-2,3-dihydro-1-benzofuran-5- | | | 621.29323; found |
| | yl}-3-pyridin-3-ylurea | | | 621.29326; |
| 253 | 1-[(2Z)-2-({1-methyl-4-[4-(pyrrolidin-1- | | 592.2 | calcd for |
| | ylcarbonyl)piperidin-1-yl]-1H-pyrrolo[2,3-b]pyridin-3- | | | C33H33N7O4 + H⁺, |
| | yl}methylene)-3-oxo-2,3-dihydro-1-benzofuran-5-yl]- | | | 592.26668; found |
| | 3-pyridin-3-ylurea | | | 592.26587; |
| 254 | N-(2-hydroxyethyl)-1-{1-methyl-3-[(Z)-{3-oxo-5- | | 582.4 | calcd for |
| | [(pyridin-3-ylcarbamoyl)amino]-1-benzofuran-2(3H)- | | | C31H31N7O5 + H⁺, |
| | ylidene}methyl]-1H-pyrrolo[2,3-b]pyridin-4- | | | 582.24594; found |
| | yl}piperidine-4-carboxamide | | | 582.24524; |
| 255 | N-methyl-1-{1-methyl-3-[(Z)-{3-oxo-5-[(pyridin-3- | decomp | 643.4 | calcd for |
| | ylcarbamoyl)amino]-1-benzofuran-2(3H)- | 125-135 | | C36H34N8O4 + H⁺, |
| | ylidene}methyl]-1H-pyrrolo[2,3-b]pyridin-4-yl}-N- | | | 643.27758; found |
| | (pyridin-4-ylmethyl)piperidine-4-carboxamide | | | 643.2776; |
| 256 | N-methyl-1-{1-methyl-3-[(Z)-{3-oxo-5-[(pyridin-3- | decomp | 643.1 | calcd for |
| | ylcarbamoyl)amino]-1-benzofuran-2(3H)- | 195-205 | | C36H34N8O4 + H⁺, |
| | ylidene}methyl]-1H-pyrrolo[2,3-b]pyridin-4-yl}-N- | | | 643.27758; found |
| | (pyridin-3-ylmethyl)piperidine-4-carboxamide | | | 643.2776; |
| 257 | N-methyl-1-{1-methyl-3-[(Z)-{3-oxo-5-[(pyridin-3- | 187-189 | 643.4 | calcd for |
| | ylcarbamoyl)amino]-1-benzofuran-2(3H)- | | | C36H34N8O4 + H⁺, |
| | ylidene}methyl]-1H-pyrrolo[2,3-b]pyridin-4-yl}-N- | | | 643.27758; found |
| | (pyridin-2-ylmethyl)piperidine-4-carboxamide | | | 643.2776; |
| 258 | 1-[(2Z)-2-({1-methyl-4-[4-(piperidin-1- | | 606.3 | calcd for |
| | ylcarbonyl)piperidin-1-yl]-1H-pyrrolo[2,3-b]pyridin-3- | | | C34H35N7O4 + H⁺, |
| | yl}methylene)-3-oxo-2,3-dihydro-1-benzofuran-5-yl]- | | | 606.28233; found |
| | 3-pyridin-3-ylurea | | | 606.28176; |
| 259 | 1-{(2Z)-2-[(4-{4-[(3-methoxypyrrolidin-1- | | 615.3 | calcd for |
| | yl)carbonyl]phenyl}-1-methyl-1H-pyrrolo[2,3- | | | C35H30N6O5 + H⁺, |
| | b]pyridin-3-yl)methylene]-3-oxo-2,3-dihydro-1- | | | 615.23505; found |
| | benzofuran-5-yl}-3-pyridin-3-ylurea | | | 615.23442; |
| 260 | N-(2-methoxyethyl)-1-{1-methyl-3-[(Z)-{3-oxo-5- | | 596.3 | calcd for |
| | [(pyridin-3-ylcarbamoyl)amino]-1-benzofuran-2(3H)- | | | C32H33N7O5 + H⁺, |
| | ylidene}methyl]-1H-pyrrolo[2,3-b]pyridin-4- | | | 596.26159; found |
| | yl}piperidine-4-carboxamide | | | 596.26152; |
| 261 | N-[2-(dimethylamino)ethyl]-1-{1-methyl-3-[(Z)-{3- | | 609.3 | calcd for |
| | oxo-5-[(pyridin-3-ylcarbamoyl)amino]-1-benzofuran- | | | C33H36N8O4 + H⁺, |
| | 2(3H)-ylidene}methyl]-1H-pyrrolo[2,3-b]pyridin-4- | | | 609.29323; found |
| | yl}piperidine-4-carboxamide | | | 609.29291; |
| 262 | 1-{1-methyl-3-[(Z)-{3-oxo-5-[(pyridin-3- | dec 180-190 | 615.3 | calcd for |
| | ylcarbamoyl)amino]-1-benzofuran-2(3H)- | | | C34H30N8O4 + H⁺, |
| | ylidene}methyl]-1H-pyrrolo[2,3-b]pyridin-4-yl}-N- | | | 615.24628; found |
| | pyridin-3-ylpiperidine-4-carboxamide | | | 615.2463; |
| 263 | 1-{1-methyl-3-[(Z)-{3-oxo-5-[(pyridin-3- | dec 230-235 | 615.3 | calcd for |
| | ylcarbamoyl)amino]-1-benzofuran-2(3H)- | | | C34H30N8O4 + H⁺, |
| | ylidene}methyl]-1H-pyrrolo[2,3-b]pyridin-4-yl}-N- | | | 615.24628; found |
| | pyridin-4-ylpiperidine-4-carboxamide | | | 615.2463; |
| 264 | 1-[(2Z)-2-({4-[4-(2,5-diazabicyclo[2.2.1]hept-2- | dec 257-262 | 619.3 | calcd for |
| | ylcarbonyl)piperidin-1-yl]-1-methyl-1H-pyrrolo[2,3- | | | C34H34N8O4 + H⁺, |
| | b]pyridin-3-yl}methylene)-3-oxo-2,3-dihydro-1- | | | 619.27758; found |
| | benzofuran-5-yl]-3-pyridin-3-ylurea | | | 619.2776; |
| 265 | 1-{(2Z)-2-[(4-{4-[(2,6-dimethylmorpholin-4- | | 636.4 | calcd for |
| | yl)carbonyl]piperidin-1-yl}-1-methyl-1H-pyrrolo[2,3- | | | C35H37N7O5 + H⁺, |
| | b]pyridin-3-yl)methylene]-3-oxo-2,3-dihydro-1- | | | 636.29289; found |
| | benzofuran-5-yl}-3-pyridin-3-ylurea | | | 636.29321; |
| 266 | N-[2-(dimethylamino)ethyl]-N-methyl-1-{1-methyl-3- | | 623.4 | calcd for |
| | [(Z)-{3-oxo-5-[(pyridin-3-ylcarbamoyl)amino]-1- | | | C34H38N8O4 + H⁺, |
| | benzofuran-2(3H)-ylidene}methyl]-1H-pyrrolo[2,3- | | | 623.30888; found |
| | b]pyridin-4-yl}piperidine-4-carboxamide | | | 623.30934; |
| 267 | N,N-dimethyl-1-(1-methyl-3-{(Z)-[3-oxo-5-({[6- | | 666.4 | calcd for |
| | (tetrahydro-2H-pyran-4-yloxy)pyridin-3- | | | C36H39N7O6 + H⁺, |
| | yl]carbamoyl}amino)-1-benzofuran-2(3H)- | | | 666.30346; found |
| | ylidene]methyl}-1H-pyrrolo[2,3-b]pyridin-4- | | | 666.30463; |
| | yl)piperidine-4-carboxamide | | | |
| 268 | N,N-dimethyl-1-(1-methyl-3-{(Z)-[5-{[(4-morpholin-4- | | | calcd for |
| | ylphenyl)carbamoyl]amino}-3-oxo-1-benzofuran- | | | C36H39N7O5 + H⁺, |
| | 2(3H)-ylidene]methyl}-1H-pyrrolo[2,3-b]pyridin-4- | | | 650.30854; found |
| | yl)piperidine-4-carboxamide | | | 650.30698; |
| 269 | N-methyl-1-{1-methyl-3-[(Z)-{3-oxo-5-[(pyridin-3- | decomp | | calcd for |
| | ylcarbamoyl)amino]-1-benzofuran-2(3H)- | 184-194 | | C35H32N8O4 + H⁺, |
| | ylidene}methyl]-1H-pyrrolo[2,3-b]pyridin-4-yl}-N- | | | 629.26193; found |
| | pyridin-3-ylpiperidine-4-carboxamide | | | 629.2619; |
| 270 | 1-{1-methyl-3-[(Z)-{3-oxo-5-[(pyridin-3- | decomp | 629.2 | calcd for |
| | ylcarbamoyl)amino]-1-benzofuran-2(3H)- | 187-194 | | C35H32N8O4 + H⁺, |
| | ylidene}methyl]-1H-pyrrolo[2,3-b]pyridin-4-yl}-N- | | | 629.26193; found |
| | (pyridin-3-ylmethyl)piperidine-4-carboxamide | | | 629.2619; |
| 271 | 1-{1-methyl-3-[(Z)-{3-oxo-5-[(pyridin-3- | decomp | 629.2 | calcd for |
| | ylcarbamoyl)amino]-1-benzofuran-2(3H)- | 208-216 | | C35H32N8O4 + H⁺, |
| | ylidene}methyl]-1H-pyrrolo[2,3-b]pyridin-4-yl}-N- | | | 629.26193; found |
| | (pyridin-4-ylmethyl)piperidine-4-carboxamide | | | 629.2619; |
| 272 | 1-{(2Z)-2-[(1-methyl-4-{4-[(5-methyl-2,5- | decomp | 633.2 | calcd for |
| | diazabicyclo[2.2.1]hept-2-yl)carbonyl]piperidin-1-yl}- | 151-164 | | C35H36N8O4 + H⁺, |
| | 1H-pyrrolo[2,3-b]pyridin-3-yl)methylidene]-3-oxo- | | | 633.29323; found |
| | 2,3-dihydro-1-benzofuran-5-yl}-3-pyridin-3-ylurea | | | 633.2932; |
| 273 | 1-[(2Z)-2-{[1-methyl-4-(8-oxa-3-azabicyclo[3.2.1]oct- | | | calcd for |
| | 3-yl)-1H-pyrrolo[2,3-b]pyridin-3-yl]methylidene}-3- | | | C34H34N6O5 + H⁺, |
| | oxo-2,3-dihydro-1-benzofuran-5-yl]-3-(4-morpholin- | | | 607.26635; found |
| | 4-ylphenyl)urea | | | 607.26552; |
| 274 | 1-{3-[(Z)-{5-[({4-[2- | | | calcd for |
| | (dimethylamino)ethoxy]phenyl}carbamoyl)amino]-3- | | | C36H41N7O5 + H⁺, |
| | oxo-1-benzofuran-2(3H)-ylidene}methyl]-1-methyl- | | | 652.32419; found |
| | 1H-pyrrolo[2,3-b]pyridin-4-yl}-N,N- | | | 652.32354; |
| | dimethylpiperidine-4-carboxamide | | | |
| 275 | 1-{(2Z)-2-[(4-{4-[(3-methoxypyrrolidin-1- | | 622.3 | calcd for |
| | yl)carbonyl]piperidin-1-yl}-1-methyl-1H-pyrrolo[2,3- | | | C34H35N7O5 + H⁺, |
| | b]pyridin-3-yl)methylidene]-3-oxo-2,3-dihydro-1- | | | 622.27724; found |
| | benzofuran-5-yl}-3-pyridin-3-ylurea | | | 622.27622; |
| 276 | 1-[(2Z)-2-{[1-methyl-4-(3-oxa-9- | dec 240-245 | 537.1 | calcd for |
| | azabicyclo[3.3.1]non-9-yl)-1H-pyrrolo[2,3-b]pyridin- | | | C30H28N6O4 + H⁺, |
| | 3-yl]methylidene}-3-oxo-2,3-dihydro-1-benzofuran-5- | | | 537.22448; found |
| | yl]-3-pyridin-3-ylurea | | | 537.2245; |
| 277 | 1-[(2Z)-2-{[1-methyl-4-(2-oxa-5-azabicyclo[2.2.2]oct- | decomp | 523.1 | calcd for |
| | 5-yl)-1H-pyrrolo[2,3-b]pyridin-3-yl]methylidene}-3- | 227-235 | | C29H26N6O4 + H⁺, |
| | oxo-2,3-dihydro-1-benzofuran-5-yl]-3-pyridin-3- | | | 523.20883; found |
| | ylurea | | | 523.2088; |
| 278 | 1-[(2Z)-2-{[1-methyl-4-(8-oxa-3-azabicyclo[3.2.1]oct- | | 619.4 | calcd for |
| | 3-yl)-1H-pyrrolo[2,3-b]pyridin-3-yl]methylidene}-3- | | | C36H38N6O4 + H⁺, |
| | oxo-2,3-dihydro-1-benzofuran-5-yl]-3-[4-(piperidin-1- | | | 619.30273; found |
| | ylmethyl)phenyl]urea | | | 619.30241; |
| 279 | 1-methyl-3-[(2Z)-2-{[1-methyl-4-(2-oxa-5- | dec 197-205 | 460.2 | calcd for |
| | azabicyclo[2.2.2]oct-5-yl)-1H-pyrrolo[2,3-b]pyridin-3- | | | C25H25N5O4 + H⁺, |
| | yl]methylidene}-3-oxo-2,3-dihydro-1-benzofuran-5- | | | 460.19793; found |
| | yl]urea | | | 460.19748; |
| 280 | 1-[(2Z)-2-{[1-methyl-4-(3-oxa-8-azabicyclo[3.2.1]oct- | dec 250-255 | 523.2 | calcd for |
| | 8-yl)-1H-pyrrolo[2,3-b]pyridin-3-yl]methylidene}-3- | | | C29H26N6O4 + H⁺, |
| | oxo-2,3-dihydro-1-benzofuran-5-yl]-3-pyridin-3- | | | 523.20883; found |
| | ylurea | | | 523.20826; |
| 281 | 1-methyl-3-[(2Z)-2-{[1-methyl-4-(3-oxa-8- | dec 226-232 | 460.2 | calcd for |
| | azabicyclo[3.2.1]oct-8-yl)-1H-pyrrolo[2,3-b]pyridin-3- | | | C25H25N5O4 + H⁺, |
| | yl]methylidene}-3-oxo-2,3-dihydro-1-benzofuran-5- | | | 460.19793; found |
| | yl]urea | | | 460.19734; |
| 282 | 1-[(2Z)-2-{[4-(6,8-dioxa-3-azabicyclo[3.2.1]oct-3-yl)- | dec 224-227 | 525.2 | calcd for |
| | 1-methyl-1H-pyrrolo[2,3-b]pyridin-3-yl]methylidene}- | | | C28H24N6O5 + H⁺, |
| | 3-oxo-2,3-dihydro-1-benzofuran-5-yl]-3-pyridin-3- | | | 525.18809; found |
| | ylurea | | | 525.18847; |
| 283 | 1-[(2Z)-2-{[1-methyl-4-(2-oxa-5- | dec 218-222 | 509.2 | calcd for |
| | azabicyclo[2.2.1]hept-5-yl)-1H-pyrrolo[2,3-b]pyridin- | | | C28H24N6O4 + H⁺, |
| | 3-yl]methylidene}-3-oxo-2,3-dihydro-1-benzofuran-5- | | | 509.19318; found |
| | yl]-3-pyridin-3-ylurea | | | 509.19403; |
| 284 | 1-methyl-3-[(2Z)-2-{[1-methyl-4-(2-oxa-5- | dec 242-247 | 446.2 | calcd for |
| | azabicyclo[2.2.1]hept-5-yl)-1H-pyrrolo[2,3-b]pyridin- | | | C24H23N5O4 + H⁺, |
| | 3-yl]methylidene}-3-oxo-2,3-dihydro-1-benzofuran-5- | | | 446.18228; found |
| | yl]urea | | | 446.18173; |
| 285 | 1-azetidin-3-yl-3-[(2Z)-2-{[1-methyl-4-(8-oxa-3- | | | calcd for |
| | azabicyclo[3.2.1]oct-3-yl)-1H-pyrrolo[2,3-b]pyridin-3- | | | C27H28N6O4 + H⁺, |
| | yl]methylidene}-3-oxo-2,3-dihydro-1-benzofuran-5- | | | 501.22448; found |
| | yl]urea | | | 501.22426; |
| 286 | 1-[(2Z)-2-{[1-methyl-4-(8-oxa-3-azabicyclo[3.2.1]oct- | | 625.3 | calcd for |
| | 3-yl)-1H-pyrrolo[2,3-b]pyridin-3-yl]methylidene}-3- | | | C31H31F3N6O5 + H⁺, |
| | oxo-2,3-dihydro-1-benzofuran-5-yl]-3-[1- | | | 625.23808; |
| | (trifluoroacetyl)piperidin-4-yl]urea | | | found 625.23779; |
| 287 | 1-{3-[(Z)-{5-[(azetidin-3-ylcarbamoyl)amino]-3-oxo-1- | | 544.3 | calcd for |
| | benzofuran-2(3H)-ylidene}methyl]-1-methyl-1H- | | | C29H33N7O4 + H⁺, |
| | pyrrolo[2,3-b]pyridin-4-yl}-N,N-dimethylpiperidine-4- | | | 544.26668; found |
| | carboxamide | | | 544.26622; |
| 288 | N,N-dimethyl-1-(1-methyl-3-{(Z)-[5-{[(1- | | 531.3 | calcd for |
| | methylethyl)carbamoyl]amino}-3-oxo-1-benzofuran- | | | C29H34N6O4 + H⁺, |
| | 2(3H)-ylidene]methyl}-1H-pyrrolo[2,3-b]pyridin-4- | | | 531.27143; found |
| | yl)piperidine-4-carboxamide | | | 531.27048; |
| 289 | 1-(1-methyl-1H-benzimidazol-2-yl)-3-[(2Z)-2-{[1- | | | calcd for |
| | methyl-4-(8-oxa-3-azabicyclo[3.2.1]oct-3-yl)-1H- | | | C32H29N7O4 + H⁺, |
| | pyrrolo[2,3-b]pyridin-3-yl]methylidene}-3-oxo-2,3- | | | 576.23538; found |
| | dihydro-1-benzofuran-5-yl]urea | | | 576.235; |
| 290 | N,N-dimethyl-1-(1-methyl-3-{(Z)-[5-{[(1-methyl-1H- | | | calcd for |
| | benzimidazol-2-yl)carbamoyl]amino}-3-oxo-1- | | | C34H34N8O4 + H⁺, |
| | benzofuran-2(3H)-ylidene]methyl}-1H-pyrrolo[2,3- | | | 619.27758; found |
| | b]pyridin-4-yl)piperidine-4-carboxamide | | | 619.27734; |
| 291 | 1-{3-[(Z)-{5-[(cyclopropylcarbamoyl)amino]-3-oxo-1- | | | calcd for |
| | benzofuran-2(3H)-ylidene}methyl]-1-methyl-1H- | | | C29H32N6O4 + H⁺, |
| | pyrrolo[2,3-b]pyridin-4-yl}-N,N-dimethylpiperidine-4- | | | 529.25578; found |
| | carboxamide | | | 529.25522; |
| 292 | 1-[(2Z)-2-({1-methyl-4-[4-(3-oxa-8- | decomp | 634.4 | calcd for |
| | azabicyclo[3.2.1]oct-8-ylcarbonyl)piperidin-1-yl]-1H- | 197-203 | | C35H35N7O5 + H⁺, |
| | pyrrolo[2,3-b]pyridin-3-yl}methylidene)-3-oxo-2,3- | | | 634.27724; found |
| | dihydro-1-benzofuran-5-yl]-3-pyridin-3-ylurea | | | 634.2772; |
| 293 | 1-[(2Z)-2-{[1-methyl-4-(9-oxa-3,7- | decomp | 538.4 | calcd for |
| | diazabicyclo[3.3.1]non-3-yl)-1H-pyrrolo[2,3- | 193-200 | | C29H27N7O4 + H⁺, |
| | b]pyridin-3-yl]methylidene}-3-oxo-2,3-dihydro-1- | | | 538.21973; found |
| | benzofuran-5-yl]-3-pyridin-3-ylurea | | | 538.2197; |
| 294 | 1-[(2Z)-2-{[4-(3,6-dihydro-2H-pyran-4-yl)-1-methyl- | | 494.2 | calcd for |
| | 1H-pyrrolo[2,3-b]pyridin-3-yl]methylidene}-3-oxo- | | | C28H23N5O4 + H⁺, |
| | 2,3-dihydro-1-benzofuran-5-yl]-3-pyridin-3-ylurea | | | 494.18228; found |
| | | | | 494.18362; |
| 295 | 1-[(2Z)-2-{[4-(2,2-diethylmorpholin-4-yl)-1-methyl- | decomp | 553 | calcd for |
| | 1H-pyrrolo[2,3-b]pyridin-3-yl]methylidene}-3-oxo- | 225-228 | | C31H32N6O4 + H⁺, |
| | 2,3-dihydro-1-benzofuran-5-yl]-3-pyridin-3-ylurea | | | 553.25578; found |
| | | | | 553.2557; |
| 296 | 1-[(2Z)-2-({4-[(2R,6S)-2,6-dimethylmorpholin-4-yl]-1- | decomp | 525.2 | calcd for |
| | methyl-1H-pyrrolo[2,3-b]pyridin-3-yl}methylidene)-3- | 255-260 | | C29H28N6O4 + H⁺, |
| | oxo-2,3-dihydro-1-benzofuran-5-yl]-3-pyridin-3- | | | 525.22448; found |
| | ylurea | | | 525.2244; |
| 297 | 1-[(2Z)-2-({4-[2-(2-methoxyethyl)morpholin-4-yl]-1- | decomp | 555 | calcd for |
| | methyl-1H-pyrrolo[2,3-b]pyridin-3-yl}methylidene)-3- | 225-260 | | C30H30N6O5 + H⁺, |
| | oxo-2,3-dihydro-1-benzofuran-5-yl]-3-pyridin-3- | | | 555.23505; found |
| | ylurea | | | 555.2350; |
| 298 | 1-(6-fluoropyridin-3-yl)-3-[(2Z)-2-{[1-methyl-4-(8-oxa- | | 541.3 | calcd for |
| | 3-azabicyclo[3.2.1]oct-3-yl)-1H-pyrrolo[2,3-b]pyridin- | | | C29H25FN6O4 + H⁺, |
| | 3-yl]methylidene}-3-oxo-2,3-dihydro-1-benzofuran-5- | | | 541.19941; |
| | yl]urea | | | found 541.19951; |
| 299 | 1-(6-chloropyridin-3-yl)-3-[(2Z)-2-{[1-methyl-4-(8- | | 577.3 | calcd for |
| | oxa-3-azabicyclo[3.2.1]oct-3-yl)-1H-pyrrolo[2,3- | | | C29H25ClN6O4 + H⁺, |
| | b]pyridin-3-yl]methylidene}-3-oxo-2,3-dihydro-1- | | | 557.16986; |
| | benzofuran-5-yl]urea | | | found 557.17029; |
| 300 | N,N-dimethyl-1-(1-methyl-3-{(Z)-[3-oxo-5-({[1- | | 668.3 | calcd for |
| | (trifluoroacetyl)piperidin-4-yl]carbamoyl}amino)-1- | | | C33H36F3N7O5 + H⁺, |
| | benzofuran-2(3H)-ylidene]methyl}-1H-pyrrolo[2,3- | | | 668.28028; |
| | b]pyridin-4-yl)piperidine-4-carboxamide | | | found 668.27901; |
| 301 | 1-[(2Z)-2-{[4-(2,2-dimethylmorpholin-4-yl)-1-methyl- | 222-226 | 525.2 | calcd for |
| | 1H-pyrrolo[2,3-b]pyridin-3-yl]methylidene}-3-oxo- | | | C29H28N6O4 + H⁺, |
| | 2,3-dihydro-1-benzofuran-5-yl]-3-pyridin-3-ylurea | | | 525.22448; found |
| | | | | 525.2244; |
| 302 | (2Z)-2-{[4-(4-{[(2R,6S)-2,6-dimethylmorpholin-4- | 162-166 | 517.4 | calcd for |
| | yl]carbonyl}piperidin-1-yl)-1-methyl-1H-pyrrolo[2,3- | | | C29H32N4O5 + H⁺, |
| | b]pyridin-3-yl]methylidene}-4-hydroxy-1-benzofuran- | | | 517.24455; found |
| | 3(2H)-one | | | 517.2445; |
| 303 | (2Z)-2-{[4-(4-{[(2R,6S)-2,6-dimethylmorpholin-4- | decomp | 510.5 | calcd for |
| | yl]carbonyl}phenyl)-1-methyl-1H-pyrrolo[2,3- | 262-270 | | C30H27N3O5 + H⁺, |
| | b]pyridin-3-yl]methylidene}-4-hydroxy-1-benzofuran- | | | 510.20235; found |
| | 3(2H)-one | | | 510.2023; |
| 304 | (2Z)-4-hydroxy-2-{[4-(4-{[4-(2- | 118-122 | 539.5 | calcd for |
| | methoxyethyl)piperazin-1-yl]carbonyl}phenyl)-1- | | | C31H30N4O5 + H⁺, |
| | methyl-1H-pyrrolo[2,3-b]pyridin-3-yl]methylidene}-1- | | | 539.22890; found |
| | benzofuran-3(2H)-one | | | 539.2288; |
| 305 | (2Z)-4-hydroxy-2-{[4-(4-{[2-(2- | 154-160 | 540.5 | calcd for |
| | methoxyethyl)morpholin-4-yl]carbonyl}phenyl)-1- | | | C31H29N3O6 + H⁺, |
| | methyl-1H-pyrrolo[2,3-b]pyridin-3-yl]methylidene}-1- | | | 540.21291; found |
| | benzofuran-3(2H)-one | | | 540.2128; |
| 306 | (2Z)-4-hydroxy-2-({1-methyl-4-[4-(8-oxa-3- | decomp | 515.3 | calcd for |
| | azabicyclo[3.2.1]oct-3-ylcarbonyl)piperidin-1-yl]-1H- | 264-268 | | C29H30N4O5 + H⁺, |
| | pyrrolo[2,3-b]pyridin-3-yl}methylidene)-1- | | | 515.22890; found |
| | benzofuran-3(2H)-one | | | 515.2288; |
| 307 | (2Z)-4-hydroxy-2-{[4-(4-{[4-(2- | 136-140 | 532.4 | calcd for |
| | hydroxyethyl)piperazin-1-yl]carbonyl}piperidin-1-yl)- | | | C29H33N5O5 + H⁺, |
| | 1-methyl-1H-pyrrolo[2,3-b]pyridin-3-yl]methylidene}- | | | 532.25545; found |
| | 1-benzofuran-3(2H)-one | | | (ESI, [M + H]+), |
| | | | | 532.2562; |
| 308 | 1-[(2Z)-2-{[4-(4-{[2-(methoxymethyl)morpholin-4- | 206-210 | 652.4 | calcd for |
| | yl]carbonyl}piperidin-1-yl)-1-methyl-1H-pyrrolo[2,3- | | | C35H37N7O6 + H⁺, |
| | b]pyridin-3-yl]methylidene}-3-oxo-2,3-dihydro-1- | | | 652.28781; found |
| | benzofuran-5-yl]-3-pyridin-3-ylurea | | | 652.2877; |
| 309 | 1-[(2Z)-2-({1-methyl-4-[4-(3-oxa-9- | 210-215 | 648.4 | calcd for |
| | azabicyclo[3.3.1]non-9-ylcarbonyl)piperidin-1-yl]-1H- | | | C36H37N7O5 + H⁺, |
| | pyrrolo[2,3-b]pyridin-3-yl}methylidene)-3-oxo-2,3- | | | 648.29289; found |
| | dihydro-1-benzofuran-5-yl]-3-pyridin-3-ylurea | | | 648.2928; |
| 310 | (2Z)-4-hydroxy-2-{[4-(4-{[2- | 222-226 | 526.3 | calcd for |
| | (methoxymethyl)morpholin-4-yl]carbonyl}phenyl)-1- | | | C30H27N3O6 + H⁺, |
| | methyl-1H-pyrrolo[2,3-b]pyridin-3-yl]methylidene}-1- | | | 526.19726; found |
| | benzofuran-3(2H)-one | | | 526.1972; |
| 311 | (2Z)-4-hydroxy-2-{[4-(4-{[2- | 112-115 | 533.4 | calcd for |
| | (methoxymethyl)morpholin-4-yl]carbonyl}piperidin-1- | | | C29H32N4O6 + H⁺, |
| | yl)-1-methyl-1H-pyrrolo[2,3-b]pyridin-3- | | | 533.23946; found |
| | yl]methylidene}-1-benzofuran-3(2H)-one | | | 533.2394; |
| 312 | (2Z)-4-hydroxy-2-({1-methyl-4-[4-(3-oxa-9- | decomp | 529.4 | calcd for |
| | azabicyclo[3.3.1]non-9-ylcarbonyl)piperidin-1-yl]-1H- | 210-218 | | C30H32N4O5 + H⁺, |
| | pyrrolo[2,3-b]pyridin-3-yl}methylidene)-1- | | | 529.24455; found |
| | benzofuran-3(2H)-one | | | 529.2445; |

### Biological Evaluation—PI3K-alpha, PI3K-beta, PI3K-gamma, and PI3K-delta Fluorescence Polarization Assay Protocols

PI3-Kinase reactions were performed in 5 mM HEPES, pH 7, 2.5 mM MgCl₂, and 25 μM ATP, with diC8-PI(4,5)P2 (Echelon, Salt Lake City Utah) as substrate. Nunc 384-well black polypropylene fluorescent plates were used for PI3K assays. Reactions were quenched by the addition of EDTA to a final concentration of 10 mM. Final reaction volumes were 10 μl. For evaluation of PI3K inhibitors, 5 ng of enzyme (PI3K-alpha, beta, gamma, or delta) and 2.5 μM of substrate was used per 10 ml reaction volume, and inhibitor concentrations ranged from 100 pM to 20 μM; the final level of DMSO in reactions never exceeded 2%. Reactions were allowed to proceed for one hour at 25° C. After 1 hour, GST-tagged GRP1 (general receptor for phosphoinositides) PH domain fusion protein was added to a final concentration of 100 nM, and BODIPY-TMRI(1,3,4,5)P4 (Echelon) was also added to a final concentration of 5 nM. Final sample volumes were 25 μl with a final DMSO concentration of 0.8%. Assay Plates were read on Perkin-Elmer Envision plate readers with appropriate filters for Tamra [BODIPY-TMRI(1,3,4,5)P4]. Data obtained were used to calculate enzymatic activity and enzyme inhibition by inhibitor compounds.

### mTOR Enzyme Assay

The routine human TOR assays with purified enzyme are performed in 96-well plates by DELFIA format as follows. Enzyme is first diluted in kinase assay buffer (10 mM HEPES (pH 7.4), 50 mM NaCl, 50 mM β-glycerophosphate, 10 mM MnCl₂, 0.5 mM DTT, 0.25 μM microcystin LR, and 100 μg/mL BSA). To each well, 12 μL of the diluted enzyme is mixed briefly with 0.5 μL test inhibitor or the control vehicle dimethylsulfoxide (DMSO). The kinase reaction is initiated by adding 12.5 μL kinase assay buffer containing ATP and His6-S6K (substrate) to give a final reaction volume of 25 μL containing 800 ng/mL FLAG-TOR, 100 μM ATP and 1.25 μM His6-S6K. The reaction plate is incubated for 2 hours (linear at 1-6 hours) at room temperature with gentle shaking and then terminated by adding 25 μL Stop buffer (20 mM HEPES, pH 7.4), 20 mM EDTA, 20 mM EGTA). The DELFIA detection of the phosphorylated His6-S6K (Thr-389) is performed at room temperature using a monoclonal anti-P(T389)-p70S6K antibody (lA5, Cell Signaling) labeled with Europium-N1-ITC (Eu) (10.4 Eu per antibody, PerkinElmer). The DELFIA Assay buffer and Enhancement solution are purchased from PerkinElmer. The terminated kinase reaction mixture (45 μL) is transferred to a MaxiSorp plate (Nunc) containing 55 μL PBS. The His6-S6K is allowed to attach for 2 hours after which the wells are aspirated and washed once with PBS. DELFIA Assay buffer (100 μL) with 40 ng/mL Eu-P(T389)-S6K antibody is added. The antibody binding is continued for 1 hour with gentle agitation. The wells are then aspirated and washed 4 times with PBS containing 0.05% Tween-20 (PBST). DELFIA Enhancement solution (100 μL) is added to each well and the plates are read in a PerkinElmer Victor model plate reader.

### In Vitro Cell Growth Assay

Cell lines used were human adenocarcinoma (LoVo), pancreatic (PC3), prostate (LNCap), breast (MDA468, MCF7), colon (HCT116), renal (HTB44 A498), and ovarian (OVCAR³) tumor cell lines. The tumor cells were plated in 96-well culture plates at approximately 3000 cells per well. One day following plating, various concentrations of inhibitors in DMSO were added to cells (final DMSO concentration in cell assays was 0.25%). Three days after drug treatment, viable cell densities were determined by cell mediated metabolic conversion of the dye MTS, a well-established indicator of cell proliferation in vitro. Cell growth assays were performed using kits purchased from Promega Corporation (Madison, Wis.), following the protocol provided by the vendor. Measuring absorbance at 490 nm generated MTS assay results. Compound effect on cell proliferation was assessed relative to untreated control cell growth. The drug concentration that conferred 50% inhibition of growth was determined as IC₅₀ (μM). IC₅₀ values of 0.7 nM to several μM were observed in the various tumor lines for compounds of this invention.

TABLE 3

| | PI3Kα | PI3Kγ | mTOR Kinase |
|---|---|---|---|
| Compound | Median IC₅₀ (nM) | Median IC₅₀ (nM) | Median IC₅₀ (nM) |
| 1 | 64 | 168 | 41.5 |
| 2 | 2337 | | |
| 3 | 506 | >10000.0 | 685.0 |
| 4 | 676 | 8502 | |
| 5 | 3927 | >10000.0 | >20000.00 |
| 6 | 5887 | >10000.0 | |
| 7 | 921 | >10000.0 | |
| 8 | 203 | 9000 | |
| 9 | 135 | 9167 | |
| 10 | >10000.0 | >10000.0 | |
| 11 | 525 | | 1200.0 |
| 12 | 167 | 270 | 2.8 |
| 13 | 116 | 79 | 1.2 |
| 14 | 188 | 220 | 1.4 |
| 15 | 183 | 139 | 1.0 |
| 16 | 24 | 247 | 5.0 |
| 17 | 7 | 57 | 0.3 |
| 18 | 19 | 106 | 2.7 |
| 19 | 14 | 35 | 0.5 |
| 20 | 6 | 44 | 0.2 |
| 21 | 34 | 140 | 3.5 |
| 22 | 109 | 276 | 4.6 |
| 23 | 381 | 2090 | 50.5 |
| 24 | 95 | 93 | 0.5 |
| 25 | 85 | 84 | 0.5 |
| 26 | 233 | 1581 | >4000.00 |
| 27 | 820 | 4000 | 50.0 |
| 28 | 5122 | 5185 | 2500.0 |
| 29 | 3672 | 8000 | 2700.0 |
| 30 | 219 | 2376 | 19.0 |
| 31 | 89 | 187 | 3.5 |
| 32 | 98 | 1050 | 46.5 |
| 33 | 8 | 38 | 0.6 |
| 34 | 6 | 38 | 0.2 |
| 35 | 11 | 51 | 0.4 |
| 36 | 7 | 36 | 0.3 |
| 37 | 6857 | 4000 | >4000.00 |
| 38 | 234 | 1568 | 37.0 |
| 39 | 106 | 104 | 5.7 |
| 40 | 68 | 282 | 0.6 |
| 41 | 24 | 141 | 0.4 |
| 42 | 129 | >10000 | 22.5 |
| 43 | 19 | 66 | 0.4 |
| 44 | 44 | 200 | 0.9 |
| 45 | 12 | 131 | 0.3 |
| 46 | 59 | 240 | 1.6 |
| 47 | 173 | 645 | 2.8 |
| 48 | 53 | 184 | 2.2 |
| 49 | 25 | 83 | 0.2 |
| 50 | 23 | 177 | 0.2 |
| 51 | 47 | 92 | 3.4 |
| 52 | 123 | 199 | 0.6 |
| 53 | 52 | 157 | 0.5 |
| 54 | 11 | 70 | 0.1 |
| 55 | 104 | 51 | 0.4 |
| 56 | 79 | 581 | 1.9 |
| 57 | 147 | 148 | 11.4 |
| 58 | 199 | 966 | 16.5 |
| 59 | 391 | 980 | 35.5 |
| 60 | >10000 | >10000 | 58.5 |
| 61 | 311 | 668 | 57.0 |
| 62 | 18 | 149 | 4.3 |
| 63 | 8 | 24 | 1.5 |
| 64 | 29 | 81 | 1.8 |
| 65 | 41 | 60 | 0.6 |
| 66 | 32 | 61 | 1.4 |
| 67 | 7 | 21 | 0.2 |
| 68 | >10000 | 1805 | >4000 |
| 69 | 64 | 281 | 24.8 |
| 70 | 271 | 242 | 115.0 |
| 71 | 62 | 249 | 16.0 |
| 72 | 5 | 66 | 0.7 |
| 73 | 23 | 75 | 3.1 |
| 74 | 14 | 132 | 1.6 |
| 75 | 92 | 319 | 10.3 |
| 76 | 41 | 132 | 8.5 |
| 77 | 2 | 22 | 0.2 |
| 78 | 31 | 315 | 5.0 |
| 79 | 8 | 49 | 1.4 |
| 80 | 23 | 69 | 1.8 |
| 81 | 28 | 79 | 1.6 |
| 82 | 20 | 70 | 1.4 |
| 83 | 124 | 951 | 9.5 |
| 84 | 4878 | >10000 | 165.0 |
| 85 | 273 | 157 | 26.5 |
| 86 | 77 | 600 | 3.4 |
| 87 | 161 | 66 | 3.6 |
| 88 | 8 | 27 | 0.2 |
| 89 | 8 | 50 | 0.5 |
| 90 | 10 | 16 | 24.0 |
| 91 | 36 | 74 | 0.7 |
| 92 | 9 | 54 | 0.2 |
| 93 | 85 | 124 | 1.3 |
| 94 | 43 | 351 | 10.5 |
| 95 | 479 | 3241 | 2000.0 |
| 96 | 301 | 2705 | 13.0 |
| 97 | 13 | 321 | 0.7 |
| 98 | 11 | 100 | 0.2 |
| 99 | >10000 | 2233 | 3500.0 |
| 100 | 806 | >10000 | 15.0 |
| 101 | 20 | 339 | 10.8 |
| 102 | 104 | 271 | 1.9 |
| 103 | 182 | 169 | 6.6 |
| 104 | 70 | 245 | 2.9 |
| 105 | >10000 | >10000 | 69.0 |
| 106 | 6014 | >10000 | 7.4 |
| 107 | 386 | 676 | 12.3 |
| 108 | 6114 | >10000 | 53.0 |
| 109 | 152 | 253 | 4.4 |
| 110 | 651 | >10000 | 82.5 |
| 111 | 36 | 66 | 0.5 |
| 112 | 120 | 576 | 39.0 |
| 113 | 101 | 626 | 20.0 |
| 114 | 29 | 127 | 0.7 |
| 115 | 155 | 208 | 17.0 |
| 116 | 8488 | >10000 | 23.0 |
| 117 | 30 | 88 | 4.4 |
| 118 | 286 | 791 | 20.0 |
| 119 | 46 | 183 | 72.0 |
| 120 | 35 | 2375 | 285.0 |
| 121 | 369 | 425 | 18.0 |
| 122 | 60 | 279 | 18.0 |
| 123 | 43 | 156 | 16.5 |
| 124 | 70 | 184 | 1.0 |
| 125 | 43 | 93 | 13.0 |
| 126 | 802 | 8643 | 6.2 |
| 127 | 107 | 829 | 8.1 |
| 128 | 49 | 54 | 0.5 |
| 129 | 47 | 167 | 14.0 |
| 130 | 154 | 263 | 3.2 |
| 131 | 580 | 557 | 9.5 |
| 132 | 33 | 83 | 1.7 |
| 133 | 37 | 30 | 1.5 |
| 134 | 55 | 89 | 3.8 |
| 135 | 156 | 1416 | 10.0 |
| 136 | 27 | 106 | 2.0 |
| 137 | 122 | 256 | 4.9 |
| 138 | 141 | 1232 | 7.8 |
| 139 | 119 | 1270 | 19.0 |
| 140 | 59 | 74 | 2.0 |
| 141 | 5898 | 1858 | 180.0 |
| 142 | 1581 | >10000 | 8.0 |
| 143 | 1324 | >10000 | 80.5 |
| 144 | 57 | 77 | 0.6 |
| 145 | 38 | 74 | 0.6 |
| 146 | 100 | 101 | 0.4 |
| 147 | 38 | 54 | 0.6 |
| 148 | 39 | 94 | 0.7 |
| 149 | 48 | 62 | 1.7 |
| 150 | 63 | 50 | 1.4 |
| 151 | 19 | 49 | 2.1 |
| 152 | 31 | 32 | 0.6 |
| 153 | 13 | 148 | 16.0 |
| 154 | 1390 | 1069 | 69.0 |
| 155 | 150 | 306 | 14.5 |
| 156 | 1939 | 6914 | 1650.0 |
| 157 | 74 | 78 | 9.4 |
| 158 | 179 | 149 | 8.6 |
| 159 | 1454 | 4151 | 70.5 |
| 160 | not tested | not tested | not tested |
| 161 | 5500 | >10000 | 397.0 |
| 162 | 5900 | 7426 | >4000 |
| 163 | 5640 | >10000 | 30.0 |
| 164 | 39 | 131 | 3.1 |
| 165 | 25 | 144 | 2.7 |
| 166 | 252 | 343 | 15.5 |
| 167 | 2080 | >10000 | 14.3 |
| 168 | 420 | 487 | 6.7 |
| 169 | >10000 | 1170 | 11.5 |
| 170 | 3111 | >10000 | 91.5 |
| 171 | 1999 | 8745 | 66.5 |
| 172 | 498 | 1123 | 145.0 |
| 173 | >10000 | >10000 | 18.0 |
| 174 | 337 | 337 | 16.5 |
| 175 | 73 | 152 | 0.9 |
| 176 | >10000 | >10000 | 445.0 |
| 177 | 2690 | 2365 | 66.0 |
| 178 | 1120 | 652 | 54.5 |
| 179 | 249 | 578 | 15.0 |
| 180 | 38 | 106 | 2.9 |
| 181 | 123 | 1212 | 58.0 |
| 182 | 16 | 38 | 1.6 |
| 183 | 367 | 479 | 755.0 |
| 184 | 632 | 484 | 83.0 |
| 185 | 275 | 348 | 71.0 |
| 186 | 445 | 550 | 4.6 |
| 187 | 34 | 99 | 195.0 |
| 188 | >10000 | >10000 | 2450.0 |
| 189 | 2030 | 2037 | 10.5 |
| 190 | 26 | 80 | 3.5 |
| 191 | 34 | 101 | 3.0 |
| 192 | 26 | 39 | 1.0 |
| 193 | 24 | 45 | 5.6 |
| 194 | 237 | 472 | 34.0 |
| 195 | 140 | 234 | 4.3 |
| 196 | 41 | 66 | 1.0 |
| 197 | 2367 | 3000 | 130.0 |
| 198 | 293 | 249 | 19.5 |
| 199 | 2239 | 2992 | 12.5 |
| 200 | >10000 | >10000 | 4.8 |
| 201 | 4234 | 6828 | 7.7 |
| 202 | 5901 | >10000 | 4.0 |
| 203 | >10000 | >10000 | 19.0 |
| 204 | 5615 | 1727 | 10.0 |
| 205 | >10000 | 9226 | 49.0 |
| 206 | >10000 | >10000 | 47.0 |
| 207 | 10000 | 4292 | 16.0 |
| 208 | >10000 | >10000 | 50.0 |
| 209 | >10000 | 4165 | 26.5 |
| 210 | >10000 | 2175 | 11.0 |
| 211 | >10000 | >10000 | 11.5 |
| 212 | 7448 | 5227 | 5.7 |
| 213 | 8362 | 7218 | 18.9 |
| 214 | >10000 | >10000 | 10.2 |
| 215 | >10000 | 9277 | 102.5 |
| 216 | 613 | 231 | 30.0 |
| 217 | 4007 | >10000 | 97.0 |
| 218 | 994 | >10000 | 445.0 |
| 219 | 2224 | >10000 | 29.0 |
| 220 | 915 | >10000 | 62.5 |
| 221 | 221 | 1013 | 175.0 |
| 222 | 2826 | 2535 | 34.0 |
| 223 | 1849 | 1271 | 23.0 |
| 224 | 2367 | 1422 | 23.5 |
| 225 | 2324 | 1920 | 49.0 |
| 226 | 921 | 1143 | 22.5 |
| 227 | 2724 | 1109 | 165.0 |
| 228 | 415 | 2753 | 170.0 |
| 229 | 2297 | 6667 | 280.0 |
| 230 | 10882 | >10000 | 74.0 |
| 231 | 703 | >10000 | 31.5 |
| 232 | 1143 | >10000 | 160.0 |
| 233 | 196 | 1726 | 38.0 |
| 234 | 253 | 2498 | >4000 |
| 235 | 845 | 2564 | 72.0 |
| 236 | 1466 | 2966 | 4000.0 |
| 237 | 289 | 2134 | 23.0 |
| 238 | 649 | 3642 | 130.0 |
| 239 | 326 | 748 | 64.0 |
| 240 | 1063 | 2183 | 105.0 |
| 241 | 53 | 34 | 10.5 |
| 242 | 243 | 781 | 64.5 |
| 243 | 167 | 219 | 90.5 |
| 244 | 308 | 3377 | 115.0 |
| 245 | 29 | 54 | 1.4 |
| 246 | 1425 | 1536 | 30.5 |
| 247 | 3296 | 484 | 30.0 |
| 248 | 976 | 479 | 1650.0 |
| 249 | 20 | 26 | 0.6 |
| 250 | 1677 | 2824 | 17.0 |
| 251 | 4053 | 5575 | 16.0 |
| 252 | 4598 | 1248 | 7.0 |
| 253 | 909 | 661 | 8.2 |
| 254 | 3330 | 1136 | 4.2 |
| 255 | 1230 | 2412 | 9.3 |
| 256 | 1025 | 1610 | 10.9 |
| 257 | 1568 | 1398 | 8.4 |
| 258 | 604 | 206 | 47.5 |
| 259 | 2062 | 529 | 17.5 |
| 260 | 744 | 1917 | 8.4 |
| 261 | >10000 | 886 | 22.5 |
| 262 | 2893 | 10500 | 14.5 |
| 263 | 15505 | 8208 | 25.0 |
| 264 | 6358 | 378 | 16.5 |
| 265 | 3747 | 1188 | 40.5 |
| 266 | >10000 | 1197 | 42.5 |
| 267 | 503 | 638 | 39.0 |
| 268 | 2588 | 6092 | 215.0 |
| 269 | 4960 | 5750 | 13.0 |
| 270 | >10000 | >10000 | 6.0 |
| 271 | 4041 | 3911 | 9.0 |
| 272 | >10000 | 2408 | 19.0 |
| 273 | 5703 | >10000 | 3350.0 |
| 274 | 778 | 934 | 21.0 |
| 275 | 1610 | 1333 | 13.0 |
| 276 | 2260 | 1117 | 31.5 |
| 277 | 296 | 2588 | 7.5 |
| 278 | 743 | 450 | 23.0 |
| 279 | 128 | 643 | 11.0 |
| 280 | 179 | 995 | 42.0 |
| 281 | 104 | 1708 | 31.0 |
| 282 | 288 | 642 | 21.0 |
| 283 | 350 | 779 | 27.0 |
| 284 | 275 | 721 | 50.0 |
| 285 | 49 | 532 | 11.4 |
| 286 | 354 | 1719 | 108.0 |
| 287 | 68 | 314 | 8.4 |
| 288 | 1488 | 3783 | 73.0 |
| 289 | 5712 | >10000 | 205.0 |
| 290 | 1251 | >10000 | 150.0 |
| 291 | 2550 | 4684 | 59.0 |
| 292 | 3477 | >10000 | 5.4 |
| 293 | >10000 | 862 | 300.0 |
| 294 | 2083 | >10000 | 27.5 |
| 295 | 721 | 1228 | 21.5 |
| 296 | 239 | 1163 | 19.0 |
| 297 | 884 | 1928 | 25.5 |
| 298 | 274 | 2120 | 26.0 |
| 299 | 747 | 2522 | 69.0 |
| 300 | 736 | 3543 | 105.0 |
| 301 | 566 | 1113 | 13.0 |
| 302 | 223 | 396 | 3.2 |
| 303 | 75 | 98 | 2.3 |
| 304 | 109 | 312 | 1.5 |
| 305 | 65 | 38 | 1.0 |
| 306 | 51 | 131 | 2.0 |
| 307 | 24 | 158 | 1.9 |
| 308 | 729 | 2802 | 37.0 |
| 309 | 164 | 1370 | 48.0 |
| 310 | 73 | 212 | 1.4 |
| 311 | 145 | 698 | 3.8 |
| 312 | 67 | 453 | 3.2 |

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A compound of Formula 1: 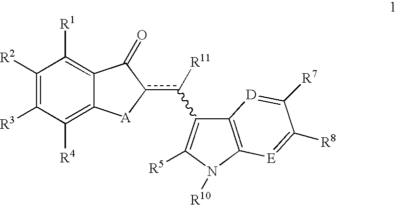
or a geometric isomer thereof or a pharmaceutically acceptable salt thereof, wherein:
A is oxygen, sulfur, or CH₂; [symbol]
represents an optional second carbon-to-carbon bond;
D is C—R⁶ or N;
E is C—R⁹ or N;
with the proviso that at least one of D and E must be N;
R¹, R², R³, and R⁴ are independently H; C₁-C₆alkoxy optionally substituted with from 1 to 3 substituents independently selected from H₂N—, (C₁-C₆alkyl)N—, and (C₁-C₆alkyl)(C₁-C₆alkyl)N—; C₁-C₆alkyl; (C₁-C₆alkoxy)carbonyl; R¹²R¹³N—; R¹²R¹³NC(O)NH—; R¹²C(O)NH—; R¹⁴OC(O)NH—; halo; or hydroxyl;
R¹² and R¹³ are each independently H; C₁-C₆alkyl optionally substituted with from 1 to 3 substituents independently selected from halo, H₂N—, (C₁-C₆alkyl)NH—, (C₁-C₆alkyl)(C₁-C₆alkyl)N-optionally substituted by C₁-C₆alkoxy, C₁-C₉heterocyclyl optionally substituted by C₁-C₆alkyl, or C₁-C₉heteroaryl optionally substituted by C₁-C₆alkyl; perfluoro(C₁-C₆)alkyl; C₁-C₉heteroaryl optionally substituted with from 1 to 3 substituents independently selected from C₁-C₆alkyl, halo, C₁-C₉heterocyclyl optionally substituted by C₁-C₆alkyl, C₁-C₆alkoxy optionally substituted with (C₁-C₆alkyl)(C₁-C₆alkyl)N—, C₁-C₉heterocyclyl-O—, heterocyclyl(C₁-C₆alkyl), and perfluoro(C₁-C₆)alkyl; C₁-C₉heterocyclyl optionally substituted by C₁-C₆alkyl or C₁-C₈acyl optionally substituted with from 1 to 3 independently selected halogens; C₆-C₁₄aryl optionally substituted with from 1 to 3 substituents independently selected from C₁-C₆alkyl, halo, C₁-C₉heterocyclyl optionally substituted by C₁-C₆alkyl, C₁-C₆alkoxy optionally substituted with (C₁-C₆alkyl)(C₁-C₆alkyl)N—, C₁-C₉heterocyclyl-O—, heterocyclyl(C₁-C₆alkyl), and perfluoro(C₁-C₆)alkyl; or C₃-C₈cycloalkyl;
R¹⁴ is independently C₁-C₆alkyl, C₁-C₆hydroxylalkyl-, or C₆-C₁₄aryl;
R⁵ is H; C₁-C₆alkyl; C₆-C₁₄aryl; C₃-C₈cycloalkyl; halo; C₁-C₉heteroaryl; C₁-C₆heterocyclylalkyl; C₁-C₆perfluoroalkyl-; R¹⁵R¹⁶NC(O)—; (C₁-C₆alkoxy)carbonyl; or CO₂H;
R¹⁵ and R¹⁶ are each independently H; C₁-C₆alkyl optionally substituted with from 1 to 3 substituents independently selected from H₂N—, (C₁-C₆alkyl)NH—, (C₁-C₆alkyl)(C₁-C₆alkyl)N—, or C₁-C₉heteroaryl; C₁-C₉heteroaryl; C₆-C₁₄aryl optionally substituted with from 1 to 3 substituents independently selected from C₁-C₆alkyl, halo, perfluoro(C₁-C₆)alkyl; C₃-C₈cycloalkyl;
or R¹⁵ and R¹⁶ when taken together with the nitrogen to which they are attached can form a 3- to 7-membered nitrogen-containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;
R⁶-R⁹ are each independently:
(a) H; (b) C₁-C₆alkoxy; (c) C₁-C₆alkyl optionally substituted by C₆-C₁₄aryl; (d) C₂-C₆alkenyl optionally substituted by C₆-C₁₄aryl; (e) C₂-C₆alkynyl optionally substituted by C₆-C₁₄aryl; (f) (C₁-C₆alkyl)amido-; (g) C₁-C₆alkylcarboxy; (h) (C₁-C₆alkyl)carboxyamido; (i) (C₁-C₆alkyl)SO₂—; (j) C₆-C₁₄aryl optionally substituted with from 1 to 3 substituents independently selected from: (i) C₁-C₈acyl, (ii) C₁-C₆alkyl, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl optionally substituted by C₁-C₆alkyl, (iii) (C₁-C₆alkyl)amido-, (iv) (C₁-C₆alkyl)carboxy, (v) (C₁-C₆alkyl)carboxyamido, (vi) C₁-C₆alkoxy- optionally substituted by C₁-C₆alkoxy- or C₁-C₉heteroaryl, (vi) (C₁-C₆alkoxy)carbonyl, (viii) (C₆-C₁₄aryl)oxy, (ix) C₃-C₈cycloalkyl, (x) halo, (xi) C₁-C₆haloalkyl-, (xii) C₁-C₉heterocyclyl optionally substituted by C₁-C₆alkyl or C₁-C₆hydroxylalkyl-, (xiii) hydroxyl, (xiv) C₁-C₆hydroxylalkyl-, (xv) C₁-C₆perfluoroalkyl-, (xvi) C₁-C₆perfluoroalkyl-O—, (xvii) R¹⁷R¹⁸N—, (xviii) CN, (xix) —COOH, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) C₁-C₉heteroaryl, (xxviii) —C(═N—(OR¹⁷))—(NR¹⁷R¹⁸), and (xxix) —NO₂; (k) (C₆-C₁₄aryl)alkyl-O—; (l) halo; (m) C₁-C₉heteroaryl optionally substituted with from 1 to 3 substituents independently selected from: (i) C₁-C₈acyl, (ii) C₁-C₆alkyl, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl optionally substituted by C₁-C₆alkyl, (iii) (C₁-C₆alkyl)amido-, (iv) (C₁-C₆alkyl)carboxy, (v) (C₁-C₆alkyl)carboxyamido, (vi) C₁-C₆alkoxy- optionally substituted by C₁-C₆alkoxy- or C₁-C₉heteroaryl, (vii) (C₁-C₆alkoxy)carbonyl, (viii) (C₆-C₁₄aryl)oxy, (ix) C₃-C₈cycloalkyl, (x) halo, (xi) C₁-C₆haloalkyl-, (xii) C₁-C₉heterocyclyl- optionally substituted by C₁-C₆alkyl or C₁-C₆hydroxylalkyl-, (xiii) hydroxyl, (xiv) C₁-C₆hydroxylalkyl-, (xv) C₁-C₆perfluoroalkyl-, (xvi) C₁-C₆perfluoroalkyl-O—, (xvii) R¹⁷R¹⁸N—, (xviii) CN, (xix) —COOH, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) C₁-C₉heteroaryl, (xxviii) —C(═N—(OR¹⁷))—(NR¹⁷R¹⁸), and (xxix) —NO₂; (n) hydroxyl; (o) C₁-C₉heterocyclyl optionally substituted with from 1 to 3 substituents independently selected from: (i) C₁-C₆alkyl, which is optionally substituted with from 1 to 3 substituents independently selected from: A) H₂N—, B) (C₁-C₆alkyl)NH—, and C) (C₁-C₆alkyl)(C₁-C₆alkyl)N—, (ii) R¹⁷R¹⁸NC(O)—, (iii) (C₁-C₆)alkyl-O—(C₁-C₆)alkylene-, (iv) hydroxyl, and (v) R¹⁷R¹⁸N—; (p) C₁-C₆perfluoroalkyl-; (q) CN; (r) (C₁-C₆alkoxy)carbonyl; (s) CO₂H; or (t) NO₂;
R¹⁷ and R¹⁸ are each independently H; C₁-C₆alkyl optionally substituted with from 1 to 3 substituents independently selected from C₁-C₆alkoxy-, hydroxyl-, H₂N—, (C₁-C₆alkyl)NH—, or (C₁-C₆alkyl)(C₁-C₆alkyl)N—; C₁-C₆alkoxy-; C₂-C₆alkenyl; C₂-C₆alkynyl; C₁-C₆-carboxyamidoalkyl-; C₁-C₉heteroaryl optionally substituted with from 1 to 3 substituents independently selected from C₁-C₆alkyl, halo, or perfluoro(C₁-C₆)alkyl; C₁-C₉heterocyclyl- optionally substituted by C₁-C₆alkyl; C₆-C₁₄aryl optionally substituted with from 1 to 3 substituents independently selected from C₁-C₆alkyl, halo, or perfluoro(C₁-C₆)alkyl; heterocyclyl(C₁-C₆alkyl); (C₆-C₁₄aryl)alkyl optionally substituted by C₁-C₆alkyl or C₁-C₆alkoxy-; (C₁-C₉heteroaryl)alkyl optionally substituted by C₁-C₆alkyl or C₁-C₆alkoxy-; or C₃-C₈cycloalkyl;
or R¹⁷ and R¹⁸ when taken together with the nitrogen to which they are attached can form a nitrogen-containing monocyclic, bicyclic, or bridged non-aromatic C₁-C₉heterocycle wherein up to two of the carbon atoms of the C₁-C₉heterocycle can be replaced with —N(H)—, —N(C₁-C₆alkyl)-, —N(C₆-C₁₄aryl)-, —S—, —SO—, —S(O)₂, or —O—, which is also optionally substituted with from 1 to 3 substituents independently selected from C₁-C₆alkyl, C₁-C₆alkoxy-, hydroxyl, C₁-C₆hydroxylalkyl-, H₂N—, (C₁-C₆alkyl)NH—, (C₁-C₆alkyl)(C₁-C₆alkyl)N—, or (C₁-C₆)alkyl-O—(C₁-C₆)alkylene-;
R¹⁹ is C₁-C₆alkyl or C₆-C₁₄aryl;
or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;
R¹⁰ is H; C₁-C₆alkyl optionally substituted with from 1 to 3 substituents independently selected from halogen, H₂N—, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)N(C₁-C₆alkyl)(C₁-C₆alkyl), —CN, hydroxyl, C₁-C₆alkoxy-, C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, or —NO₂; C₂-C₁₀ alkenyl; C₆-C₁₄aryl; C₃-C₈cycloalkyl; C₁-C₉heteroaryl; or C₁-C₆heterocyclylalkyl group optionally substituted with from 1 to 3 substituents independently selected from halogen, H₂N—, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy-, C₁-C₆alkyl, —C(O)OH, —C(O)O(C₁-C₆alkyl), —C(O)(C₁-C₆alkyl), 4- to 7-membered monocyclic heterocycle, C₆-C₁₄aryl, C₁-C₉heteroaryl, C₁-C₆heterocyclylalkyl, or C₃-C₈cycloalkyl;
R^{X} is H or C₁-C₆alkyl.

2) A compound of claim 1 of the Formula 2: 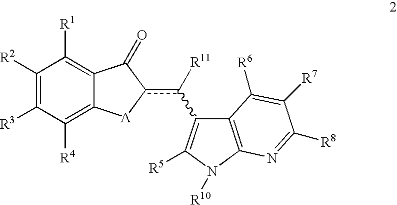
or a geometric isomer thereof or a pharmaceutically acceptable salt thereof.

3) A compound of claim 1 of the Formula 3: 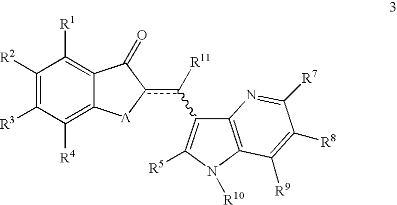
or a geometric isomer thereof or a pharmaceutically acceptable salt thereof.

4) A compound of claim 2 of the Formula 4: 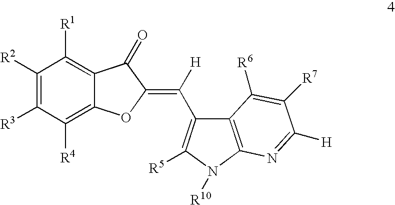
or a geometric isomer thereof or a pharmaceutically acceptable salt thereof.

5) A compound of claim 3 of the Formula 5: 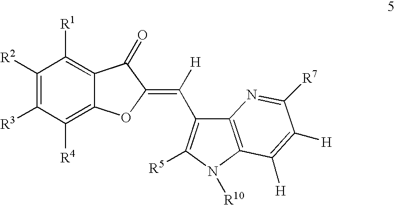
or a geometric isomer thereof or a pharmaceutically acceptable salt thereof.

6) A compound of claim 4, wherein R¹ is hydroxyl.

7) A compound of claim 6 wherein R² is H.

8) A compound of claim 7, wherein R³ is hydroxyl.

9) A compound of claim 8, wherein R⁴ is H.

10) A compound of claim 9, wherein R⁵ is H.

11) A compound of claim 10, wherein R⁶ is C₆-C₁₄aryl, optionally independently substituted or C₁-C₉heterocyclyl.

12) A compound of claim 11, wherein R⁷ is H.

13) A compound of claim 12, wherein R¹⁰ is C₁-C₆alkyl.

14) A compound of claim 13, wherein R¹⁰ is methyl.

15) A compound selected from the group consisting of:
4,6-dihydroxy-2-[(1-methyl-1H-pyrrolo[2,3-b]pyridin-3-yl)methylene]-1-benzofuran-3(2H)-one;
6,7-dihydroxy-2-[(1-methyl-1H-pyrrolo[2,3-b]pyridin-3-yl)methylene]-1-benzofuran-3(2H)-one;
4,6-dihydroxy-2-({1-[3-(4-methylpiperazin-1-yl)propyl]-1H-pyrrolo[2,3-b]pyridin-3-yl}methylene)-1-benzofuran-3(2H)-one;
4,6-dihydroxy-2-{[1-(3-morpholin-4-ylpropyl)-1H-pyrrolo[2,3-b]pyridin-3-yl]methylene}-1-benzofuran-3(2H)-one;
6,7-dihydroxy-2-({1-[3-(4-methylpiperazin-1-yl)propyl]-1H-pyrrolo[2,3-b]pyridin-3-yl}methylene)-1-benzofuran-3(2H)-one;
6,7-dihydroxy-2-{[1-(3-morpholin-4-ylpropyl)-1H-pyrrolo[2,3-b]pyridin-3-yl]methylene}-1-benzofuran-3(2H)-one;
6,7-dihydroxy-2-[(1-{3-[4-(2-hydroxyethyl)piperazin-1-yl]propyl}-1H-pyrrolo[2,3-b]pyridin-3-yl)methylene]-1-benzofuran-3(2H)-one;
4,6-dihydroxy-2-(1H-pyrrolo[2,3-b]pyridin-3-ylmethylene)-1-benzofuran-3(2H)-one;
(2Z)-2-{[4-(2-chlorophenyl)-1-methyl-1H-pyrrolo[2,3-b]pyridin-3-yl]methylene}-4,6-dihydroxy-1-benzofuran-3(2H)-one;
(2Z)-2-{[4-(3-chlorophenyl)-1-methyl-1H-pyrrolo[2,3-b]pyridin-3-yl]methylene}-4,6-dihydroxy-1-benzofuran-3(2H)-one;
(2Z)-4,6-dihydroxy-2-{[4-(2-methoxyphenyl)-1-methyl-1H-pyrrolo[2,3-b]pyridin-3-yl]methylene}-1-benzofuran-3(2H)-one;
(2Z)-4,6-dihydroxy-2-({1-methyl-4-[4-(trifluoromethoxy)phenyl]-1H-pyrrolo[2,3-b]pyridin-3-yl}methylene)-1-benzofuran-3(2H)-one;
(2Z)-4,6-dihydroxy-2-[(4-{4-[2-(1H-imidazol-1-yl)ethoxy]phenyl}-1-methyl-1H-pyrrolo[2,3-b]pyridin-3-yl)methylene]-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}benzenesulfonamide;
N-(3-{3-[(Z)-(4,6-dihydroxy-3-oxo-1-benzofuran-2(3H)-ylidene)methyl]-1-methyl-1H-pyrrolo[2,3-b]pyridin-4-yl}phenyl)methanesulfonamide;
(2Z)-4,6-dihydroxy-2-{[4-(4-hydroxyphenyl)-1-methyl-1H-pyrrolo[2,3-b]pyridin-3-yl]methylene}-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}-N′-hydroxybenzenecarboximidamide;
3-{3-[(Z)-(4-hydroxy-3-oxo-1-benzofuran-2(3H)-ylidene)methyl]-1-methyl-1H-pyrrolo[2,3-b]pyridin-4-yl}benzenesulfonamide;
(2Z)-2-({4-[4-(5,6-dihydro-4H-1,3-oxazin-2-yl)phenyl]-1-methyl-1H-pyrrolo[2,3-b]pyridin-3-yl}methylidene)-4-hydroxy-1-benzofuran-3(2H)-one;
(2Z)-4-hydroxy-2-{[1-methyl-4-(1H-pyrrol-2-yl)-1H-pyrrolo[2,3-b]pyridin-3-yl]methylidene}-1-benzofuran-3(2H)-one;
(2Z)-4,6-dihydroxy-2-[(1-methyl-4-phenyl-1H-pyrrolo[2,3-b]pyridin-3-yl)methylene]-1-benzofuran-3(2H)-one;
(2Z)-2-({4-[4-(dimethylamino)phenyl]-1-methyl-1H-pyrrolo[2,3-b]pyridin-3-yl}methylene)-4,6-dihydroxy-1-benzofuran-3(2H)-one;
(2Z)-4,6-dihydroxy-2-[(1-methyl-2-phenyl-1H-pyrrolo[2,3-b]pyridin-3-yl)methylene]-1-benzofuran-3(2H)-one;
(2Z)-6-hydroxy-2-[(1-methyl-2-phenyl-1H-pyrrolo[2,3-b]pyridin-3-yl)methylene]-1-benzofuran-3(2H)-one;
(2Z)-4-hydroxy-2-[(1-methyl-2-phenyl-1H-pyrrolo[2,3-b]pyridin-3-yl)methylene]-1-benzofuran-3(2H)-one;
(2Z)-6-hydroxy-2-[(1-methyl-4-phenyl-1H-pyrrolo[2,3-b]pyridin-3-yl)methylene]-1-benzofuran-3(2H)-one;
(2Z)-4-hydroxy-2-[(1-methyl-4-phenyl-1H-pyrrolo[2,3-b]pyridin-3-yl)methylene]-1-benzofuran-3(2H)-one;
(2Z)-4-fluoro-6-hydroxy-2-[(1-methyl-4-phenyl-1H-pyrrolo[2,3-b]pyridin-3-yl)methylene]-1-benzofuran-3(2H)-one;
(2Z)-4,6-dihydroxy-2-[(1-methyl-4-pyridin-3-yl-1H-pyrrolo[2,3-b]pyridin-3-yl)methylene]-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}benzamide;
N-(4-{3-[(Z)-(4,6-dihydroxy-3-oxo-1-benzofuran-2(3H)-ylidene)methyl]-1-methyl-1H-pyrrolo[2,3-b]pyridin-4-yl}phenyl)acetamide;
N-(4-{3-[(Z)-(4,6-dihydroxy-3-oxo-1-benzofuran-2(3H)-ylidene)methyl]-1-methyl-1H-pyrrolo[2,3-b]pyridin-4-yl}phenyl)methanesulfonamide;
(2Z)-5-bromo-2-[(1-methyl-4-phenyl-1H-pyrrolo[2,3-b]pyridin-3-yl)methylene]-1-benzofuran-3(2H)-one;
1-methyl-3-{(2Z)-2-[(1-methyl-4-phenyl-1H-pyrrolo[2,3-b]pyridin-3-yl)methylene]-3-oxo-2,3-dihydro-1-benzofuran-5-yl}urea;
(2Z)-4,6-dihydroxy-2-{[1-methyl-4-(phenylethynyl)-1H-pyrrolo[2,3-b]pyridin-3-yl]methylene}-1-benzofuran-3(2H)-one;
(2Z)-4,6-dihydroxy-2-[(1-methyl-4-piperidin-1-yl-1H-pyrrolo[2,3-b]pyridin-3-yl)methylene]-1-benzofuran-3(2H)-one;
(2Z)-2-{[4-(4-aminophenyl)-1-methyl-1H-pyrrolo[2,3-b]pyridin-3-yl]methylene}-4,6-dihydroxy-1-benzofuran-3(2H)-one;
(2Z)-5-hydroxy-2-[(1-methyl-4-phenyl-1H-pyrrolo[2,3-b]pyridin-3-yl)methylene]-1-benzofuran-3(2H)-one;
(2Z)-4,6-dihydroxy-2-({1-methyl-4-[4-(methylamino)phenyl]-1H-pyrrolo[2,3-b]pyridin-3-yl}methylene)-1-benzofuran-3(2H)-one;
(2Z)-4,6-dihydroxy-2-{[1-methyl-4-(4-methylpiperazin-1-yl)-1H-pyrrolo[2,3-b]pyridin-3-yl]methylene}-1-benzofuran-3(2H)-one;
(2Z)-4,6-dihydroxy-2-[(1-methyl-4-morpholin-4-yl-1H-pyrrolo[2,3-b]pyridin-3-yl)methylene]-1-benzofuran-3(2H)-one;
(2Z)-4,6-dihydroxy-2-[(1-methyl-4-pyrrolidin-1-yl-1H-pyrrolo[2,3-b]pyridin-3-yl)methylene]-1-benzofuran-3(2H)-one;
(2Z)-4-hydroxy-2-{[1-methyl-4-(4-methylpiperazin-1-yl)-1H-pyrrolo[2,3-b]pyridin-3-yl]methylene}-1-benzofuran-3(2H)-one;
(2Z)-4-hydroxy-2-[(1-methyl-4-morpholin-4-yl-1H-pyrrolo[2,3-b]pyridin-3-yl)methylene]-1-benzofuran-3(2H)-one;
(2Z)-4,6-dihydroxy-2-{[1-methyl-4-(4-morpholin-4-ylphenyl)-1H-pyrrolo[2,3-b]pyridin-3-yl]methylene}-1-benzofuran-3(2H)-one;
(2Z)-4,6-dihydroxy-2-{[4-(4-hydroxypiperidin-1-yl)-1-methyl-1H-pyrrolo[2,3-b]pyridin-3-yl]methylene}-1-benzofuran-3(2H)-one;
(2Z)-4,6-dihydroxy-2-({1-methyl-4-[(E)-2-phenylvinyl]-1H-pyrrolo[2,3-b]pyridin-3-yl}methylene)-1-benzofuran-3(2H)-one;
(2Z)-2-({4-[3-(dimethylamino)phenyl]-1-methyl-1H-pyrrolo[2,3-b]pyridin-3-yl}methylene)-4,6-dihydroxy-1-benzofuran-3(2H)-one;
(2Z)-4,6-dihydroxy-2-{[4-(4-methoxyphenyl)-1-methyl-1H-pyrrolo[2,3-b]pyridin-3-yl]methylene}-1-benzofuran-3(2H)-one;
(2Z)-4,6-dihydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one;
(2Z)-4-hydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one;
(2Z)-4,6-dihydroxy-2-({1-methyl-4-[4-(4-methylpiperazin-1-yl)phenyl]-1H-pyrrolo[2,3-b]pyridin-3-yl}methylene)-1-benzofuran-3(2H)-one;
(2Z)-4-hydroxy-2-({1-methyl-4-[4-(4-methylpiperazin-1-yl)phenyl]-1H-pyrrolo[2,3-b]pyridin-3-yl}methylene)-1-benzofuran-3(2H)-one;
(2Z)-5-amino-2-[(1-methyl-4-phenyl-1H-pyrrolo[2,3-b]pyridin-3-yl)methylene]-1-benzofuran-3(2H)-one;
(2Z)-5-bromo-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one;
2-hydroxyethyl {(2Z)-2-[(1-methyl-4-phenyl-1H-pyrrolo[2,3-b]pyridin-3-yl)methylene]-3-oxo-2,3-dihydro-1-benzofuran-5-yl}carbamate;
1-ethyl-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
(2Z)-4,6-dihydroxy-2-[(1-methyl-4-pyrimidin-5-yl-1H-pyrrolo[2,3-b]pyridin-3-yl)methylene]-1-benzofuran-3(2H)-one;
(2Z)-2-{[4-(6-chloropyridin-3-yl)-1-methyl-1H-pyrrolo[2,3-b]pyridin-3-yl]methylene}-4,6-dihydroxy-1-benzofuran-3(2H)-one;
(2Z)-2-{[4-(4-chlorophenyl)-1-methyl-1H-pyrrolo[2,3-b]pyridin-3-yl]methylene}-4,6-dihydroxy-1-benzofuran-3(2H)-one;
(2Z)-2-[(4-azepan-1-yl-1-methyl-1H-pyrrolo[2,3-b]pyridin-3-yl)methylene]-4,6-dihydroxy-1-benzofuran-3(2H)-one;
(2Z)-4,6-dihydroxy-2-{[4-(3-methoxyphenyl)-1-methyl-1H-pyrrolo[2,3-b]pyridin-3-yl]methylene}-1-benzofuran-3(2H)-one;
(2Z)-4,6-dihydroxy-2-({4-[4-(hydroxymethyl)phenyl]-1-methyl-1H-pyrrolo[2,3-b]pyridin-3-yl}methylene)-1-benzofuran-3(2H)-one;
2,2-dimethyl-N-{(2Z)-2-[(1-methyl-4-phenyl-1H-pyrrolo[2,3-b]pyridin-3-yl)methylene]-3-oxo-2,3-dihydro-1-benzofuran-4-yl}propanamide;
(2Z)-4-hydroxy-2-{[1-methyl-4-(6-morpholin-4-ylpyridin-3-yl)-1H-pyrrolo[2,3-b]pyridin-3-yl]methylene}-1-benzofuran-3(2H)-one;
(2Z)-4-hydroxy-2-{[1-methyl-4-(6-piperidin-1-ylpyridin-3-yl)-1H-pyrrolo[2,3-b]pyridin-3-yl]methylene}-1-benzofuran-3(2H)-one;
(2Z)-4-hydroxy-2-{[4-(6-methoxypyridin-3-yl)-1-methyl-1H-pyrrolo[2,3-b]pyridin-3-yl]methylene}-1-benzofuran-3(2H)-one;
(2Z)-4,6-dihydroxy-2-({1-methyl-4-[4-(methylsulfonyl)phenyl]-1H-pyrrolo[2,3-b]pyridin-3-yl}methylene)-1-benzofuran-3(2H)-one;
(2Z)-4-hydroxy-2-({1-methyl-4-[4-(methylsulfonyl)phenyl]-1H-pyrrolo[2,3-b]pyridin-3-yl}methylene)-1-benzofuran-3(2H)-one;
(2Z)-4,6-dihydroxy-2-({1-methyl-4-[3-(methylsulfonyl)phenyl]-1H-pyrrolo[2,3-b]pyridin-3-yl}methylene)-1-benzofuran-3(2H)-one;
(2Z)-4-hydroxy-2-({1-methyl-4-[3-(methylsulfonyl)phenyl]-1H-pyrrolo[2,3-b]pyridin-3-yl}methylene)-1-benzofuran-3(2H)-one;
(2Z)-4-hydroxy-2-({4-[6-(hydroxymethyl)pyridin-3-yl]-1-methyl-1H-pyrrolo[2,3-b]pyridin-3-yl}methylene)-1-benzofuran-3(2H)-one;
4-{3-[(Z)-(4,6-dihydroxy-3-oxo-1-benzofuran-2(3H)-ylidene)methyl]-1-methyl-1H-pyrrolo[2,3-b]pyridin-4-yl}-N-methylbenzenesulfonamide;
(2Z)-4,6-dihydroxy-2-{[1-methyl-4-(1,2,3,6-tetrahydropyridin-4-yl)-1H-pyrrolo[2,3-b]pyridin-3-yl]methylene}-1-benzofuran-3(2H)-one;
(2Z)-4,6-dihydroxy-2-({1-methyl-4-[3-(pyrrolidin-1-ylcarbonyl)phenyl]-1H-pyrrolo[2,3-b]pyridin-3-yl}methylene)-1-benzofuran-3(2H)-one;
(2Z)-4,6-dihydroxy-2-({1-methyl-4-[3-(piperidin-1-ylsulfonyl)phenyl]-1H-pyrrolo[2,3-b]pyridin-3-yl}methylene)-1-benzofuran-3(2H)-one;
(2Z)-4-hydroxy-2-({4-[4-methoxy-3-(morpholin-4-ylsulfonyl)phenyl]-1-methyl-1H-pyrrolo[2,3-b]pyridin-3-yl}methylene)-1-benzofuran-3(2H)-one;
N-cyclopropyl-4-{3-[(Z)-(4-hydroxy-3-oxo-1-benzofuran-2(3H)-ylidene)methyl]-1-methyl-1H-pyrrolo[2,3-b]pyridin-4-yl}benzenesulfonamide;
N-(4-fluorophenyl)-4-{3-[(Z)-(4-hydroxy-3-oxo-1-benzofuran-2(3H)-ylidene)methyl]-1-methyl-1H-pyrrolo[2,3-b]pyridin-4-yl}benzamide;
(2Z)-4-hydroxy-2-{[1-methyl-4-(4-phenoxyphenyl)-1H-pyrrolo[2,3-b]pyridin-3-yl]methylene}-1-benzofuran-3(2H)-one;
(2Z)-2-{[4-(1-benzofuran-2-yl)-1-methyl-1H-pyrrolo[2,3-b]pyridin-3-yl]methylene}-4-hydroxy-1-benzofuran-3(2H)-one;
(2Z)-4-hydroxy-2-({4-[4-methoxy-3-(pyrrolidin-1-ylsulfonyl)phenyl]-1-methyl-1H-pyrrolo[2,3-b]pyridin-3-yl}methylene)-1-benzofuran-3(2H)-one;
(2Z)-4,6-dihydroxy-2-{[1-methyl-4-(4-phenoxyphenyl)-1H-pyrrolo[2,3-b]pyridin-3-yl]methylene}-1-benzofuran-3(2H)-one;
(2Z)-4,6-dihydroxy-2-({4-[4-methoxy-3-(morpholin-4-ylsulfonyl)phenyl]-1-methyl-1H-pyrrolo[2,3-b]pyridin-3-yl}methylene)-1-benzofuran-3(2H)-one;
(2Z)-4,6-dihydroxy-2-({4-[4-methoxy-3-(pyrrolidin-1-ylsulfonyl)phenyl]-1-methyl-1H-pyrrolo[2,3-b]pyridin-3-yl}methylene)-1-benzofuran-3(2H)-one;
N-benzyl-4-{3-[(Z)-(4-hydroxy-3-oxo-1-benzofuran-2(3H)-ylidene)methyl]-1-methyl-1H-pyrrolo[2,3-b]pyridin-4-yl}benzenesulfonamide;
(2Z)-4-hydroxy-2-({1-methyl-4-[4-(pyrrolidin-1-ylcarbonyl)phenyl]-1H-pyrrolo[2,3-b]pyridin-3-yl}methylene)-1-benzofuran-3(2H)-one;
(2Z)-4,6-dihydroxy-2-({1-methyl-4-[4-(pyrrolidin-1-ylcarbonyl)phenyl]-1H-pyrrolo[2,3-b]pyridin-3-yl}methylene)-1-benzofuran-3(2H)-one;
(2Z)-2-{[4-(2,6-dimethylmorpholin-4-yl)-1-methyl-1H-pyrrolo[2,3-b]pyridin-3-yl]methylene}-4-hydroxy-1-benzofuran-3(2H)-one;
(2Z)-4,6-dihydroxy-2-[(1-methyl-4-phenoxy-1H-pyrrolo[2,3-b]pyridin-3-yl)methylene]-1-benzofuran-3(2H)-one;
(2Z)-4-hydroxy-2-[(4-hydroxy-1-methyl-1H-pyrrolo[2,3-b]pyridin-3-yl)methylene]-1-benzofuran-3(2H)-one;
N-(4-fluorophenyl)-3-{3-[(Z)-(4-hydroxy-3-oxo-1-benzofuran-2(3H)-ylidene)methyl]-1-methyl-1H-pyrrolo[2,3-b]pyridin-4-yl}benzamide;
3-{3-[(Z)-(4,6-dihydroxy-3-oxo-1-benzofuran-2(3H)-ylidene)methyl]-1-methyl-1H-pyrrolo[2,3-b]pyridin-4-yl}-N-(4-fluorophenyl)benzamide;
5-{3-[(Z)-(4,6-dihydroxy-3-oxo-1-benzofuran-2(3H)-ylidene)methyl]-1-methyl-1H-pyrrolo[2,3-b]pyridin-4-yl}-2-fluoro-N-phenylbenzamide;
1-methyl-3-{(2Z)-2-[(1-methyl-4-phenyl-1H-pyrrolo[2,3-b]pyridin-3-yl)methylene]-3-oxo-2,3-dihydro-1-benzofuran-4-yl}urea;
(2Z)-2-{[4-(3,5-dimethylpiperidin-1-yl)-1-methyl-1H-pyrrolo[2,3-b]pyridin-3-yl]methylene}-4-hydroxy-1-benzofuran-3(2H)-one;
(2Z)-2-{[4-(2,5-diazabicyclo[2.2.1]hept-2-yl)-1-methyl-1H-pyrrolo[2,3-b]pyridin-3-yl]methylene}-4-hydroxy-1-benzofuran-3(2H)-one;
(2Z)-4-hydroxy-2-[(1-methyl-4-thiomorpholin-4-yl-1H-pyrrolo[2,3-b]pyridin-3-yl)methylene]-1-benzofuran-3(2H)-one;
(2Z)-4-hydroxy-2-({1-methyl-4-[3-(pyrrolidin-1-ylcarbonyl)phenyl]-1H-pyrrolo[2,3-b]pyridin-3-yl}methylene)-1-benzofuran-3(2H)-one;
(2Z)-4-hydroxy-2-({1-methyl-4-[4-(pyrrolidin-1-ylcarbonyl)piperazin-1-yl]-1H-pyrrolo[2,3-b]pyridin-3-yl}methylene)-1-benzofuran-3(2H)-one;
(2Z)-4-hydroxy-2-{[1-methyl-4-(1,3,3-trimethyl-6-azabicyclo[3.2.1]oct-6-yl)-1H-pyrrolo[2,3-b]pyridin-3-yl]methylene}-1-benzofuran-3(2H)-one;
(2Z)-4-hydroxy-2-({1-methyl-4-[4-(trifluoromethyl)phenyl]-1H-pyrrolo[2,3-b]pyridin-3-yl}methylene)-1-benzofuran-3(2H)-one;
(2Z)-4-hydroxy-2-({1-methyl-4-[3-(trifluoromethyl)phenyl]-1H-pyrrolo[2,3-b]pyridin-3-yl}methylene)-1-benzofuran-3(2H)-one;
(2Z)-4-amino-2-[(1-methyl-4-phenyl-1H-pyrrolo[2,3-b]pyridin-3-yl)methylene]-1-benzofuran-3(2H)-one;
(2Z)-4-hydroxy-2-({1-methyl-4-[3-(pyrrolidin-1-ylcarbonyl)piperidin-1-yl]-1H-pyrrolo[2,3-b]pyridin-3-yl}methylene)-1-benzofuran-3(2H)-one;
(2Z)-4-hydroxy-2-({1-methyl-4-[3-(trifluoromethoxy)phenyl]-1H-pyrrolo[2,3-b]pyridin-3-yl}methylene)-1-benzofuran-3(2H)-one;
4-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzamide;
(2Z)-2-({4-[3-(dimethylamino)pyrrolidin-1-yl]-1-methyl-1H-pyrrolo[2,3-b]pyridin-3-yl}methylene)-4-hydroxy-1-benzofuran-3(2H)-one;
(2Z)-2-[(4-{4-[(dimethylamino)methyl]phenyl}-1-methyl-1H-pyrrolo[2,3-b]pyridin-3-yl)methylene]-4-hydroxy-1-benzofuran-3(2H)-one;
1-{3-[(Z)-(4-hydroxy-3-oxo-1-benzofuran-2(3H)-ylidene)methyl]-1-methyl-1H-pyrrolo[2,3-b]pyridin-4-yl}piperidine-4-carboxamide;
(2Z)-2-({4-[4-(aminomethyl)piperidin-1-yl]-1-methyl-1H-pyrrolo[2,3-b]pyridin-3-yl}methylene)-4-hydroxy-1-benzofuran-3(2H)-one;
(2Z)-2-[(1-ethyl-4-phenyl-1H-pyrrolo[2,3-b]pyridin-3-yl)methylene]-4-hydroxy-1-benzofuran-3(2H)-one;
(2Z)-4-hydroxy-2-{[1-methyl-4-(2-oxa-5-azabicyclo[2.2.1]hept-5-yl)-1H-pyrrolo[2,3-b]pyridin-3-yl]methylene}-1-benzofuran-3(2H)-one;
(2Z)-2-[(4-{4-[3-(dimethylamino)propyl]piperazin-1-yl}-1-methyl-1H-pyrrolo[2,3-b]pyridin-3-yl)methylene]-4-hydroxy-1-benzofuran-3(2H)-one;
1-{(2Z)-2-[(5-methoxy-1H-pyrrolo[2,3-b]pyridin-3-yl)methylene]-3-oxo-2,3-dihydro-1-benzofuran-5-yl}-3-methylurea;
1-[(2Z)-2-({5-methoxy-1-[2-(4-methylpiperazin-1-yl)ethyl]-1H-pyrrolo[2,3-b]pyridin-3-yl}methylene)-3-oxo-2,3-dihydro-1-benzofuran-5-yl]-3-methylurea;
3-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzamide;
(2Z)-2-[(4-{3-[(dimethylamino)methyl]phenyl}-1-methyl-1H-pyrrolo[2,3-b]pyridin-3-yl)methylene]-4-hydroxy-1-benzofuran-3(2H)-one;
(2Z)-4-hydroxy-2-({4-[4-methoxy-3-(piperidin-1-ylsulfonyl)phenyl]-1-methyl-1H-pyrrolo[2,3-b]pyridin-3-yl}methylene)-1-benzofuran-3(2H)-one;
(2Z)-4-hydroxy-2-[(1-methyl-4-{4-[(4-methylpiperazin-1-yl)carbonyl]phenyl}-1H-pyrrolo[2,3-b]pyridin-3-yl)methylene]-1-benzofuran-3(2H)-one;
(2Z)-4-hydroxy-2-{[4-(3-hydroxyphenyl)-1-methyl-1H-pyrrolo[2,3-b]pyridin-3-yl]methylene}-1-benzofuran-3(2H)-one;
(2Z)-4-hydroxy-2-{[1-methyl-4-(4-methyl-3,4-dihydro-2H-1,4-benzoxazin-7-yl)-1H-pyrrolo[2,3-b]pyridin-3-yl]methylene}-1-benzofuran-3(2H)-one;
(2Z)-2-({4-[4-(2-furyl)phenyl]-1-methyl-1H-pyrrolo[2,3-b]pyridin-3-yl}methylene)-4-hydroxy-1-benzofuran-3(2H)-one;
(2Z)-4-hydroxy-2-({1-methyl-4-[4-(morpholin-4-ylcarbonyl)phenyl]-1H-pyrrolo[2,3-b]pyridin-3-yl}methylene)-1-benzofuran-3(2H)-one;
(2Z)-4-hydroxy-2-({1-methyl-4-[4-(4-methylpiperazin-1-yl)piperidin-1-yl]-1H-pyrrolo[2,3-b]pyridin-3-yl}methylene)-1-benzofuran-3(2H)-one;
(2Z)-4-hydroxy-2-({1-methyl-4-[4-(morpholin-4-ylmethyl)phenyl]-1H-pyrrolo[2,3-b]pyridin-3-yl}methylene)-1-benzofuran-3(2H)-one;
1-methyl-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
4-{3-[(Z)-(4-hydroxy-3-oxo-1-benzofuran-2(3H)-ylidene)methyl]-1-methyl-1H-pyrrolo[2,3-b]pyridin-4-yl}-N-methylbenzamide;
1-(4-{3-[(Z)-(4-hydroxy-3-oxo-1-benzofuran-2(3H)-ylidene)methyl]-1-methyl-1H-pyrrolo[2,3-b]pyridin-4-yl}phenyl)-3-methylurea;
(2Z)-2-{[4-(4-acetylphenyl)-1-methyl-1H-pyrrolo[2,3-b]pyridin-3-yl]methylene}-4-hydroxy-1-benzofuran-3(2H)-one;
(2Z)-4-hydroxy-2-{[1-methyl-4-(4-piperazin-1-ylphenyl)-1H-pyrrolo[2,3-b]pyridin-3-yl]methylene}-1-benzofuran-3(2H)-one;
(2Z)-4-hydroxy-2-({1-methyl-4-[4-(piperazin-1-ylcarbonyl)phenyl]-1H-pyrrolo[2,3-b]pyridin-3-yl}methylene)-1-benzofuran-3(2H)-one;
2-fluoro-5-{3-[(Z)-(4-hydroxy-3-oxo-1-benzofuran-2(3H)-ylidene)methyl]-1-methyl-1H-pyrrolo[2,3-b]pyridin-4-yl}-N-phenylbenzamide;
3-{3-[(Z)-(4-hydroxy-3-oxo-1-benzofuran-2(3H)-ylidene)methyl]-1-methyl-1H-pyrrolo[2,3-b]pyridin-4-yl}-N-phenylbenzamide;
(2Z)-2-{[4-(4-aminopiperidin-1-yl)-1-methyl-1H-pyrrolo[2,3-b]pyridin-3-yl]methylene}-4-hydroxy-1-benzofuran-3(2H)-one;
3-(4-{3-[(Z)-(4-hydroxy-3-oxo-1-benzofuran-2(3H)-ylidene)methyl]-1-methyl-1H-pyrrolo[2,3-b]pyridin-4-yl}phenyl)-1,1-dimethylurea;
2-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzamide;
(2Z)-4-hydroxy-2-{[1-methyl-4-(4-piperidin-1-ylphenyl)-1H-pyrrolo[2,3-b]pyridin-3-yl]methylidene}-1-benzofuran-3(2H)-one;
(2Z)-4-hydroxy-2-{[1-methyl-4-(4-pyrrolidin-1-ylphenyl)-1H-pyrrolo[2,3-b]pyridin-3-yl]methylidene}-1-benzofuran-3(2H)-one;
(2Z)-4-hydroxy-2-({1-methyl-4-[4-(8-oxa-3-azabicyclo[3.2.1]oct-3-ylcarbonyl)phenyl]-1H-pyrrolo[2,3-b]pyridin-3-yl}methylidene)-1-benzofuran-3(2H)-one;
1-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylpiperidine-4-carboxamide;
(2Z)-4-hydroxy-2-{[4-(4-{[4-(2-hydroxyethyl)piperazin-1-yl]carbonyl}phenyl)-1-methyl-1H-pyrrolo[2,3-b]pyridin-3-yl]methylidene}-1-benzofuran-3(2H)-one;
4-{3-[(Z)-(4-hydroxy-3-oxo-1-benzofuran-2(3H)-ylidene)methyl]-1-methyl-1H-pyrrolo[2,3-b]pyridin-4-yl}-N-(3-hydroxypropyl)benzamide;
N-(2-hydroxyethyl)-1-{3-[(Z)-(4-hydroxy-3-oxo-1-benzofuran-2(3H)-ylidene)methyl]-1-methyl-1H-pyrrolo[2,3-b]pyridin-4-yl}piperidine-4-carboxamide;
4-{3-[(Z)-(4-hydroxy-3-oxo-1-benzofuran-2(3H)-ylidene)methyl]-1-methyl-1H-pyrrolo[2,3-b]pyridin-4-yl}-N-(2-methoxyethyl)benzamide;
(2Z)-4-hydroxy-2-({1-methyl-4-[4-(morpholin-4-ylcarbonyl)piperidin-1-yl]-1H-pyrrolo[2,3-b]pyridin-3-yl}methylidene)-1-benzofuran-3(2H)-one;
(2Z)-4-hydroxy-2-{[1-methyl-4-(3-oxa-8-azabicyclo[3.2.1]oct-8-yl)-1H-pyrrolo[2,3-b]pyridin-3-yl]methylidene}-1-benzofuran-3(2H)-one;
(2Z)-4-hydroxy-2-({1-methyl-4-[4-(pyrrolidin-1-ylcarbonyl)piperidin-1-yl]-1H-pyrrolo[2,3-b]pyridin-3-yl}methylidene)-1-benzofuran-3(2H)-one;
(2Z)-4-hydroxy-2-{[1-methyl-4-(5-methyl-2,5-diazabicyclo[2.2.1]hept-2-yl)-1H-pyrrolo[2,3-b]pyridin-3-yl]methylidene}-1-benzofuran-3(2H)-one;
N-{(2Z)-2-[(1-methyl-4-phenyl-1H-pyrrolo[2,3-b]pyridin-3-yl)methylidene]-3-oxo-2,3-dihydro-1-benzofuran-5-yl}acetamide;
N-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]acetamide;
2,2,2-trifluoro-N-{(2Z)-2-[(1-methyl-4-phenyl-1H-pyrrolo[2,3-b]pyridin-3-yl)methylidene]-3-oxo-2,3-dihydro-1-benzofuran-5-yl}acetamide;
(2Z)-4-hydroxy-2-({1-methyl-4-[4-(8-oxa-3-azabicyclo[3.2.1]oct-3-yl)phenyl]-1H-pyrrolo[2,3-b]pyridin-3-yl}methylidene)-1-benzofuran-3(2H)-one;
N-[2-(dimethylamino)ethyl]-4-{3-[(Z)-(4-hydroxy-3-oxo-1-benzofuran-2(3H)-ylidene)methyl]-1-methyl-1H-pyrrolo[2,3-b]pyridin-4-yl}benzamide;
(2Z)-4-[2-(dimethylamino)ethoxy]-2-[(1-methyl-4-phenyl-1H-pyrrolo[2,3-b]pyridin-3-yl)methylidene]-1-benzofuran-3(2H)-one;
ethyl 4-{3-[(Z)-(4-hydroxy-3-oxo-1-benzofuran-2(3H)-ylidene)methyl]-1-methyl-1H-pyrrolo[2,3-b]pyridin-4-yl}benzoate;
1,1-dimethyl-3-{(2Z)-2-[(1-methyl-4-phenyl-1H-pyrrolo[2,3-b]pyridin-3-yl)methylidene]-3-oxo-2,3-dihydro-1-benzofuran-5-yl}urea;
N-{(2Z)-2-[(1-methyl-4-phenyl-1H-pyrrolo[2,3-b]pyridin-3-yl)methylidene]-3-oxo-2,3-dihydro-1-benzofuran-5-yl}methanesulfonamide;
N,N-dimethyl-1-{1-methyl-3-[(Z)-{3-oxo-5-[(trifluoroacetyl)amino]-1-benzofuran-2(3H)-ylidene}methyl]-1H-pyrrolo[2,3-b]pyridin-4-yl}piperidine-4-carboxamide;
(2Z)-4-hydroxy-2-[(1-methyl-4-{4-[(4-methylpiperazin-1-yl)carbonyl]piperidin-1-yl}-1H-pyrrolo[2,3-b]pyridin-3-yl)methylidene]-1-benzofuran-3(2H)-one;
(2Z)-2-{[4-(6,8-dioxa-3-azabicyclo[3.2.1]oct-3-yl)-1-methyl-1H-pyrrolo[2,3-b]pyridin-3-yl]methylidene}-4-hydroxy-1-benzofuran-3(2H)-one;
1-[(2Z)-2-{[4-(4-{[4-(2-hydroxyethyl)piperazin-1-yl]carbonyl}phenyl)-1-methyl-1H-pyrrolo[2,3-b]pyridin-3-yl]methylidene}-3-oxo-2,3-dihydro-1-benzofuran-5-yl]-3-methylurea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-pyridin-3-ylurea;
N,N-dimethyl-1-{1-methyl-3-[(Z)-{5-[(methylcarbamoyl)amino]-3-oxo-1-benzofuran-2(3H)-ylidene}methyl]-1H-pyrrolo[2,3-b]pyridin-4-yl}piperidine-4-carboxamide;
1-{3-[(Z)-(5-bromo-3-oxo-1-benzofuran-2(3H)-ylidene)methyl]-1-methyl-1H-pyrrolo[2,3-b]pyridin-4-yl}-N,N-dimethylpiperidine-4-carboxamide;
N-[(2Z)-2-({4-[4-(dimethylcarbamoyl)piperidin-1-yl]-1-methyl-1H-pyrrolo[2,3-b]pyridin-3-yl}methylidene)-3-oxo-2,3-dihydro-1-benzofuran-5-yl]morpholine-4-carboxamide;
ethyl[(2Z)-2-({4-[4-(dimethylcarbamoyl)piperidin-1-yl]-1-methyl-1H-pyrrolo[2,3-b]pyridin-3-yl}methylidene)-3-oxo-2,3-dihydro-1-benzofuran-5-yl]carbamate;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-phenylurea;
1-[(2Z)-2-({1-methyl-4-[4-(morpholin-4-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
1-methyl-3-[(2Z)-2-({1-methyl-4-[4-(morpholin-4-ylcarbonyl)piperidin-1-yl]-1H-pyrrolo[2,3-b]pyridin-3-yl}methylidene)-3-oxo-2,3-dihydro-1-benzofuran-5-yl]urea;
4-{3-[(Z)-(4-hydroxy-3-oxo-1-benzofuran-2(3H)-ylidene)methyl]-1-methyl-1H-pyrrolo[2,3-b]pyridin-4-yl}-N-(2-methoxyethyl)-N-methylbenzamide;
(2Z)-2-[(1-methyl-4-phenyl-1H-pyrrolo[2,3-b]pyridin-3-yl)methylidene]-3-oxo-2,3-dihydro-1-benzofuran-5-carboxylic acid;
1-[(2Z)-2-{[1-ethyl-4-(4-{[4-(2-hydroxyethyl)piperazin-1-yl]carbonyl}phenyl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-({1-methyl-4-[4-(morpholin-4-ylcarbonyl)phenyl]-1H-pyrrolo[2,3-b]pyridin-3-yl}methylidene)-3-oxo-2,3-dihydro-1-benzofuran-5-yl]-3-pyridin-3-ylurea;
1-methyl-3-[(2Z)-2-({1-methyl-4-[4-(morpholin-4-ylcarbonyl)phenyl]-1H-pyrrolo[2,3-b]pyridin-3-yl}methylidene)-3-oxo-2,3-dihydro-1-benzofuran-5-yl]urea;
4-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylbenzenesulfonamide;
(2Z)-4-hydroxy-2-({1-methyl-4-[4-(piperidin-1-ylsulfonyl)phenyl]-1H-pyrrolo[2,3-b]pyridin-3-yl}methylidene)-1-benzofuran-3(2H)-one;
N-(2-hydroxyethyl)-4-{3-[(Z)-(4-hydroxy-3-oxo-1-benzofuran-2(3H)-ylidene)methyl]-1-methyl-1H-pyrrolo[2,3-b]pyridin-4-yl}benzamide;
1-{(2Z)-2-[(1-methyl-4-phenyl-1H-pyrrolo[2,3-b]pyridin-3-yl)methylidene]-3-oxo-2,3-dihydro-1-benzofuran-5-yl}-3-pyridin-3-ylurea;
1-[(2Z)-2-{[1-methyl-4-(4-methylpiperazin-1-yl)-1H-pyrrolo[2,3-b]pyridin-3-yl]methylidene}-3-oxo-2,3-dihydro-1-benzofuran-5-yl]-3-pyridin-3-ylurea;
1-{(2Z)-2-[(1-methyl-4-morpholin-4-yl-1H-pyrrolo[2,3-b]pyridin-3-yl)methylidene]-3-oxo-2,3-dihydro-1-benzofuran-5-yl}-3-pyridin-3-ylurea;
(2Z)-2-{[1-ethyl-4-(4-{[4-(2-hydroxyethyl)piperazin-1-yl]carbonyl}phenyl)-1H-pyrrolo[2,3-b]pyridin-3-yl]methylidene}-4-hydroxy-1-benzofuran-3(2H)-one;
(2Z)-N-[2-(dimethylamino)ethyl]-2-[(1-methyl-4-phenyl-1H-pyrrolo[2,3-b]pyridin-3-yl)methylidene]-3-oxo-2,3-dihydro-1-benzofuran-5-carboxamide;
4-methyl-N-{(2Z)-2-[(1-methyl-4-phenyl-1H-pyrrolo[2,3-b]pyridin-3-yl)methylidene]-3-oxo-2,3-dihydro-1-benzofuran-5-yl}piperazine-1-carboxamide;
N,N-dimethyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
(2Z)-4-hydroxy-2-({1-methyl-4-[4-(pyrrolidin-1-ylsulfonyl)phenyl]-1H-pyrrolo[2,3-b]pyridin-3-yl}methylidene)-1-benzofuran-3(2H)-one;
(2Z)-4-hydroxy-2-({1-methyl-4-[4-(morpholin-4-ylsulfonyl)phenyl]-1H-pyrrolo[2,3-b]pyridin-3-yl}methylidene)-1-benzofuran-3(2H)-one;
4-{3-[(Z)-(4-hydroxy-3-oxo-1-benzofuran-2(3H)-ylidene)methyl]-1-methyl-1H-pyrrolo[2,3-b]pyridin-4-yl}-N-methoxybenzamide;
1-[2-(dimethylamino)ethyl]-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[2-(4-methylpiperazin-1-yl)ethyl]urea;
N-(2-hydroxyethyl)-4-{3-[(Z)-(4-hydroxy-3-oxo-1-benzofuran-2(3H)-ylidene)methyl]-1-methyl-1H-pyrrolo[2,3-b]pyridin-4-yl}-N-methylbenzamide;
4-{3-[(Z)-(4-hydroxy-3-oxo-1-benzofuran-2(3H)-ylidene)methyl]-1-methyl-1H-pyrrolo[2,3-b]pyridin-4-yl}-N-methoxy-N-methylbenzamide;
(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-carboxylic acid;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-pyridin-4-ylurea;
N-(3-methoxypropyl)-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-(3-methoxypropyl)-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-[2-(dimethylamino)-2-oxoethyl]-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-(2-amino-2-oxoethyl)-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(2-pyrrolidin-1-ylethyl)piperidine-4-carboxamide;
1-[2-({4-[4-(2,5-dihydro-1H-pyrrol-1-ylcarbonyl)piperidin-1-yl]-1-methyl-1H-pyrrolo[2,3-b]pyridin-3-yl}methylidene)-3-oxo-2,3-dihydro-1-benzofuran-5-yl]-3-pyridin-3-ylurea;
N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(1-methylpyrrolidin-3-yl)piperidine-4-carboxamide;
N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-[(3R)-pyrrolidin-3-yl]piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-[(3R)-pyrrolidin-3-yl]piperidine-4-carboxamide;
N-methyl-1-[1-methyl-3-({3-oxo-5-[(pyridin-3-ylcarbamoyl)amino]-1-benzofuran-2(3H)-ylidene}methyl)-H-pyrrolo[2,3-b]pyridin-4-yl]-N-[(3S)-pyrrolidin-3-yl]piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-[(3S)-pyrrolidin-3-yl]piperidine-4-carboxamide;
1-(2-{[4-(4-{[3-(dimethylamino)pyrrolidin-1-yl]carbonyl}piperidin-1-yl)-1-methyl-1H-pyrrolo[2,3-b]pyridin-3-yl]methylidene}-3-oxo-2,3-dihydro-1-benzofuran-5-yl)-3-pyridin-3-ylurea;
N-(1,1-dioxidotetrahydrothiophen-3-yl)-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-(1,1-dioxidotetrahydrothiophen-3-yl)-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(tetrahydro-2H-pyran-4-yl)piperidine-4-carboxamide;
1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]-N-(tetrahydro-2H-pyran-4-yl)piperidine-4-carboxamide;
N-benzyl-N-methyl-1-[1-methyl-3-({3-oxo-5-[(pyridin-3-ylcarbamoyl)amino]-1-benzofuran-2(3H)-ylidene}methyl)-1H-pyrrolo[2,3-b]pyridin-4-yl]piperidine-4-carboxamide;
N,N-dimethyl-1-(1-methyl-3-{(Z)-[3-oxo-5-({[4-(piperidin-1-ylmethyl)phenyl]carbamoyl}amino)-1-benzofuran-2(3H)-ylidene]methyl}-1H-pyrrolo[2,3-b]pyridin-4-yl)piperidine-4-carboxamide;
N,N-dimethyl-1-(1-methyl-3-{(Z)-[5-{[(6-morpholin-4-ylpyridin-3-yl)carbamoyl]amino}-3-oxo-1-benzofuran-2(3H)-ylidene]methyl}-1H-pyrrolo[2,3-b]pyridin-4-yl)piperidine-4-carboxamide;
N,N-dimethyl-1-(1-methyl-3-{(Z)-[5-({[4-(4-methylpiperazin-1-yl)phenyl]carbamoyl}amino)-3-oxo-1-benzofuran-2(3H)-ylidene]methyl}-1H-pyrrolo[2,3-b]pyridin-4-yl)piperidine-4-carboxamide;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-(6-morpholin-4-ylpyridin-3-yl)urea;
1-{4-[2-(dimethylamino)ethoxy]phenyl}-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-(4-methylpiperazin-1-yl)urea;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-[(3-methylpyridin-4-yl)methyl]piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-[(2-methylpyridin-4-yl)methyl]piperidine-4-carboxamide;
N-[(4-methoxypyridin-2-yl)methyl]-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-[(6-methylpyridin-2-yl)methyl]piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-prop-2-yn-1-ylpiperidine-4-carboxamide;
N-cyclopentyl-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N,N-dimethyl-1-(1-methyl-3-{(Z)-[5-{[(4-methylpiperazin-1-yl)carbamoyl]amino}-3-oxo-1-benzofuran-2(3H)-ylidene]methyl}-1H-pyrrolo[2,3-b]pyridin-4-yl)piperidine-4-carboxamide;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[4-(4-methylpiperazin-1-yl)phenyl]urea;
N,N-dimethyl-1-(1-methyl-3-{(Z)-[5-({[6-(4-methylpiperazin-1-yl)pyridin-3-yl]carbamoyl}amino)-3-oxo-1-benzofuran-2(3H)-ylidene]methyl}-1H-pyrrolo[2,3-b]pyridin-4-yl)piperidine-4-carboxamide;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[6-(4-methylpiperazin-1-yl)pyridin-3-yl]urea;
1-[(1-methyl-1H-imidazol-5-yl)methyl]-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-({1-methyl-4-[4-(2-oxa-5-azabicyclo[2.2.2]oct-5-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
1-[(2Z)-2-{[1-methyl-4-(piperidin-1-ylcarbonyl)-1H-pyrrolo[2,3-b]pyridin-3-yl]methylidene}-3-oxo-2,3-dihydro-1-benzofuran-5-yl]-3-pyridin-3-ylurea;
1-(1-methylethyl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-cyclohexyl-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-cyclopropyl-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-({1-methyl-4-[(3S)-3-methylmorpholin-4-yl]-1H-pyrrolo[2,3-b]pyridin-3-yl}methylidene)-3-oxo-2,3-dihydro-1-benzofuran-5-yl]-3-pyridin-3-ylurea;
1-{(2Z)-2-[(4-{4-[(2R,6S)-2,6-dimethylmorpholin-4-yl]piperidin-1-yl}-1-methyl-1H-pyrrolo[2,3-b]pyridin-3-yl)methylidene]-3-oxo-2,3-dihydro-1-benzofuran-5-yl}-3-pyridin-3-ylurea;
1-(3-chloropropyl)-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
1-[3-(dimethylamino)propyl]-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
1-{3-[(2-methoxyethyl)(methyl)amino]propyl}-3-[(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-5-yl]urea;
N-ethyl-N-(2-hydroxyethyl)-4-{3-[(Z)-(4-hydroxy-3-oxo-1-benzofuran-2(3H)-ylidene) methyl]-1-methyl-1H-pyrrolo[2,3-b]pyridin-4-yl}benzamide;
N,N-diethyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
(2Z)-4-hydroxy-2-[(4-{4-[(3-hydroxypyrrolidin-1-yl)carbonyl]phenyl}-1-methyl-1H-pyrrolo[2,3-b]pyridin-3-yl)methylene]-1-benzofuran-3(2H)-one;
N-(2-methoxyethyl)-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-(2-methoxyethyl)-N-methyl-4-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}benzamide;
N-methoxy-4-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}benzamide;
(2Z)-4-hydroxy-2-[(4-{4-[(3-methoxypyrrolidin-1-yl)carbonyl]phenyl}-1-methyl-1H-pyrrolo[2,3-b]pyridin-3-yl)methylene]-1-benzofuran-3(2H)-one;
1-[(2Z)-2-({1-methyl-4-[4-(8-oxa-3-azabicyclo[3.2.1]oct-3-ylcarbonyl)piperidin-1-yl]-1H-pyrrolo[2,3-b]pyridin-3-yl}methylene)-3-oxo-2,3-dihydro-1-benzofuran-5-yl]-3-pyridin-3-ylurea;
N,N-bis(2-methoxyethyl)-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
1-{(2Z)-2-[(1-methyl-4-{4-[(4-methylpiperazin-1-yl)carbonyl]piperidin-1-yl}-1H-pyrrolo[2,3-b]pyridin-3-yl)methylidene]-3-oxo-2,3-dihydro-1-benzofuran-5-yl}-3-pyridin-3-ylurea;
1-[(2Z)-2-({1-methyl-4-[4-(pyrrolidin-1-ylcarbonyl)piperidin-1-yl]-1H-pyrrolo[2,3-b]pyridin-3-yl}methylene)-3-oxo-2,3-dihydro-1-benzofuran-5-yl]-3-pyridin-3-ylurea;
N-(2-hydroxyethyl)-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-4-ylmethyl)piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-3-ylmethyl)piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-2-ylmethyl)piperidine-4-carboxamide;
1-[(2Z)-2-({1-methyl-4-[4-(piperidin-1-ylcarbonyl)piperidin-1-yl]-1H-pyrrolo[2,3-b]pyridin-3-yl}methylene)-3-oxo-2,3-dihydro-1-benzofuran-5-yl]-3-pyridin-3-ylurea;
1-{(2Z)-2-[(4-{4-[(3-methoxypyrrolidin-1-yl)carbonyl]phenyl}-1-methyl-1H-pyrrolo[2,3-b]pyridin-3-yl)methylene]-3-oxo-2,3-dihydro-1-benzofuran-5-yl}-3-pyridin-3-ylurea;
N-(2-methoxyethyl)-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N-[2-(dimethylamino)ethyl]-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-pyridin-3-ylpiperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-pyridin-4-ylpiperidine-4-carboxamide;
1-[(2Z)-2-({4-[4-(2,5-diazabicyclo[2.2.1]hept-2-ylcarbonyl)piperidin-1-yl]-1-methyl-1H-pyrrolo[2,3-b]pyridin-3-yl}methylene)-3-oxo-2,3-dihydro-1-benzofuran-5-yl]-3-pyridin-3-ylurea;
1-{(2Z)-2-[(4-{4-[(2,6-dimethylmorpholin-4-yl)carbonyl]piperidin-1-yl}-1-methyl-1H-pyrrolo[2,3-b]pyridin-3-yl)methylene]-3-oxo-2,3-dihydro-1-benzofuran-5-yl}-3-pyridin-3-ylurea;
N-[2-(dimethylamino)ethyl]-N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
N,N-dimethyl-1-(1-methyl-3-{(Z)-[3-oxo-5-({[6-(tetrahydro-2H-pyran-4-yloxy)pyridin-3-yl]carbamoyl}amino)-1-benzofuran-2(3H)-ylidene]methyl}-1H-pyrrolo[2,3-b]pyridin-4-yl)piperidine-4-carboxamide;
N,N-dimethyl-1-(1-methyl-3-{(Z)-[5-{[(4-morpholin-4-ylphenyl)carbamoyl]amino}-3-oxo-1-benzofuran-2(3H)-ylidene]methyl}-1H-pyrrolo[2,3-b]pyridin-4-yl)piperidine-4-carboxamide;
N-methyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-pyridin-3-ylpiperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-3-ylmethyl)piperidine-4-carboxamide;
1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}-N-(pyridin-4-ylmethyl)piperidine-4-carboxamide;
1-{(2Z)-2-[(1-methyl-4-{4-[(5-methyl-2,5-diazabicyclo[2.2.1]hept-2-yl)carbonyl]piperidin-1-yl}-1H-pyrrolo[2,3-b]pyridin-3-yl)methylidene]-3-oxo-2,3-dihydro-1-benzofuran-5-yl}-3-pyridin-3-ylurea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-(4-morpholin-4-ylphenyl)urea;
1-{3-[(Z)-{5-[({4-[2-(dimethylamino)ethoxy]phenyl}carbamoyl)amino]-3-oxo-1-benzofuran-2(3H)-ylidene}methyl]-1-methyl-1H-pyrrolo[2,3-b]pyridin-4-yl}-N,N-dimethylpiperidine-4-carboxamide;
1-{(2Z)-2-[(4-{4-[(3-methoxypyrrolidin-1-yl)carbonyl]piperidin-1-yl}-1-methyl-1H-pyrrolo[2,3-b]pyridin-3-yl)methylidene]-3-oxo-2,3-dihydro-1-benzofuran-5-yl}-3-pyridin-3-ylurea;
1-[(2Z)-2-{[1-methyl-4-(3-oxa-9-azabicyclo[3.3.1]non-9-yl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[1-methyl-4-(2-oxa-5-azabicyclo[2.2.2]oct-5-yl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[4-(piperidin-1-ylmethyl)phenyl]urea;
1-methyl-3-[(2Z)-2-{[1-methyl-4-(2-oxa-5-azabicyclo[2.2.2]oct-5-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(3-oxa-8-azabicyclo[3.2.1]oct-8-yl)-1H-pyrrolo[2,3-b]pyridin-3-yl]methylidene}-3-oxo-2,3-dihydro-1-benzofuran-5-yl]-3-pyridin-3-ylurea;
1-methyl-3-[(2Z)-2-{[1-methyl-4-(3-oxa-8-azabicyclo[3.2.1]oct-8-yl)-1H-pyrrolo[2,3-b]pyridin-3-yl]methylidene}-3-oxo-2,3-dihydro-1-benzofuran-5-yl]urea;
1-[(2Z)-2-{[4-(6,8-dioxa-3-azabicyclo[3.2.1]oct-3-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[1-methyl-4-(2-oxa-5-azabicyclo[2.2.1]hept-5-yl)-1H-pyrrolo[2,3-b]pyridin-3-yl]methylidene}-3-oxo-2,3-dihydro-1-benzofuran-5-yl]-3-pyridin-3-ylurea;
1-methyl-3-[(2Z)-2-{[1-methyl-4-(2-oxa-5-azabicyclo[2.2.1]hept-5-yl)-1H-pyrrolo[2,3-b]pyridin-3-yl]methylidene}-3-oxo-2,3-dihydro-1-benzofuran-5-yl]urea;
1-azetidin-3-yl-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-[1-(trifluoroacetyl)piperidin-4-yl]urea;
1-{3-[(Z)-{5-[(azetidin-3-ylcarbamoyl)amino]-3-oxo-1-benzofuran-2(3H)-ylidene}methyl]-1-methyl-1H-pyrrolo[2,3-b]pyridin-4-yl}-N,N-dimethylpiperidine-4-carboxamide;
N,N-dimethyl-1-(1-methyl-3-{(Z)-[5-{[(1-methylethyl)carbamoyl]amino}-3-oxo-1-benzofuran-2(3H)-ylidene]methyl}-1H-pyrrolo[2,3-b]pyridin-4-yl)piperidine-4-carboxamide;
1-(1-methyl-1H-benzimidazol-2-yl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
N,N-dimethyl-1-(1-methyl-3-{(Z)-[5-{[(1-methyl-1H-benzimidazol-2-yl)carbamoyl]amino}-3-oxo-1-benzofuran-2(3H)-ylidene]methyl}-1H-pyrrolo[2,3-b]pyridin-4-yl)piperidine-4-carboxamide;
1-{3-[(Z)-{5-[(cyclopropylcarbamoyl)amino]-3-oxo-1-benzofuran-2(3H)-ylidene}methyl]-1-methyl-1H-pyrrolo[2,3-b]pyridin-4-yl}-N,N-dimethylpiperidine-4-carboxamide;
1-[(2Z)-2-({1-methyl-4-[4-(3-oxa-8-azabicyclo[3.2.1]oct-8-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
1-[(2Z)-2-{[1-methyl-4-(9-oxa-3,7-diazabicyclo[3.3.1]non-3-yl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[4-(3,6-dihydro-2H-pyran-4-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[4-(2,2-diethylmorpholin-4-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-({4-[(2R,6S)-2,6-dimethylmorpholin-4-yl]-1-methyl-1H-pyrrolo[2,3-b]pyridin-3-yl}methylidene)-3-oxo-2,3-dihydro-1-benzofuran-5-yl]-3-pyridin-3-ylurea;
1-[(2Z)-2-({4-[2-(2-methoxyethyl)morpholin-4-yl]-1-methyl-1H-pyrrolo[2,3-b]pyridin-3-yl}methylidene)-3-oxo-2,3-dihydro-1-benzofuran-5-yl]-3-pyridin-3-ylurea;
1-(6-fluoropyridin-3-yl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
1-(6-chloropyridin-3-yl)-3-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]urea;
N,N-dimethyl-1-(1-methyl-3-{(Z)-[3-oxo-5-({[1-(trifluoroacetyl)piperidin-4-yl]carbamoyl}amino)-1-benzofuran-2(3H)-ylidene]methyl}-1H-pyrrolo[2,3-b]pyridin-4-yl)piperidine-4-carboxamide;
1-[(2Z)-2-{[4-(2,2-dimethylmorpholin-4-yl)-1-methyl-1H-pyrrolo[2,3-b]pyridin-3-yl]methylidene}-3-oxo-2,3-dihydro-1-benzofuran-5-yl]-3-pyridin-3-ylurea;
(2Z)-2-{[4-(4-{[(2R,6S)-2,6-dimethylmorpholin-4-yl]carbonyl}piperidin-1-yl)-1-methyl-1H-pyrrolo[2,3-b]pyridin-3-yl]methylidene}-4-hydroxy-1-benzofuran-3(2H)-one;
(2Z)-2-{[4-(4-{[(2R,6S)-2,6-dimethylmorpholin-4-yl]carbonyl}phenyl)-1-methyl-1H-pyrrolo[2,3-b]pyridin-3-yl]methylidene}-4-hydroxy-1-benzofuran-3(2H)-one;
(2Z)-4-hydroxy-2-{[4-(4-{[4-(2-methoxyethyl)piperazin-1-yl]carbonyl}phenyl)-1-methyl-1H-pyrrolo[2,3-b]pyridin-3-yl]methylidene}-1-benzofuran-3(2H)-one;
(2Z)-4-hydroxy-2-{[4-(4-{[2-(2-methoxyethyl)morpholin-4-yl]carbonyl}phenyl)-1-methyl-1H-pyrrolo[2,3-b]pyridin-3-yl]methylidene}-1-benzofuran-3(2H)-one;
(2Z)-4-hydroxy-2-({1-methyl-4-[4-(8-oxa-3-azabicyclo[3.2.1]oct-3-ylcarbonyl)piperidin-1-yl]-1H-pyrrolo[2,3-b]pyridin-3-yl}methylidene)-1-benzofuran-3(2H)-one;
(2Z)-4-hydroxy-2-{[4-(4-{[4-(2-hydroxyethyl)piperazin-1-yl]carbonyl}piperidin-1-yl)-1-methyl-1H-pyrrolo[2,3-b]pyridin-3-yl]methylidene}-1-benzofuran-3(2H)-one;
1-[(2Z)-2-{[4-(4-{[2-(methoxymethyl)morpholin-4-yl]carbonyl}piperidin-1-yl)-1-methyl-1H-pyrrolo[2,3-b]pyridin-3-yl]methylidene}-3-oxo-2,3-dihydro-1-benzofuran-5-yl]-3-pyridin-3-ylurea;
1-[(2Z)-2-({1-methyl-4-[4-(3-oxa-9-azabicyclo[3.3.1]non-9-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea;
(2Z)-4-hydroxy-2-{[4-(4-{[2-(methoxymethyl)morpholin-4-yl]carbonyl}phenyl)-1-methyl-1H-pyrrolo[2,3-b]pyridin-3-yl]methylidene}-1-benzofuran-3(2H)-one;
(2Z)-4-hydroxy-2-{[4-(4-{[2-(methoxymethyl)morpholin-4-yl]carbonyl}piperidin-1-yl)-1-methyl-1H-pyrrolo[2,3-b]pyridin-3-yl]methylidene}-1-benzofuran-3(2H)-one; and
(2Z)-4-hydroxy-2-({1-methyl-4-[4-(3-oxa-9-azabicyclo[3.3.1]non-9-ylcarbonyl)piperidin-1-yl]-1H-pyrrolo[2,3-b]pyridin-3-yl}methylidene)-1-benzofuran-3(2H)-one.

16) A compound of claim 15 selected from the group consisting of:
1-[(2Z)-2-{[1-methyl-4-(2-oxa-5-azabicyclo[2.2.2]oct-5-yl)-1H-pyrrolo[2,3-b]pyridin-3-yl]methylidene}-3-oxo-2,3-dihydro-1-benzofuran-5-yl]-3-pyridin-3-ylurea;
1-[(2Z)-2-{[1-methyl-4-(8-oxa-3-azabicyclo[3.2.1]oct-3-yl)-1H-pyrrolo[2,3-b]pyridin-3-yl]methylidene}-3-oxo-2,3-dihydro-1-benzofuran-5-yl]-3-pyridin-3-ylurea;
(2Z)-4-hydroxy-2-{[4-(4-{[4-(2-hydroxyethyl)piperazin-1-yl]carbonyl}phenyl)-1-methyl-1H-pyrrolo[2,3-b]pyridin-3-yl]methylidene}-1-benzofuran-3(2H)-one;
1-{3-[(Z)-(4-hydroxy-3-oxo-1-benzofuran-2(3H)-ylidene)methyl]-1-methyl-1H-pyrrolo[2,3-b]pyridin-4-yl}-N,N-dimethylpiperidine-4-carboxamide;
N,N-dimethyl-1-{1-methyl-3-[(Z)-{3-oxo-5-[(pyridin-3-ylcarbamoyl)amino]-1-benzofuran-2(3H)-ylidene}methyl]-1H-pyrrolo[2,3-b]pyridin-4-yl}piperidine-4-carboxamide;
(2Z)-4-hydroxy-2-({1-methyl-4-[4-(morpholin-4-ylcarbonyl)piperidin-1-yl]-1H-pyrrolo[2,3-b]pyridin-3-yl}methylidene)-1-benzofuran-3(2H)-one; and
1-[(2Z)-2-({1-methyl-4-[4-(3-oxa-8-azabicyclo[3.2.1]oct-8-ylcarbonyl)piperidin-1-yl]-1H-pyrrolo[2,3-b]pyridin-3-yl}methylidene)-3-oxo-2,3-dihydro-1-benzofuran-5-yl]-3-pyridin-3-ylurea.

17) A composition comprising a compound of claim 1 and a pharmaceutically acceptable carrier.

18) The composition of claim 17, wherein the pharmaceutically acceptable carrier is suitable for oral administration and the composition comprises an oral dosage form.

19) A composition comprising a compound of claim 1; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

20) The composition of claim 19, wherein the second compound is Avastin.

21) A method of treating a PI3K-related disorder, comprising administering to a mammal in need thereof a compound of claim 1 in an amount effective to treat a PI3K-related disorder.

22) The method of claim 21, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

23) The method of claim 22, wherein the PI3K-related disorder is cancer.

24) The method of claim 23, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

25) A method of treating an mTOR-related disorder, comprising administering to a mammal in need thereof a compound of claim 1 in an amount effective to treat an mTOR-related disorder.

26) The method of claim 25, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

27) The method of claim 26, wherein the mTOR-related disorder is cancer.

28) The method of claim 27, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

29) A method of treating advanced renal cell carcinoma, comprising administering to a mammal in need thereof a compound of claim 1 in an amount effective to treat advanced renal cell carcinoma.

30) A method of treating acute lymphoblastic leukemia, comprising administering to a mammal in need thereof a compound of claim 1 in an amount effective to treat acute lymphoblastic leukemia.

31) A method of treating acute malignant melanoma, comprising administering to a mammal in need thereof a compound of claim 1 in an amount effective to treat malignant melanoma.

32) A method of treating soft-tissue or bone sarcoma, comprising administering to a mammal in need thereof a compound of claim 1 in an amount effective to treat soft-tissue or bone sarcoma.

33) A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 20 in an amount effective to treat the cancer.

34) A method of inhibiting mTOR in a subject, comprising administering to a subject in need thereof a compound of claim 1 in an amount effective to inhibit mTOR.

35) A method of inhibiting PI3K in a subject, comprising administering to a subject in need thereof a compound of claim 1 in an amount effective to inhibit PI3K.

36) A method of inhibiting mTOR, PI3K, and hSMG-1 together in a subject, comprising administering to a subject in need thereof a compound of claim 1 in an amount effective to inhibit mTOR, PI3K, and hSMG-1.

37) A method of synthesizing a compound of Formula 1′, comprising:
a) condensing a compound of the formula XVIII with a compound of formula XIX: 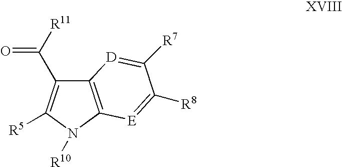
under acidic conditions, and A, D, E, and R¹-R¹¹ are as defined in claim 1 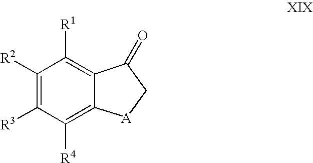
thereby producing a compound of formula 1′: 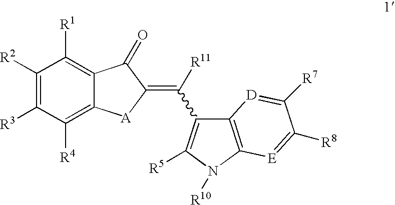
b) optionally reducing the compound of formula 1′ and thereby producing a compound of formula 1″: 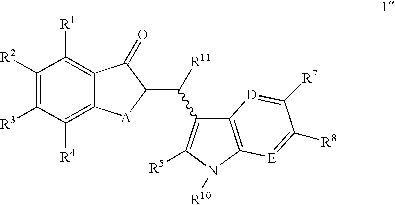
or a pharmaceutically acceptable salt thereof.

38) The method of claim 37 further comprising:
a) acylation with R¹¹C(O)X, wherein X is halogen, or Vilsmeier-Haack formylation, of a compound of formula XVI: 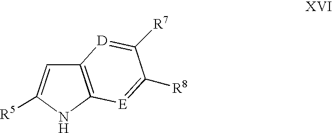
thereby producing a compound of formula XVII: 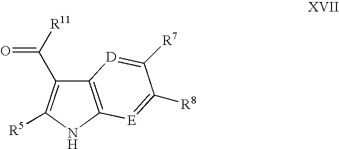
b) optionally alkylating the compound of formula XVII with R¹⁰Cl, thereby producing a compound of Formula XVIII.
